SEQUENCE LISTING (417 sequences)

SEQ ID NO: 1 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 2 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 3 - protein, 100 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 4 - protein, 512 aa, Artificial Sequence
  Description of Artificial Sequence: Synthetic polypeptide
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 5 - protein, 115 aa, Artificial Sequence
  Description of Artificial Sequence: Synthetic polypeptide
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 6 - protein, 100 aa, Artificial Sequence
  Description of Artificial Sequence: Synthetic polypeptide
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 7 - DNA, 1536 bp, Homo sapiens
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatc

SEQ ID NO: 8 - DNA, 345 bp, Homo sapiens
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccacc

SEQ ID NO: 9 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 10 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 11 - protein, 100 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 12 - DNA, 1539 bp, Homo sapiens
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 13 - DNA, 345 bp, Homo sapiens
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 14 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 17 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 18 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 3 aa, Artificial Sequence
  Description of Artificial Sequence: Synthetic peptide
GGG

SEQ ID NO: 20 - protein, 4 aa, Artificial Sequence
  Description of Artificial Sequence: Synthetic peptide
GGGG

SEQ ID NO: 21 - protein, 5 aa, Artificial Sequence
  Description of Artificial Sequence: Synthetic peptide
TGGGG

SEQ ID NO: 22 - protein, 5 aa, Artificial Sequence
  Description of Artificial Sequence: Synthetic peptide
SGGGG

SEQ ID NO: 23 - protein, 4 aa, Artificial Sequence
  Description of Artificial Sequence: Synthetic peptide
TGGG

SEQ ID NO: 24 - protein, 4 aa, Artificial Sequence
  Description of Artificial Sequence: Synthetic peptide
SGGG

SEQ ID NO: 25 - protein, 5 aa, Artificial Sequence
  Description of Artificial Sequence: Synthetic peptide
GGGGS

SEQ ID NO: 26-31 - skipped (6 entries)

SEQ ID NO: 32 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 21 aa, Apis sp.
MKFLVNVALVFMVVYISYIYA

SEQ ID NO: 34 - protein, 22 aa, Unknown
  Description of Unknown: Tissue plasminogen activator peptide
MDAMKRGLCCVLLLCGAVFVSP

SEQ ID NO: 35 - protein, 20 aa, Unknown
  Description of Unknown: Native peptide
MGAAAKLAFAVFLISCSSGA

SEQ ID NO: 36 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1114 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 38 - protein, 9 aa, Artificial Sequence
  Description of Artificial Sequence: Synthetic peptide
ILGRSETQE

SEQ ID NO: 39 - protein, 329 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 20 aa, Unknown
  Description of Unknown: Native peptide
MGAAAKLAFAVFLISCSSGA

SEQ ID NO: 42 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 44 - protein, 6 aa, Artificial Sequence
  Description of Artificial Sequence: Synthetic peptide
GRGEAE

SEQ ID NO: 45 - protein, 335 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 115 aa, Artificial Sequence
  Description of Artificial Sequence: Synthetic polypeptide
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPT

SEQ ID NO: 48 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggacgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 50 - protein, 141 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEGPWASTTIPSGGPEATAAAGDQGSGALWLCLEGPAHE

SEQ ID NO: 51 - protein, 141 aa, Artificial Sequence
  Description of Artificial Sequence: Synthetic polypeptide
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEGPWASTTIPSGGPEATAAAGDQGSGALWLCLEGPAHE

SEQ ID NO: 52 - protein, 370 aa, Artificial Sequence
  Description of Artificial Sequence: Synthetic polypeptide
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEGPWASTTIPSGGPEATAAAGDQGSGALWLCLEGPAHETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 150 aa, Rattus sp.
MTAPWAALALLWGSLCAGSGRGEAETRECIYYNANWELERTNQSGLERCEGEQDKRLHCYASWPNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 54 - protein, 150 aa, Sus sp.
MTAPWAALALLWGSLCVGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 55 - protein, 150 aa, Mus sp.
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 56 - protein, 150 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 57 - protein, 150 aa, Bos taurus
MTAPWAALALLWGSLCAGSGRGEAETRECIYYNANWELERTNQSGLERCEGERDKRLHCYASWRNSSGTIELVKKGCWLDDFNCYDRQECVATEENPQVYFCCCEGNFCNERFTHLPEAGGPEVTYEPPPTAPTLLTVLAYSLLPVGGLS

SEQ ID NO: 58 - protein, 150 aa, Xenopus sp.
MGASVALTFLLLLATFRAGSGHDEVETRECIYYNANWELEKTNQSGVERLVEGKKDKRLHCYASWRNNSGFIELVKKGCWLDDFNCYDRQECIAKEENPQVFFCCCEGNYCNKKFTHLPEVETFDPKPQPSASVLNILIYSLLPIVGLSM

SEQ ID NO: 59 - protein, 150 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLI

SEQ ID NO: 60 - protein, 154 aa, Artificial Sequence
  Description of Artificial Sequence: Synthetic polypeptide
  MOD_RES (8)..(8): T, A or absent
  MOD_RES (121)..(121): P, A, V or M
MTAPWAAXLALLWGSLCAGSGRGEAETRECIYYNANWELERTNQSGLERLCEGEQDKRLHCYASWRNSSGTLELVKKGCWLDDFNCYDRQECVATEENPQVYFCCCEGNFCNERFTHLPEXGGPEVTYEPKPPTAPTLLTVLAYSLLPIGGLSM

SEQ ID NO: 61 - protein, 115 aa, Homo sapiens; identical to SEQ ID NO: 10

SEQ ID NO: 62 - protein, 115 aa, Ovis aries
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 63 - protein, 115 aa, Unknown
  Description of Unknown: Talpidae family peptide sequence
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKAP

SEQ ID NO: 64 - protein, 115 aa, Mus sp.
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPP

SEQ ID NO: 65 - protein, 115 aa, Gallus gallus
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 66 - protein, 115 aa, Bos taurus; identical to SEQ ID NO: 62

SEQ ID NO: 67 - protein, 115 aa, Tyto alba; identical to SEQ ID NO: 65

SEQ ID NO: 68 - protein, 115 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 69 - protein, 360 aa, Artificial Sequence
  Description of Artificial Sequence: Synthetic polypeptide
MDAMKRGLCCVLLLCGAVFVSPGAAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1083 bp, Artificial Sequence
  Description of Artificial Sequence: Synthetic polynucleotide
  CDS (73)..(396)
atggatgcaatgaagagagggctctgctgtgtgctgctgctgtgtggagcagtcttcgtttcgcccggcgccgctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacaggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 71 - DNA, 1083 bp, Artificial Sequence
  Description of Artificial Sequence: Synthetic polynucleotide
tacctacgttacttctctcccgagacgacacacgacgacgacacacctcgtcagaagcaaagcgggccgcggcgactctgtgccctcacgtagatgatgttgcggttgaccctcgacctcgcgtggttggtctcgccggacctcgcgacgcttccgctcgtcctgttcgccgacgtgacgatgcggaggaccgcgttgtcgagaccgtggtagctcgagcacttcttcccgacgaccgatctactgaagttgacgatgctatccgtcctcacacaccggtgactcctcttgggggtccacatgaagacgacgacacttccgttgaagacgttgctcgcgaagtgagtaaacggtctccgacccccgggccttcagtgcatgctcggtgggggctgtccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 72 - DNA, 1083 bp, Artificial Sequence
  Description of Artificial Sequence: Synthetic polynucleotide
  CDS (73)..(396)
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctggctggacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 73 - DNA, 1083 bp, Artificial Sequence
  Description of Artificial Sequence: Synthetic polynucleotide
tacctacgttacttctctcccgagacgacacacgacgacgacacacctcgtcagaagcaaagcgggccgcggcggctttgggcgcttacataaataatgttacgattaacccttgagcttgcctgcttggttaggcccgagcttgccacactcccccttgtcctatttgcggaggtaacgatacgcagcacctccttgaggaggccctgctaacttgaccagttctttcccacgaccgacctgctaaagttaacaatactggcggtccttacacagcgctggcttctcttaggcgtccagataaagacaacaacgctccccttaaagacattacttgccaaatgggtggaggggcttcggccgcccgggctccactggatacttgggggcgggtggccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 74 - protein, 368 aa, Artificial Sequence; identical to SEQ ID NO: 48
  Description of Artificial Sequence: Synthetic polypeptide

SEQ ID NO: 75 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggatgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccaccaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 76 - DNA, 1107 bp, Artificial Sequence
  Description of Artificial Sequence: Synthetic polynucleotide
tacctacgttacttctctcccgagacgacacacgacgacgacacacctcgtcagaagcaaagcgggccgcggagacccgcacccctccgactctgtgccctcacgtagatgatgttgcggttgaccctcgacctcgcgtggttggtctcgccggacctcgcgacgcttccgctcgtcctgttcgccgacgtgacgatgcggaggaccgcgttgtcgagaccgtggtagctcgagcacttcttcccgacgaccctactactgaagttgacgatgctatccgtcctcacacaccggtgactcctcttgggggtccacatgaagacgacgacacttccgttgaagacgttgctcgcgaagtgagtaaacggtctccgacccccgggccttcagtgcatgctcggtgggggctgtcgggggtggccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 77 - protein, 360 aa, Artificial Sequence
  Description of Artificial Sequence: Synthetic polypeptide
MDAMKRGLCCVLLLCGAVFVSPGAAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 335 aa, Artificial Sequence
  Description of Artificial Sequence: Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPRVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 107 aa, Artificial Sequence
  Description of Artificial Sequence: Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 80 - DNA, 1083 bp, Artificial Sequence
  Description of Artificial Sequence: Synthetic polynucleotide
  CDS (76)..(396)
atggatgcaatgaagagagggctctgctgtgtgctgctgctgtgtggagcagtcttcgtttcgcccggcgccgctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggacgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacaggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 81 - DNA, 1083 bp, Artificial Sequence
  Description of Artificial Sequence: Synthetic polynucleotide
tacctacgttacttctctcccgagacgacacacgacgacgacacacctcgtcagaagcaaagcgggccgcggcgactctgtgccctcacgtagatgatgttgcggttgaccctcgacctcgcgtggttggtctcgccggacctcgcgacgcttccgctcgtcctgttcgccgacgtgacgatgcggaggaccgcgttgtcgagaccgtggtagctcgagcacttcttcccgacgaccctgctactgaagttgacgatgctatccgtcctcacacaccggtgactcctcttgggggtccacatgaagacgacgacacttccgttgaagacgttgctcgcgaagtgagtaaacggtctccgacccccgggccttcagtgcatgctcggtgggggctgtccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccctgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 82 - DNA, 1083 bp, Artificial Sequence
  Description of Artificial Sequence: Synthetic polynucleotide
  CDS (76)..(396)
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctgggacgacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 83 - DNA, 1083 bp, Artificial Sequence
  Description of Artificial Sequence: Synthetic polynucleotide
tacctacgttacttctctcccgagacgacacacgacgacgacacacctcgtcagaagcaaagcgggccgcggcggctttgggcgcttacataaataatgttacgattaacccttgagcttgcctgcttggttaggcccgagcttgccacactcccccttgtcctatttgcggaggtaacgatacgcagcacctccttgaggaggccctgctaacttgaccagttctttcccacgaccctgctgctaaagttaacaatactggcggtccttacacagcgctggcttctcttaggcgtccagataaagacaacaacgctccccttaaagacattacttgccaaatgggtggaggggcttcggccgcccgggctccactggatacttgggggcgggtggccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 84 - DNA, 321 bp, Artificial Sequence
  Description of Artificial Sequence: Synthetic polynucleotide
gaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctgggacgacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccacc

SEQ ID NO: 85 - protein, 10 aa, Artificial Sequence
  Description of Artificial Sequence: Synthetic peptide
GGGGSGGGGS

SEQ ID NO: 86 - protein, 11 aa, Artificial Sequence
  Description of Artificial Sequence: Synthetic peptide
TGGGGSGGGGS

SEQ ID NO: 87 - protein, 16 aa, Artificial Sequence
  Description of Artificial Sequence: Synthetic peptide
TGGGGSGGGGSGGGGS

SEQ ID NO: 88 - protein, 21 aa, Artificial Sequence
  Description of Artificial Sequence: Synthetic peptide
TGGGGSGGGGSGGGGSGGGGS

SEQ ID NO: 89 - protein, 26 aa, Artificial Sequence
  Description of Artificial Sequence: Synthetic peptide
TGGGGSGGGGSGGGGSGGGGSGGGGS

SEQ ID NO: 90 - protein, 31 aa, Artificial Sequence
  Description of Artificial Sequence: Synthetic polypeptide
TGGGGSGGGGSGGGGSGGGGSGGGGSGGGGS

SEQ ID NO: 91 - protein, 36 aa, Artificial Sequence
  Description of Artificial Sequence: Synthetic polypeptide
TGGGGSGGGGSGGGGSGGGGSGGGGSGGGGSGGGGS

SEQ ID NO: 92 - protein, 10 aa, Artificial Sequence
  Description of Artificial Sequence: Synthetic peptide
TGGGPKSCDK

SEQ ID NO: 93 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 94 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 95 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 96 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98-99 - skipped (2 entries)

SEQ ID NO: 100 - protein, 505 aa, Homo sapiens
MAESAGASSFFPLVVLLLAGSGGSGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVELVGIIAGPVFLLFLIIIIVFLVINYHQRVYHNRQRLDMEDPSCEMCLSKDKTLQDLVYDLSTSGSGSGLPLFVQRTVARTIVLQEIIGKGRFGEVWRGRWRGGDVAVKIFSSREERSWFREAEIYQTVMLRHENILGFIAADNKDNGTWTQLWLVSDYHEHGSLFDYLNRYTVTIEGMIKLALSAASGLAHLHMEIVGTQGKPGIAHRDLKSKNILVKKNGMCAIADLGLAVRHDAVTDTIDIAPNQRVGTKRYMAPEVLDETINMKHFDSFKCADIYALGLVYWEIARRCNSGGVHEEYQLPYYDLVPSDPSIEEMRKVVCDQKLRPNIPNWWQSYEALRVMGKMMRECWYANGAARLTALRIKKTLSQLSVQEDVKI

SEQ ID NO: 101 - protein, 103 aa, Homo sapiens
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVE

SEQ ID NO: 102 - DNA, 1515 bp, Homo sapiens
atggcggagtcggccggagcctcctccttcttcccccttgttgtcctcctgctcgccggcagcggcgggtccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatc

SEQ ID NO: 103 - DNA, 309 bp, Homo sapiens
tccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggag

SEQ ID NO: 104 - protein, 453 aa, Homo sapiens
MVSIFNLDGMEHHVRTCIPKVELVPAGKPFYCLSSEDLRNTHCCYTDYCNRIDLRVPSGHLKEPEHPSMWGPVELVGIIAGPVFLLFLIIIIVFLVINYHQRVYHNRQRLDMEDPSCEMCLSKDKTLQDLVYDLSTSGSGSGLPLFVQRTVARTIVLQEIIGKGRFGEVWRGRWRGGDVAVKIFSSREERSWFREAEIYQTVMLRHENILGFIAADNKDNGTWTQLWLVSDYHEHGSLFDYLNRYTVTIEGMIKLALSAASGLAHLHMEIVGTQGKPGIAHRDLKSKNILVKKNGMCAIADLGLAVRHDAVTDTIDIAPNQRVGTKRYMAPEVLDETINMKHFDSFKCADIYALGLVYWEIARRCNSGGVHEEYQLPYYDLVPSDPSIEEMRKVVCDQKLRPNIPNWWQSYEALRVMGKMMRECWYANGAARLTALRIKKTLSQLSVQEDVKI

SEQ ID NO: 105 - protein, 74 aa, Homo sapiens
MVSIFNLDGMEHHVRTCIPKVELVPAGKPFYCLSSEDLRNTHCCYTDYCNRIDLRVPSGHLKEPEHPSMWGPVE

SEQ ID NO: 106 - DNA, 1362 bp, Homo sapiens
atggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatctaa

SEQ ID NO: 107 - DNA, 230 bp, Homo sapiens
atggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtagg

SEQ ID NO: 108 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 109 - DNA, 1104 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 110 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 111 - protein, 355 aa, Artificial Sequence
  Description of Artificial Sequence: Synthetic polypeptide
MDAMKRGLCCVLLLCGAVFVSPGASGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 112 - DNA, 1065 bp, Artificial Sequence
  Description of Artificial Sequence: Synthetic polynucleotide
atggatgcaatgaagagagggctctgctgtgtgctgctgctgtgtggagcagtcttcgtttcgcccggcgcctccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 113 - protein, 331 aa, Artificial Sequence
  Description of Artificial Sequence: Synthetic polypeptide
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 114 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 356 aa, Artificial Sequence
  Description of Artificial Sequence: Synthetic polypeptide
MDAMKRGLCCVLLLCGAVFVSPGASGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 117 - protein, 332 aa, Artificial Sequence
  Description of Artificial Sequence: Synthetic polypeptide
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 118 - protein, 367 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTENQVSLWCLVKGFYPSDIAVEWESNGQPENNYKTTPPVLDSDGSFFLYSKLTVDKSRWQQGNVFSCSVMHEALHNHYTQDSLSLSPG

SEQ ID NO: 119 - DNA, 1101 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggaggagatgaccgagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcaggacagcctctccctgtctccgggt

SEQ ID NO: 120 - protein, 342 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTENQVSLWCLVKGFYPSDIAVEWESNGQPENNYKTTPPVLDSDGSFFLYSKLTVDKSRWQQGNVFSCSVMHEALHNHYTQDSLSLSPG

SEQ ID NO: 121 - DNA, 1026 bp, Artificial Sequence
  Description of Artificial Sequence: Synthetic polynucleotide
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggaggagatgaccgagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcaggacagcctctccctgtctccgggt

SEQ ID NO: 122 - protein, 356 aa, Artificial Sequence
  Description of Artificial Sequence: Synthetic polypeptide
MDAMKRGLCCVLLLCGAVFVSPGASGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESRGQPENNYKTTPPVLDSRGSFFLVSKLTVDKSRWQQGNVFSCSVMHEALHNHYTQKSLSLSPGK

SEQ ID NO: 123 - DNA, 1068 bp, Artificial Sequence
  Description of Artificial Sequence: Synthetic polynucleotide
atggatgcaatgaagagagggctctgctgtgtgctgctgctgtgtggagcagtcttcgtttcgcccggcgcctccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggaggagatgaccaagaaccaggtcagcctgtcctgcgccgtcaaaggcttctatcccagcgacatcgccgtggagtgggagagccgcgggcagccggagaacaactacaagaccacgcctcccgtgctggactcccgcggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 124 - protein, 332 aa, Artificial Sequence
  Description of Artificial Sequence: Synthetic polypeptide
SGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESRGQPENNYKTTPPVLDSRGSFFLVSKLTVDKSRWQQGNVFSCSVMHEALHNHYTQKSLSLSPGK

SEQ ID NO: 125 - DNA, 996 bp, Artificial Sequence
  Description of Artificial Sequence: Synthetic polynucleotide
tccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggaggagatgaccaagaaccaggtcagcctgtcctgcgccgtcaaaggcttctatcccagcgacatcgccgtggagtgggagagccgcgggcagccggagaacaactacaagaccacgcctcccgtgctggactcccgcggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 126 - protein, 103 aa, Homo sapiens; identical to SEQ ID NO: 101

SEQ ID NO: 127 - protein, 103 aa, Unknown
  Description of Unknown: Talpidae family peptide sequence
SGPRGIQALLCACTSCLQANLTCETDGACMVSIFNLDGLEHHVRTCIPKVELVPAGKPFYCLSSEDLRNTHCCYTDFCNKIDLRVPSGHVKEPERPSVWGPVE

SEQ ID NO: 128 - protein, 103 aa, Erinaceus europaeus
SGPRGIQALLCACTSCLQTNYTCETDGACMVSIFNLDGMEHHVRTCIPKVELVPAGKPFYCLSSEDLRNTHCCYTDFCNRIDLRVPSGHPKESEQASMWGPVE

SEQ ID NO: 129 - protein, 102 aa, Gallus gallus
APGGARALTCLCSDCKQANSTCETDGACMVSVFNLDGVKHHVRTCIPEAKLIPAGKPFYCLSSEDLRNTHCCYSDFCNKIDLMVPSGHLKDNEPPSSWGPVE

SEQ ID NO: 130 - protein, 103 aa, Mus sp.
AGPRGIQALLCACTSCLQTNYTCETDGACMVSIFNLDGVEHHVRTCIPKVELVPAGKPFYCLSSEDLRNTHCCYIDFCNKIDLRVPSGHLKEPAHPSMWGPVE

SEQ ID NO: 131 - protein, 103 aa, Sus sp.
SGPRGIQALLCACTSCLQANYTCETDGACMVSIFNLDGMEHHVRTCIPKVELVPAGKPFYCLSSEDLRNTHCCYTDFCNKIDLRVPSGHLKEPEHPSMWGPVE

SEQ ID NO: 132 - protein, 103 aa, Rattus sp.
SGPRGIQALLCACTSCLQTNYTCETDGACMVSIFNLDGMEHHVRTCIPKVELVPAGKPFYCLSSEDLRNTHCCYIDFCNKIDLRVPSGHLKEPEHPSMWGPVE

SEQ ID NO: 133 - protein, 225 aa, Homo sapiens; identical to SEQ ID NO: 14

SEQ ID NO: 134 - protein, 232 aa, Homo sapiens; identical to SEQ ID NO: 16

SEQ ID NO: 135 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 223 aa, Homo sapiens; identical to SEQ ID NO: 15

SEQ ID NO: 137 - protein, 6 aa, Artificial Sequence
  Description of Artificial Sequence: Synthetic 6xHis tag
HHHHHH

SEQ ID NO: 138 - protein, 108 aa, Artificial Sequence
  Description of Artificial Sequence: Synthetic polypeptide
AETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPT

SEQ ID NO: 139 - protein, 109 aa, Artificial Sequence
  Description of Artificial Sequence: Synthetic polypeptide
  MOD_RES (14)..(14): F or Y
  MOD_RES (15)..(15): F or Y
  MOD_RES (20)..(20): E or A
  MOD_RES (21)..(21): K or L
  MOD_RES (22)..(22): D or E
  MOD_RES (23)..(23): R or A
  MOD_RES (31)..(31): P or R
  MOD_RES (33)..(33): Y or E
  MOD_RES (35)..(35): D or E
  MOD_RES (36)..(36): K or Q
  MOD_RES (37)..(37): D or A
  MOD_RES (38)..(38): K or A
  MOD_RES (39)..(39): R or A
  MOD_RES (40)..(40): R or L
  MOD_RES (43)..(43): F or Y
  MOD_RES (47)..(47): K, R, or A
  MOD_RES (57)..(57): K, A, Y, F, or I
  MOD_RES (58)..(58): Q or K
  MOD_RES (61)..(61): W or A
  MOD_RES (62)..(62): L or A
  MOD_RES (63)..(63): D, K, R, A, F, G, M, N, or I
  MOD_RES (65)..(65): I, F, or A
  MOD_RES (76)..(76): K or T
  MOD_RES (77)..(77): K or E
  MOD_RES (78)..(78): D or E
  MOD_RES (79)..(79): S or N
  MOD_RES (81)..(81): E or Q
  See specification as filed for detailed description of substitutions and preferred embodiments
GAILGRSETQECLXXNANWXXXXTNQTGVEXCXGXXXXXXHCXATWXNISGSIEIVXXGCXXXDXNCYDRTDCVEXXXXPXVYFCCCEGNMCNEKFSYFPEMEVTQPTS

SEQ ID NO: 140 - protein, 109 aa, Artificial Sequence
  Description of Artificial Sequence: Synthetic polypeptide
  MOD_RES (15)..(15): F, A, V, I, L, M, F, Y or W
  MOD_RES (20)..(20): D, E or A
  MOD_RES (21)..(21): K or L
  MOD_RES (22)..(22): D, E, or A
  MOD_RES (23)..(23): R, H, K, or A
  MOD_RES (31)..(31): P or R
  MOD_RES (33)..(33): Y or E
  MOD_RES (35)..(35): D, E, or A
  MOD_RES (37)..(37): D, E, or A
  MOD_RES (38)..(38): K, R, H, or A
  MOD_RES (39)..(39): R, H, K, or A
  MOD_RES (40)..(40): R or L
  MOD_RES (43)..(43): F, A, V, I, L, M, F, Y or W
  MOD_RES (47)..(47): K, R, H, or A
  MOD_RES (57)..(57): K, A, Y, F, I, V, L, M, or W
  MOD_RES (58)..(58): Q or K
  MOD_RES (61)..(61): W or A
  MOD_RES (62)..(62): L or A
  MOD_RES (63)..(63): D, K, R, H, A, F, G, M, N, or I
  MOD_RES (65)..(65): I, F, A, V, L, M, Y or W
  MOD_RES (76)..(76): K or T
  MOD_RES (77)..(77): K, R, H, D, or E
  MOD_RES (78)..(78): D, E, or A
  MOD_RES (79)..(79): S, N, T, or Q
  MOD_RES (81)..(81): E or Q
  See specification as filed for detailed description of substitutions and preferred embodiments
GAILGRSETQECLFXNANWXXXXTNQTGVEXCXGXKXXXXHCXATWXNISGSIEIVXXGCXXXDXNCYDRTDCVEXXXXPXVYFCCCEGNMCNEKFSYFPEMEVTQPTS

SEQ ID NO: 141 - protein, 109 aa, Artificial Sequence
  Description of Artificial Sequence: Synthetic polypeptide
  MOD_RES (15)..(15): F, A, V, I, L, M, F, Y or W
  MOD_RES (21)..(21): K or L
  MOD_RES (22)..(22): D, E, or A
  MOD_RES (31)..(31): P or R
  MOD_RES (33)..(33): Y or E
  MOD_RES (35)..(35): D, E, or A
  MOD_RES (40)..(40): R or L
  MOD_RES (43)..(43): F, A, V, I, L, M, F, Y or W
  MOD_RES (47)..(47): K, R, H, or A
  MOD_RES (58)..(58): Q or K
  MOD_RES (65)..(65): I, F, A, V, L, M, Y or W
  MOD_RES (76)..(76): K or T
  MOD_RES (77)..(77): K, R, H, D, or E
  MOD_RES (78)..(78): D, E, or A
  MOD_RES (79)..(79): S, N, T, or Q
  MOD_RES (81)..(81): E or Q
  See specification as filed for detailed description of substitutions and preferred embodiments
GAILGRSETQECLFXNANWEXXRTNQTGVEXCXGXKDKRXHCXATWXNISGSIEIVKXGCWLDDXNCYDRTDCVEXXXXPXVYFCCCEGNMCNEKFSYFPEMEVTQPTS

SEQ ID NO: 142 - protein, 109 aa, Artificial Sequence
  Description of Artificial Sequence: Synthetic polypeptide
  MOD_RES (15)..(15): F, A, V, I, L, M, F, Y or W
  MOD_RES (21)..(21): K or L
  MOD_RES (31)..(31): P or R
  MOD_RES (33)..(33): Y or E
  MOD_RES (35)..(35): D, E, or A
  MOD_RES (40)..(40): R or L
  MOD_RES (43)..(43): F, A, V, I, L, M, F, Y or W
  MOD_RES (47)..(47): K, R, H, or A
  MOD_RES (58)..(58): Q or K
  MOD_RES (65)..(65): I, F, A, V, L, M, Y or W
  MOD_RES (76)..(76): K or T
  MOD_RES (78)..(78): D, E, or A
  MOD_RES (79)..(79): S, N, T, or Q
  MOD_RES (81)..(81): E or Q
  See specification as filed for detailed description of substitutions and preferred embodiments
GAILGRSETQECLFXNANWEXDRTNQTGVEXCXGXKDKRXHCXATWXNISGSIEIVKXGCWLDDXNCYDRTDCVEXKXXPXVYFCCCEGNMCNEKFSYFPEMEVTQPTS

SEQ ID NO: 143 - protein, 109 aa, Artificial Sequence
  Description of Artificial Sequence: Synthetic polypeptide
  MOD_RES (15)..(15): F, A, V, I, L, M, F, Y or W
  MOD_RES (21)..(21): K or L
  MOD_RES (33)..(33): Y or E
  MOD_RES (35)..(35): D, E, or A
  MOD_RES (40)..(40): R or L
  MOD_RES (58)..(58): Q or K
  MOD_RES (76)..(76): K or T
  MOD_RES (78)..(78): D, E, or A
  MOD_RES (79)..(79): S, N, T, or Q
  MOD_RES (81)..(81): E or Q
  See specification as filed for detailed description of substitutions and preferred embodiments
GAILGRSETQECLFXNANWEXDRTNQTGVEPCXGXKDKRXHCFATWKNISGSIEIVKXGCWLDDINCYDRTDCVEXKXXPXVYFCCCEGNMCNEKFSYFPEMEVTQPTS

SEQ ID NO: 144 - protein, 109 aa, Artificial Sequence
  Description of Artificial Sequence: Synthetic polypeptide
GAILGRSETQECLFYNANWELDRTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 145 - protein, 109 aa, Artificial Sequence
  Description of Artificial Sequence: Synthetic polypeptide
GAILGRSETQECLFYNANWELERTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 146 - protein, 109 aa, Artificial Sequence
  Description of Artificial Sequence: Synthetic polypeptide
GAILGRSETQECLFYNANWELDRTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 147 - protein, 109 aa, Artificial Sequence
  Description of Artificial Sequence: Synthetic polypeptide
GAILGRSETQECLFYNANWELDRTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 148 - protein, 109 aa, Artificial Sequence
  Description of Artificial Sequence: Synthetic polypeptide
GAILGRSETQECLFYNANWELDRTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 149 - protein, 109 aa, Artificial Sequence
  Description of Artificial Sequence: Synthetic polypeptide
GAILGRSETQECLFYNANWELDRTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 150 - protein, 109 aa, Artificial Sequence
  Description of Artificial Sequence: Synthetic polypeptide
GAILGRSETQECLFYNANWELDRTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 151 - protein, 109 aa, Artificial Sequence
  Description of Artificial Sequence: Synthetic polypeptide
GAILGRSETQECLFYNANWELERTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 152 - protein, 109 aa, Artificial Sequence
  Description of Artificial Sequence: Synthetic polypeptide
GAILGRSETQECLFYNANWELERTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 153 - protein, 109 aa, Artificial Sequence
  Description of Artificial Sequence: Synthetic polypeptide
GAILGRSETQECLFYNANWELERTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 154 - protein, 109 aa, Artificial Sequence
  Description of Artificial Sequence: Synthetic polypeptide
GAILGRSETQECLFYNANWELERTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 155 - protein, 109 aa, Artificial Sequence
  Description of Artificial Sequence: Synthetic polypeptide
GAILGRSETQECLFYNANWELERTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 156 - protein, 109 aa, Artificial Sequence
  Description of Artificial Sequence: Synthetic polypeptide
GAILGRSETQECLFYNANWELDRTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 157 - protein, 109 aa, Artificial Sequence
  Description of Artificial Sequence: Synthetic polypeptide
GAILGRSETQECLFYNANWELDRTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 158 - protein, 109 aa, Artificial Sequence
  Description of Artificial Sequence: Synthetic polypeptide
GAILGRSETQECLFYNANWELDRTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 159 - protein, 109 aa, Artificial Sequence
  Description of Artificial Sequence: Synthetic polypeptide
GAILGRSETQECLFYNANWELDRTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 160 - protein, 109 aa, Artificial Sequence
  Description of Artificial Sequence: Synthetic polypeptide
GAILGRSETQECLFYNANWELDRTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 161 - protein, 109 aa, Artificial Sequence
  Description of Artificial Sequence: Synthetic polypeptide
GAILGRSETQECLFYNANWELDRTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 162 - protein, 109 aa, Artificial Sequence
  Description of Artificial Sequence: Synthetic polypeptide
GAILGRSETQECLFYNANWELDRTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 163 - protein, 109 aa, Artificial Sequence
  Description of Artificial Sequence: Synthetic polypeptide
GAILGRSETQECLFYNANWELDRTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 164 - protein, 109 aa, Artificial Sequence
  Description of Artificial Sequence: Synthetic polypeptide
GAILGRSETQECLFYNANWELDRTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 165 - protein, 109 aa, Artificial Sequence
  Description of Artificial Sequence: Synthetic polypeptide
GAILGRSETQECLFYNANWELDRTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 166 - protein, 109 aa, Artificial Sequence
  Description of Artificial Sequence: Synthetic polypeptide
GAILGRSETQECLFYNANWELERTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 167 - protein, 109 aa, Artificial Sequence
  Description of Artificial Sequence: Synthetic polypeptide
GAILGRSETQECLFYNANWELERTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 168 - protein, 109 aa, Artificial Sequence
  Description of Artificial Sequence: Synthetic polypeptide
GAILGRSETQECLFYNANWELERTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 169 - protein, 109 aa, Artificial Sequence
  Description of Artificial Sequence: Synthetic polypeptide
GAILGRSETQECLFYNANWELERTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 170 - protein, 109 aa, Artificial Sequence
  Description of Artificial Sequence: Synthetic polypeptide
GAILGRSETQECLFYNANWELERTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 171 - protein, 109 aa, Artificial Sequence
  Description of Artificial Sequence: Synthetic polypeptide
GAILGRSETQECLFYNANWELERTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 172 - protein, 109 aa, Artificial Sequence
  Description of Artificial Sequence: Synthetic polypeptide
GAILGRSETQECLFYNANWELERTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 173 - protein, 109 aa, Artificial Sequence
  Description of Artificial Sequence: Synthetic polypeptide
GAILGRSETQECLFYNANWELERTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 174 - protein, 109 aa, Artificial Sequence
  Description of Artificial Sequence: Synthetic polypeptide
GAILGRSETQECLFYNANWELERTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 175 - protein, 109 aa, Artificial Sequence
  Description of Artificial Sequence: Synthetic polypeptide
GAILGRSETQECLFYNANWELERTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 176 - protein, 109 aa, Artificial Sequence
  Description of Artificial Sequence: Synthetic polypeptide
GAILGRSETQECLFYNANWELDRTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 177 - protein, 109 aa, Artificial Sequence
  Description of Artificial Sequence: Synthetic polypeptide
GAILGRSETQECLFYNANWELDRTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 178 - protein, 109 aa, Artificial Sequence
  Description of Artificial Sequence: Synthetic polypeptide
GAILGRSETQECLFYNANWELDRTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 179 - protein, 109 aa, Artificial Sequence
  Description of Artificial Sequence: Synthetic polypeptide
GAILGRSETQECLFYNANWELDRTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 180 - protein, 109 aa, Artificial Sequence
  Description of Artificial Sequence: Synthetic polypeptide
GAILGRSETQECLFYNANWELDRTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 181 - protein, 109 aa, Artificial Sequence
  Description of Artificial Sequence: Synthetic polypeptide
GAILGRSETQECLFYNANWELDRTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 182 - protein, 109 aa, Artificial Sequence
  Description of Artificial Sequence: Synthetic polypeptide
GAILGRSETQECLFYNANWELDRTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 183 - protein, 109 aa, Artificial Sequence
  Description of Artificial Sequence: Synthetic polypeptide
GAILGRSETQECLFYNANWELDRTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 184 - protein, 109 aa, Artificial Sequence
  Description of Artificial Sequence: Synthetic polypeptide
GAILGRSETQECLFYNANWELDRTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 185 - protein, 109 aa, Artificial Sequence
  Description of Artificial Sequence: Synthetic polypeptide
GAILGRSETQECLFYNANWELDRTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 186 - protein, 109 aa, Artificial Sequence
  Description of Artificial Sequence: Synthetic polypeptide
GAILGRSETQECLFYNANWELERTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 187 - protein, 109 aa, Artificial Sequence
  Description of Artificial Sequence: Synthetic polypeptide
GAILGRSETQECLFYNANWELERTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 188 - protein, 109 aa, Artificial Sequence
  Description of Artificial Sequence: Synthetic polypeptide
GAILGRSETQECLFYNANWELERTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 189 - protein, 109 aa, Artificial Sequence
  Description of Artificial Sequence: Synthetic polypeptide
GAILGRSETQECLFYNANWELERTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 190 - protein, 109 aa, Artificial Sequence
  Description of Artificial Sequence: Synthetic polypeptide
GAILGRSETQECLFYNANWELERTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 191 - protein, 109 aa, Artificial Sequence
  Description of Artificial Sequence: Synthetic polypeptide
GAILGRSETQECLFYNANWELERTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 192 - protein, 109 aa, Artificial Sequence
  Description of Artificial Sequence: Synthetic polypeptide
GAILGRSETQECLFYNANWELERTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 193 - protein, 109 aa, Artificial Sequence
  Description of Artificial Sequence: Synthetic polypeptide
GAILGRSETQECLFYNANWELERTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 194 - protein, 109 aa, Artificial Sequence
  Description of Artificial Sequence: Synthetic polypeptide
GAILGRSETQECLFYNANWELERTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 195 - protein, 109 aa, Artificial Sequence
  Description of Artificial Sequence: Synthetic polypeptide
GAILGRSETQECLFYNANWELERTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 196 - protein, 109 aa, Artificial Sequence
  Description of Artificial Sequence: Synthetic polypeptide
GAILGRSETQECLFYNANWELDRTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 197 - protein, 109 aa, Artificial Sequence
  Description of Artificial Sequence: Synthetic polypeptide
GAILGRSETQECLFYNANWELDRTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 198 - protein, 109 aa, Artificial Sequence
  Description of Artificial Sequence: Synthetic polypeptide
GAILGRSETQECLFYNANWELDRTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 199 - protein, 109 aa, Artificial Sequence
  Description of Artificial Sequence: Synthetic polypeptide
GAILGRSETQECLFYNANWELDRTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 200 - protein, 109 aa, Artificial Sequence
  Description of Artificial Sequence: Synthetic polypeptide
GAILGRSETQECLFYNANWELDRTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 201 - protein, 109 aa, Artificial Sequence
  Description of Artificial Sequence: Synthetic polypeptide
GAILGRSETQECLFYNANWELERTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 202 - protein, 109 aa, Artificial Sequence
  Description of Artificial Sequence: Synthetic polypeptide
GAILGRSETQECLFYNANWELERTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 203 - protein, 109 aa, Artificial Sequence
  Description of Artificial Sequence: Synthetic polypeptide
GAILGRSETQECLFYNANWELERTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 204 - protein, 109 aa, Artificial Sequence
  Description of Artificial Sequence: Synthetic polypeptide
GAILGRSETQECLFYNANWELERTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 205 - protein, 109 aa, Artificial Sequence
  Description of Artificial Sequence: Synthetic polypeptide
GAILGRSETQECLFYNANWELDRTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 206 - protein, 109 aa, Artificial Sequence
  Description of Artificial Sequence: Synthetic polypeptide
GAILGRSETQECLFYNANWELERTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 207 - protein, 109 aa, Artificial Sequence
  Description of Artificial Sequence: Synthetic polypeptide
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 208 - protein, 109 aa, Artificial Sequence
  Description of Artificial Sequence: Synthetic polypeptide
GAILGRSETQECLYYNANWELERTNQTGVERCEGEQDKRLHCYATWRNISGSIEIVKKGCWLDDFNCYDRTDCVETEENPQVYFCCCEGNMCNEKFSYFPEMEVTQPTS

SEQ ID NO: 209 - protein, 111 aa, Artificial Sequence
  Description of Artificial Sequence: Synthetic polypeptide
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNP

SEQ ID NO: 210 - protein, 115 aa, Artificial Sequence
  Description of Artificial Sequence: Synthetic polypeptide
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNPVTPK

SEQ ID NO: 211 - protein, 109 aa, Unknown
  Description of Unknown: wild-type extracellular ActRIIA sequence
GAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 212 - protein, 109 aa, Artificial Sequence
  Description of Artificial Sequence: Synthetic polypeptide
GAILGRSETQECLFFNANWEKDRTNQTGVEPCYGDKDKRRHCFATWANISGSIEIVKQGCWLDDINCYDRTDCVEKKDSPEVYFCCCEGNMCNEKFSYFPEMEVTQPTS

SEQ ID NO: 213 - protein, 109 aa, Artificial Sequence
  Description of Artificial Sequence: Synthetic polypeptide
GAILGRSETQECLFFNANWAKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 214 - protein, 109 aa, Artificial Sequence
  Description of Artificial Sequence: Synthetic polypeptide
GAILGRSETQECLFFNANWEKDATNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 215 - protein, 109 aa, Artificial Sequence
  Description of Artificial Sequence: Synthetic polypeptide
GAILGRSETQECLFFNANWEKDRTNQTGVEPCYGDKAKRRHCFATWKNISGSIEIVKQGCWLDDINCYDRTDCVEKKDSPEVYFCCCEGNMCNEKFSYFPEMEVTQPTS

SEQ ID NO: 216 - protein, 109 aa, Artificial Sequence
  Description of Artificial Sequence: Synthetic polypeptide
GAILGRSETQECLFFNANWEKDRTNQTGVEPCYGDKDARRHCFATWKNISGSIEIVKQGCWLDDINCYDRTDCVEKKDSPEVYFCCCEGNMCNEKFSYFPEMEVTQPTS

SEQ ID NO: 217 - protein, 109 aa, Artificial Sequence
  Description of Artificial Sequence: Synthetic polypeptide
GAILGRSETQECLFFNANWEKDRTNQTGVEPCYGDKDKARHCFATWKNISGSIEIVKQGCWLDDINCYDRTDCVEKKDSPEVYFCCCEGNMCNEKFSYFPEMEVTQPTS

SEQ ID NO: 218 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVAQGCWLDDINCYDRTDCVEKKDSPEVYFCCCEGNMCNEKFSYFPEMEVTQPTS

SEQ ID NO: 219 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVYQGCWLDDINCYDRTDCVEKKDSPEVYFCCCEGNMCNEKFSYFPEMEVTQPTS

SEQ ID NO: 220 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVFQGCWLDDINCYDRTDCVEKKDSPEVYFCCCEGNMCNEKFSYFPEMEVTQPTS

SEQ ID NO: 221 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVIQGCWLDDINCYDRTDCVEKKDSPEVYFCCCEGNMCNEKFSYFPEMEVTQPTS

SEQ ID NO: 222 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCALDDINCYDRTDCVEKKDSPEVYFCCCEGNMCNEKFSYFPEMEVTQPTS

SEQ ID NO: 223 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWADDINCYDRTDCVEKKDSPEVYFCCCEGNMCNEKFSYFPEMEVTQPTS

SEQ ID NO: 224 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLKDINCYDRTDCVEKKDSPEVYFCCCEGNMCNEKFSYFPEMEVTQPTS

SEQ ID NO: 225 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLRDINCYDRTDCVEKKDSPEVYFCCCEGNMCNEKFSYFPEMEVTQPTS

SEQ ID NO: 226 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLADINCYDRTDCVEKKDSPEVYFCCCEGNMCNEKFSYFPEMEVTQPTS

SEQ ID NO: 227 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLFDINCYDRTDCVEKKDSPEVYFCCCEGNMCNEKFSYFPEMEVTQPTS

SEQ ID NO: 228 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLGDINCYDRTDCVEKKDSPEVYFCCCEGNMCNEKFSYFPEMEVTQPTS

SEQ ID NO: 229 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLMDINCYDRTDCVEKKDSPEVYFCCCEGNMCNEKFSYFPEMEVTQPTS

SEQ ID NO: 230 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLNDINCYDRTDCVEKKDSPEVYFCCCEGNMCNEKFSYFPEMEVTQPTS

SEQ ID NO: 231 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLIDINCYDRTDCVEKKDSPEVYFCCCEGNMCNEKFSYFPEMEVTQPTS

SEQ ID NO: 232 - protein, 109 aa, Artificial Sequence
  Description of Artificial Sequence: Synthetic polypeptide
GAILGRSETQECLFFNANWEKDRTNQTGVEPCYGDKDKRRHCFATWKNISGSIEIVKQGCWLDDANCYDRTDCVEKKDSPEVYFCCCEGNMCNEKFSYFPEMEVTQPTS

SEQ ID NO: 233 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 234 - protein, 4 aa, Artificial Sequence
  Description of Artificial Sequence: Synthetic peptide
GGGA

SEQ ID NO: 235 - protein, 4 aa, Artificial Sequence
  Description of Artificial Sequence: Synthetic peptide
GGGS

SEQ ID NO: 236 - protein, 5 aa, Artificial Sequence
  Description of Artificial Sequence: Synthetic peptide
GGGGA

SEQ ID NO: 237 - protein, 5 aa, Artificial Sequence
  Description of Artificial Sequence: Synthetic peptide
GGGGG

SEQ ID NO: 238 - protein, 4 aa, Artificial Sequence
  Description of Artificial Sequence: Synthetic peptide
GGAG

SEQ ID NO: 239 - protein, 4 aa, Artificial Sequence
  Description of Artificial Sequence: Synthetic peptide
GGSG

SEQ ID NO: 240 - protein, 4 aa, Artificial Sequence
  Description of Artificial Sequence: Synthetic peptide
AGGG

SEQ ID NO: 241 - protein, 4 aa, Artificial Sequence
  Description of Artificial Sequence: Synthetic peptide
GAGA

SEQ ID NO: 242 - protein, 4 aa, Artificial Sequence
  Description of Artificial Sequence: Synthetic peptide
GSGS

SEQ ID NO: 243 - protein, 6 aa, Artificial Sequence
  Description of Artificial Sequence: Synthetic peptide
GAGAGA

SEQ ID NO: 244 - protein, 6 aa, Artificial Sequence
  Description of Artificial Sequence: Synthetic peptide
GSGSGS

SEQ ID NO: 245 - protein, 8 aa, Artificial Sequence
  Description of Artificial Sequence: Synthetic peptide
GAGAGAGA

SEQ ID NO: 246 - protein, 8 aa, Artificial Sequence
  Description of Artificial Sequence: Synthetic peptide
GSGSGSGS

SEQ ID NO: 247 - protein, 10 aa, Artificial Sequence
  Description of Artificial Sequence: Synthetic peptide
GAGAGAGAGA

SEQ ID NO: 248 - protein, 10 aa, Artificial Sequence
  Description of Artificial Sequence: Synthetic peptide
GSGSGSGSGS

SEQ ID NO: 249 - protein, 12 aa, Artificial Sequence
  Description of Artificial Sequence: Synthetic peptide
GAGAGAGAGAGA

SEQ ID NO: 250 - protein, 12 aa, Artificial Sequence
  Description of Artificial Sequence: Synthetic peptide
GSGSGSGSGSGS

SEQ ID NO: 251 - protein, 6 aa, Artificial Sequence
  Description of Artificial Sequence: Synthetic peptide
GGAGGA

SEQ ID NO: 252 - protein, 6 aa, Artificial Sequence
  Description of Artificial Sequence: Synthetic peptide
GGSGGS

SEQ ID NO: 253 - protein, 9 aa, Artificial Sequence
  Description of Artificial Sequence: Synthetic peptide
GGAGGAGGA

SEQ ID NO: 254 - protein, 9 aa, Artificial Sequence
  Description of Artificial Sequence: Synthetic peptide
GGSGGSGGS

SEQ ID NO: 255 - protein, 12 aa, Artificial Sequence
  Description of Artificial Sequence: Synthetic peptide
GGAGGAGGAGGA

SEQ ID NO: 256 - protein, 12 aa, Artificial Sequence
  Description of Artificial Sequence: Synthetic peptide
GGSGGSGGSGGS

SEQ ID NO: 257 - protein, 8 aa, Artificial Sequence
  Description of Artificial Sequence: Synthetic peptide
GGAGGGAG

SEQ ID NO: 258 - protein, 8 aa, Artificial Sequence
  Description of Artificial Sequence: Synthetic peptide
GGSGGGSG

SEQ ID NO: 259 - protein, 12 aa, Artificial Sequence
  Description of Artificial Sequence: Synthetic peptide
GGAGGGAGGGAG

SEQ ID NO: 260 - protein, 12 aa, Artificial Sequence
  Description of Artificial Sequence: Synthetic peptide
GGSGGGSGGGSG

SEQ ID NO: 261 - protein, 15 aa, Artificial Sequence
  Description of Artificial Sequence: Synthetic peptide
GGGGAGGGGAGGGGA

SEQ ID NO: 262 - protein, 15 aa, Artificial Sequence
  Description of Artificial Sequence: Synthetic peptide
GGGGSGGGGSGGGGS

SEQ ID NO: 263 - protein, 5 aa, Artificial Sequence
  Description of Artificial Sequence: Synthetic peptide
GGGAG

SEQ ID NO: 264 - protein, 6 aa, Artificial Sequence
  Description of Artificial Sequence: Synthetic peptide
GGGAGG

SEQ ID NO: 265 - protein, 7 aa, Artificial Sequence
  Description of Artificial Sequence: Synthetic peptide
GGGAGGG

SEQ ID NO: 266 - protein, 4 aa, Artificial Sequence
  Description of Artificial Sequence: Synthetic peptide
AAAL

SEQ ID NO: 267 - protein, 4 aa, Artificial Sequence
  Description of Artificial Sequence: Synthetic peptide
AAAK

SEQ ID NO: 268 - protein, 4 aa, Artificial Sequence
  Description of Artificial Sequence: Synthetic peptide
AAAR

SEQ ID NO: 269 - protein, 14 aa, Artificial Sequence
  Description of Artificial Sequence: Synthetic peptide
EGKSSGSGSESKST

SEQ ID NO: 270 - protein, 12 aa, Artificial Sequence
  Description of Artificial Sequence: Synthetic peptide
GSAGSAAGSGEF

SEQ ID NO: 271 - protein, 12 aa, Artificial Sequence
  Description of Artificial Sequence: Synthetic peptide
AEAAAKEAAAKA

SEQ ID NO: 272 - protein, 18 aa, Artificial Sequence
  Description of Artificial Sequence: Synthetic peptide
KESGSVSSEQLAQFRSLD

SEQ ID NO: 273 - protein, 10 aa, Artificial Sequence
  Description of Artificial Sequence: Synthetic peptide
GENLYFQSGG

SEQ ID NO: 274 - protein, 8 aa, Artificial Sequence
  Description of Artificial Sequence: Synthetic peptide
SACYCELS

SEQ ID NO: 275 - protein, 5 aa, Artificial Sequence
  Description of Artificial Sequence: Synthetic peptide
RSIAT

SEQ ID NO: 276 - protein, 17 aa, Artificial Sequence
  Description of Artificial Sequence: Synthetic peptide
RPACKIPNDLKQKVMNH

SEQ ID NO: 277 - protein, 36 aa, Artificial Sequence
  Description of Artificial Sequence: Synthetic polypeptide
GGSAGGSGSGSSGGSSGASGTGTAGGTGSGSGTGSG

SEQ ID NO: 278 - protein, 17 aa, Artificial Sequence
  Description of Artificial Sequence: Synthetic peptide
AAANSSIDLISVPVDSR

SEQ ID NO: 279 - protein, 36 aa, Artificial Sequence
  Description of Artificial Sequence: Synthetic polypeptide
GGSGGGSEGGGSEGGGSEGGGSEGGGSEGGGSGGGS

SEQ ID NO: 280 - protein, 5 aa, Artificial Sequence
  Description of Artificial Sequence: Synthetic peptide
EAAAK

SEQ ID NO: 281 - protein, 5 aa, Artificial Sequence
  Description of Artificial Sequence: Synthetic peptide
PAPAP

SEQ ID NO: 282 - protein, 115 aa, Artificial Sequence
  Description of Artificial Sequence: Synthetic polypeptide
GRGEAETRECIFYNANWEKDRTNQSGLEPCYGDQDKRRHCFASWKNSSGTIELVKQGCWLDDINCYDRQECVAKKDSPEVYFCCCEGNFCNERFTHLPEAGGPEVTYEPPPTAPT

SEQ ID NO: 283 - protein, 109 aa, Artificial Sequence
  Description of Artificial Sequence: Synthetic polypeptide
GAILGRSETQECLFYNANWELERTNQTGVERCEGEKDKRLHCYATWRNISGSIEIVAKGCWLDDFNCYDRTDCVETEENPQVYFCCCEGNMCNEKFSYFPEMEVTQPTS

SEQ ID NO: 284 - protein, 246 aa, Unknown
  Description of Unknown: wild-type Fc domain
MEWSWVFLFFLSVTTGVHSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 11 aa, Unknown
  Description of Unknown: albumin binding peptide
DICLPRWGCLW

SEQ ID NO: 286 - protein, 93 aa, Unknown
  Description of Unknown: fibronectin domain
GPVEVFITETPSQPNSHPIQWNAPQPSHISKYILRWRPKNSVGRWKEATIPGHLNSYTIKGLKPGVVYEGQLISIQQYGHQEVTRFDFTTTST

SEQ ID NO: 287 - protein, 609 aa, Homo sapiens [sequence omitted]

SEQ ID NO: 288 - protein, 6 aa, Artificial Sequence
  Description of Artificial Sequence: Synthetic peptide
NPVTPK

SEQ ID NO: 289 - protein, 115 aa, Artificial Sequence
  Description of Artificial Sequence: Synthetic polypeptide
  MOD_RES (11)..(11): I or L
  MOD_RES (12)..(12): F or Y
  MOD_RES (19)..(19): L or K
  MOD_RES (20)..(20): D or E
  MOD_RES (25)..(25): T or S
  MOD_RES (27)..(27): L or V
  MOD_RES (29)..(29): P or R
  MOD_RES (31)..(31): Y or E
  MOD_RES (33)..(33): D or E
  MOD_RES (34)..(34): K or Q
  MOD_RES (38)..(38): R or L
  MOD_RES (41)..(41): Y or F
  MOD_RES (45)..(45): R or K
  MOD_RES (47)..(47): S or I
  MOD_RES (48)..(48): S or T
  MOD_RES (50)..(50): S or T
  MOD_RES (51)..(51): I or L
  MOD_RES (53)..(53): I or L
  MOD_RES (56)..(56): K or Q
  MOD_RES (63)..(63): F or I
  MOD_RES (69)..(69): Q, T, or D
  MOD_RES (70)..(70): E, D, or T
  MOD_RES (74)..(74): K or T
  MOD_RES (75)..(75): K or E
  MOD_RES (76)..(76): D or E
  MOD_RES (77)..(77): S or N
  MOD_RES (79)..(79): E or Q
  MOD_RES (89)..(89): F or M
  SITE (1)..(115): Either Xaa at position 75 is E and/or either Xaa at position 69 is T and Xaa at position 70 is D or Xaa at position 69 is D and Xaa at position 70 is T
  See specification as filed for detailed description of substitutions and preferred embodiments
GRGEAETRECXXYNANWEXXRTNQXGXEXCXGXXDKRXHCXASWXNXXGXXEXVKXGCWLDDXNCYDRXXCVAXXXXPXVYFCCCEGNXCNERFTHLPEAGGPEVTYEPPPTAPT

SEQ ID NO: 290 - protein, 115 aa, Artificial Sequence
  Description of Artificial Sequence: Synthetic polypeptide
GRGEAETRECIYYNANWELDRTNQSGLERCEGEQDKRLHCYASWRNSSGTIELVKKGCWLDDINCYDRQECVATKENPQVYFCCCEGNFCNERFTHLPEAGGPEVTYEPPPTAPT

SEQ ID NO: 291 - protein, 115 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 292 - protein, 115 aa, Artificial Sequence
  Description of Artificial Sequence: Synthetic polypeptide
GRGEAETRECIYYNANWELERTNQTGLERCEGEQDKRLHCYASWRNISGTIELVKKGCWLDDFNCYDRQECVAKKDSPEVYFCCCEGNFCNERFTHLPEAGGPEVTYEPPPTAPT

SEQ ID NO: 293 - protein, 115 aa, Artificial Sequence
  Description of Artificial Sequence: Synthetic polypeptide
GRGEAETRECIYYNANWELERTNQTGLERCEGEQDKRLHCYASWRNITGTIELVKKGCWLDDFNCYDRQECVAKKDSPEVYFCCCEGNFCNERFTHLPEAGGPEVTYEPPPTAPT

SEQ ID NO: 294 - protein, 115 aa, Artificial Sequence
  Description of Artificial Sequence: Synthetic polypeptide
GRGEAETRECIYYNANWELERTNQSGLEPCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 295 - protein, 115 aa, Artificial Sequence
  Description of Artificial Sequence: Synthetic polypeptide
GRGEAETRECIYYNANWELERTNQSGLERCYGDKDKRLHCFASWKNSSGTIELVKQGCWLDDFNCYDRQECVAKKDSPEVYFCCCEGNFCNERFTHLPEAGGPEVTYEPPPTAPT

SEQ ID NO: 296 - protein, 115 aa, Artificial Sequence; identical to SEQ ID NO: 291
  Description of Artificial Sequence: Synthetic polypeptide

SEQ ID NO: 297 - protein, 115 aa, Artificial Sequence
  Description of Artificial Sequence: Synthetic polypeptide
GRGEAETRECIFYNANWEKDRTNQSGLERCYGDQDKRRHCYASWRNSSGTIELVKKGCWLDDFNCYDRQECVAKKDSPEVYFCCCEGNFCNERFTHLPEAGGPEVTYEPPPTAPT

SEQ ID NO: 298 - protein, 115 aa, Artificial Sequence; identical to SEQ ID NO: 291
  Description of Artificial Sequence: Synthetic polypeptide

SEQ ID NO: 299 - protein, 115 aa, Artificial Sequence
  Description of Artificial Sequence: Synthetic polypeptide
GRGEAETRECLYYNANWELERTNQSGVERCEGEKDKRLHCYASWRNSSGSLEIVKKGCWLDDFNCYDRTDCVATEENPQVYFCCCEGNMCNERFTHLPEAGGPEVTYEPPPTAPT

SEQ ID NO: 300 - protein, 115 aa, Artificial Sequence
  Description of Artificial Sequence: Synthetic polypeptide
GRGEAETRECLYYNANWELERTNQSGVERCEGEKDKRLHCYASWRNSSGSLEIVKKGCWLDDFNCYDRDTCVATEENPQVYFCCCEGNMCNERFTHLPEAGGPEVTYEPPPTAPT

SEQ ID NO: 301 - protein, 115 aa, Artificial Sequence
  Description of Artificial Sequence: Synthetic polypeptide
GRGEAETRECLYYNANWELERTNQSGVERCEGEKDKRLHCYASWRNSSGSLEIVKKGCWLDDFNCYDRTDCVATKENPQVYFCCCEGNMCNERFTHLPEAGGPEVTYEPPPTAPT

SEQ ID NO: 302 - protein, 115 aa, Artificial Sequence
  Description of Artificial Sequence: Synthetic polypeptide
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPT

SEQ ID NO: 303 - protein, 110 aa, Unknown
  Description of Unknown: extracellular domain of ActRIIB
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 304 - protein, 110 aa, Unknown
  Description of Unknown: extracellular domain of ActRIIA
ETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 305 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDINCYDRTDCVEKKDSPEVYFCCCEGNFCNERFTHLPEAGGPEVTYEPPPTAPT

SEQ ID NO: 306 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDFNCYDRTDCVEKKDSPEVYFCCCEGNFCNERFTHLPEAGGPEVTYEPPPTAPT

SEQ ID NO: 307 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDINCYDRTDCVEKKDSPEVYFCCCEGNFCNERFTHLPEAGGPEVTYEPPPTAPT

SEQ ID NO: 308 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDINCYDRTDCVEKKDSPQVYFCCCEGNFCNERFTHLPEAGGPEVTYEPPPTAPT

SEQ ID NO: 309 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRTDCVEKKDSPQVYFCCCEGNFCNERFTHLPEAGGPEVTYEPPPTAPT

SEQ ID NO: 310 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRTDCVEKKDSPEVYFCCCEGNFCNERFTHLPEAGGPEVTYEPPPTAPT

SEQ ID NO: 311 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVEKKDSPEVYFCCCEGNFCNERFTHLPEAGGPEVTYEPPPTAPT

SEQ ID NO: 312 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVEKKDSPQVYFCCCEGNFCNERFTHLPEAGGPEVTYEPPPTAPT

SEQ ID NO: 313 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDINCYDRQECVEKKDSPEVYFCCCEGNFCNERFTHLPEAGGPEVTYEPPPTAPT

SEQ ID NO: 314 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRTDCVEKKDSPEVYFCCCEGNMCNERFTHLPEAGGPEVTYEPPPTAPT

SEQ ID NO: 315 - protein, 110 aa, Artificial Sequence
  Description of Artificial Sequence: Synthetic polypeptide
ETRECIYYNANWELERTNQSGLERCEGDQDKRLHCYASWRNSSGTIELVKKGCWLDDFNCYDRQECVEKKDSPEVYFCCCEGNMCNERFTHLPEAGGPEVTYEPPPTAPT

SEQ ID NO: 316 - protein, 110 aa, Artificial Sequence
  Description of Artificial Sequence: Synthetic polypeptide
ETQECIYYNANWEKDRTNQTGVEPCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 317 - protein, 110 aa, Artificial Sequence
  Description of Artificial Sequence: Synthetic polypeptide
ETQECIYYNANWEKDRTNQTGVEPCEGDQDKRLHCYASWRNSSGTIELVKKGCWLDDFNCYDRQECVATEENPQVYFCCCEGNFCNERFTHLPEAGGPEVTYEPPPTAPT

SEQ ID NO: 318 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 319 - protein, 110 aa, Artificial Sequence
  Description of Artificial Sequence: Synthetic polypeptide
ETRECIYYNANWEKDRTNQTGVEPCEGDQDKRLHCYASWRNSSGTIELVKKGCWLDDFNCYDRQECVATEENPQVYFCCCEGNFCNERFTHLPEAGGPEVTYEPPPTAPT

SEQ ID NO: 320 - protein, 110 aa, Artificial Sequence; identical to SEQ ID NO: 317
  Description of Artificial Sequence: Synthetic polypeptide

SEQ ID NO: 321 - protein, 110 aa, Artificial Sequence
  Description of Artificial Sequence: Synthetic polypeptide
ETQECIYYNANWEKDRTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 322 - protein, 110 aa, Artificial Sequence
  Description of Artificial Sequence: Synthetic polypeptide
ETQECIYYNANWEKDRTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 323 - protein, 110 aa, Artificial Sequence
  Description of Artificial Sequence: Synthetic polypeptide
ETRECIYYNANWEKDRTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 324 - protein, 110 aa, Artificial Sequence
  Description of Artificial Sequence: Synthetic polypeptide
ETQECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 325 - protein, 110 aa, Artificial Sequence
  Description of Artificial Sequence: Synthetic polypeptide
ETRECIYYNANWELERTNQSGLERCEGEQDKRLHCFATWRNSSGTIELVKQGCWLDDFNCYDRQECVATEENPQVYFCCCEGNFCNERFTHLPEAGGPEVTYEPPPTAPT

SEQ ID NO: 326 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDINCYDRQECVATEENPQVYFCCCEGNFCNERFTHLPEAGGPEVTYEPPPTAPT

SEQ ID NO: 327 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATKENPQVYFCCCEGNFCNERFTHLPEAGGPEVTYEPPPTAPT

SEQ ID NO: 328 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDINCYDRQECVATEENPQVYFCCCEGNFCNERFTHLPEAGGPEVTYEPPPTAPT

SEQ ID NO: 329 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDINCYDRQECVATKENPQVYFCCCEGNFCNERFTHLPEAGGPEVTYEPPPTAPT

SEQ ID NO: 330 - protein, 110 aa, Artificial Sequence
  Description of Artificial Sequence: Synthetic polypeptide
ETRECIYYNANWELERTNQSGLERCEGDKDKRLHCYASWRNSSGTIELVKKGCWLDDINCYDRQECVATKENPQVYFCCCEGNFCNERFTHLPEAGGPEVTYEPPPTAPT

SEQ ID NO: 331 - protein, 110 aa, Artificial Sequence
  Description of Artificial Sequence: Synthetic polypeptide
ETRECIYYNANWELERTNQSGLERCEGDQDKRLHCYASWRNSSGTIELVKKGCWLDDINCYDRQECVATKENPQVYFCCCEGNFCNERFTHLPEAGGPEVTYEPPPTAPT

SEQ ID NO: 332 - protein, 110 aa, Artificial Sequence
  Description of Artificial Sequence: Synthetic polypeptide
ETRECIYYNANWELERTNQSGLERCEGDQDKRLHCYASWRNSSGTIELVKKGCWLDDFNCYDRQECVATKENPQVYFCCCEGNFCNERFTHLPEAGGPEVTYEPPPTAPT

SEQ ID NO: 333 - protein, 110 aa, Artificial Sequence
  Description of Artificial Sequence: Synthetic polypeptide
ETRECIYYNANWELERTNQSGLERCEGDQDKRLHCYASWRNSSGTIELVKKGCWLDDFNCYDRQECVATEENPQVYFCCCEGNFCNERFTHLPEAGGPEVTYEPPPTAPT

SEQ ID NO: 334 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDINCYDRQECVEKKDSPEVYFCCCEGNFCNERFTHLPEAGGPEVTYEPPPTAPT

SEQ ID NO: 335 - protein, 110 aa, Artificial Sequence
  Description of Artificial Sequence: Synthetic polypeptide
ETRECIFFNANWEKDRTNQTGVEPCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 336 - protein, 110 aa, Artificial Sequence
  Description of Artificial Sequence: Synthetic polypeptide
ETRECIYYNANWELERTNQSGLERCEGEQDKRLHCFATWKNISGSIELVKQGCWLDDFNCYDRQECVATEENPQVYFCCCEGNFCNERFTHLPEAGGPEVTYEPPPTAPT

SEQ ID NO: 337 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRTDCVATEENPQVYFCCCEGNFCNERFTHLPEAGGPEVTYEPPPTAPT

SEQ ID NO: 338 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEENPQVYFCCCEGNMCNERFTHLPEAGGPEVTYEPPPTAPT

SEQ ID NO: 339 - protein, 110 aa, Artificial Sequence
  Description of Artificial Sequence: Synthetic polypeptide
ETRECIYYNANWELERTNQSGLERCEGDQDKRLHCYASWRNSSGTIELVKKGCWLDDINCYDRQECVATEENPQVYFCCCEGNFCNERFTHLPEAGGPEVTYEPPPTAPT

SEQ ID NO: 340 - protein, 110 aa, Artificial Sequence
  Description of Artificial Sequence: Synthetic polypeptide
ETQECLFFNANWEKDRTNQSGVEPCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 341 - protein, 110 aa, Artificial Sequence
  Description of Artificial Sequence: Synthetic polypeptide
ETQECLFFNANWEKDRTNQSGVEPCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 342 - protein, 110 aa, Artificial Sequence
  Description of Artificial Sequence: Synthetic polypeptide
ETRECLFFNANWEKDRTNQSGVEPCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 343 - protein, 110 aa, Artificial Sequence
  Description of Artificial Sequence: Synthetic polypeptide
ETQECLFFNANWEKDRTNQSGVEPCYGEQDKRLHCYASWRNSSGTIELVKKGCWLDDFNCYDRQECVATEENPQVYFCCCEGNFCNERFTHLPEAGGPEVTYEPPPTAPT

SEQ ID NO: 344 - protein, 110 aa, Artificial Sequence
  Description of Artificial Sequence: Synthetic polypeptide
ETRECLFFNANWEKDRTNQSGVEPCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 345 - protein, 110 aa, Artificial Sequence
  Description of Artificial Sequence: Synthetic polypeptide
ETRECLFFNANWEKDRTNQTGVEPCYGDKDKRRHCFATWKNISGSIEIVKQGCWLDDFNCYDRQECVATEENPQVYFCCCEGNFCNERFTHLPEAGGPEVTYEPPPTAPT

SEQ ID NO: 346 - protein, 110 aa, Artificial Sequence
  Description of Artificial Sequence: Synthetic polypeptide
ETRECLFFNANWEKDRTNQTGVEPCYGDKDKRRHCFATWKNISGSIEIVKQGCWLDDINCYDRQECVATEENPQVYFCCCEGNFCNERFTHLPEAGGPEVTYEPPPTAPT

SEQ ID NO: 347 - protein, 110 aa, Artificial Sequence
  Description of Artificial Sequence: Synthetic polypeptide
ETRECLFFNANWEKDRTNQTGVEPCYGDKDKRRHCFATWKNISGSIEIVKQGCWLDDINCYDRTDCVEKKDSPEVYFCCCEGNFCNERFTHLPEAGGPEVTYEPPPTAPT

SEQ ID NO: 348 - protein, 110 aa, Artificial Sequence
  Description of Artificial Sequence: Synthetic polypeptide
ETQECIYYNANWELERTNQSGLERCYGDKDKRRHCFATWKNISGSIEIVKQGCWLDDINCYDRQECVATEENPQVYFCCCEGNFCNERFTHLPEAGGPEVTYEPPPTAPT

SEQ ID NO: 349 - protein, 110 aa, Artificial Sequence
  Description of Artificial Sequence: Synthetic polypeptide
ETRECIYYNANWELERTNQSGLERCYGDKDKRRHCFATWKNISGSIEIVKQGCWLDDINCYDRTDCVEKKDSPEVYFCCCEGNFCNERFTHLPEAGGPEVTYEPPPTAPT

SEQ ID NO: 350 - protein, 110 aa, Artificial Sequence
  Description of Artificial Sequence: Synthetic polypeptide
ETRECIYYNANWELERTNQSGLERCYGDKDKRRHCFATWKNISGSIEIVKQGCWLDDINCYDRTDCVEKKDSPEVYFCCCEGNMCNERFTHLPEAGGPEVTYEPPPTAPT

SEQ ID NO: 351 - protein, 110 aa, Artificial Sequence
  Description of Artificial Sequence: Synthetic polypeptide
ETRECIYYNANWELERTNQSGLERCEGEQDKRLHCFATWKNISGSIEIVKQGCWLDDINCYDRTDCVEKKDSPEVYFCCCEGNFCNERFTHLPEAGGPEVTYEPPPTAPT

SEQ ID NO: 352 - protein, 110 aa, Artificial Sequence
  Description of Artificial Sequence: Synthetic polypeptide
ETRECIYYNANWELERTNQSGLERCEGEQDKRLHCFATWKNISGSIEIVKQGCWLDDINCYDRTDCVATEENPQVYFCCCEGNFCNERFTHLPEAGGPEVTYEPPPTAPT

SEQ ID NO: 353 - protein, 110 aa, Artificial Sequence
  Description of Artificial Sequence: Synthetic polypeptide
ETRECIYYNANWELERTNQSGLERCEGEQDKRLHCFATWKNISGSIEIVKQGCWLDDFNCYDRTDCVATEENPQVYFCCCEGNFCNERFTHLPEAGGPEVTYEPPPTAPT

SEQ ID NO: 354 - protein, 110 aa, Artificial Sequence
  Description of Artificial Sequence: Synthetic polypeptide
ETRECIYYNANWELERTNQSGLERCEGEQDKRLHCFATWKNISGSIEIVKQGCWLDDFNCYDRQECVATEENPQVYFCCCEGNFCNERFTHLPEAGGPEVTYEPPPTAPT

SEQ ID NO: 355 - protein, 110 aa, Artificial Sequence
  Description of Artificial Sequence: Synthetic polypeptide
ETQECIYYNANWELERTNQSGLERCYGDKDKRRHCYASWRNSSGTIELVKKGCWLDDINCYDRTDCVEKKDSPEVYFCCCEGNFCNERFTHLPEAGGPEVTYEPPPTAPT

SEQ ID NO: 356 - protein, 110 aa, Artificial Sequence
  Description of Artificial Sequence: Synthetic polypeptide
ETQECIYYNANWELERTNQSGLERCYGDKDKRRHCYASWRNSSGTIELVKKGCWLDDINCYDRTDCVEKKDSPEVYFCCCEGNMCNERFTHLPEAGGPEVTYEPPPTAPT

SEQ ID NO: 357 - protein, 110 aa, Artificial Sequence
  Description of Artificial Sequence: Synthetic polypeptide
ETQECIYYNANWELERTNQSGLERCYGDKDKRRHCFATWKNISGSIEIVKQGCWLDDINCYDRTDCVEKKDSPEVYFCCCEGNMCNERFTHLPEAGGPEVTYEPPPTAPT

SEQ ID NO: 358 - protein, 110 aa, Artificial Sequence
  Description of Artificial Sequence: Synthetic polypeptide
ETQECIYYNANWELERTNQSGLERCYGDKDKRRHCFATWKNISGSIEIVKQGCWLDDINCYDRTDCVEKKDSPEVYFCCCEGNFCNERFTHLPEAGGPEVTYEPPPTAPT

SEQ ID NO: 359 - protein, 110 aa, Artificial Sequence
  Description of Artificial Sequence: Synthetic polypeptide
ETRECLFFNANWEKDRTNQTGVEPCEGEQDKRLHCFATWKNISGSIEIVKQGCWLDDINCYDRTDCVEKKDSPEVYFCCCEGNFCNERFTHLPEAGGPEVTYEPPPTAPT

SEQ ID NO: 360 - protein, 110 aa, Artificial Sequence
  Description of Artificial Sequence: Synthetic polypeptide
ETRECLFFNANWEKDRTNQSGVEPCEGEQDKRLHCYASWRNSSGTIELVKKGCWLDDINCYDRTDCVEKKDSPEVYFCCCEGNFCNERFTHLPEAGGPEVTYEPPPTAPT

SEQ ID NO: 361 - protein, 110 aa, Artificial Sequence
  Description of Artificial Sequence: Synthetic polypeptide
ETRECLFFNANWEKDRTNQSGVEPCYGDKDKRRHCYASWRNSSGTIELVKKGCWLDDINCYDRTDCVEKKDSPEVYFCCCEGNFCNERFTHLPEAGGPEVTYEPPPTAPT

SEQ ID NO: 362 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDFNCYDRTDCVATEENPQVYFCCCEGNFCNERFTHLPEAGGPEVTYEPPPTAPT

SEQ ID NO: 363 - protein, 110 aa, Artificial Sequence
  Description of Artificial Sequence: Synthetic polypeptide
ETRECIYYNANWELERTNQSGLERCYGDKDKRRHCYASWRNSSGTIELVKQGCWLDDFNCYDRTDCVATEENPQVYFCCCEGNFCNERFTHLPEAGGPEVTYEPPPTAPT

SEQ ID NO: 364 - protein, 110 aa, Artificial Sequence
  Description of Artificial Sequence: Synthetic polypeptide
ETRECIYYNANWELERTNQSGLERCYGDKDKRRHCYASWRNSSGTIEIVKKGCWLDDFNCYDRTDCVATEENPQVYFCCCEGNFCNERFTHLPEAGGPEVTYEPPPTAPT

SEQ ID NO: 365 - protein, 110 aa, Artificial Sequence
  Description of Artificial Sequence: Synthetic polypeptide
ETRECIYYNANWELERTNQSGLERCYGDKDKRRHCYASWRNSSGSIELVKKGCWLDDFNCYDRTDCVATEENPQVYFCCCEGNFCNERFTHLPEAGGPEVTYEPPPTAPT

SEQ ID NO: 366 - protein, 110 aa, Artificial Sequence
  Description of Artificial Sequence: Synthetic polypeptide
ETRECIYYNANWELERTNQSGLERCYGDKDKRRHCYASWRNSSGSIEIVKKGCWLDDFNCYDRTDCVATEENPQVYFCCCEGNFCNERFTHLPEAGGPEVTYEPPPTAPT

SEQ ID NO: 367 - protein, 110 aa, Artificial Sequence
  Description of Artificial Sequence: Synthetic polypeptide
ETRECIYYNANWELERTNQSGLERCYGDKDKRRHCYASWRNSSGSIEIVKQGCWLDDFNCYDRTDCVATEENPQVYFCCCEGNFCNERFTHLPEAGGPEVTYEPPPTAPT

SEQ ID NO: 368 - protein, 110 aa, Artificial Sequence; identical to SEQ ID NO: 337
  Description of Artificial Sequence: Synthetic polypeptide

SEQ ID NO: 369 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEENPQVYFCCCEGNFCNEKFSYFPEMEVTQPTSNPVTPKPP

SEQ ID NO: 370 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEENPQVYFCCCEGNFCNEKFSYFPQMEVTQPTSNPVTPKPP

SEQ ID NO: 371 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDINCYDRTDCVEKKDSPEVYFCCCEGNFCNEKFSYFPEMEVTQPTSNPVTPKPP

SEQ ID NO: 372 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDINCYDRTDCVEKKDSPEVYFCCCEGNFCNEKFSYFPQMEVTQPTSNPVTPKPP

SEQ ID NO: 373 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDFNCYDRQECVATEENPQVYFCCCEGNFCNEKFSYFPQMEVTQPTSNPVTPKPP

SEQ ID NO: 374 - protein, 110 aa, Artificial Sequence
  Description of Artificial Sequence: Synthetic polypeptide
ETRECIYYNANWELERTNQSGLERCYGDKDKRRHCYASWRNSSGTIELVKQGCWLDDFNCYDRQECVATEENPQVYFCCCEGNFCNEKFSYFPQMEVTQPTSNPVTPKPP

SEQ ID NO: 375 - protein, 110 aa, Artificial Sequence
  Description of Artificial Sequence: Synthetic polypeptide
ETRECIYYNANWELERTNQSGLERCYGDKDKRRHCYASWRNSSGTIEIVKKGCWLDDFNCYDRQECVATKENPQVYFCCCEGNFCNEKFSYFPQMEVTQPTSNPVTPKPP

SEQ ID NO: 376 - protein, 110 aa, Artificial Sequence
  Description of Artificial Sequence: Synthetic polypeptide
ETRECIYYNANWELERTNQSGLERCYGDKDKRRHCYASWRNSSGSIELVKKGCWLDDFNCYDRQECVATKENPQVYFCCCEGNFCNEKFSYFPQMEVTQPTSNPVTPKPP

SEQ ID NO: 377 - protein, 110 aa, Artificial Sequence
  Description of Artificial Sequence: Synthetic polypeptide
ETRECIYYNANWELERTNQSGLERCEGEQDKRLHCYASWRNSSGSIELVKKGCWLDDFNCYDRQECVATEENPQVYFCCCEGNFCNEKFSYFPQMEVTQPTSNPVTPKPP

SEQ ID NO: 378 - protein, 110 aa, Artificial Sequence
  Description of Artificial Sequence: Synthetic polypeptide
ETRECIYYNANWELERTNQSGLERCEGEQDKRLHCYASWRNSSGTIEIVKKGCWLDDFNCYDRQECVATEENPQVYFCCCEGNFCNEKFSYFPQMEVTQPTSNPVTPKPP

SEQ ID NO: 379 - protein, 110 aa, Artificial Sequence
  Description of Artificial Sequence: Synthetic polypeptide
ETRECIYYNANWELERTNQSGLERCEGEQDKRLHCYASWRNSSGTIELVKQGCWLDDFNCYDRQECVATEENPQVYFCCCEGNFCNEKFSYFPQMEVTQPTSNPVTPKPP

SEQ ID NO: 380 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVETEENPQVYFCCCEGNFCNEKFSYFPQMEVTQPTSNPVTPKPP

SEQ ID NO: 381 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVAKEENPQVYFCCCEGNFCNEKFSYFPQMEVTQPTSNPVTPKPP

SEQ ID NO: 382 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATKENPQVYFCCCEGNFCNEKFSYFPQMEVTQPTSNPVTPKPP

SEQ ID NO: 383 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEDNPQVYFCCCEGNFCNEKFSYFPQMEVTQPTSNPVTPKPP

SEQ ID NO: 384 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEESPQVYFCCCEGNFCNEKFSYFPQMEVTQPTSNPVTPKPP

SEQ ID NO: 385 - protein, 110 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEENPEVYFCCCEGNFCNEKFSYFPQMEVTQPTSNPVTPKPP

SEQ ID NO: 386 - protein, 110 aa, Artificial Sequence
  Description of Artificial Sequence: Synthetic polypeptide
ETRECIYYNANWELERTNQSGLERCEGDKDKRLHCYASWRNSSGTIELVKKGCWLDDFNCYDRQECVETEENPQVYFCCCEGNFCNEKFSYFPQMEVTQPTSNPVTPKPP

SEQ ID NO: 387 - protein, 110 aa, Artificial Sequence
  Description of Artificial Sequence: Synthetic polypeptide
ETRECIYYNANWELERTNQSGLERCEGEKDKRLHCYASWRNSSGTIELVKKGCWLDDFNCYDRQECVAKEENPQVYFCCCEGNFCNEKFSYFPQMEVTQPTSNPVTPKPP

SEQ ID NO: 388 - protein, 110 aa, Artificial Sequence
  Description of Artificial Sequence: Synthetic polypeptide
ETRECIYYNANWELERTNQSGLERCEGDQDKRLHCYASWRNSSGTIELVKKGCWLDDFNCYDRQECVATKENPQVYFCCCEGNFCNEKFSYFPQMEVTQPTSNPVTPKPP

SEQ ID NO: 389 - protein, 110 aa, Artificial Sequence
  Description of Artificial Sequence: Synthetic polypeptide
ETRECIYYNANWELERTNQSGLERCEGDKDKRLHCYASWRNSSGTIELVKQGCWLDDFNCYDRQECVAKKENPQVYFCCCEGNFCNEKFSYFPQMEVTQPTSNPVTPKPP

SEQ ID NO: 390 - protein, 110 aa, Artificial Sequence
  Description of Artificial Sequence: Synthetic polypeptide
ETRECIYYNANWELERTNQSGLERCEGDKDKRLHCYASWRNSSGTIEIVKQGCWLDDFNCYDRQECVAEKENPQVYFCCCEGNFCNEKFSYFPQMEVTQPTSNPVTPKPP

SEQ ID NO: 391 - protein, 110 aa, Artificial Sequence
  Description of Artificial Sequence: Synthetic polypeptide
ETRECIYYNANWELERTNQSGLERCYGDQDKRLHCYASWRNSSGSIEIVKQGCWLDDFNCYDRQECVAKKENPQVYFCCCEGNFCNEKFSYFPQMEVTQPTSNPVTPKPP

SEQ ID NO: 392 - protein, 110 aa, Artificial Sequence
  Description of Artificial Sequence: Synthetic polypeptide
ETRECIYYNANWELERTNQSGLERCEGEKDKRRHCYASWRNSSGTIEIVKKGCWLDDFNCYDRQECVATKENPQVYFCCCEGNFCNEKFSYFPQMEVTQPTSNPVTPKPP

SEQ ID NO: 393 - protein, 110 aa, Artificial Sequence
  Description of Artificial Sequence: Synthetic polypeptide
ETRECIYYNANWELERTNQSGLERCYGDQDKRRHCYASWRNSSGTIELVKKGCWLDDFNCYDRQECVATEENPEVYFCCCEGNFCNEKFSYFPQMEVTQPTSNPVTPKPP

SEQ ID NO: 394 - protein, 110 aa, Artificial Sequence
  Description of Artificial Sequence: Synthetic polypeptide
ETRECIYYNANWELERTNQSGLERCEGEQDKRRHCYASWRNSSGTIELVKKGCWLDDFNCYDRQECVATEENPQVYFCCCEGNFCNEKFSYFPQMEVTQPTSNPVTPKPP

SEQ ID NO: 395 - protein, 110 aa, Artificial Sequence
  Description of Artificial Sequence: Synthetic polypeptide
ETRECIYYNANWELERTNQSGLERCYGEQDKRLHCYASWRNSSGSIEIVKKGCWLDDFNCYDRTDCVATEENPQVYFCCCEGNFCNEKFSYFPQMEVTQPTSNPVTPKPP

SEQ ID NO: 396 - protein, 110 aa, Artificial Sequence
  Description of Artificial Sequence: Synthetic polypeptide
ETRECIYYNANWELERTNQSGLERCEGEQDKRRHCYASWRNSSGSIELVKKGCWLDDFNCYDRQECVAKEENPQVYFCCCEGNFCNEKFSYFPQMEVTQPTSNPVTPKPP

SEQ ID NO: 397 - protein, 110 aa, Artificial Sequence
  Description of Artificial Sequence: Synthetic polypeptide
ETRECIYYNANWELERTNQSGLERCEGEQDKRRHCYASWRNSSGTIEIVKKGCWLDDFNCYDRQECVAKEENPQVYFCCCEGNFCNEKFSYFPQMEVTQPTSNPVTPKPP

SEQ ID NO: 398 - protein, 110 aa, Artificial Sequence
  Description of Artificial Sequence: Synthetic polypeptide
ETRECIYYNANWELERTNQSGLERCEGEQDKRRHCYASWRNSSGSIEIVKKGCWLDDFNCYDRQECVATKENPQVYFCCCEGNFCNEKFSYFPQMEVTQPTSNPVTPKPP

SEQ ID NO: 399 - protein, 110 aa, Artificial Sequence
  Description of Artificial Sequence: Synthetic polypeptide
ETRECIYYNANWELERTNQSGLERCEGDQDKRLHCYASWRNSSGTIEIVKKGCWLDDFNCYDRQECVATKENPQVYFCCCEGNFCNEKFSYFPQMEVTQPTSNPVTPKPP

SEQ ID NO: 400 - protein, 110 aa, Artificial Sequence
  Description of Artificial Sequence: Synthetic polypeptide
ETRECIYYNANWELERTNQSGLERCEGDQDKRLHCYASWRNSSGSIELVKKGCWLDDFNCYDRQECVATKENPQVYFCCCEGNFCNEKFSYFPQMEVTQPTSNPVTPKPP

SEQ ID NO: 401 - protein, 110 aa, Artificial Sequence; identical to SEQ ID NO: 388
  Description of Artificial Sequence: Synthetic polypeptide

SEQ ID NO: 402 - protein, 110 aa, Artificial Sequence
  Description of Artificial Sequence: Synthetic polypeptide
ETRECIYYNANWELERTNQSGLERCEGEQDKRLHCYASWRNSSGTIEIVKKGCWLDDFNCYDRQECVATKENPQVYFCCCEGNFCNEKFSYFPQMEVTQPTSNPVTPKPP

SEQ ID NO: 403 - protein, 110 aa, Artificial Sequence; identical to SEQ ID NO: 382
  Description of Artificial Sequence: Synthetic polypeptide

SEQ ID NO: 404 - protein, 110 aa, Artificial Sequence
  Description of Artificial Sequence: Synthetic polypeptide
ETRECIYYNANWELERTNQSGLERCEGDQDKRLHCYASWRNSSGTIEIVKKGCWLDDFNCYDRQECVAKKENPQVYFCCCEGNFCNEKFSYFPQMEVTQPTSNPVTPKPP

SEQ ID NO: 405 - protein, 110 aa, Artificial Sequence
  Description of Artificial Sequence: Synthetic polypeptide
ETQECLFFNANWEKDRTNQTGVEPCYGDKDKRRHCFATWKNISGSIEIVKQGCWLDDINCYDRTDCVEKKDSPEVYFCCCEGNMCNERFTHLPEAGGPEVTYEPPPTAPT

SEQ ID NO: 406 - protein, 110 aa, Artificial Sequence
  Description of Artificial Sequence: Synthetic polypeptide
ETRECIYYNANWELERTNQSGLERCYGDKDKRRHCYASWRNSSGTIELVKKGCWLDDINCYDRQECVATKENPQVYFCCCEGNFCNERFTHLPEAGGPEVTYEPPPTAPT

SEQ ID NO: 407 - protein, 116 aa, Homo sapiens
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 408 - protein, 513 aa, Homo sapiens
MGAATKLAFAVFLISCSSGAILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPPLFNTLLYSLVPIMGIAVIVLFSFWMYRHHKLAYPPVLVPTQDPGPPPPSPLMGLKPLQLLEIKARGRFGCVWKAQLLNEYVAVKIFPIQDKQSWQNEYEIYSLPGMKHDNILQFIGAEKRGTSIDVDLWLITAFHEKGSLTDFLKANVVSWNELCHIAQTMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTASDGPVDEYMLPFEEEIGQHPSLEDMQEVVVHKKKRPVLRECWQKHSGMAMLCETIEECWDHDAEARLSAGCVEERIIQMQKLTNIITTEDIVTVVTMVTNVDFPPKESSL

SEQ ID NO: 409 - protein, 116 aa, Homo sapiens
AILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 410 - protein, 109 aa, Artificial Sequence; identical to SEQ ID NO: 139
  Description of Artificial Sequence: Synthetic polypeptide
  MOD_RES (14)..(14): F, A, V, I, L, M, F, Y or W
  MOD_RES (15)..(15): F, A, V, I, L, M, F, Y or W
  MOD_RES (20)..(20): D, E or A
  MOD_RES (21)..(21): K or L
  MOD_RES (22)..(22): D, E, or A
  MOD_RES (23)..(23): R, H, K, or A
  MOD_RES (31)..(31): P or R
  MOD_RES (33)..(33): Y or E
  MOD_RES (35)..(35): D, E, or A
  MOD_RES (36)..(36): K or Q
  MOD_RES (37)..(37): D, E, or A
  MOD_RES (38)..(38): K, R, H, or A
  MOD_RES (39)..(39): R, H, K, or A
  MOD_RES (40)..(40): R or L
  MOD_RES (43)..(43): F, A, V, I, L, M, F, Y or W
  MOD_RES (47)..(47): K, R, H, or A
  MOD_RES (57)..(57): K, A, Y, F, I, V, L, M, or W
  MOD_RES (58)..(58): Q or K
  MOD_RES (61)..(61): W or A
  MOD_RES (62)..(62): L or A
  MOD_RES (63)..(63): D, K, R, H, A, F, G, M, N, or I
  MOD_RES (65)..(65): I, F, A, V, L, M, Y or W
  MOD_RES (76)..(76): K or T
  MOD_RES (77)..(77): K, R, H, D, or E
  MOD_RES (78)..(78): D, E, or A
  MOD_RES (79)..(79): S, N, T, or Q
  MOD_RES (81)..(81): E or Q
  See specification as filed for detailed description of substitutions and preferred embodiments

SEQ ID NO: 411 - protein, 115 aa, Artificial Sequence; identical to SEQ ID NO: 289
  Description of Artificial Sequence: Synthetic polypeptide
  MOD_RES (11)..(11): I or L
  MOD_RES (12)..(12): F, Y, A, V, I, L, M, or W
  MOD_RES (19)..(19): L or K
  MOD_RES (20)..(20): D, E, or A
  MOD_RES (25)..(25): T, S, N, or Q
  MOD_RES (27)..(27): L, V, A, I, M, F, Y, or W
  MOD_RES (29)..(29): P or R
  MOD_RES (31)..(31): Y or E
  MOD_RES (33)..(33): D, E, or A
  MOD_RES (34)..(34): K or Q
  MOD_RES (38)..(38): R or L
  MOD_RES (41)..(41): A, V, I, L, M, Y, W or F
  MOD_RES (45)..(45): R, H, or K
  MOD_RES (47)..(47): S or I
  MOD_RES (48)..(48): S, N, Q, or T
  MOD_RES (50)..(50): S, N, Q, or T
  MOD_RES (51)..(51): L, V, A, I, M, F, Y, or W
  MOD_RES (53)..(53): L, V, A, I, M, F, Y, or W
  MOD_RES (56)..(56): K or Q
  MOD_RES (63)..(63): L, V, A, I, M, F, Y, or W
  MOD_RES (69)..(69): Q, T, S, N or D
  MOD_RES (70)..(70): E, D, A, or T
  MOD_RES (74)..(74): K or T
  MOD_RES (75)..(75): K or E
  MOD_RES (76)..(76): D, A, or E
  MOD_RES (77)..(77): T, S, N, or Q
  MOD_RES (79)..(79): E or Q
  MOD_RES (89)..(89): L, V, A, I, M, F, Y, or W
  SITE (1)..(115): Either Xaa at position 75 is E and/or either Xaa at position 69 is T and Xaa at position 70 is D or Xaa at position 69 is D and Xaa at position 70 is T
  See specification as filed for detailed description of substitutions and preferred embodiments

SEQ ID NO: 412 - protein, 4 aa, Artificial Sequence
  Description of Artificial Sequence: Synthetic peptide
TEEN

SEQ ID NO: 413 - protein, 4 aa, Artificial Sequence
  Description of Artificial Sequence: Synthetic peptide
TKEN

SEQ ID NO: 414 - protein, 20 aa, Artificial Sequence
  Description of Artificial Sequence: Synthetic peptide
  SITE (1)..(20): This sequence may encompass 1-4 “Gly Gly Gly Gly Ser” repeating units
GGGGSGGGGSGGGGSGGGGS

SEQ ID NO: 415 - protein, 10 aa, Artificial Sequence
  Description of Artificial Sequence: Synthetic peptide
  MOD_RES (1)..(1): Any amino acid
  MOD_RES (3)..(3): Any amino acid
  MOD_RES (5)..(5): Any amino acid
  MOD_RES (7)..(7): Any amino acid
  MOD_RES (9)..(9): Any amino acid
  SITE (1)..(10): This sequence may encompass 1-5 “Xaa Pro” repeating units
  See specification as filed for detailed description of substitutions and preferred embodiments
XPXPXPXPXP

SEQ ID NO: 416 - protein, 10 aa, Artificial Sequence
  Description of Artificial Sequence: Synthetic peptide
  See specification as filed for detailed description of substitutions and preferred embodiments
GGGGSGGGGS

SEQ ID NO: 417 - DNA, 48 bp, Artificial Sequence
  Description of Artificial Sequence: Synthetic oligonucleotide
cagacagacagacagacagacagacagacagacagacagacagacaga

(19) United States
(12) Patent Application Publication
(10) Pub. No.: US 2023/0129812 A1
(43) Pub. Date: Apr. 27, 2023

(54) COMPOSITIONS AND METHODS FOR TREATING PULMONARY HYPERTENSION

(71) Applicant: Acceleron Pharma Inc., Cambridge, MA (US)

(72) Inventor: Ravindra Kumar, Cambridge, MA (US)

(21) Appl. No.: 17/797,229

(22) PCT Filed: Feb. 3, 2021
(86) PCT No.: PCT/US2021/016461

§ 371 Date: Aug. 3, 2022

Related U.S. Application Data
(60) Provisional application No. 62/969,519, filed on Feb. 3, 2020.

(51) Int. Cl. A61K 38/17; A61K 47/68; A61P 9/12; C07K 14/71

(52) U.S. Cl. CPC A61K 38/1796; A61K 38/1709; A61K 47/68; A61P 9/12; C07K 14/71; C07K 2319/30

(57) ABSTRACT

In some aspects, the disclosure relates to ActRII antagonists and methods of using ActRII antagonists to treat, prevent, or reduce the progression rate and/or severity of pulmonary hypertension (PH), particularly treating, preventing or reducing the progression rate and/or severity of one or more PH-associated complications. The disclosure also provides methods of using an ActRII antagonist to treat, prevent, or reduce the progression rate and/or severity of a variety of conditions including, but not limited to, pulmonary vascular remodeling, pulmonary fibrosis, and right ventricular hypertrophy. The disclosure further provides methods of using an ActRII antagonist to reduce right ventricular systolic pressure in a subject in need thereof.

### CROSS-REFERENCE TO RELATED APPLICATIONS

This application claims the benefit of priority from U.S. Provisional Application No. 62/969,519, filed Feb. 3, 2020. The specification of the foregoing application is incorporated herein by reference in its entirety.

### BACKGROUND OF THE INVENTION

Pulmonary hypertension (PH) is a disease characterized by high blood pressure in lung vasculature, including pulmonary arteries, pulmonary veins, and pulmonary capillaries. In general, PH is defined as a mean pulmonary arterial (PA) pressure ≥25 mm Hg at rest or ≥30 mm Hg with exercise [Hill et al., Respiratory Care 54(7):958-68 (2009)]. The main PH symptom is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches [Simonneau et al., JACC 54(1):S44-54 (2009)]. Clinical classification of PH was first proposed in 1973, and a recent updated clinical classification was endorsed by the World Health Organization (WHO) in 2008. According to the updated PH clinical classification, there are five main groups of PH: pulmonary arterial hypertension (PAH), characterized by a PA wedge pressure ≤15 mm Hg; PH owing to a left heart disease (also known as pulmonary venous hypertension or congestive heart failure), characterized by a PA wedge pressure >15 mm Hg; PH owing to lung diseases and/or hypoxia; chronic thromboemboli PH; and PH with unclear or multifactorial etiologies [Simonneau et al., JACC 54(1):S44-54 (2009); Hill et al., Respiratory Care 54(7):958-68 (2009)]. PAH is further classified into idiopathic PAH (IPAH), a sporadic disease in which there is neither a family history of PAH nor an identified risk factor; heritable PAH; PAH induced by drugs and toxins; PAH associated with connective tissue diseases, HIV infection, portal hypertension, congenital heart diseases, schistosomiasis, and chronic hemolytic anemia; and persistent PH of newborns [Simonneau et al., JACC 54(1):S44-54 (2009)]. Diagnosis of various types of PH requires a series of tests.

In general, PH treatment depends on the cause or classification of the PH. Where PH is caused by a known medicine or medical condition, it is known as a secondary PH, and its treatment is usually directed at the underlying disease. Treatment of pulmonary venous hypertension generally involves optimizing left ventricular function by administering diuretics, beta blockers, and ACE inhibitors, or repairing or replacing a mitral valve or aortic valve. PAH therapies include pulmonary vasodilators, digoxin, diuretics, anticoagulants, and oxygen therapy. Pulmonary vasodilators target different pathways, including prostacyclin pathway (e.g., prostacyclins, including intravenous epoprostenol, subcutaneous or intravenous treprostinil, and inhaled iloprost), nitric oxide pathway (e.g., phosphodiesterase-5 inhibitors, including sildenafil and tadalafil), and endotheline-1 pathway (e.g., endothelin receptor antagonists, including oral bosentan and oral ambrisentan) [Humbert, M. Am. J. Respir. Crit. Care Med. 179:650-6 (2009); Hill et al., Respiratory Care 54(7):958-68 (2009)]. However, current therapies provide no cure for PH, and they do not directly treat the underling vascular remodeling and muscularization of blood vessels observed in many PH patients.

Thus, there is a high, unmet need for effective therapies for treating pulmonary hypertension. Accordingly, it is an object of the present disclosure to provide methods for treating, preventing, or reducing the progression rate and/or severity of PH, particular treating, preventing or reducing the progression rate and/or severity of one or more PH-associated complications.

### SUMMARY OF THE INVENTION

In part, the data presented herein demonstrates that ActRII antagonists or heteromultimers comprising the same can be used to treat pulmonary hypertension. For example, it was previously shown that a soluble ActRIIA polypeptide and an ALK4:ActRIIB heterodimer can be used, individually, to reduce blood pressure, cardiac hypertrophy, and lung weight in a monocrotaline-induced pulmonary arterial hypertension (PAH) model. Similar positive effects were observed for the ActRIIA polypeptide in the Sugen hypoxia PAH model. Histological analysis further revealed that the ActRIIA polypeptide had surprising and significant effects on decreasing vascular remodeling and muscularization of blood vessels in both the monocrotaline-induced and Sugen hypoxia models of PAH. Moreover, both the ActRIIA polypeptide and ALK4:ActRIIB heterodimer surprisingly had a greater effect on ameliorating various complications of PAH compared to sildenafil, which is a drug approved for the treatment of PAH. Thus, the disclosure establishes that antagonists of the ActRII (ActRIIA and ActRIIB) signaling pathways may be used to reduce the severity of pulmonary hypertension. While soluble ActRIIA polypeptides and ALK4:ActRIIB heteromultimers may affect pulmonary hypertension through a mechanism other than ActRIIA/B ligand antagonisms, the disclosure nonetheless demonstrates that desirable therapeutic agents may be selected on the basis of ActRII signaling antagonist activity. Therefore, in some embodiments, the disclosure provides methods for using various ActRII signaling antagonists for treating hypertension, particularly pulmonary hypertension, including, for example, antagonists that inhibit one or more TGF-beta family ligands [e.g., activin A, activin B, activin AB, activin AC, BMP6, BMP7, BMP9, BMP10, GDF3, GDF8, and GDF11]; antagonists that inhibit ActRIIA or ActRIIB; and antagonists that inhibit one or more downstream signaling components (e.g., Smad proteins). As used herein, such signaling antagonists are collectively referred to as “ActRII antagonists” or “ActRII inhibitors”. Accordingly, the disclosure provides, in part, ActRII antagonist compositions and methods for treating pulmonary hypertension (e.g., PAH), particularly treating one or more complications of pulmonary hypertension (e.g., elevated blood pressure, cardiac hypertrophy, vascular remodeling, and muscularization of vessels). ActRII antagonists to be used in accordance with the methods and uses of the disclosure include, for example, ligand traps (e.g., soluble ActRIIA polypeptides, ActRIIB polypeptides, ALK4 polypeptides, ALK4:ActRIIB heteromultimer polypeptides, and ALK4:ActRIIA heteromultimer polypeptides). Optionally, ActRII antagonists may be used in combination with one or more supportive therapies and/or additional active agents for treating pulmonary hypertension.

In certain aspects, the present disclosure relates to methods of treating pulmonary hypertension, comprising administering to a patient in need thereof an effective amount of an ActRIIA variant polypeptide. In certain aspects, the present disclosure relates to methods of treating, preventing, or reducing the progression rate and/or severity of one or more complications of pulmonary hypertension, comprising administering to a patient in need thereof an effective amount of an ActRIIA variant polypeptide. In certain aspects, the present disclosure relates to methods of treating pulmonary hypertension, comprising administering to a patient in need thereof an effective amount of an ActRIIB variant polypeptide. In certain aspects, the present disclosure relates to methods of treating, preventing, or reducing the progression rate and/or severity of one or more complications of pulmonary hypertension, comprising administering to a patient in need thereof an effective amount of an ActRIIB variant polypeptide. In some embodiments, the one or more complications of pulmonary hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis. In some embodiments, the pulmonary hypertension is pulmonary arterial hypertension.

In certain aspects, the present disclosure relates to methods of treating, preventing, or reducing the progression rate and/or severity of one or more complications of an interstitial lung disease, comprising administering to a patient in need thereof an effective amount of an ActRIIA variant polypeptide. In certain aspects, the present disclosure relates to methods of treating, preventing, or reducing the progression rate and/or severity of one or more complications of an interstitial lung disease, comprising administering to a patient in need thereof an effective amount of an ActRIIB variant polypeptide. In some embodiments, the interstitial lung disease is idiopathic pulmonary fibrosis. In some embodiments, the ActRIIA variant polypeptide comprises the sequence of GAILGRSETQECLX₁X₂NANWX₃X₄X₅X₆TNQTGVEX₇CX₈GX₉X₁₀X₁₁X₁₂X₁₃X₁₄HCX₁₅A TWX₁₆NISGSIEIVX₁₇X₁₈GCX₁₉X₂₀X₂₁DX₂₂NCYDRTDCVEX₂₃X₂₄X₂₅X₂₆PX₂₇VYFCCCE GNMCNEKFSYFPEMEVTQPTS (SEQ ID NO: 139), wherein X₁ is F or Y; X₂ is F or Y; X₃ is E or A; X₄ is K or L; X₅ is D or E; X₆ is R or A; X₇ is P or R; X₅ is Y or E; X₉ is D or E; X₁₀ is K or Q; X₁₁ is D or A; X₁₂ is K or A; X₁₃ is R or A; X₁₄ is R or L; X₁₅ is F or Y; X₁₆ is K, R, or A; X₁₇ is K, A, Y, F, or I; X₁₈ is Q or K; X₁₉ is W or A; X₂₀ is L or A; X₂₁ is D, K, R, A, F, G, M, N, or I; X₂₂ is I, F, or A; X₂₃ is K or T; X₂₄ is K or E; X₂₅ is D or E; X₂₆ is S or N; and X₂₇ is E or Q, and wherein the ActRIIA variant polypeptide has at least one amino acid substitution relative to a wild-type extracellular ActRIIA having the sequence of SEQ ID NO: 211 or an extracellular ActRIIA having any one of the sequences of SEQ ID NOs: 212-232. In some embodiments, the ActRIIA variant polypeptide has a sequence of GAILGRSETQECLFX₂NANWX₃X₄X₅X₆TNQTGVEX₇CX₈GX₉KX₁₁X₁₂X₁₃X₁₄HCX₁₅AT WX₁₆NISGSIEIVX₁₇X₁₈GCX₁₉X₂₀X₂₁DX₂₂NCYDRTDCVEX₂₃X₂₄X₂₅X₂₆PX₂₇VYFCCCEG NMCNEKFSYFPEMEVTQPTS (SEQ ID NO: 140). In some embodiments, the ActRIIA variant polypeptide has a sequence of GAILGRSETQECLFX₂NANWEX₄X₅RTNQTGVEX₇CX₈GX₉KDKRX₁₄HCX₁₅ATWX₁₆NI SGSIEIVKX₁₈GCWLDDX₂₂NCYDRTDCVEX₂₃X₂₄X₂₅X₂₆PX₂₇VYFCCCEGNMCNEKFSY FPEMEVTQPTS (SEQ ID NO: 141). In some embodiments, the ActRIIA variant polypeptide has a sequence of GAILGRSETQECLFX₂NANWEX₄DRTNQTGVEX₇CX₈GX₉KDKRX₁₄HCX₁₅ATWX₁₆NIS GSIEIVKX₁₈GCWLDDX₂₂NCYDRTDCVEX₂₃KX₂₅X₂₆PX₂₇VYFCCCEGNMCNEKFSYFP EMEVTQPTS (SEQ ID NO: 142). In some embodiments, the ActRIIA variant polypeptide has a sequence of GAILGRSETQECLFX₂NANWEX₄DRTNQTGVEPCX₈GX₉KDKRX₁₄HCFATWKNISGSI EIVKX₁₈GCWLDDINCYDRTDCVEX₂₃KX₂₅X₂₆PX₂₇VYFCCCEGNMCNEKFSYFPEMEV TQPTS (SEQ ID NO: 143). In some embodiments, X₁ is F. In some embodiments, X₁ is Y. In some embodiments, X₁₀ is K. In some embodiments, X₁₀ is Q. In some embodiments, X₂ is F. In some embodiments, X₂ is Y. In some embodiments, X₃ is E. In some embodiments, X₃ is A. In some embodiments, X₄ is K. In some embodiments, X₄ is L. In some embodiments, X₅ is D. In some embodiments, X₅ is E. In some embodiments, X₆ is R. In some embodiments, X₆ is A. In some embodiments, X₇ is P. In some embodiments, X₇ is R. In some embodiments, X₈ is Y. In some embodiments, X₈ is E. In some embodiments, X₉ is D. In some embodiments, X₉ is E. In some embodiments, X₁₁ is D. In some embodiments, X₁₁ is A. In some embodiments, X₁₂ is K. In some embodiments, X₁₂ is A. In some embodiments, X₁₃ is R. In some embodiments, X₁₃ is A. In some embodiments, X₁₄ is R. In some embodiments, X₁₄ is L. In some embodiments, X₁₅ is F. In some embodiments, X₁₅ is Y. In some embodiments, X₁₆ is K. In some embodiments, X₁₆ is R. In some embodiments, X₁₆ is A. In some embodiments, X₁₇ is K. In some embodiments, X₁₇ is A. In some embodiments, X₁₇ is Y. In some embodiments, X₁₇ is F. In some embodiments, X₁₇ is I. In some embodiments, X₁₈ is Q. In some embodiments, X₁₈ is K. In some embodiments, X₁₉ is W. In some embodiments, X₁₉ is A. In some embodiments, X₂₀ is L. In some embodiments, X₂₀ is A. In some embodiments, X₂₁ is D. In some embodiments, X₂₁ is K. In some embodiments, X₂₁ is R. In some embodiments, X₂₁ is A. In some embodiments, X₂₁ is F. In some embodiments, X₂₁ is G. In some embodiments, X₂₁ is M. In some embodiments, X₂₁ is N. In some embodiments, X₂₁ is I. In some embodiments, X₂₂ is I. In some embodiments, X₂₂ is F. In some embodiments, X₂₂ is A. In some embodiments, X₂₃ is K. In some embodiments, X₂₃ is T. In some embodiments, X₂₄ is K. In some embodiments, X₂₄ is E. In some embodiments, X₂₅ is D. In some embodiments, X₂₅ is E. In some embodiments, X₂₆ is S. In some embodiments, X₂₆ is N. In some embodiments, X₂₇ is E. In some embodiments, X₂₇ is Q. In some embodiments, X₂₃ is T, X₂₄ is E, X₂₅ is E, and X₂₆ is N. In some embodiments, X₂₃ is T, X₂₄ is E, X₂₅ is E, and X₂₆ is N. In some embodiments, X₁₇ is K. In some embodiments, the ActRIIA variant polypeptide has the sequence of any one of SEQ ID NOs: 145-210. In some embodiments, the amino acid at position X₂₄ is replaced with the amino acid K. In some embodiments, the amino acid at position X₂₄ is replaced with the amino acid E. In some embodiments, the ActRIIA variant polypeptide further comprises a C-terminal extension of one or more amino acids. In some embodiments, the C-terminal extension is NP. In some embodiments, the C-terminal extension is NPVTPK.

In some embodiments, the ActRIIB variant polypeptide comprises the amino acid sequence of SEQ ID NO: 303, wherein at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 303 is substituted with another amino acid, and wherein said ActRIIB variant polypeptide is capable of binding myostatin and activin A, but demonstrates a decreased binding affinity for BMP9 relative to a wild-type ActRIIB polypeptide. In some embodiments, the at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 303 is substituted with the amino acid at the corresponding position of SEQ ID NO: 304, and wherein the ActRIIB variant polypeptide is capable of binding myostatin and activin A, but demonstrates a decreased binding affinity for BMP9 relative to a wild-type ActRIIB polypeptide. In some embodiments, the ActRIIB variant polypeptide comprises the amino acid sequence selected from the group consisting of: SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 138, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338 and SEQ ID NO: 339. In some embodiments, the ActRIIB variant polypeptide comprises the amino acid sequence selected from the group consisting of: SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, and SEQ ID NO: 406. In some embodiments, the ActRIIB variant polypeptide comprises an amino acid sequence selected from the group consisting of SEQ ID NO: 318 and SEQ ID NO: 331. In some embodiments, the ActRIIB variant polypeptide is selected from the group consisting of: a polypeptide comprising an amino acid sequence that is at least 90% identical to SEQ ID NO: 318; a polypeptide comprising an amino acid sequence that is at least 95% identical to SEQ ID NO: 318; a polypeptide comprising an amino acid sequence that is at least 99% identical to SEQ ID NO: 318; and a polypeptide comprising the amino acid sequence of SEQ ID NO: 318. In some embodiments, the ActRIIB variant polypeptide is selected from the group consisting of: a polypeptide comprising an amino acid sequence that is at least 90% identical to SEQ ID NO: 331; a polypeptide comprising an amino acid sequence that is at least 95% identical to SEQ ID NO: 331; a polypeptide comprising an amino acid sequence that is at least 99% identical to SEQ ID NO: 331; and a polypeptide comprising the amino acid sequence of SEQ ID NO: 331. In some embodiments, the patient is administered an additional active agent and/or supportive therapy for treating pulmonary hypertension. In some embodiments, the additional active agent and/or supportive therapy for treating pulmonary hypertension is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-β-O-D-glucopyranosyl-oleanolic acid; 3-β-O-D-glucopyranosyl (1→3)-β-D-glucopyranosiduronic acid (CS1); oleanolic acid 3-β-O-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); lung and/or heart transplantation. In some embodiments, the patient has resting pulmonary arterial pressure (PAP) of at least 25 mm Hg (e.g., 25, 30, 35, 40, 45, or 50 mm Hg). In some embodiments, the method reduces PAP in the patient. In some embodiments, the method reduces PAP by at least 3 mmHg (e.g., at least 3, 5, 7, 10, 12, 15, 20, or 25 mm Hg) in the patient. In some embodiments, the method reduces pulmonary vascular resistance in the patient. In some embodiments, the method increases pulmonary capillary wedge pressure. In some embodiments, the method increases left ventricular end-diastolic pressure. In some embodiments, the method increases exercise capacity of the patient. In some embodiments, the method increases the patient's 6-minute walk distance. In some embodiments, the method increases the patient's 6-minute walk distance by at least 10 meters (e.g., at least 10, 20, 30, 40, 50, 60, 70, 80, 90, 100 or more meters). In some embodiments, the method reduces the patient's Borg dyspnea index (BDI). In some embodiments, the method reduces the patient's BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points). In some embodiments, the patient has Functional Class I, Class II, Class III, or Class IV pulmonary hypertension as recognized by the World Health Organization. In some embodiments, the method prevents or delays pulmonary hypertension Functional Class progression (e.g., prevents or delays progression from Functional Class I to Class II, Class II to Class III, or Class III to Class IV pulmonary hypertension as recognized by the World Health Organization). In some embodiments, the method promotes or increases pulmonary hypertension Functional Class regression (e.g., promotes or increases regression from Class IV to Class III, Class III to Class II, or Class II to Class I pulmonary hypertension as recognized by the World Health Organization). In some embodiments, the ActRIIB variant polypeptide is part of a homodimer protein complex. In some embodiments, the ActRIIB variant polypeptide is part of a heteromultimer protein complex.

In some embodiments, the heteromultimer protein complex comprises an ALK4 polypeptide and an ActRIIB polypeptide. In some embodiments, the ALK4 polypeptide comprises a polypeptide selected from: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids of 24, 25, 26, 27, 28, 29, 30, 31, 32, 33, or 34 SEQ ID NO: 100, and ends at any one of amino acids 101, 102, 103, 104, 105, 106, 107, 108, 109, 110, 111, 112, 113, 114, 115, 116, 117, 118, 119, 120, 121, 122, 123, 124, 125, or 126 of SEQ ID NO: 100; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 34-101 of SEQ ID NO: 100; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 24-126 of SEQ ID NO: 100; d. a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 101; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 105. In some embodiments, the ActRIIB polypeptide comprises a polypeptide selected from: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids of 20, 21, 22, 23, 24, 25, 26, 27, 28, or 29 SEQ ID NO: 1, and ends at any one of amino acids 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 29-109 of SEQ ID NO: 1; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 25-131 of SEQ ID NO: 1; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 2; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 5; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 6; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOs: 4, 50-60, 69, 74, 138, 282, 289-303, and 305-407. In some embodiments, the ActRIIB polypeptide does not comprise an acidic amino acid at the position corresponding to L79 of SEQ ID NO: 1. In some embodiments, the ALK4 polypeptide is a fusion protein further comprising a heterologous domain that comprises a first or second member of an interaction pair. In some embodiments, the ActRIIB polypeptide is a fusion protein further comprising a heterologous domain that comprises a first or second member of an interaction pair. In some embodiments, the heterologous domain is an Fc immunoglobulin domain. In some embodiments, the ALK4 polypeptide and/or ActRIIB polypeptide comprise one or more amino acid modifications that promote heteromultimer formation. In some embodiments, the ALK4 and/or ActRIIB fusion protein further comprises a linker domain positioned between the ALK4 and/or ActRIIB domain and the heterologous domain. In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ALK4 fusion protein comprises a polypeptide selected from selected from the group consisting of: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 111; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 113; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 116; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 117; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 122; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 124. In some embodiments, the ActRIIB fusion protein comprises a polypeptide selected from selected from the group consisting of: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 108; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 110; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 114; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 115; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 118; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 120. In some embodiments, the ALK4 and/or ActRIIB polypeptide or fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety. In some embodiments, the ALK4 and/or ActRIIB polypeptide or fusion protein is glycosylated and has a mammalian glycosylation pattern. In some embodiments, the ALK4 and/or ActRIIB polypeptide or fusion protein has a glycosylation pattern obtainable from a Chinese hamster ovary cell line. In some embodiments, the heteromultimer binds to one or more ligands selected from the group consisting of: activin A, activin B, GDF11, GDF8, and BMP6. In some embodiments, the heteromultimer binds to activin A. In some embodiments, the heteromultimer inhibits one or more TGFβ superfamily ligands selected from the group consisting of: activin A, activin B, GDF11, GDF8, and BMP6. In some embodiments, the heteromultimer inhibits activin A. In some embodiments, the heteromultimer does not bind or does not substantially bind to one or more ligands selected from the group consisting of: BMP10, BMP9, and GDF3. In some embodiments, the heteromultimer binds to one or more of BMP10, BMP9, or GDF3 with lower affinity compared to a corresponding ActRIIB homomultimer. In some embodiments, the heteromultimer is in a pharmaceutical preparation. In some embodiments, the pharmaceutical preparation comprises less than about 10%, 9%, 8%, 7%, 6%, 5%, 4%, 3%, 2%, or less than about 1% ALK4 homomultimers. In some embodiments, the pharmaceutical preparation comprises less than about 10%, 9%, 8%, 7%, 6%, 5%, 4%, 3%, 2%, or less than about 1% ActRIIB homomultimers. In some embodiments, the heteromultimer is an ALK4:ActRIIB heterodimer. In some embodiments, the ActRIIA variant polypeptide is part of a homodimer protein complex. In some embodiments, the ActRIIA variant polypeptide is part of a heteromultimer protein complex.

In some embodiments, the heteromultimer protein complex comprises an ALK4 polypeptide and an ActRIIA polypeptide. In some embodiments, the ALK4 polypeptide comprises a polypeptide selected from: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids of 24, 25, 26, 27, 28, 29, 30, 31, 32, 33, or 34 SEQ ID NO: 100, and ends at any one of amino acids 101, 102, 103, 104, 105, 106, 107, 108, 109, 110, 111, 112, 113, 114, 115, 116, 117, 118, 119, 120, 121, 122, 123, 124, 125, or 126 of SEQ ID NO: 100; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 34-101 of SEQ ID NO: 100; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 24-126 of SEQ ID NO: 100; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 101; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 105. In some embodiments, the ActRIIA polypeptide comprises a polypeptide selected from: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical SEQ ID NO: 10; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical SEQ ID NO: 11; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 21-135 of SEQ ID NO: 9; a polypeptide comprising an amino acid sequence that begins at any one of amino acids 21, 22, 23, 24, 25, 26, 27, 28, 29, or 30 SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOs: 9-11, 32, 36, 39, 61-68, 93, 95, 96, 139-211, 283, 304, and 408-409. In some embodiments, the ALK4 polypeptide is a fusion protein further comprising a heterologous domain that comprises a first or second member of an interaction pair. In some embodiments, the ActRIIA polypeptide is a fusion protein further comprising a heterologous domain that comprises a first or second member of an interaction pair. In some embodiments, the heterologous domain is an Fc immunoglobulin domain. In some embodiments, the ALK4 polypeptide and/or ActRIIA polypeptide comprise one or more amino acid modifications that promote heteromultimer formation. In some embodiments, the ALK4 and/or ActRIIA fusion protein further comprises a linker domain positioned between the ALK4 and/or ActRIIA domain and the heterologous domain. In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ALK4 fusion protein comprises a polypeptide selected from selected from the group consisting of: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 111; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 113; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 116; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 117; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 122; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 124. In some embodiments, the ActRIIA fusion protein comprises a polypeptide selected from selected from the group consisting of: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 32; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 36; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 39; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 93; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 95; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 96. In some embodiments, the ALK4 and/or ActRIIA polypeptide or fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety. In some embodiments, the ALK4 and/or ActRIIA polypeptide or fusion protein is glycosylated and has a mammalian glycosylation pattern. In some embodiments, the ALK4 and/or ActRIIA polypeptide or fusion protein has a glycosylation pattern obtainable from a Chinese hamster ovary cell line. In some embodiments, the heteromultimer binds to one or more ligands selected from the group consisting of: activin A, activin B, GDF11, GDF8, and BMP6. In some embodiments, the heteromultimer binds to activin A. In some embodiments, the heteromultimer inhibits one or more TGFβ superfamily ligands selected from the group consisting of: activin A, activin B, GDF11, GDF8, and BMP6. In some embodiments, the heteromultimer inhibits activin A. In some embodiments, the heteromultimer does not bind or does not substantially bind to one or more ligands selected from the group consisting of: BMP10, BMP9, and GDF3. In some embodiments, the heteromultimer binds to one or more of BMP10, BMP9, or GDF3 with lower affinity compared to a corresponding ActRIIA homomultimer. In some embodiments, the heteromultimer is in a pharmaceutical preparation. In some embodiments, the pharmaceutical preparation comprises less than about 10%, 9%, 8%, 7%, 6%, 5%, 4%, 3%, 2%, or less than about 1% ALK4 homomultimers. In some embodiments, the pharmaceutical preparation comprises less than about 10%, 9%, 8%, 7%, 6%, 5%, 4%, 3%, 2%, or less than about 1% ActRIIA homomultimers. In some embodiments, the heteromultimer is an ALK4:ActRIIA heterodimer. In some embodiments, the ActRIIA polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 9 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 9. In some embodiments, the ActRIIA polypeptide is selected from the group consisting of: a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of amino acids corresponding to residues 30-110 of SEQ ID NO: 9; a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 10; and a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 11. In some embodiments, the polypeptide is a fusion protein further comprising an Fc domain of an immunoglobulin. In some embodiments, the Fc domain of the immunoglobulin is an Fc domain of an IgG1 immunoglobulin. In some embodiments, the Fc fusion protein further comprises a linker domain positioned between the ActRIIA polypeptide domain and the Fc domain of the immunoglobulin. In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 32. In some embodiments, the polypeptide is part of a homodimer protein complex. In some embodiments, the polypeptide is glycosylated. In some embodiments, the polypeptide has a glycosylation pattern obtainable by expression in a Chinese hamster ovary cell.

In some embodiments, the method decreases pulmonary arterial pressure in the patient. In some embodiments, the method decreases pulmonary arterial pressure in the patient by at least 10% (e.g., 10%, 15%, 20%, 25%, 30%, 35%, 40%, 45%, 50%, 55%, 60%, 65%, 70%, 75%, or at least 80%). In some embodiments, the method decreases ventricle hypertrophy in the patient. In some embodiments, the method decreases ventricle hypertrophy in the patient by at least 10% (e.g., 10%, 15%, 20%, 25%, 30%, 35%, 40%, 45%, 50%, 55%, 60%, 65%, 70%, 75%, or at least 80%). In some embodiments, the method decreases smooth muscle hypertrophy in the patient. In some embodiments, the method decreases smooth muscle hypertrophy in the patient by at least 10% (e.g., 10%, 15%, 20%, 25%, 30%, 35%, 40%, 4%5% 50%, 55%, 60, 65%, 70%, 75% or at least 80%). In some embodiments, the method decreases pulmonary arteriole muscularity in the patient. In some embodiments, the method decreases pulmonary arteriole muscularity in the patient by at least 10% (e.g., 10%, 15%, 20%, 25%, 30%, 35%, 40%, 45%, 50%, 55%, 60%, 65%, 70%, 75%, or at least 80%). In some embodiments, the method decreases pulmonary vascular resistance in the patient. In some embodiments, the method decreases pulmonary vascular resistance in the patient by at least 10% (e.g., 10%, 15%, 20%, 25%, 30%, 35%, 40%, 45%, 50%, 55%, 60%, 65%, 70%, 75%, or at least 80%). In some embodiments, the patient has pulmonary arterial hypertension and has Functional Class II or Class III pulmonary hypertension in accordance with the World Health Organization's functional classification system for pulmonary hypertension. In some embodiments, the patient has pulmonary arterial hypertension that is classified as one or more subtypes selected from the group consisting of: idiopathic or heritable pulmonary arterial hypertension, drug- and/or toxin-induced pulmonary hypertension, pulmonary hypertension associated with connective tissue disease, and pulmonary hypertension associated with congenital systemic-to-pulmonary shunts at least 1 year following shunt repair. In some embodiments, the patient has been treated with one or more vasodilators. In some embodiments, the patient has been treated with one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, the method further comprises administration of one or more vasodilators. In some embodiments, the method further comprises the administration of one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, the patient has a 6-minute walk distance from 150 to 400 meters. In some embodiments, the method increases the patient's 6-minute walk distance by at least 10 meters (e.g., at least 10, 20, 30, 40, 50, 60, 70, 80, 90, 100, 125, 150, 175, 200, 250, 300, or more than 400 meters). In some embodiments, the patient has a hemoglobin level from >8 and <15 g/dl. In some embodiments, the method delays clinical worsening of pulmonary arterial hypertension. In some embodiments, the method delays clinical worsening of pulmonary hypertension in accordance with the World Health Organization's functional classification system for pulmonary hypertension. In some embodiments, the method reduces the risk of hospitalization for one or more complications associated with pulmonary arterial hypertension. In some embodiments, the ActRIIA polypeptide binds to one or more ligands selected from the group consisting of: activin A, activin B, and GDF11. In some embodiments, the ActRIIA polypeptide further binds to one or more ligands selected from the group consisting of: BMP10, GDF8, and BMP6.

### BRIEF DESCRIPTION OF THE DRAWINGS

FIG. 1 shows an alignment of extracellular domains of human ActRIIB (SEQ ID NO: 2) and human ActRIIA (SEQ ID NO: 10) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIB proteins (SEQ ID NOs: 53-58) and human ActRIIA (SEQ ID NO: 59) as well as a consensus ActRII sequence derived from the alignment (SEQ ID NO: 60).

FIG. 3 shows a multiple sequence alignment of various vertebrate ActRIIA proteins and human ActRIIA (SEQ ID NOs: 61-68).

FIG. 4 shows multiple sequence alignment of Fc domains from human IgG isotypes using Clustal 2.1. Hinge regions are indicated by dotted underline. Double underline indicates examples of positions engineered in IgG1 (SEQ ID NO: 133) Fc to promote asymmetric chain pairing and the corresponding positions with respect to other isotypes IgG2 (SEQ ID NO: 135), IgG3 (SEQ ID NO: 136) and IgG4 (SEQ ID NO: 134).

FIG. 5 shows the purification of ActRIIA-hFc expressed in CHO cells. The protein purifies as a single, well-defined peak as visualized by sizing column (top panel) and Coomassie stained SDS-PAGE (bottom panel) (left lane: molecular weight standards; right lane: ActRIIA-hFc).

FIG. 6 shows the binding of ActRIIA-hFc to activin (top panel) and GDF-11 (bottom panel), as measured by Biacore™ assay.

FIG. 7 shows the full, unprocessed amino acid sequence for ActRIIB(25-131)-hFc (SEQ ID NO: 69). The TPA leader (residues 1-22) and double-truncated ActRIIB extracellular domain (residues 24-131, using numbering based on the native sequence in SEQ ID NO: 1) are each underlined. Boxed is the glutamate revealed by sequencing to be the N-terminal amino acid of the mature fusion protein, which is at position 25 relative to SEQ ID NO: 1.

FIGS. 8A and 8B show a nucleotide sequence encoding ActRIIB(25-131)-hFc (the coding strand is shown at top, SEQ ID NO: 70, and the complement shown at bottom 3′-5′, SEQ ID NO: 71). Sequences encoding the TPA leader (nucleotides 1-66) and ActRIIB extracellular domain (nucleotides 73-396) are underlined. The corresponding amino acid sequence for ActRIIB(25-131) (SEQ ID NO: 138) is also shown.

FIGS. 9A and 9B show an alternative nucleotide sequence encoding ActRIIB(25-131)-hFc (the coding strand is shown at top, SEQ ID NO: 72, and the complement shown at bottom 3′-5′, SEQ ID NO: 73). This sequence confers a greater level of protein expression in initial transformants, making cell line development a more rapid process. Sequences encoding the TPA leader (nucleotides 1-66) and ActRIIB extracellular domain (nucleotides 73-396) are underlined, and substitutions in the wild type nucleotide sequence of the ECD (see FIG. 8) are boxed. The corresponding amino acid sequence for ActRIIB(25-131) (SEQ ID NO: 138) is also shown.

FIG. 10 shows the full amino acid sequence for the ActRIIB(L79D 20-134)-hFc (SEQ ID NO: 74), including the TPA leader sequence (double underline, ActRIIB extracellular domain (residues 20-134 in SEQ ID NO: 1; single underline), and hFc domain. The aspartate substituted at position 79 in the native sequence is double underlined and boxed, as is the glycine revealed by sequencing to be the N-terminal residue in the mature fusion protein.

FIGS. 11A and 11B show a nucleotide sequence encoding ActRIIB(L79D 20-134)-hFc. SEQ ID NO: 75 corresponds to the sense strand, and SEQ ID NO: 76 corresponds to the antisense strand. The TPA leader (nucleotides 1-66) is double underlined, and the ActRIIB extracellular domain (nucleotides 76-420) is single underlined.

FIG. 12 shows the full amino acid sequence for the truncated ActRIIB(L79D 25-131)-hFc (SEQ ID NO: 77), including the TPA leader (double underline, truncated ActRIIB extracellular domain (residues 25-131 in SEQ ID NO:1; single underline), and hFc domain. The aspartate substituted at position 79 in the native sequence is double underlined and boxed, as is the glutamate revealed by sequencing to be the N-terminal residue in the mature fusion protein.

FIG. 13 shows the amino acid sequence for the truncated ActRIIB(L79D 25-131)-hFc without a leader (SEQ ID NO: 78). The truncated ActRIIB extracellular domain (residues 25-131 in SEQ ID NO: 1) is underlined. The aspartate substituted at position 79 in the native sequence is double underlined and boxed, as is the glutamate revealed by sequencing to be the N-terminal residue in the mature fusion protein.

FIG. 14 shows the amino acid sequence for the truncated ActRIIB(L79D 25-131) without the leader, hFc domain, and linker (SEQ ID NO: 79). The aspartate substituted at position 79 in the native sequence is underlined and boxed, as is the glutamate revealed by sequencing to be the N-terminal residue in the mature fusion protein.

FIGS. 15A and 15B show a nucleotide sequence encoding ActRIIB(L79D 25-131)-hFc. SEQ ID NO: 80 corresponds to the sense strand, and SEQ ID NO: 81 corresponds to the antisense strand. The TPA leader (nucleotides 1-66) is double underlined, and the truncated ActRIIB extracellular domain (nucleotides 76-396) is single underlined. The amino acid sequence for the ActRIIB extracellular domain (SEQ ID NO: 79) is also shown.

FIGS. 16A and 16B show an alternative nucleotide sequence encoding ActRIIB(L79D 25-131)-hFc. SEQ ID NO: 82 corresponds to the sense strand, and SEQ ID NO: 83 corresponds to the antisense strand. The TPA leader (nucleotides 1-66) is double underlined, the truncated ActRIIB extracellular domain (nucleotides 76-396) is underlined, and substitutions in the wild-type nucleotide sequence of the extracellular domain are double underlined and boxed (compare with SEQ ID NO: 81, FIG. 15). The amino acid sequence for the ActRIIB extracellular domain (SEQ ID NO: 79) is also shown.

FIG. 17 shows nucleotides 76-396 (SEQ ID NO: 84) of the alternative nucleotide sequence shown in FIG. 16 (SEQ ID NO: 82). The same nucleotide substitutions indicated in FIG. 16 are also underlined and boxed here. SEQ ID NO: 84 encodes only the truncated ActRIIB extracellular domain (corresponding to residues 25-131 in SEQ ID NO: 1) with a L79D substitution, e.g., ActRIIB(L79D 25-131).

FIG. 18 shows a multiple sequence alignment of various vertebrate ALK4 proteins and human ALK4 (SEQ ID NOs: 126-132).

FIG. 19 shows comparative ligand binding data for an ALK4-Fc:ActRIIB-Fc heterodimeric protein complex compared to ActRIIB-Fc homodimer and ALK4-Fc homodimer. For each protein complex, ligands are ranked by k_{off}, a kinetic constant that correlates well with ligand signaling inhibition, and listed in descending order of binding affinity (ligands bound most tightly are listed at the top). At left, yellow, red, green, and blue lines indicate magnitude of the off-rate constant. Solid black lines indicate ligands whose binding to heterodimer is enhanced or unchanged compared with homodimer, whereas dashed red lines indicate substantially reduced binding compared with homodimer. As shown, the ALK4-Fc:ActRIIB-Fc heterodimer displays enhanced binding to activin B compared with either homodimer, retains strong binding to activin A, GDF8, and GDF11 as observed with ActRIIB-Fc homodimer, and exhibits substantially reduced binding to BMP9, BMP10, and GDF3. Like ActRIIB-Fc homodimer, the heterodimer retains intermediate-level binding to BMP6.

FIG. 20 shows comparative ALK4-Fc:ActRIIB-Fc heterodimer/ActRIIB-Fc:ActRIIB-Fc homodimer IC₅₀ data as determined by an A-204 Reporter Gene Assay as described herein. ALK4-Fc:ActRIIB-Fc heterodimer inhibits activin A, activin B, GDF8, and GDF11 signaling pathways similarly to the ActRIIB-Fc:ActRIIB-Fc homodimer. However, ALK4-Fc:ActRIIB-Fc heterodimer inhibition of BMP9 and BMP10 signaling pathways is significantly reduced compared to the ActRIIB-Fc:ActRIIB-Fc homodimer. These data demonstrate that ALK4:ActRIIB heterodimers are more selective antagonists of activin A, activin B, GDF8, and GDF11 compared to corresponding ActRIIB:ActRIIB homodimers.

FIGS. 21A and 21B show two schematic examples of heteromeric protein complexes comprising type I receptor and type II receptor polypeptides. FIG. 21A depicts a heterodimeric protein complex comprising one type I receptor fusion polypeptide and one type II receptor fusion polypeptide, which can be assembled covalently or noncovalently via a multimerization domain contained within each polypeptide chain. Two assembled multimerization domains constitute an interaction pair, which can be either guided or unguided. FIG. 21B depicts a heterotetrameric protein complex comprising two heterodimeric complexes as depicted in FIG. 21A. Complexes of higher order can be envisioned.

FIG. 22 shows a schematic example of a heteromeric protein complex comprising a type I receptor polypeptide (indicated as “I”) (e.g. a polypeptide that is at least 70%, 75%, 80%, 85%, 90%, 91%, 92%, 93%, 94%, 95%, 97%, 98%, 99% or 100% identical to an extracellular domain of an ALK4 protein from humans or other species such as those described herein) and a type II receptor polypeptide (indicated as “II”) (e.g. a polypeptide that is at least 70%, 75%, 80%, 85%, 90%, 91%, 92%, 93%, 94%, 95%, 97%, 98%, 99% or 100% identical to an extracellular domain of an ActRIIB protein from humans or other species as such as those described herein). In the illustrated embodiments, the type I receptor polypeptide is part of a fusion polypeptide that comprises a first member of an interaction pair (“C₁”), and the type II receptor polypeptide is part of a fusion polypeptide that comprises a second member of an interaction pair (“C₂”). In each fusion polypeptide, a linker may be positioned between the type I or type II receptor polypeptide and the corresponding member of the interaction pair. The first and second members of the interaction pair may be a guided (asymmetric) pair, meaning that the members of the pair associate preferentially with each other rather than self-associate, or the interaction pair may be unguided, meaning that the members of the pair may associate with each other or self-associate without substantial preference and may have the same or different amino acid sequences. Traditional Fc fusion proteins and antibodies are examples of unguided interaction pairs, whereas a variety of engineered Fc domains have been designed as guided (asymmetric) interaction pairs [e.g., Spiess et al (2015) Molecular Immunology 67(2A): 95-106].

FIGS. 23A-23D show schematic examples of heteromeric protein complexes comprising an ALK4 polypeptide (e.g. a polypeptide that is at least 70%, 75%, 80%, 85%, 90%, 91%, 92%, 93%, 94%, 95%, 97%, 98%, 99% or 100% identical to an extracellular domain of an ALK4 protein from humans or other species such as those described herein) and an ActRIIB polypeptide (e.g. a polypeptide that is at least 70%, 75%, 80%, 85%, 90%, 91%, 92%, 93%, 94%, 95%, 97%, 98%, 99% or 100% identical to an extracellular domain of an ActRIIB protein from humans or other species such as those described herein). In the illustrated embodiments, the ALK4 polypeptide is part of a fusion polypeptide that comprises a first member of an interaction pair (“C₁”), and the ActRIIB polypeptide is part of a fusion polypeptide that comprises a second member of an interaction pair (“C₂”). Suitable interaction pairs included, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the ALK4 or ActRIIB polypeptide and the corresponding member of the interaction pair. The first and second members of the interaction pair may be unguided, meaning that the members of the pair may associate with each other or self-associate without substantial preference, and they may have the same or different amino acid sequences. See FIG. 23A. Alternatively, the interaction pair may be a guided (asymmetric) pair, meaning that the members of the pair associate preferentially with each other rather than self-associate. See FIG. 23B. Complexes of higher order can be envisioned. See FIGS. 23C and 23D.

FIG. 24 shows ligand binding data for an ActRIIA-Fc:ALK4-Fc heterodimeric protein complex as compared to ActRIIA-Fc homodimer and ALK4-Fc homodimer For each protein complex, ligands are ranked by k_{off}, a kinetic constant that correlates well with ligand signaling inhibition, and listed in descending order of binding affinity (ligands bound most tightly are listed at the top). At left, yellow, red, green, and blue lines indicate magnitude of the off-rate constant. Solid black lines indicate ligands whose binding to heterodimer is enhanced or unchanged compared with homodimer, whereas dashed red lines indicate substantially reduced binding compared with homodimer. As shown, the ActRIIA-Fc:ALK4-Fc heterodimer exhibits enhanced binding to activin A, and particularly enhanced binding to activin AC, compared to ActRIIA-Fc homodimer, while retaining strong binding to activin AB and GDF11. In addition, the ligand with highest affinity for ActRIIA-Fc homodimer, activin B, displays reduced affinity (albeit still within the high-affinity range) for the ActRIIA-Fc:ALK4-Fc heterodimer. The ActRIIA-Fc:ALK4-Fc heterodimer also exhibits markedly reduced binding to BMP10 compared to ActRIIA-Fc homodimer.

### DETAILED DESCRIPTION OF THE INVENTION

### 1. Overview

The TGF-β superfamily is comprised of over 30 secreted factors including TGF-betas, activins, nodals, bone morphogenetic proteins (BMPs), growth and differentiation factors (GDFs), and anti-Mullerian hormone (AMH) [Weiss et al. (2013) Developmental Biology, 2(1): 47-63]. Members of the superfamily, which are found in both vertebrates and invertebrates, are ubiquitously expressed in diverse tissues and function during the earliest stages of development throughout the lifetime of an animal. Indeed, TGF-β superfamily proteins are key mediators of stem cell self-renewal, gastrulation, differentiation, organ morphogenesis, and adult tissue homeostasis. Consistent with this ubiquitous activity, aberrant TGF-beta superfamily signaling is associated with a wide range of human pathologies including, for example, autoimmune disease, cardiovascular disease, fibrotic disease, and cancer.

Ligands of the TGF-beta superfamily share the same dimeric structure in which the central 3½ turn helix of one monomer packs against the concave surface formed by the beta-strands of the other monomer. The majority of TGF-beta family members are further stabilized by an intermolecular disulfide bond. This disulfide bonds traverses through a ring formed by two other disulfide bonds generating what has been termed a ‘cysteine knot’ motif [Lin et al. (2006) Reproduction 132: 179-190; and Hinck et al. (2012) FEBS Letters 586: 1860-1870].

TGF-beta superfamily signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation [Massague (2000) Nat. Rev. Mol. Cell Biol. 1:169-178]. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGF-beta family can be divided into two phylogenetic branches based on the type I receptors they bind and the Smad proteins they activate. One is the more recently evolved branch, which includes, e.g., the TGF-betas, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8.

Activins are members of the TGF-beta superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84; and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a β_{A}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the β_{B} subunit of the complex). Similarly, agents disclosed herein that antagonize (inhibit) “activin A” are agents that inhibit one or more activities as mediated by a β_{A} subunit, whether in the context of an isolated β_{A} subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB” are agents that inhibit one or more activities as mediated by the JA subunit and one or more activities as mediated by the OB subunit.

The BMPs and GDFs together form a family of cysteine-knot cytokines sharing the characteristic fold of the TGF-beta superfamily [Rider et al. (2010) Biochem J., 429(1):1-12]. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGF-beta superfamily ligands.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle [McPherron et al. Nature (1997) 387:83-90]. Similar increases in skeletal muscle mass are evident in naturally occurring mutations of GDF8 in cattle and, strikingly, in humans [Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461; Kambadur et al. Genome Res. (1997) 7:910-915; and Schuelke et al. (2004) N Engl J Med, 350:2682-8]. Studies have also shown that muscle wasting associated with HIV-infection in humans is accompanied by increases in GDF8 protein expression [Gonzalez-Cadavid et al., PNAS (1998) 95:14938-43]. In addition, GDF8 can modulate the production of muscle-specific enzymes (e.g., creatine kinase) and modulate myoblast cell proliferation [International Patent Application Publication No. WO 00/43781]. The GDF8 propeptide can noncovalently bind to the mature GDF8 domain dimer, inactivating its biological activity [Miyazono et al. (1988) J. Biol. Chem., 263: 6407-6415; Wakefield et al. (1988) J. Biol. Chem., 263; 7646-7654; and Brown et al. (1990) Growth Factors, 3: 35-43]. Other proteins which bind to GDF8 or structurally related proteins and inhibit their biological activity include follistatin, and potentially, follistatin-related proteins [Gamer et al. (1999) Dev. Biol., 208: 222-232].

GDF11, also known as BMP11, is a secreted protein that is expressed in the tail bud, limb bud, maxillary and mandibular arches, and dorsal root ganglia during mouse development [McPherron et al. (1999) Nat. Genet., 22: 260-264; and Nakashima et al. (1999) Mech. Dev., 80: 185-189]. GDF11 plays a unique role in patterning both mesodermal and neural tissues [Gamer et al. (1999) Dev Biol., 208:222-32]. GDF11 was shown to be a negative regulator of chondrogenesis and myogenesis in developing chick limb [Gamer et al. (2001) Dev Biol., 229:407-20]. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium [Wu et al. (2003) Neuron., 37:197-207]. Hence, GDF11 may have in vitro and in vivo applications in the treatment of diseases such as muscle diseases and neurodegenerative diseases (e.g., amyotrophic lateral sclerosis).

As demonstrated herein, a soluble ActRIIA polypeptide and ALK4:ActRIIB heterodimer, which both bind to various ActRIIA and ActRIIB-interacting ligands, is effective in decreasing blood pressure and cardiac hypertrophy in a PAH model. While not wishing to be bound to any particular mechanism, it is expected that the effects of these agents is caused primarily by an ActRII (ActRIIA and/or ActRIIB) signaling antagonist effect. Regardless of the mechanism, it is apparent from the data presented herein that ActRII antagonists decrease blood pressure, decrease cardiac hypertrophy, and have other positivity effects in treating pulmonary hypertension. It should be noted that blood pressure and hypertrophy are dynamic, with changes depending on a balance of factors that increase blood pressure and hypertrophy and factors that decrease blood pressure and hypertrophy. Blood pressure and cardiac hypertrophy can be decreased by increasing factors that reduce blood pressure and cardiac hypertrophy, decreasing factors that promote elevated blood pressure and cardiac hypertrophy, or both. The terms decreasing blood pressure or decreasing cardiac hypertrophy refer to the observable physical changes in blood pressure and cardiac tissue and are intended to be neutral as to the mechanism by which the changes occur.

The rat models for PAH that were used in the studies described herein are considered to be predicative of efficacy in humans, and therefore, this disclosure provides methods for using ActRIIA polypeptides, ActRIIB polypeptides, ALK4:ActRIIB heteromultimers, ALK4:ActRIIA heteromultimers, and other ActRII antagonists to treat pulmonary hypertension (e.g., PAH), particularly treating, preventing, or reducing the severity or duration of one or more complications of pulmonary hypertension, in humans. As disclosed herein, the term ActRII antagonists refers a variety of agents that may be used to antagonize ActRII signaling including, for example, antagonists that inhibit one or more TGF-beta family ligands [e.g., activin A, activin B, activin AB, activin AC, BMP6, BMP7, BMP9, BMP10, GDF3, GDF8, and GDF11]; antagonists that inhibit ActRIIA or ActRIIB; and antagonists that inhibit one or more downstream signaling components (e.g., Smad proteins). The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is +10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII Polypeptides, ALK4 Polypeptides, ALK4:ActRIIB Heteromultimers, ALK4:ActRIIA Heteromultimers, and Variants Thereof

In certain aspects, the disclosure relates ActRII polypeptides and uses thereof (e.g., of treating, preventing, or reducing the progression rate and/or severity of pulmonary hypertension or one or more complications of pulmonary hypertension), a kidney-associated disease (e.g. Alport syndrome, focal segmental glomerulosclerosis (FSGS), polycystic kidney disease, or chronic kidney disease), and/or an interstitial lung disease (e.g., idiopathic pulmonary fibrosis). As used herein, the term “ActRII” refers to the family of type II activin receptors. This family includes activin receptor type IIA (ActRIIA) and activin receptor type IIB (ActRIIB).

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627, WO 2008/097541, WO 2010/151426, WO 2011/020045, WO2019140283, WO2018/089706, WO2018/089715 WO2019/094751, WO2016/171948, and WO2018/075747 which are incorporated herein by reference in their entirety. Numbering of amino acids for all ActRIIB-related polypeptides described herein is based on the numbering of the human ActRIIB precursor protein sequence provided below (SEQ ID NO: 1), unless specifically designated otherwise.

The human ActRIIB precursor protein sequence is as follows:

(SEQ ID NO: 1)

| | |
|---|---|
| 1 | MTAPWVALAL LWGSLCAGSG RGEAETRECI YYNANWELER |
| | TNQSGLERCE |
| 51 | GEQDKRLHCY ASWRNSSGTI ELVKKGCWLD DFNCYDRQEC |
| | VATEENPQVY |
| 101 | FCCCEGNFCN ERFTHLPEAG GPEVTYEPPP TAPTLLTVLA |
| | YSLLPIGGLS |
| 151 | LIVLLAFWMY RHRKPPYGHV DIHEDPGPPP PSPLVGLKPL |
| | QLLEIKARGR |
| 201 | FGCVWKAQLM NDFVAVKIFP LQDKQSWQSE REIFSTPGMK |
| | HENLLQFIAA |
| 251 | EKRGSNLEVE LWLITAFHDK GSLTDYLKGN IITWNELCHV |
| | AETMSRGLSY |
| 301 | LHEDVPWCRG EGHKPSIAHR DFKSKNVLLK SDLTAVLADF |
| | GLAVRFEPGK |
| 351 | PPGDTHGQVG TRRYMAPEVL EGAINFQRDA FLRIDMYAMG |
| | LVLWELVSRC |
| 401 | KAADGPVDEY MLPFEEEIGQ HPSLEELQEV VVHKKMRPTI |
| | KDHWLKHPGL |
| 451 | AQLCVTIEEC WDHDAEARLS AGCVEERVSL IRRSVNGTTS |
| | DCLVSLVTSV |
| 501 | TNVDLPPKES SI |

The signal peptide is indicated with a single underline; the extracellular domain is indicated in bold font; and the potential, endogenous N-linked glycosylation sites are indicated with a double underline.

The processed (mature) extracellular ActRIIB polypeptide sequence is as follows:

(SEQ ID NO: 2)
GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPT.

In some embodiments, the protein may be produced with an “SGR . . . ” sequence at the N-terminus. The C-terminal “tail” of the extracellular domain is indicated by a single underline. The sequence with the “tail” deleted (a A15 sequence) is as follows:

(SEQ ID NO: 3)
GRGEAETRECIYYNANWELERTNQSGLERCEGEQDKRLHCYASWRNSSGT
IELVKKGCWLDDFNCYDRQECVATEENPQVYFCCCEGNFCNERFTHLPE
A.

A form of ActRIIB with an alanine at position 64 of SEQ ID NO: 1 (A64) is also reported in the literature. See, e.g., Hilden et al. (1994) Blood, 83(8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB with an alanine at position 64 is as follows:

(SEQ ID NO: 4)

| | |
|---|---|
| 1 | MTAPWVALAL LWGSLCAGSG RGEAETRECI YYNANWELER |
| | TNQSGLERCE |
| 51 | GEQDKRLHCY ASWANSSGTI ELVKKGCWLD DFNCYDRQEC |
| | VATEENPQVY |
| 101 | FCCCEGNFCN ERFTHLPEAG GPEVTYEPPP TAPTLLTVLA |
| | YSLLPIGGLS |
| 151 | LIVLLAFWMY RHRKPPYGHV DIHEDPGPPP PSPLVGLKPL |
| | QLLEIKARGR |
| 201 | FGCVWKAQLM NDFVAVKIFP LQDKQSWQSE REIFSTPGMK |
| | HENLLQFIAA |
| 251 | EKRGSNLEVE LWLITAFHDK GSLTDYLKGN IITWNELCHV |
| | AETMSRGLSY |
| 301 | LHEDVPWCRG EGHKPSIAHR DFKSKNVLLK SDLTAVLADF |
| | GLAVRFEPGK |
| 351 | PPGDTHGQVG TRRYMAPEVL EGAINFQRDA FLRIDMYAMG |
| | LVLWELVSRC |
| 401 | KAADGPVDEY MLPFEEEIGQ HPSLEELQEV VVHKKMRPTI |
| | KDHWLKHPGL |
| 451 | AQLCVTIEEC WDHDAEARLS AGCVEERVSL IRRSVNGTTS |
| | DCLVSLVTSV |
| 501 | TNVDLPPKES SI |

The signal peptide is indicated by single underline and the extracellular domain is indicated by bold font.

The processed (mature) extracellular ActRIIB polypeptide sequence of the alternative A64 form is as follows:

(SEQ ID NO: 5)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA
GGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a A15 sequence) is as follows:

(SEQ ID NO: 6)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA

A nucleic acid sequence encoding the human ActRIIB precursor protein is shown below (SEQ ID NO: 7), representing nucleotides 25-1560 of Genbank Reference Sequence NM_001106.3, which encode amino acids 1-513 of the ActRIIB precursor. The sequence as shown provides an arginine at position 64 and may be modified to provide an alanine instead. The signal sequence is underlined.

(SEQ ID NO: 7)

| | |
|---|---|
| 1 | ATGACGGCGC CCTGGGTGGC CCTCGCCCTC CTCTGGGGAT |
| | CGCTGTGCGC |
| 51 | CGGCTCTGGG CGTGGGGAGG CTGAGACACG GGAGTGCATC |
| | TACTACAACG |
| 101 | CCAACTGGGA GCTGGAGCGC ACCAACCAGA GCGGCCTGGA |
| | GCGCTGCGAA |
| 151 | GGCGAGCAGG ACAAGCGGCT GCACTGCTAC GCCTCCTGGC |
| | GCAACAGCTC |
| 201 | TGGCACCATC GAGCTCGTGA AGAAGGGCTG CTGGCTAGAT |
| | GACTTCAACT |
| 251 | GCTAGGATAG GCAGGAGTGT GTGGCCACTG AGGAGAACCC |
| | CCAGGTGTAC |
| 301 | TTCTGCTGCT GTGAAGGCAA CTTCTGCAAC GAACGCTTCA |
| | CTCATTTGCC |
| 351 | AGAGGCTGGG GGCCCGGAAG TCACGTACGA GCCACCCCCG |
| | ACAGCCCCCA |
| 401 | CCCTGCTCAC GGTGCTGGCC TACTCACTGC TGCCCATCGG |
| | GGGCCTTTCC |
| 451 | CTCATCGTCC TGCTGGCCTT TTGGATGTAC CGGCATCGCA |
| | AGCCCCCCTA |
| 501 | CGGTCATGTG GACATCCATG AGGACCCTGG GCCTCCACCA |
| | CCATCCCCTC |
| 551 | TGGTGGGCCT GAAGCCACTG CAGCTGCTGG AGATCAAGGC |
| | TCGGGGGCGC |
| 601 | TTTGGCTGTG TCTGGAAGGC CCAGCTCATG AATGACTTTG |
| | TAGCTGTCAA |
| 651 | GATCTTCCCA CTCCAGGACA AGCAGTCGTG GCAGAGTGAA |
| | CGGGAGATCT |
| 701 | TCAGCACACC TGGCATGAAG CACGAGAACC TGCTACAGTT |
| | CATTGCTGCC |
| 751 | GAGAAGCGAG GCTCCAACCT CGAAGTAGAG CTGTGGCTCA |
| | TCACGGCCTT |
| 801 | CCATGACAAG GGCTCCCTCA CGGATTACCT CAAGGGGAAC |
| | ATCATCACAT |
| 851 | GGAACGAACT GTGTCATGTA GCAGAGACGA TGTCACGAGG |
| | CCTCTCATAC |
| 901 | CTGCATGAGG ATGTGCCCTG GTGCCGTGGC GAGGGCCACA |
| | AGCCGTCTAT |
| 951 | TGCCCACAGG GACTTTAAAA GTAAGAATGT ATTGCTGAAG |
| | AGCGACCTCA |
| 1001 | CAGCCGTGCT GGCTGACTTT GGCTTGGCTG TTCGATTTGA |
| | GCCAGGGAAA |
| 1051 | CCTCCAGGGG ACACCCACGG ACAGGTAGGC ACGAGACGGT |
| | ACATGGCTCC |
| 1101 | TGAGGTGCTC GAGGGAGCCA TCAACTTCCA GAGAGATGCC |
| | TTCCTGCGCA |
| 1151 | TTGACATGTA TGCCATGGGG TTGGTGCTGT GGGAGCTTGT |
| | GTCTCGCTGC |
| 1201 | AAGGCTGCAG ACGGACCCGT GGATGAGTAC ATGCTGCCCT |
| | TTGAGGAAGA |
| 1251 | GATTGGCCAG CACCCTTCGT TGGAGGAGCT GCAGGAGGTG |
| | GTGGTGCACA |
| 1301 | AGAAGATGAG GCCCACCATT AAAGATCACT GGTTGAAACA |
| | CCCGGGCCTG |
| 1351 | GCCCAGCTTT GTGTGACCAT CGAGGAGTGC TGGGACCATG |
| | ATGCAGAGGC |
| 1401 | TCGCTTGTCC GCGGGCTGTG TGGAGGAGCG GGTGTCCCTG |
| | ATTCGGAGGT |
| 1451 | CGGTCAACGG CACTACCTCG GACTGTCTCG TTTCCCTGGT |
| | GACCTCTGTC |
| 1501 | ACCAATGTGG ACCTGCCCCC TAAAGAGTCA AGCATC |

A nucleic acid sequence encoding processed extracellular human ActRIIB polypeptide is as follows (SEQ ID NO: 8). The sequence as shown provides an arginine at position 64, and may be modified to provide an alanine instead.

(SEQ ID NO: 8)

| | |
|---|---|
| 1 | GGGCGTGGGG AGGCTGAGAC ACGGGAGTGC ATGTAGTACA |
| | ACGCCAACTG |
| 51 | GGAGCTGGAG CGCACCAACC AGAGCGGCCT GGAGCGCTGC |
| | GAAGGCGAGC |
| 101 | AGGACAAGCG GCTGCACTGC TACGCCTCCT GGCGCAACAG |
| | CTCTGGCACC |
| 151 | ATCGAGCTCG TGAAGAAGGG CTGCTGGCTA GATGACTTCA |
| | ACTGCTACGA |
| 201 | TAGGCAGGAG TGTGTGGCCA CTGAGGAGAA CCCCCAGGTG |
| | TACTTCTGCT |
| 251 | GCTGTGAAGG CAACTTCTGC AACGAACGCT TCACTCATTT |
| | GCCAGAGGCT |
| 301 | GGGGGCCCGG AAGTCAGGTA CGAGCCACCC CCGACAGCCC |
| | CCACC |

In some embodiments the ActRIIB polypeptide comprises the accession number NP_001097.2 (SEQ ID NO: 1 herein), and variants thereof. In some embodiments, the term “wild-type ActRIIB” refers to the extracellular domain of ActRIIB, amino acids 1 to 134 (with signal sequence), or amino acids 19 through 134 of SEQ ID NO: 1 (without signal sequence) (referred to herein as SEQ ID NO: 407).

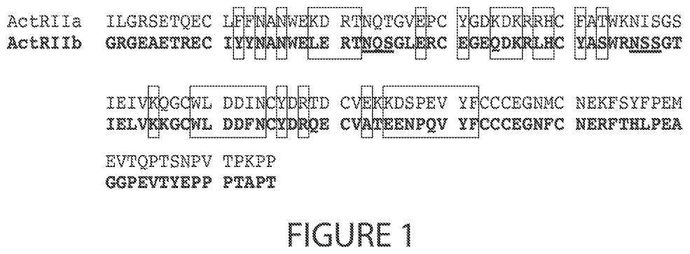

An alignment of the amino acid sequences of human ActRIIB extracellular domain and human ActRIIA extracellular domain are illustrated in FIG. 1. This alignment indicates amino acid residues within both receptors that are believed to directly contact ActRII ligands. For example, the composite ActRII structures indicated that the ActRIIB-ligand binding pocket is defined, in part, by residues Y31, N33, N35, L38 through T41, E47, E50, Q53 through K55, L57, H58, Y60, S62, K74, W78 through N83, Y85, R87, A92, and E94 through F101 (based on the numbering of SEQ ID NO: 1). At these positions, it is expected that conservative mutations will be tolerated.

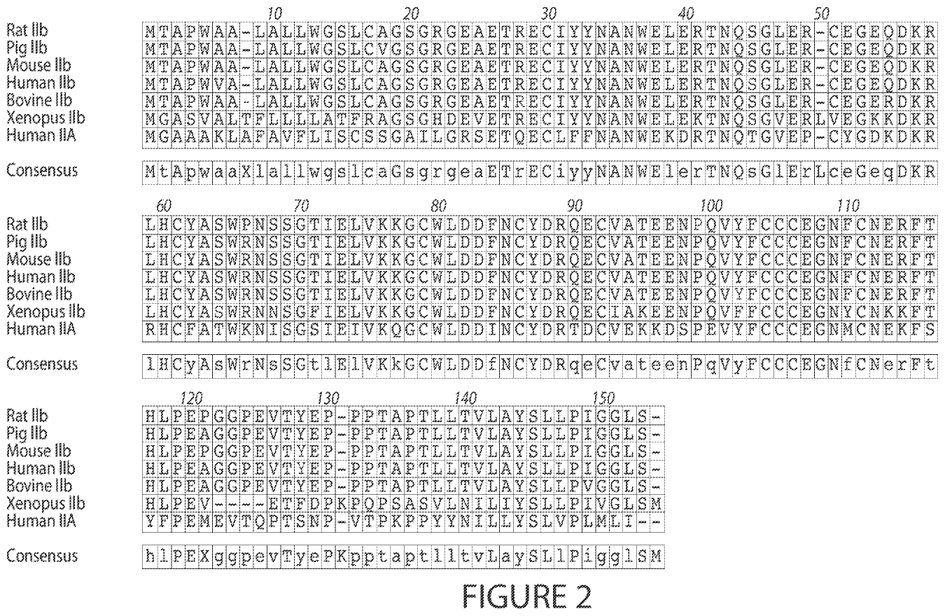

In addition, ActRIIB is well-conserved among vertebrates, with large stretches of the extracellular domain completely conserved. For example, FIG. 2 depicts a multi-sequence alignment of a human ActRIIB extracellular domain compared to various ActRIIB orthologs. Many of the ligands that bind to ActRIIB are also highly conserved. Accordingly, from these alignments, it is possible to predict key amino acid positions within the ligand-binding domain that are important for normal ActRIIB-ligand binding activities as well as to predict amino acid positions that are likely to be tolerant to substitution without significantly altering normal ActRIIB-ligand binding activities. Therefore, an active, human ActRIIB variant polypeptide useful in accordance with the presently disclosed methods may include one or more amino acids at corresponding positions from the sequence of another vertebrate ActRIIB, or may include a residue that is similar to that in the human or other vertebrate sequences. Without meaning to be limiting, the following examples illustrate this approach to defining an active ActRIIB variant. L46 in the human extracellular domain (SEQ ID NO: 2) is a valine in Xenopus ActRIIB (SEQ ID NO: 58), and so this position may be altered, and optionally may be altered to another hydrophobic residue, such as V, I or F, or a non-polar residue such as A. E52 in the human extracellular domain is a K in Xenopus, indicating that this site may be tolerant of a wide variety of changes, including polar residues, such as E, D, K, R, H, S, T, P, G, Y and probably A. T93 in the human extracellular domain is a K in Xenopus, indicating that a wide structural variation is tolerated at this position, with polar residues favored, such as S, K, R, E, D, H, G, P, G and Y. F108 in the human extracellular domain is a Y in Xenopus, and therefore Y or other hydrophobic group, such as I, V or L should be tolerated. E111 in the human extracellular domain is K in Xenopus, indicating that charged residues will be tolerated at this position, including D, R, K and H, as well as Q and N. R112 in the human extracellular domain is K in Xenopus, indicating that basic residues are tolerated at this position, including R and H. A at position 119 in the human extracellular domain is relatively poorly conserved, and appears as P in rodents and V in Xenopus, thus essentially any amino acid should be tolerated at this position.

Moreover, ActRII proteins have been characterized in the art in terms of structural and functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103 (20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide amply guidance for how to generate ActRIIB variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIB, as demarcated by the outermost of these conserved cysteines, corresponds to positions 29-109 of SEQ ID NO: 1 (ActRIIB precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, or 28 residues at the N-terminus and/or 1, 2, 3, 4, 5, 6, 7, 8, 9, 10, 11, 12, 13, 14, 15, 16, 17, 18, 19, 20, 21, or 22 residues a the C-terminus without necessarily altering ligand binding. Exemplary ActRIIB extracellular domains for N-terminal and/or C-terminal truncation include SEQ ID NOs: 2, 3, 5, 6, 318, and 331.

Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 1, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar, but somewhat reduced activity, relative to the wild-type, even though the proline knot region is disrupted.

Thus, ActRIIB extracellular domains that stop at amino acid 134, 133, 132, 131, 130 and 129 (with respect to SEQ ID NO: 1) are all expected to be active, but constructs stopping at 134 or 133 may be most active. Similarly, mutations at any of residues 129-134 (with respect to SEQ ID NO: 1) are not expected to alter ligand-binding affinity by large margins. In support of this, it is known in the art that mutations of P129 and P130 (with respect to SEQ ID NO: 1) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO:1) is poorly conserved and so is readily altered or truncated. ActRIIB polypeptides ending at 128 (with respect to SEQ ID NO: 1) or later should retain ligand-binding activity. ActRIIB polypeptides ending at or between 119 and 127 (e.g., 119, 120, 121, 122, 123, 124, 125, 126, or 127), with respect to SEQ ID NO: 1, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 1) will retain ligand-binding activity. Amino acid 29 represents the initial cysteine. An alanine-to-asparagine mutation at position 24 (with respect to SEQ ID NO: 1) introduces an N-linked glycosylation sequence without substantially affecting ligand binding [U.S. Pat. No. 7,842,663]. This confirms that mutations in the region between the signal cleavage peptide and the cysteine cross-linked region, corresponding to amino acids 20-29, are well tolerated. In particular, ActRIIB polypeptides beginning at position 20, 21, 22, 23, and 24 (with respect to SEQ ID NO: 1) should retain general ligand-biding activity, and ActRIIB polypeptides beginning at positions 25, 26, 27, 28, and 29 (with respect to SEQ ID NO: 1) are also expected to retain ligand-biding activity. It has been demonstrated, e.g., U.S. Pat. No. 7,842,663, that, surprisingly, an ActRIIB construct beginning at 22, 23, 24, or 25 will have the most activity.

Taken together, a general formula for an active portion (e.g., ligand-binding portion) of ActRIIB comprises amino acids 29-109 of SEQ ID NO: 1. Therefore ActRIIB polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to any one of amino acids 20-29 (e.g., beginning at any one of amino acids 20, 21, 22, 23, 24, 25, 26, 27, 28, or 29) of SEQ ID NO: 1 and ending at a position corresponding to any one amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. Other examples include polypeptides that begin at a position from 20-29 (e.g., any one of positions 20, 21, 22, 23, 24, 25, 26, 27, 28, or 29) or 21-29 (e.g., any one of positions 21, 22, 23, 24, 25, 26, 27, 28, or 29) of SEQ ID NO: 1 and end at a position from 119-134 (e.g., any one of positions 119, 120, 121, 122, 123, 124, 125, 126, 127, 128, 129, 130, 131, 132, 133, or 134), 119-133 (e.g., any one of positions 119, 120, 121, 122, 123, 124, 125, 126, 127, 128, 129, 130, 131, 132, or 133), 129-134 (e.g., any one of positions 129, 130, 131, 132, 133, or 134), or 129-133 (e.g., any one of positions 129, 130, 131, 132, or 133) of SEQ ID NO: 1. Other examples include constructs that begin at a position from 20-24 (e.g., any one of positions 20, 21, 22, 23, or 24), 21-24 (e.g., any one of positions 21, 22, 23, or 24), or 22-25 (e.g., any one of positions 22, 22, 23, or 25) of SEQ ID NO: 1 and end at a position from 109-134 (e.g., any one of positions 109, 110, 111, 112, 113, 114, 115, 116, 117, 118, 119, 120, 121, 122, 123, 124, 125, 126, 127, 128, 129, 130, 131, 132, 133, or 134), 119-134 (e.g., any one of positions 119, 120, 121, 122, 123, 124, 125, 126, 127, 128, 129, 130, 131, 132, 133, or 134) or 129-134 (e.g., any one of positions 129, 130, 131, 132, 133, or 134) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1.

The variations described herein may be combined in various ways. In some embodiments, ActRIIB variants comprise no more than 1, 2, 5, 6, 7, 8, 9, 10 or 15 conservative amino acid changes in the ligand-binding pocket, optionally zero, one or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 1). An asparagine-to-alanine alteration at position 65 (N65A) does not appear to decrease ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 1, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 1.

It has been previously demonstrated that the addition of a further N-linked glycosylation site (N-X-S/T) into the ActRIIB extracellular domain is well-tolerated (see, e.g., U.S. Pat. No. 7,842,663). Therefore, N-X-S/T sequences may be generally introduced at positions outside the ligand binding pocket defined in FIG. 1 in ActRIIB polypeptide of the present disclosure. Particularly suitable sites for the introduction of non-endogenous N-X-S/T sequences include amino acids 20-29, 20-24, 22-25, 109-134, 120-134 or 129-134 (with respect to SEQ ID NO: 1). N-X-S/T sequences may also be introduced into the linker between the ActRIIB sequence and an Fc domain or other fusion component as well as optionally into the fusion component itself. Such a site may be introduced with minimal effort by introducing an N in the correct position with respect to a pre-existing S or T, or by introducing an S or T at a position corresponding to a pre-existing N. Thus, desirable alterations that would create an N-linked glycosylation site are: A24N, R64N, S67N (possibly combined with an N65A alteration), E105N, R112N, G120N, E123N, P129N, A132N, R112S and R112T (with respect to SEQ ID NO: 1). Any S that is predicted to be glycosylated may be altered to a T without creating an immunogenic site, because of the protection afforded by the glycosylation. Likewise, any T that is predicted to be glycosylated may be altered to an S. Thus the alterations S67T and S44T (with respect to SEQ ID NO: 1) are contemplated. Likewise, in an A24N variant, an S26T alteration may be used. Accordingly, an ActRIIB polypeptide of the present disclosure may be a variant having one or more additional, non-endogenous N-linked glycosylation consensus sequences as described above.

In certain embodiments, the disclosure relates to ActRII antagonists (inhibitors) that comprise a ActRIIB polypeptide, which includes fragments, functional variants, and modified forms thereof as well as uses thereof (e.g., treating or preventing PH or one or more PH-associated complication, treating a kidney-associated disease (e.g., Alport syndrome, focal segmental glomerulosclerosis (FSGS), polycystic kidney disease, or chronic kidney disease), and/or treating an interstitial lung disease). Preferably, ActRIIB polypeptides are soluble (e.g., comprise an extracellular domain of ActRIIB). In some embodiments, ActRIIB polypeptides antagonize activity (e.g., Smad signaling) of one or more TGF-beta family ligands [e.g., activin A, activin B, BMP6, BMP9, BMP10, GDF3, GDF8, and/or GDF11]. Therefore, in some embodiments, ActRIIB polypeptides bind to one or more TGF-beta family ligands [e.g., activin A, activin B, BMP6, BMP9, BMP10, GDF3, GDF8, and/or GDF11]. In some embodiments, ActRIIB polypeptides of the disclosure demonstrate a decreased binding affinity for BMP9. In some embodiments, ActRIIB polypeptides of the disclosure do not bind BMP9. In some embodiments, ActRIIB polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to amino acids 20-29 (e.g., beginning at any one of amino acids 20, 21, 22, 23, 24, 25, 26, 27, 28, or 29) of SEQ ID NO: 1 and ending at a position corresponding to amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. In some embodiments, ActRIIB polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1. In some embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1, wherein the position corresponding to L79 of SEQ ID NO: 1 is an acidic amino acid (naturally occurring acidic amino acids D and E or an artificial acidic amino acid). In certain embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1. In certain embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1, wherein the position corresponding to L79 of SEQ ID NO: 1 is an acidic amino acid. In some embodiments, ActRIIB polypeptide of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 108, 110, 114, 115, 118, 120, 121, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 1. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 2. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 3. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 4. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 5. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 6. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 40. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 42. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 45. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 46. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 47. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 48. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 69. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 74. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 77. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 78. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 79. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 108. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 110. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 114. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 115. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 118. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 120. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 121. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 138. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 282. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 289. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 290. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 291. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 292. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 293. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 294. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 295. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 296. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 297. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 298. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 299. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 300. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 301. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 302. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 303. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 305. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 306. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 307. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 308. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 309. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 310. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 311. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 312. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 313. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 314. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 315. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 316. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 317. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 318. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 319. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 320. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 321. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 322. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 323. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 324. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 325. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 326. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 327. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 328. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 329. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 330. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 331. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 332. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 333. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 334. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 335. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 336. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 337. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 338. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 339. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 340. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 341. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 342. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 343. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 344. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 345. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 346. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 347. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 348. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 349. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 350. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 351. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 352. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 353. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 354. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 355. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 356. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 357. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 358. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 359. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 360. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 361. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 362. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 363. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 364. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 365. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 366. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 367. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 368. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 369. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 370. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 371. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 372. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 373. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 374. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 375. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 376. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 377. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 378. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 379. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 380. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 381. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 382. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 383. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 384. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 385. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 386. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 387. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 388. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 389. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 390. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 391. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 392. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 393. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 394. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 395. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 396. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 397. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 398. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 399. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 400. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 401. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 402. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 403. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 404. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 405. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 406. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 407. In some embodiments, ActRIIB polypeptide of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 108, 110, 114, 115, 118, 120, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407, wherein the position corresponding to L79 of SEQ ID NO: 1 is an acidic amino acid. In some embodiments, ActRIIB polypeptides of the disclosure comprise, consist, or consist essentially of, at least one ActRIIB polypeptide wherein the position corresponding to L79 of SEQ ID NO: 1 is not an acidic amino acid (i.e., is not naturally occurring acid amino acids D or E or an artificial acidic amino acid residue).

In some embodiments, the ActRIIB polypeptide of the disclosure comprises an alternate, souble form of ActRIIB (designated ActRIIB5), in which exon 4, including the ActRIIB transmembrane domain, has been replaced by a different C-terminal sequence (see, e.g., WO 2007/053775). In some embodiments, ActRIIB5 polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a polypeptide selected from the group consisting of SEQ ID NOs: 50, 51, or 52.

In some embodiments, ActRIIB polypeptides of the disclosure comprise, consist, or consist essentially of, at least one extracellular ActRIIB variant polypeptide having the sequence of SEQ ID NO: 282 shown below:

(SEQ ID NO: 282)
GRGEAETRECIFYNANWEKDRTNQSGLEPCYGDQDKRRHCFASWKNSSGT
IELVKQGCWLDDINCYDRQECVAKKDSPEVYFCCCEGNFCNERFTHLPEA
GGPEVTYEPPPTAPT.

In some embodiments, ActRIIB polypeptides of the disclosure comprise, consist, or consist essentially of, at least one extracellular ActRIIB variant polypeptide having the sequence of any one of SEQ ID NOs: 282, 289, or 290-302. In some embodiments, ActRIIB polypeptides of the disclosure comprise, consist, or consist essentially of, at least one extracellular ActRIIB variant polypeptide having the sequence of any one of SEQ ID NOs: 282 or 290-302 (Table 3).

In some embodiments, ActRIIB polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the processed (mature) extracellular ActRIIB polypeptide sequence (SEQ ID NO: 2).

Polypeptides described herein include an extracellular ActRIIB variant having at least one amino acid substitution relative to the processed (mature) extracellular ActRIIB polypeptide sequence having the sequence of SEQ ID NO: 2. Possible amino acid substitutions at 28 different positions may be introduced to an extracellular ActRIIB variant (Table 1). An extracellular ActRIIB variant may have one or more (e.g., 1-28, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a processed (mature) extracellular ActRIIB polypeptide sequence (SEQ ID NO: 2). In some embodiments, an extracellular ActRIIB variant (e.g., an extracellular ActRIIB variant having a sequence of SEQ ID NO: 289) may include amino acid substitutions at all of the 28 positions as listed in Table 1. In some embodiments, an extracellular ActRIIB variant may include amino acid substitutions at a number of positions, e.g., at 3, 4, 5, 6, 7, 8, 9, 10, 12, 15, 16, 18, 20, 22, 24, 26, or 27 out of the 28 positions, as listed in Table 1. In some embodiments, the substitutions are substitutions of an amino acid from an ActRIIA polypeptide sequence into the same position in an ActRIIB polypeptide sequence. In some embodiments, the substitutions are novel changes (e.g., substitutions of amino acids that are not in the corresponding position of ActRIIA, e.g., S48T, 151 L, Q69D, or E70T).

Amino acid substitutions can worsen or improve the activity and/or binding affinity of the ActRIIB variants disclosed herein (e.g., an extracellular ActRIIB variant having the sequence of any one of SEQ ID NOs: 282, 289, and 290-30 (e.g., SEQ ID NOs: 282 and 290-302)). In some embodiments, the amino acid substitutions worsen the binding affinity of the ActRIIB variants to BMP9 (e.g., the variants have reduced binding to BMP9 relative to wild-type extracellular ActRIIB, or have lower binding to BMP9 than to other ActRIIB ligands (e.g., activin A or B, myostatin, or GDF-11)). In some embodiments, the ActRIIB variants have reduced or no substantial binding to BMP9. In some embodiments, the amino acid substitutions improve the binding affinity of ActRIIB to myostatin, activin A or B, and/or GDF-11 (e.g., the variants have improved binding affinity relative to wild-type extracellular ActRIIB, or bind more strongly to myostatin, activin A or B, or GDF-11 than to BMP9). In some embodiments, the amino acid substitutions reduce the binding affinity of ActRIIB to myostatin, activin A or B, and/or GDF-11 (e.g., the variants have decreased binding affinity relative to wild-type extracellular ActRIIB, or have reduced binding to myostatin, activin A or B, or GDF-11 as compared to BMP9). In some embodiments, the amino acid substitutions do not substantially change extracellular ActRIIB function (e.g., the ActRIIB variants increase lean mass, muscle, mass, or bone mineral density, or reduce or prevent fibrosis, by a similar amount as wild-type extracellular ActRIIB, e.g., the ActRIIB variants are functionally equivalent to the wild-type extracellular ActRIIB). In some embodiments, the amino acid substitutions confer a property or activity of an ActRIIA polypeptide on an ActRIIB variant polypeptide (e.g., the ActRIIB variant polypeptide has a longer half-life than wild-type extracellular ActRIIB). In some embodiments, the ActRIIB variant polypeptides have one or more, two or more, or three or more of the above properties (e.g., reduced BMP9 binding and improved binding to activin A or B, myostatin, and/or GDF-11, or reduced BMP9 binding and functional equivalence to wild-type ActRIIB).

In some embodiments, ActRIIB polypeptides of the disclosure (e.g., an extracellular ActRIIB variant having the sequence of any one of SEQ ID NOs: 282, 289, and 290-30 (e.g., SEQ ID NOs: 282 and 290-302)) have one or more amino acid substitutions that reduce BMP9 binding. In some embodiments, the amino acid substitution that reduces BMP9 binding is E75K (e.g., X₂₄ is K in SEQ ID NO: 289). In some embodiments, the amino acid substitutions that reduce BMP9 binding are Q69T and E70D (e.g., X₂₁ is T and X₂₂ is D in SEQ ID NO: 289). In some embodiments, the amino acid substitutions that reduce BMP9 binding are Q69D and E70T (e.g., X₂₁ is D and X₂₂ is T in SEQ ID NO: 289). In some embodiments, the amino acid substitutions that reduce BMP9 binding are T74K, E75K, E76D, N77S, and Q79E (e.g., X₂₃, X₂₄, X₂₅, X₂₆, and X₂₈ are K, K, D, S, and E, respectively, in SEQ ID NO: 289). In some embodiments, the ActRIIB variants have more than one of the aforementioned amino acid substitutions that reduce BMP9 binding (e.g., substitution E75K and substitutions Q69D and E70T, or substitution E75K and substitutions Q69T and E70D). In some embodiments, the ActRIIB variants disclosed herein have one or more amino acid substitutions that reduce BMP9 binding, and one or more additional amino acid substitutions. The additional amino acid substitutions may confer other beneficial properties, such as altered binding to activins or myostatin or improved activity. For example, amino acid substitutions T74K, E75K, E76D, N77S, and Q79E lead to a reduction in ActRIIB variant activity, but including additional substitutions S25T and S47I; E31Y, E33D, and Q34K; or Y41F, R45K, and K56Q improves the ActRIIB variant activity. The additional amino acid substitutions may include one or more of substitutions I11L, Y12F, L19K, E20D, S25T, L27V, R29P, E31Y, E33D, Q34K, L38R, Y41F, R45K, S47I, S48T, T50S, I51L, L53I, K56Q, F63I, T74K, E76D, N77S, Q79E, or F89M.

In some embodiments, variant ActRIIB polypeptides of the disclosure comprise one or more amino acid substitutions relative to the sequence of SEQ ID NO: 2, in which the variant contains one or more amino acid substitutions that impart reduced BMP9 binding relative to wild type extracellular ActRIIB, and one or more additional amino acid substitutions, wherein the substitutions that reduce BMP9 binding are one or more of: (a) amino acid substitution E75K; (b) amino acid substitutions Q69T and E70D; or (c) amino acid substitutions Q69D and E70T. In some embodiments, the one or more additional amino acid substitutions are selected from the group consisting of I11L, Y12F, L19K, E20D, S25T, L27V, R29P, E31Y, E33D, Q34K, L38R, Y41F, R45K, S47I, S48T, T50S, I51L, L53I, K56Q, F63I, T74K, E76D, N77S, Q79E, and F89M. In some embodiments, the variant contains amino acid substitution E75K and additional amino acid substitutions E20D and F63I. In some embodiments, the variant polypeptide further comprises amino acid substitution E75K. In some embodiments, the variant contains amino acid substitution E75K and additional amino acid substitutions that reduce BMP9 binding. In some embodiments of any of the above embodiments, the additional amino acid substitutions that reduce BMP9 binding are T74K, E76D, N77S, and Q79E. In some embodiments, the variant further contains one or more additional amino acid substitutions. In some embodiments, the variant contains additional amino acid substitutions Y41F, R45K, and K56Q. In some embodiments, the variant further contains additional amino acid substitutions Y12F, L19K, E20D, R29P, E31Y, E33D, L38R, and F631. In some embodiments, the variant contains additional amino acid substitutions S25T and S47I. In some embodiments, the variant contains additional amino acid substitution S48T. In some embodiments, the variant contains additional amino acid substitution R29P. In some embodiments, the variant contains additional amino acid substitutions E31Y, E33D, and Q34K. In some embodiments, the variant contains additional amino acid substitutions Y12F, L19K, and E20D. In some embodiments, the variant contains additional amino acid substitutions E31Y, E33D, and L38R. In some embodiments, the variant contains amino acid substitutions Q69T and E70D, and additional amino acid substitutions I11L, L27V, Q34K, T50S, I51L, L53I, and F89M. In some embodiments, the variant contains amino acid substitutions Q69D and E70T, and additional amino acid substitutions I11L, L27V, Q34K, T5S, 151L, L53X, and F89M. In some embodiments, the variant further contains amino acid substitution E75K. In some embodiments, the variant polypeptide comprises the sequence of any one of SEQ TD NOs: 282 or 290-302. See, e.g., Table 3.

In some embodiments, a polypeptide described herein includes an extracellular ActRIIB variant having the sequence of SEQ ID NO: 289.

TABLE 1
Amino acid substitutions in an extracellular
ActRIIB variant having a sequence of
SEQ ID NO: 289

GRGEAETRECX₁X₂YNANWEX₃X₄RTNQX₅GX₆EX₇CX₈GX₉X₁₀DKR
X₁₁HCX₁₂ASWX₁₃NX₁₄X₁₅GX₁₆X₁₇EX₁₈VKX₁₉GCWLDDX₂₀NC
YDRX₂₁X₂₂CVAX₂₃X₂₄X₂₅X₂₆PX₂₇VYFCCCEGNX₂₈CNERFTHL
PEAGGPEVTYEPPPTAPT (SEQ ID NO: 289)

| | |
|---|---|
| X₁ | I or L |
| X₂ | F, Y, A, V, I, L, M, or W |
| X₃ | L or K |
| X₄ | D, E, or A |
| X₅ | T, S, N, or Q |
| X₆ | L, V, A, I, M, F, Y, or W |
| X₇ | P or R |
| X₈ | Y or E |
| X₉ | D, E, or A |
| X₁₀ | K or Q |
| X₁₁ | R or L |
| X₁₂ | A, V, I, L, M, Y, W or F |
| X₁₃ | R, H, or K |
| X₁₄ | S or I |
| X₁₅ | S, N, Q, or T |
| X₁₆ | S, N, Q, or T |
| X₁₇ | L, V, A, I, M, F, Y, or W |
| X₁₈ | L, V, A, I, M, F, Y, or W |
| X₁₉ | K or Q |
| X₂₀ | L, V, A, I, M, F, Y, or W |
| X₂₁ | Q, T, S, N or D |
| X₂₂ | E, D, A, or T |
| X₂₃ | K or T |
| X₂₄ | K or E |
| X₂₅ | D, E, or A |
| X₂₆ | T, S, N, or Q |
| X₂₇ | E or Q |
| X₂₈ | L, V, A, I, M, F, Y, or W |

TABLE 2
Compositions that can be administered to a subject
according to the methods described herein.

| Row | Composition |
|---|---|
| 1 | A polypeptide containing an ActRIIB variant, the variant |
| | having a sequence of |
| | GRGEAETRECX₁X₂YNANWEX₃X₄RTNQX₅GX₆EX₇CX₈GX₉X₁₀DKRX₁₁HCX₁₂ |
| | ASWX₁₃NX₁₄X₁₅GX₁₆X₁₇EX₁₈VKX₁₉GCWLDDX₂₀NCYDRX₂₁X₂₂CVAX₂₃ |
| | X₂₄X₂₅X₂₆PX₂₇VYFCCCEGNX₂₈CNERFTHLPEAGGPEVTYEPPPTAPT |
| | (SEQ ID NO: 289), |
| | wherein X₁ is I or L; X₂ is F or Y; X₃ is L or K; X₄ is |
| | D or E; X₅ is T or S; X₆ is L or V; X₇ is P or R; X₈ is |
| | Y or E; X₉ is D or E; X₁₀ is K or Q; X₁₁ is R or L; X₁₂ |
| | is Y or F; X₁₃ is R or K; X₁₄ is S or I; X₁₅ is S or T; |
| | X₁₆ is S or T; X₁₇ is I or L; X₁₈ is I or L; X₁₉ is K or |
| | Q; X₂₀ is F or I; X₂₁ is Q, T, or D; X₂₂ is E, D, or T; |
| | X₂₃ is K or T; X₂₄ is K or E; X₂₅ is D or E; X₂₆ is S or |
| | N; X₂₇ is E or Q; and X₂₈ is F or M, and wherein X₂₄ is |
| | E and/or either X₂₁ is T and X₂₂ is D or X₂₁ is D and |
| | X₂₂ is T. |
| 2 | A polypeptide containing an ActRIIB variant, the variant |
| | having a sequence of GRGEAETRECX₁X₂YNANWEX₃X₄RTNQX₅GX₆E |
| | X₇CX₈GX₉X₁₀DKRX₁₁HCX₁₂ASWX₁₃NX₁₄X₁₅GX₁₆X₁₇EX₁₈VKX₁₉GCWL |
| | DDX₂₀NCYDRX₂₁X₂₂CVAX₂₃X₂₄X₂₅X₂₆PX₂₇VYFCCCEGNX₂₈CNERFTHL |
| | PEAGGPEVTYEPPPTAPT (SEQ ID NO: 289), |
| | wherein X₁ is I or L; X₂ is F, Y, A, V, I, L, M, or W; |
| | X₃ is L or K; X₄ is D, E, or A; X₅ is T, S, N, or Q; X₆ |
| | is L, V, A, I, M, F, Y, or W; X₇ is P or R; X₈ is Y or |
| | E; X₉ is D, E, or A; X₁₀ is K or Q; X₁₁ is R or L; X₁₂ |
| | is A, V, I, L, M, Y, W or F; X₁₃ is R, H, or K; X₁₄ is S |
| | or I; X₁₅ is S, N, Q, or T; Xie is S, N, Q, or T; X₁₇ is |
| | L, V, A, I, M, F, Y, or W; X₁₈ is L, V, A, I, M, F, Y, |
| | or W; X₁₉ is K or Q; X₂₀ is L, V, A, I, M, F, Y, or W; |
| | X₂₁ is Q, T, S, N or D; X₂₂ is E, D, A, or T; X₂₃ is K |
| | or T; X₂₄ is K or E; X₂₅ is D, A, or E; X₂₆ is T, S, N, |
| | or Q; X₂₇ is E or Q; and X₂₈ is L, V, A, I, M, F, Y, or |
| | W, and wherein X₂₄ is E and/or either X₂₁ is T and X₂₂ |
| | is D or X₂₁ is D and X₂₂ is T. |
| 3 | The variant of row 1 or 2, wherein X₁ is I. |
| 4 | The variant of row 1 or 2, wherein X₁ is L. |
| 5 | The variant of any one of rows 1-4, wherein X₂ is F. |
| 6 | The variant of any one of rows 1-4, wherein X₂ is Y. |
| 7 | The variant of any one of rows 1-6, wherein X₃ is L. |
| 8 | The variant of any one of rows 1-6, wherein X₃ is K. |
| 9 | The variant of any one of rows 1-8, wherein X₄ is D. |
| 10 | The variant of any one of rows 1-8, wherein X₄ is E. |
| 11 | The variant of any one of rows 1-10, wherein X₅ is T. |
| 12 | The variant of any one of rows 1-10, wherein X₅ is S. |
| 13 | The variant of any one of rows 1-12, wherein X₆ is L. |
| 14 | The variant of any one of rows 1-12, wherein X₆ is V. |
| 15 | The variant of any one of rows 1-14, wherein X₇ is P. |
| 16 | The variant of any one of rows 1-14, wherein X₇ is R. |
| 17 | The variant of any one of rows 1-16, wherein X₈ is Y. |
| 18 | The variant of any one of rows 1-16, wherein X₈ is E. |
| 19 | The variant of any one of rows 1-18, wherein X₉ is D. |
| 20 | The variant of any one of rows 1-18, wherein X₉ is E. |
| 21 | The variant of any one of rows 1-20, wherein X₁₀ is K. |
| 22 | The variant of any one of rows 1-20, wherein X₁₀ is Q. |
| 23 | The variant of any one of rows 1-22, wherein X₁₁ is R. |
| 24 | The variant of any one of rows 1-22, wherein X₁₁ is L. |
| 25 | The variant of any one of rows 1-24, wherein X₁₂ is Y. |
| 26 | The variant of any one of rows 1-24, wherein X₁₂ is F. |
| 27 | The variant of any one of rows 1-26, wherein X₁₃ is R. |
| 28 | The variant of any one of rows 1-26, wherein X₁₃ is K. |
| 29 | The variant of any one of rows 1-28, wherein X₁₄ is S. |
| 30 | The variant of any one of rows 1-28, wherein X₁₄ is I. |
| 31 | The variant of any one of rows 1-30, wherein X₁₅ is S. |
| 32 | The variant of any one of rows 1-30, wherein X₁₅ is T. |
| 33 | The variant of any one of rows 1-32, wherein X₁₆ is S. |
| 34 | The variant of any one of rows 1-32, wherein X₁₆ is T. |
| 35 | The variant of any one of rows 1-34, wherein X₁₇ is I. |
| 36 | The variant of any one of rows 1-34, wherein X₁₇ is L. |
| 37 | The variant of any one of rows 1-36, wherein X₁₈ is I. |
| 38 | The variant of any one of rows 1-36, wherein X₁₈ is L. |
| 39 | The variant of any one of rows 1-38, wherein X₁₉ is K. |
| 40 | The variant of any one of rows 1-38, wherein X₁₉ is Q. |
| 41 | The variant of any one of rows 1-40, wherein X₂₀ is F. |
| 42 | The variant of any one of rows 1-40, wherein X₂₀ is I. |
| 43 | The variant of any one of rows 1-42, wherein X₂₁ is Q. |
| 44 | The variant of any one of rows 1-42, wherein X₂₁ is T. |
| 45 | The variant of any one of rows 1-42, wherein X₂₁ is D. |
| 46 | The variant of any one of rows 1-43, wherein X₂₂ is E. |
| 47 | The variant of any one of rows 1-42 and row 45, wherein |
| | X₂₂ is D. |
| 48 | The variant of any one of rows 1-42 and row 45, wherein |
| | X₂₂ is T. |
| 49 | The variant of any one of rows 1-48, wherein X₂₃ is K. |
| 50 | The variant of any one of rows 1-48, wherein X₂₃ is T. |
| 51 | The variant of any one of rows 1-50, wherein X₂₄ is K. |
| 52 | The variant of any one of rows 1-42, row 44, row 45, row |
| | 47, and row 48-50, wherein X₂₄ is E. |
| 53 | The variant of any one of rows 1-52, wherein X₂₅ is D. |
| 54 | The variant of any one of rows 1-52, wherein X₂₅ is E. |
| 55 | The variant of any one of rows 1-54, wherein X₂₆ is S. |
| 56 | The variant of any one of rows 1-54, wherein X₂₆ is N. |
| 57 | The variant of any one of rows 1-56, wherein X₂₇ is E. |
| 58 | The variant of any one of rows 1-56, wherein X₂₇ is Q. |
| 59 | The variant of any one of rows 1-58, wherein X₂₈ is F. |
| 60 | The variant of any one of rows 1-58, wherein X₂₈ is M. |

In some embodiments, a polypeptide described herein includes an extracellular ActRIIB variant having a sequence of any one of SEQ ID NOs: 282 and 290-302 (Table 3).

TABLE 3
Extracellular ActRIIB variants having the sequences of
SEQ ID NOs: 282 and 290-302

| SEQ ID NO | Amino Acid Sequence |
|---|---|
| 282 | GRGEAETRECIFYNANWEKDRTNQSGLEPCYGDQDKRRHCFASWKNSSGT |
| | IELVKQGCWLDDINCYDRQECVAKKDSPEVYFCCCEGNFCNERFTHLPEA |
| | GGPEVTYEPPPTAPT |
| 290 | GRGEAETRECIYYNANWELDRTNQSGLERCEGEQDKRLHCYASWRNSSGT |
| | IELVKKGCWLDDINCYDRQECVATKENPQVYFCCCEGNFCNERFTHLPEA |
| | GGPEVTYEPPPTAPT |
| 291 | GRGEAETRECIYYNANWELERTNQSGLERCEGEQDKRLHCYASWRNSSGT |
| | IELVKKGCWLDDFNCYDRQECVAKKDSPEVYFCCCEGNFCNERFTHLPEA |
| | GGPEVTYEPPPTAPT |
| 292 | GRGEAETRECIYYNANWELERTNQTGLERCEGEQDKRLHCYASWRNISGT |
| | IELVKKGCWLDDFNCYDRQECVAKKDSPEVYFCCCEGNFCNERFTHLPEA |
| | GGPEVTYEPPPTAPT |
| 293 | GRGEAETRECIYYNANWELERTNQTGLERCEGEQDKRLHCYASWRNITGT |
| | IELVKKGCWLDDFNCYDRQECVAKKDSPEVYFCCCEGNFCNERFTHLPEA |
| | GGPEVTYEPPPTAPT |
| 294 | GRGEAETRECIYYNANWELERTNQSGLEPCEGEQDKRLHCYASWRNSSGT |
| | IELVKKGCWLDDFNCYDRQECVAKKDSPEVYFCCCEGNFCNERFTHLPEA |
| | GGPEVTYEPPPTAPT |
| 295 | GRGEAETRECIYYNANWELERTNQSGLERCYGDKDKRLHCFASWKNSSGT |
| | IELVKQGCWLDDFNCYDRQECVAKKDSPEVYFCCCEGNFCNERFTHLPEA |
| | GGPEVTYEPPPTAPT |
| 296 | GRGEAETRECIYYNANWELERTNQSGLERCEGEQDKRLHCYASWRNSSGT |
| | IELVKKGCWLDDFNCYDRQECVAKKDSPEVYFCCCEGNFCNERFTHLPEA |
| | GGPEVTYEPPPTAPT |
| 297 | GRGEAETRECIFYNANWEKDRTNQSGLERCYGDQDKRRHCYASWRNSSGT |
| | IELVKKGCWLDDFNCYDRQECVAKKDSPEVYFCCCEGNFCNERFTHLPEA |
| | GGPEVTYEPPPTAPT |
| 298 | GRGEAETRECIYYNANWELERTNQSGLERCEGEQDKRLHCYASWRNSSGT |
| | IELVKKGCWLDDFNCYDRQECVAKKDSPEVYFCCCEGNFCNERFTHLPEA |
| | GGPEVTYEPPPTAPT |
| 299 | GRGEAETRECLYYNANWELERTNQSGVERCEGEKDKRLHCYASWRNSSGS |
| | LEIVKKGCWLDDFNCYDRTDCVATEENPQVYFCCCEGNMCNERFTHLPEA |
| | GGPEVTYEPPPTAPT |
| 300 | GRGEAETRECLYYNANWELERTNQSGVERCEGEKDKRLHCYASWRNSSGS |
| | LEIVKKGCWLDDFNCYDRDTCVATEENPQVYFCCCEGNMCNERFTHLPEA |
| | GGPEVTYEPPPTAPT |
| 301 | GRGEAETRECLYYNANWELERTNQSGVERCEGEKDKRLHCYASWRNSSGS |
| | LEIVKKGCWLDDFNCYDRTDCVATKENPQVYFCCCEGNMCNERFTHLPEA |
| | GGPEVTYEPPPTAPT |
| 302 | GRGEAETRECLYYNANWELERTNQSGVERCEGEKDKRLHCYASWRNSSGS |
| | LEIVKKGCWLDDFNCYDRDTCVATKENPQVYFCCCEGNMCNERFTHLPEA |
| | GGPEVTYEPPPTAPT |

In one aspect, the present disclosure provides isolated variant ActRIIB polypeptides comprising hybrid soluble ActRIIB polypeptides which retain myostatin- and activin A-neutralizing activities, but demonstrate dramatically reduced BMP9-neutralization. In various embodiments, the variant ActRIIB polypeptides comprise a hybrid soluble ActRIIB polypeptide having the amino acid sequence of SEQ ID NO: 303 wherein at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least two of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least three of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least four of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least five of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least six of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least seven of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least eight of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least nine of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least ten of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least fifteen of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least twenty of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least twenty-five of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least thirty of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide.

In various embodiments, the variant ActRIIB polypeptides comprise hybrid soluble ActRIIB polypeptides having an amino acid sequence set forth in any one of SEQ ID NOs: 305-339 (see, e.g., Table 15), wherein the hybrid ActRIIB polypeptide is capable of binding myostatin and activin A, but demonstrates a decreased binding affinity for BMP9 relative to a wild-type ActRIIB polypeptide. In various embodiments, the hybrid soluble ActRIIB polypeptides are hybrid soluble ActRIIB polypeptides having an amino acid sequence that is at least 70%, 75%, 80%, 85%, 90%, 91%, 92%, 93%, 94%, 95%, 96%, 97%, 98%, 99% or 100% identical to an amino acid sequence selected from SEQ ID NOs: 305-339 (see, e.g., Table 15), wherein the hybrid ActRIIB polypeptide is capable of binding myostatin and activin A, but demonstrates a decreased binding affinity for BMP9 relative to a wild-type ActRIIB polypeptide.

In various embodiments, the variant ActRIIB polypeptide comprises an amino acid sequence set forth in any one of SEQ ID NOs: 340-406, wherein the variant ActRIIB polypeptide is capable of binding myostatin and activin A, but demonstrates a decreased binding affinity for BMP9 relative to a wild-type ActRIIB polypeptide. In various embodiments, the variant ActRIIB polypeptides are hybrid soluble ActRIIB polypeptides having an amino acid sequence that is at least 70%, 75%, 80%, 85%, 90%, 91%, 92%, 93%, 94%, 95%, 96%, 97%, 98%, 99% or 100% identical to an amino acid sequence selected from SEQ ID NOs: 340-406, wherein the hybrid ActRIIB polypeptide is capable of binding myostatin and activin A, but demonstrates a decreased binding affinity for BMP9 relative to a wild-type ActRIIB polypeptide.

In another aspect, the present disclosure provides isolated nucleic acid molecules comprising a polynucleotide encoding a hybrid soluble ActRIIB polypeptide of the present disclosure. In various embodiments, the polynucleotides encodes one of the polypeptide sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having an amino acid sequence at least 70%, 75%, 80%, 85%, 90%, 91%, 92%, 93%, 94%, 95%, 96%, 97%, 98%, 99% or 100% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having at least 90% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having an amino acid sequence at least 95% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide.

In some embodiments, an ActRIIB polypeptide of the disclosure comprises a hybrid soluble ActRIIB polypeptide that is derived from wild-type ActRIIB and wild-type ActRIIA. The hybrid soluble ActRIIB polypeptides are specifically engineered by replacing one or more amino acids of a truncated wild-type ActRIIB polypeptide with the amino acids from a truncated wild-type ActRIIA polypeptide at corresponding positions based on sequence alignment between the two truncated ActRII polypeptide extracellular domains at the amino acid level. The one or more amino acid replacements are specifically selected for purposes of providing hybrid soluble ActRIIB polypeptides which demonstrate a reduction of BMP9-neutralization as compared to wild-type ActRIIB polypeptide, while retaining myostatin- and activin A-neutralization.

In various embodiments, the truncated extracellular domain of ActRIIB used to prepare the hybrid soluble ActRIIB polypeptides has the 110 amino acid sequence set forth in SEQ ID NO: 303:

(SEQ ID NO: 303)
ETRECIYYNANWELERTNQSGLERCEGEQDKRLHCYASWRNSSGTIELVK
KGCWLDDFNCYDRQECVATEENPQVYFCCCEGNFCNERFTHLPEAGGPEV
TYEPPPTAPT

In various embodiments, the truncated extracellular domain of ActRIIA used to prepare the hybrid soluble ActRIIB polypeptides has the 110 amino acid sequence set forth in SEQ ID NO: 304:

(SEQ ID NO: 304)
ETQECLFFNANWEKDRTNQTGVEPCYGDKDKRRHCFATWKNISGSIEIVK
QGCWLDDINCYDRTDCVEKKDSPEVYFCCCEGNMCNEKFSYFPEMEVTQP
TSNPVTPKPP

In various embodiments, the variant ActRIIB polypeptides comprise a hybrid soluble ActRIIB polypeptide having the amino acid sequence of SEQ ID NO: 303 wherein at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with the amino acid at the corresponding position of wild-type ActRIIA sequence (SEQ ID NO: 304), and wherein the hybrid soluble ActRIIB polypeptide is capable of binding myostatin and activin A, but demonstrates a decreased binding affinity for BMP9 relative to a wild-type ActRIIB polypeptide.

In various embodiments, the hybrid soluble ActRIIB polypeptide comprises the amino acid sequence of SEQ ID NO: 305, wherein amino acid residues E26, E28, Q29, L33, F58, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 306, wherein amino acid residues E26, E28, Q29, L33, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 307, wherein amino acid residues F58, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 308, wherein amino acid residues F58, Q64, E65, A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 309, wherein amino acid residues Q64, E65, A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 310, wherein amino acid residues Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 311, wherein amino acid residues A68, T69, E70, E71, N72 and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 312, wherein amino acid residues A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 313, wherein amino acid residues F58, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 314, wherein amino acid residues Q64, E65, A68, T69, E70, E71, N72, Q74, and F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 315, wherein amino acid residues A68, T69, E70, E71, N72, Q74, and F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 316, wherein amino acid residues R3, L14, E15, S20, L22, R24, E26, E28, Q29, and L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 317, wherein amino acid residues R3, L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 318, wherein amino acid residues E26, E28, Q29, and L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 319, wherein amino acid residues L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 320, wherein amino acid residues R3, L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 321, wherein amino acid residues R3, L14, E15, and S20 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 322, wherein amino acid residues R3, L14, and E15 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 323, wherein amino acid residues L14 and E15 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 324, wherein amino acid residue R3 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 325, wherein amino acid residues Y36, S38, and K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 326, wherein amino acid residues E26, E28, Q29, L33, and F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 327, wherein amino acid residue E70 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 328, wherein amino acid residue F58 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 329, wherein amino acid residues F58 and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 330, wherein amino acid residues E28, Q29, F58, and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 331, wherein amino acid residues E28, F58, and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 332, wherein amino acid residues E28 and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9.

In some embodiments, the hybrid soluble ActRIIB polypeptide binds myostatin and/or activin A. In various embodiments, the hybrid soluble ActRIIB polypeptide comprises the amino acid sequence of SEQ ID NO: 333, wherein amino acid residue E28 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 334, wherein amino acid residues E26, E28, Q29, L33, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 335, wherein amino acid residues Y7, Y8, L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 336, wherein amino acid residues Y36, S38, R40, S42, T45, and K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 337, wherein amino acid residues Q64 and E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 338, wherein amino acid residue F84 of SEQ ID NO: 303 have been replaced by the amino acid residue in the corresponding position of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 339, wherein amino acid residues E28 and F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 340, wherein amino acid residues R3, I6, Y7, Y8, L14, E15, L22, R24, E26, E28, Q29, L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 341, wherein amino acid residues R3, I6, Y7, Y8, L14, E15, L22, R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 342, wherein amino acid residues 16, Y7, Y8, L14, E15, L22, R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 343, wherein amino acid residues 16, Y7, Y8, L14, E15, L22, R24, E26 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 344, wherein amino acid residues 16, Y7, Y8, L14, E15, L22, R24, E26, E28, Q29, L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 345, wherein amino acid residues 16, Y7, Y8, L14, E15, L22, R24, E26, E28, Q29, L33, Y36, S38, R40, S42, T45, L48, K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 346, wherein amino acid residues 16, Y7, Y8, L14, E15, L22, R24, E26, E28, Q29, L33, Y36, S38, R40, S42, T45, L48, K51, F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 347, wherein amino acid residues 16, Y7, Y8, L14, E15, L22, R24, E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 348, wherein amino acid residues R3, E26, E28, Q29, L33, Y36, S38, R40, S42, T45, L48, K51, F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 349, wherein amino acid residues E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 350, wherein amino acid residues E26, E28, Q29, L33, Y36, S38, R40, S42, T45, L48, K51, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 351, wherein amino acid residues Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 352, wherein amino acid residues Y36, S38, R40, S42, T45, L48, K51, F58, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 353, wherein amino acid residues Y36, S38, R40, S42, T45, L48, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 354, wherein amino acid residues Y36, S38, R40, S42, T45, L48, K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 355, wherein amino acid residues R3, E26, E28, Q29, L33, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 356, wherein amino acid residues R3, E26, E28, Q29, L33, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 357, wherein amino acid residues R3, E26, E28, Q29, L33, Y36, S38, R40, S42, T45, L48, K51, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 358, wherein amino acid residues R3, E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 359, wherein amino acid residues 16, Y7, Y8, L14, E15, L22, R24, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 360, wherein amino acid residues 16, Y7, Y8, L14, E15, L22, R24, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 361, wherein amino acid residues 16, Y7, Y8, L14, E15, L22, R24, E26, E28, Q29, L33, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 362, wherein amino acid residues E26, E28, Q29, L33, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 363, wherein amino acid residues E26, E28, Q29, L33, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 364, wherein amino acid residues E26, E28, Q29, L33, L48, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 365, wherein amino acid residues E26, E28, Q29, L33, T45, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 366, wherein amino acid residues E26, E28, Q29, L33, T45, L48, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 367, wherein amino acid residues E26, E28, Q29, L33, T45, L48, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 368, wherein amino acid residues Q64, E65, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 369, wherein amino acid residues R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 370, wherein amino acid residues R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 371, wherein amino acid residues E26, E28, Q29, L33, F58, Q64, E65, A68, T69, E70, E71, N72, Q74, R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 372, wherein amino acid residues E26, E28, Q29, L33, Q64, E65, A68, T69, E70, E71, N72, Q74, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 373, wherein amino acid residues E26, E28, Q29, L33, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 374, wherein amino acid residues E26, E28, Q29, L33, K51, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 375, wherein amino acid residues E26, E28, Q29, L33, L48, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 376, wherein amino acid residues E26, E28, Q29, L33, T45, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 377, wherein amino acid residues T45, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 378, wherein amino acid residues L48, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 379, wherein amino acid residues K51, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 380, wherein amino acid residues A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 381, wherein amino acid residues A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 382, wherein amino acid residues E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 383, wherein amino acid residues E71, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 384, wherein amino acid residues N72, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 385, wherein amino acid residues Q74, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 386, wherein amino acid residues E28, Q29, A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 387, wherein amino acid residues Q29, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 388, wherein amino acid residues E28, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 389, wherein amino acid residues E28, Q29, K51, T69, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 390, wherein amino acid residues E28, Q29, L48, K51, T69E, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 391, wherein amino acid residues E26, E28, T45, L48, K51, T69, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 392, wherein amino acid residues Q29, L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 393, wherein amino acid residues E26, E28, L33, Q70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 394, wherein amino acid residues L33, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 395, wherein amino acid residues E26, T45, L48, Q64, E65, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 396, wherein amino acid residues L33, T45, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 397, wherein amino acid residues L33, L48, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 398, wherein amino acid residues L33, T45, L48, E70, R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 399, wherein amino acid residues E28, L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 400, wherein amino acid residues E28, T45, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 401, wherein amino acid residues E28, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 402, wherein amino acid residues L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 403, wherein amino acid residues E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 404, wherein amino acid residues E28, L48, T79, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 405, wherein amino acid residues R3, I6, Y7, Y8, L14, E15, S20, L22, R24, E26, E28, Q29, L33, Y36, S38, R40, S42, T45, L48, K51, F58, Q64, E65, A68, T69, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 406, wherein amino acid residues E26, E28, Q29, L33, F56, E68 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In certain embodiments, the present disclosure relates to ActRIIA polypeptides. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Patent Application Publication Nos. WO 2006/012627, WO 2007/062188, WO2018/089706, WO2018/089715, and WO2019/094751 which are incorporated herein by reference in their entirety. Numbering of amino acids for all ActRIIA-related polypeptides described herein is based on the numbering of the human ActRIIA precursor protein sequence provided below (SEQ ID NO: 9), unless specifically designated otherwise.

The canonical human ActRIIA precursor protein sequence is as follows:

(SEQ ID NO: 9)

| | |
|---|---|
| 1 | MGAAAKLAFA VELISCSSGA ILGRSETQEC LFFNANWEKD |
| | RTNQTGVEPC |
| 51 | YGDKDKRRHC FATWKNISGS IEIVKQGCWL DDINCYDRTD |
| | CVEKKDSPEV |
| 101 | YFCCCEGNMC NEKFSYFPEM EVTQPTSNPV TPKPPYYNIL |
| | LYSLVPLMLI |
| 151 | AGIVICAFWV YRHHKMAYPP VLVPTQDPGP PPPSPLLGLK |
| | PLQLLEVKAR |
| 201 | GRFGCVWKAQ LLNEYVAVKI FPIQDKQSWQ NEYEVYSLPG |
| | MKHENILQFI |
| 251 | GAEKRGTSVD VDLWLITAFH EKGSLSDFLK ANVVSWNELC |
| | HIAETMARGL |
| 301 | AYLHEDIPGL KDGHKPAISH RDIKSKNVLL KNNLTACIAD |
| | FGLALKFEAG |
| 351 | KSAGDTHGQV GTRRYMAPEV LEGAINFQRD AFLRIDMYAM |
| | GLVLWELASR |
| 401 | CTAADGPVDE YMLPFEEEIG QHPSLEDMQE VVVHKKKRPV |
| | LRDYWQKHAG |
| 451 | MAMLCETIEE CWDHDAEARL SAGCVGERIT QMQRLTNIIT |
| | TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A processed (mature) extracellular human ActRIIA polypeptide sequence is as follows:

(SEQ ID NO: 10)
ILGRSETQECLFENANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

The C-terminal “tail” of the extracellular domain is indicated by single underline. The sequence with the “tail” deleted (a A15 sequence) is as follows:

(SEQ ID NO: 11)
ILGRSETQECLFENANWEKDRTNQTGVEPCYGDKDKRRHCFATWKNISGS
IEIVKQGCWLDDINCYDRTDCVEKKDSPEVYFCCCEGNMCNEKFSYFPEM

A nucleic acid sequence encoding the human ActRIIA precursor protein (SEQ ID NO: 9) is shown below (SEQ ID NO: 12), as follows nucleotides 159-1700 of Genbank Reference Sequence NM_001616.4. The signal sequence is underlined.

(SEQ ID NO: 12

| | |
|---|---|
| 1 | ATGGGAGCTG CTGCAAAGTT GGCGTTTGCC GTCTTTCTTA |
| | TCTCCTGTTC |
| 51 | TTCAGGTGCT ATACTTGGTA GATCAGAAAC TCAGGAGTGT |
| | CTTTTCTTTA |
| 101 | ATGCTAATTG GGAAAAAGAC AGAACCAATC AAACTGGTGT |
| | TGAACCGTGT |
| 151 | TATGGTGACA AAGATAAACG GCGGCATTGT TTTGCTACCT |
| | GGAAGAATAT |
| 201 | TTCTGGTTCC ATTGAAATAG TGAAACAAGG TTGTTGGCTG |
| | GATGATATCA |
| 251 | ACTGCTATGA CAGGACTGAT TGTGTAGAAA AAAAAGACAG |
| | CCCTGAAGTA |
| 301 | TATTTTTGTT GCTGTGAGGG CAATATGTGT AATGAAAAGT |
| | TTTCTTATTT |
| 351 | TCCGGAGATG GAAGTCACAC AGCCCACTTC AAATCCAGTT |
| | ACACCTAAGC |
| 401 | CACCCTATTA CAACATCCTG CTCTATTCCT TGGTGCCACT |
| | TATGTTAATT |
| 451 | GCGGGGATTG TCATTTGTGC ATTTTGGGTG TACAGGCATC |
| | ACAAGATGGC |
| 501 | CTACCCTCCT GTACTTGTTC CAACTCAAGA CCCAGGACCA |
| | CCCCCACCTT |
| 551 | CTCCATTACT AGGTTTGAAA CCACTGCAGT TATTAGAAGT |
| | GAAAGCAAGG |
| 601 | GGAAGATTTG GTTGTGTCTG GAAAGCCCAG TTGCTTAACG |
| | AATATGTGGC |
| 651 | TGTCAAAATA TTTCCAATAC AGGACAAACA GTCATGGCAA |
| | AATGAATACG |
| 701 | AAGTCTACAG TTTGCCTGGA ATGAAGCATG AGAACATATT |
| | ACAGTTCATT |
| 751 | GGTGCAGAAA AACGAGGCAC CAGTGTTGAT GTGGATCTTT |
| | GGCTGATCAC |
| 801 | AGCATTTCAT GAAAAGGGTT CACTATCAGA CTTTCTTAAG |
| | GCTAATGTGG |
| 851 | TCTCTTGGAA TGAACTGTGT CATATTGCAG AAACCATGGC |
| | TAGAGGATTG |
| 901 | GCATATTTAC ATGAGGATAT ACCTGGCCTA AAAGATGGCC |
| | ACAAACCTGC |
| 951 | CATATCTCAC AGGGACATCA AAAGTAAAAA TGTGCTGTTG |
| | AAAAACAACC |
| 1001 | TGACAGCTTG CATTGCTGAC TTTGGGTTGG CCTTAAAATT |
| | TGAGGCTGGC |
| 1051 | AAGTCTGCAG GCGATACCCA TGGACAGGTT GGTACCCGGA |
| | GGTACATGGC |
| 1101 | TCCAGAGGTA TTAGAGGGTG CTATAAACTT CCAAAGGGAT |
| | GCATTTTTGA |
| 1151 | GGATAGATAT GTATGCCATG GGATTAGTCC TATGGGAACT |
| | GGCTTCTCGC |
| 1201 | TGTACTGCTG CAGATGGACC TGTAGATGAA TACATGTTGC |
| | CATTTGAGGA |
| 1251 | GGAAATTGGC CAGCATCCAT CTCTTGAAGA CATGCAGGAA |
| | GTTGTTGTGC |
| 1301 | ATAAAAAAAA GAGGCCTGTT TTAAGAGATT ATTGGCAGAA |
| | ACATGCTGGA |
| 1351 | ATGGCAATGC TCTGTGAAAC CATTGAAGAA TGTTGGGATC |
| | AGGACGCAGA |
| 1401 | AGCCAGGTTA TCAGCTGGAT GTGTAGGTGA AAGAATTACC |
| | CAGATGCAGA |
| 1451 | GACTAACAAA TATTATTACC ACAGAGGACA TTGTAACAGT |
| | GGTCACAATG |
| 1501 | GTGACAAATG TTGACTTTCC TCCCAAAGAA TCTAGTCTA |

A nucleic acid sequence encoding the processed soluble (extracellular) human ActRIIA polypeptide (SEQ ID NO: 10) is as follows:

(SEQ ID NO: 13)

| | |
|---|---|
| 1 | ATACTTGGTA GATCAGAAAC TCAGGAGTGT CTTTTCTTTA |
| | ATGCTAATTG |
| 51 | GGAAAAAGAC AGAACCAATC AAACTGGTGT TGAACCGTGT |
| | TATGGTGACA |
| 101 | AAGATAAACG GCGGCATTGT TTTGCTACCT GGAAGAATAT |
| | TTCTGGTTCC |
| 151 | ATTGAAATAG TGAAACAAGG TTGTTGGCTG GATGATATCA |
| | ACTGCTATGA |
| 201 | CAGGACTGAT TGTGTAGAAA AAAAAGACAG CCCTGAAGTA |
| | TATTTTTGTT |
| 251 | GCTGTGAGGG CAATATGTGT AATGAAAAGT TTTCTTATTT |
| | TCCGGAGATG |
| 301 | GAAGTCACAC AGCCCACTTC AAATCCAGTT ACACCTAAGC |
| | CACCC |

In some embodiments, the ActRIIA polypeptide sequence comprises accession number UniProtKB/Swiss-Prot P27037.1 (SEQ ID NO: 408 herein), and variants thereof. In some embodiments, the term “wild-type ActRIIA polypeptide” refers to the extracellular domain of ActRIIA, amino acids 1 to 135 (with signal sequence), or amino acids 20 through 135 of SEQ ID NO: 407 (without signal sequence) (referred to herein as SEQ ID NO: 409).

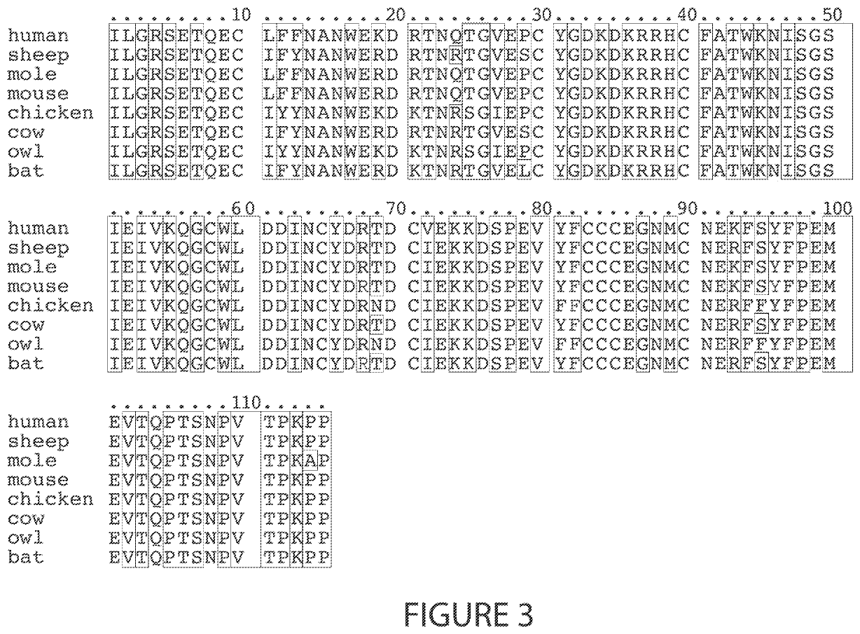

ActRIIA is well-conserved among vertebrates, with large stretches of the extracellular domain completely conserved. For example, FIG. 3 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant polypeptide useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 3, F13 in the human extracellular domain (SEQ ID NO: 10) is Y in Ovis aries (SEQ ID NO: 62), Gallus gallus (SEQ ID NO: 65), Bos Taurus (SEQ ID NO: 66), Tyto alba (SEQ ID NO: 67), and Myotis davidii (SEQ ID NO: 68) ActRIIA, indicating that aromatic residues are tolerated at this position, including F, W, and Y. Q24 in the human extracellular domain (SEQ ID NO: 10) is R in Bos Taurus ActRIIA, indicating that charged residues will be tolerated at this position, including D, R, K, H, and E. S95 in the human extracellular domain (SEQ ID NO: 10) is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain (SEQ ID NO: 10) is D in Ovis aries ActRIIA, indicating that acidic residues are tolerated at this position, including D and E. P29 in the human extracellular (SEQ ID NO: 10) domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103 (20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide amply guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIA, as demarcated by the outermost of these conserved cysteines, corresponds to positions 30-110 of SEQ ID NO: 9 (ActRIIA precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIA extracellular domains truncations include SEQ ID NOs: 10 and 11.

Accordingly, a general formula for an active portion (e.g., ligand binding) of ActRIIA is a polypeptide that comprises, consists essentially of, or consists of amino acids 30-110 of SEQ ID NO: 9. Therefore ActRIIA polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 9. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 9, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 9. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 9. Thus, in some embodiments, an ActRIIA polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9. Optionally, ActRIIA polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9, and comprising no more than 1, 2, 5, 10 or 15 conservative amino acid changes in the ligand-binding pocket.

In certain embodiments, the disclosure relates to ActRII antagonists (inhibitors) that comprise an ActRIIA polypeptide, which includes fragments, functional variants, and modified forms thereof as well as uses thereof (e.g., increasing an immune response in a patient in need thereof and treating cancer). Preferably, ActRIIA polypeptides are soluble (e.g., an extracellular domain of ActRIIA). In some embodiments, ActRIIA polypeptides inhibit (e.g., Smad signaling) of one or more ligands [e.g., GDF11, GDF8, activin (activin A, activin B, activin AB, activin AC, BMP6, BMP7, BMP10, GDF3, GDF8, and/or GDF11]. In some embodiments, ActRIIA polypeptides bind to one or more ligands [e.g., activin A, activin B, activin AB, activin AC, BMP6, BMP7, BMP10, GDF3, GDF8, and/or GDF11]. In some embodiments, ActRIIA polypeptides of the disclosure demonstrate a decreased binding affinity for BMP9. In some embodiments, ActRIIA polypeptides of the disclosure do not bind BMP9. In some embodiments, ActRIIA polypeptide of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 9. In certain embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, 11, 32, 36, 39, 93, 95, 96, 97, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 10. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 11. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 32. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 36. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 39. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 93. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 95. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 96. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 97. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 139. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 140. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 141. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 142. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 143. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 144. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 145. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 146. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 147. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 148. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 149. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 150. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 151. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 152. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 153. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 154. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 155. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 156. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 157. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 158. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 159. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 160. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 161. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 162. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 163. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 164. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 165. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 166. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 167. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 168. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 169. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 170. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 171. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 172. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 173. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 174. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 175. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 176. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 177. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 178. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 179. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 180. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 181. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 182. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 183. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 184. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 185. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 186. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 187. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 188. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 189. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 190. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 191. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 192. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 193. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 194. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 195. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 196. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 197. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 198. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 199. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 200. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 201. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 202. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 203. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 204. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 205. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 206. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 207. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 208. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 209. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 210. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 211. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 283. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 304. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 408. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 409.

In some embodiments, an extracellular ActRIIA variant polypeptide may have a sequence of any one of SEQ ID NOs: 139-210. In some embodiments, an extracellular ActRIIA variant polypeptide has a sequence of any one of SEQ ID NOs: 144-210 (Table 5). In some embodiments, an extracellular ActRIIA variant polypeptide may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of a wild-type extracellular ActRIIA polypeptide (SEQ ID NO: 211).

In some embodiments, polypeptides described herein include an extracellular ActRIIA variant having at least one amino acid substitution relative to the wild-type extracellular ActRIIA having the sequence of SEQ ID NO: 211 or the extracellular ActRIIA having any one of the sequences of SEQ ID NOs: 212-232. Possible amino acid substitutions at 27 different positions may be introduced to an extracellular ActRIIA variant (Table 4). An extracellular ActRIIA variant may have one or more (e.g., 1-27, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a wild-type extracellular ActRIIA (SEQ ID NO: 211). In some embodiments, an extracellular ActRIIA variant (e.g., an extracellular ActRIIA variant having a sequence of SEQ ID NO: 139) may include amino acid substitutions at all of the 27 positions as listed in Table 4. In some embodiments, an extracellular ActRIIA variant may include amino acid substitutions at a number of positions, e.g., at 2, 4, 6, 8, 10, 12, 14, 16, 18, 20, 22, 24, or 26 out of the 27 positions, as listed in Table 4.

Amino acid substitutions can worsen or improve the activity and/or binding affinity of the ActRIIA variants disclosed herein. In some embodiments, to maintain polypeptide function, it is important that the lysine (K) at position X₁₇ in the sequences shown in Tables 4 and 5 (SEQ ID NOs: 139-210 (e.g., SEQ ID NOs: 144-210)) be retained. Substitutions at that position can lead to a loss of activity. For example, an ActRIIA variant having the sequence

(SEQ ID NO: 283)
GAILGRSETQECLFYNANWELERTNQTGVERCEGEKDKRLHCYATWRNIS
GSIEIVAKGCWLDDFNCYDRTDCVETEENPQVYFCCCEGNMCNEKFSYFP
EMEVTQPTS

has reduced activity in vivo, indicating that the substitution of alanine (A) for lysine (K) at X₁₇ is not tolerated. ActRIIA variants disclosed herein, including variants in Tables 4 and 5 (e.g., SEQ ID NOs: 139-210 (e.g., SEQ ID NOs: 144-210), therefore, may retain amino acid K at the position corresponding to X₁₇ in SEQ ID NO: 139 or SEQ ID NO: 140.

In some embodiments, the ActRIIA variants disclosed herein have reducedor no substantial binding to BMP9. In some embodiments, BMP9 binding is reduced in ActRIIA variants containing the amino acid sequence TEEN at positions X₂₃, X₂₄, X₂₅, and X₂₆, as well as in variants that maintain the amino acid K at position X₂₄ and have the amino acid sequence TKEN at positions X₂₃, X₂₄, X₂₅, and X₂₆. The sequences TEEN and TKEN can be employed interchangeably in the ActRIIA variants (e.g., the variants in Tables 4 and 5, e.g., SEQ ID NOs: 139-210 (e.g., SEQ ID NOs: 144-210)) disclosed herein to provide reduced BMP9 binding.

In some embodiments, the ActRIIA variants disclosed herein may further include a C-terminal extension (e.g., additional amino acids at the C-terminus). The C-terminal extension can add one to six additional amino acids at the C-terminus (e.g., 1, 2, 3, 4, 5, 6 or more additional amino acids) to any of the variant polypeptides shown in Tables 4 and 5 (e.g., SEQ ID NOs: 139-208 (e.g., SEQ ID NOs: 144-208)). One potential C-terminal extension that can be included in the ActRIIA variant polypeptides disclosed herein is amino acid sequence NP. For example, the sequence including the C-terminal extension is SEQ ID NO: 209 (e.g., SEQ ID NO: 207 with a C-terminal extension of NP). Another exemplary C-terminal extension that can be included in the ActRIIA variant polypeptides disclosed herein is amino acid sequence NPVTPK (SEQ ID NO: 288). For example, the sequence including the C-terminal extension is SEQ ID NO: 210 (e.g., SEQ ID NO: 207 with a C-terminal extension of NPVTPK).

TABLE 4
Amino acid substitutions in an extracellular
ActRIIA variant having a sequence of any one of
SEQ ID NOs: 139-143

GAILGRSETQECLX₁X₂NANWX₃X₄X₅X₆TNQTGVEX₇CX₈GX₉X₁₀X₁₁
X₁₂X₁₃X₁₄HCX₁₅ATWX₁₆NISGSIEIVX₁₇X₁₈GCX₁₉X₂₀X₂₁DX₂₂
NCYDRTDCVEX₂₃X₂₄X₂₅X₂₆PX₂₇VYFCCCEGNMCNEKFSYFPEMEVT
QPTS (SEQ ID NO: 139)
GAILGRSETQECLFX₂NANWX₃X₄X₅X₆TNQTGVEX₇CX₈GX₉KX₁₁X₁₂
X₁₃X₁₄HCX₁₅ATWX₁₆NISGSIEIVX₁₇X₁₈GCX₁₉X₂₀X₂₁DX₂₂NCY
DRTDCVEX₂₃X₂₄X₂₅X₂₆PX₂₇VYFCCCEGNMCNEKFSYFPEMEVTQPT
S (SEQ ID NO: 140)
GAILGRSETQECLFX₂NANWEX₄X₅RTNQTGVEX₇CX₈GX₉KDKRX₁₄HC
X₁₅ATWX₁₆NISGSIEIVKX₁₈GCWLDDX₂₂NCYDRTDCVEX₂₃X₂₄X₂₅
X₂₆PX₂₇VYFCCCEGNMCNEKFSYFPEMEVTQPTS (SEQ ID NO:
141)
GAILGRSETQECLFX₂NANWEX₄DRTNQTGVEX₇CX₈GX₉KDKRX₁₄HC
X₁₅ATWX₁₆NISGSIEIVKX₁₈GCWLDDX₂₂NCYDRTDCVEX₂₃KX₂₅
X₂₆PX₂₇VYFCCCEGNMCNEKFSYFPEMEVTQPTS (SEQ ID NO:
142)
GAILGRSETQECLFX₂NANWEX₄DRTNQTGVEPCX₈GX₉KDKRX₁₄HCFA
TWKNISGSIEIVKX₁₈GCWLDDINCYDRTDCVEX₂₃KX₂₅X₂₆PX₂₇VYF
CCCEGNMCNEKFSYFPEMEVTQPTS (SEQ ID NO: 143)
X₁ F, A, V, I, L, M, F, Y or W
X₂ F, A, V, I, L, M, F, Y or W
X₃ D, E or A
X₄ K or L
X₅ D, E, or A
X₆ R, H, K, or A
X₇ P or R
X₈ Y or E
X₉ D, E, or A
X₁₀ K or Q
X₁₁ D, E, or A
X₁₂ K, R, H, or A
X₁₃ R, H, K, or A
X₁₄ R or L
X₁₅ F, A, V, I, L, M, F, Y or W
X₁₆ K, R, H, or A
X₁₇ K, A, Y, F, I, V, L, M, or W
X₁₈ Q or K
X₁₉ W or A
X₂₀ L or A
X₂₁ D, K, R, H, A, F, G, M, N, or I
X₂₂ I, F, A, V, L, M, Y or W
X₂₃ K or T
X₂₄ K, R, H, D, or E
X₂₅ D, E, or A
X₂₆ S, N, T, or Q
X₂₇ E or Q

In some embodiments, an extracellular ActRIIA variant comprising the sequence of SEQ ID NO: 140 has the following amino acid substitutions: X₃ is E, X₆ is R, X₁₁ is D, X₁₂ is K, X₁₃ is R, X₁₆ is K or R, X₁₇ is K, X₁₉ is WV, X₂₀ is L, X₂₁ is D, and X₂₂ is I or F. In some embodiments, an extracellular ActRIIA variant comprising the sequence of SEQ ID NO: 139 or 140 has the following amino acid substitutions: X₁₇ is K. In some embodiments, an extracellular ActRIIA variant comprising the sequence of SEQ ID NOs: 139-141 has the following amino acid substitutions: X₁₇ is K, X₂₃ is T, X₂₄ is E, X₂₅ is E, and X₂₆ is N. In some embodiments, an extracellular ActRIIA variant comprising the sequence of any one of SEQ ID NOs: 139-143 has the following amino acid substitutions: X₁₇ is K, X₂₃ is T, X₂₄ is K, X₂₅ is E, and X₂₆ is N.

In some embodiments, a polypeptide described herein includes an extracellular ActRIIA variant having a sequence of any one of SEQ TD NOs: 144-210 (Table 5).

TABLE 5
Extracellular ActRIIA variant polypeptides having the sequences of SEQ ID
NOs: 144-210

| SEQ ID | |
|---|---|
| NO | Amino Acid Sequence |
| 144 | GAILGRSETQECLFYNANWELDRTNQTGVEPCEGEKDKRLHCFATWKNIS |
| | GSIEIVKKGCWLDDINCYDRTDCVETKENPQVYFCCCEGNMCNEKFSYFPE |
| | MEVTQPTS |
| 145 | GAILGRSETQECLFYNANWELERTNQTGVEPCEGEKDKRLHCFATWKNIS |
| | GSIEIVKKGCWLDDINCYDRTDCVETKENPQVYFCCCEGNMCNEKFSYFPE |
| | MEVTQPTS |
| 146 | GAILGRSETQECLFYNANWELDRTNQTGVERCEGEKDKRLHCFATWKNIS |
| | GSIEIVKKGCWLDDINCYDRTDCVETKENPQVYFCCCEGNMCNEKFSYFPE |
| | MEVTQPTS |
| 147 | GAILGRSETQECLFYNANWELDRTNQTGVEPCEGEKDKRLHCYATWKNIS |
| | GSIEIVKKGCWLDDINCYDRTDCVETKENPQVYFCCCEGNMCNEKFSYFPE |
| | MEVTQPTS |
| 148 | GAILGRSETQECLFYNANWELDRTNQTGVEPCEGEKDKRLHCFATWRNIS |
| | GSIEIVKKGCWLDDINCYDRTDCVETKENPQVYFCCCEGNMCNEKFSYFPE |
| | MEVTQPTS |
| 149 | GAILGRSETQECLFYNANWELDRTNQTGVEPCEGEKDKRLHCFATWKNIS |
| | GSIEIVKKGCWLDDFNCYDRTDCVETKENPQVYFCCCEGNMCNEKFSYFP |
| | EMEVTQPTS |
| 150 | GAILGRSETQECLFYNANWELDRTNQTGVEPCEGEKDKRLHCFATWKNIS |
| | GSIEIVKKGCWLDDINCYDRTDCVETEENPQVYFCCCEGNMCNEKFSYFPE |
| | MEVTQPTS |
| 151 | GAILGRSETQECLFYNANWELERTNQTGVERCEGEKDKRLHCFATWKNIS |
| | GSIEIVKKGCWLDDINCYDRTDCVETKENPQVYFCCCEGNMCNEKFSYFPE |
| | MEVTQPTS |
| 152 | GAILGRSETQECLFYNANWELERTNQTGVEPCEGEKDKRLHCYATWKNIS |
| | GSIEIVKKGCWLDDINCYDRTDCVETKENPQVYFCCCEGNMCNEKFSYFPE |
| | MEVTQPTS |
| 153 | GAILGRSETQECLFYNANWELERTNQTGVEPCEGEKDKRLHCFATWRNIS |
| | GSIEIVKKGCWLDDINCYDRTDCVETKENPQVYFCCCEGNMCNEKFSYFPE |
| | MEVTQPTS |
| 154 | GAILGRSETQECLFYNANWELERTNQTGVEPCEGEKDKRLHCFATWKNIS |
| | GSIEIVKKGCWLDDFNCYDRTDCVETKENPQVYFCCCEGNMCNEKFSYFP |
| | EMEVTQPTS |
| 155 | GAILGRSETQECLFYNANWELERTNQTGVEPCEGEKDKRLHCFATWKNIS |
| | GSIEIVKKGCWLDDINCYDRTDCVETEENPQVYFCCCEGNMCNEKFSYFPE |
| | MEVTQPTS |
| 156 | GAILGRSETQECLFYNANWELDRTNQTGVERCEGEKDKRLHCYATWKNIS |
| | GSIEIVKKGCWLDDINCYDRTDCVETKENPQVYFCCCEGNMCNEKFSYFPE |
| | MEVTQPTS |
| 157 | GAILGRSETQECLFYNANWELDRTNQTGVERCEGEKDKRLHCFATWRNIS |
| | GSIEIVKKGCWLDDINCYDRTDCVETKENPQVYFCCCEGNMCNEKFSYFPE |
| | MEVTQPTS |
| 158 | GAILGRSETQECLFYNANWELDRTNQTGVERCEGEKDKRLHCFATWKNIS |
| | GSIEIVKKGCWLDDFNCYDRTDCVETKENPQVYFCCCEGNMCNEKFSYFP |
| | EMEVTQPTS |
| 159 | GAILGRSETQECLFYNANWELDRTNQTGVERCEGEKDKRLHCFATWKNIS |
| | GSIEIVKKGCWLDDINCYDRTDCVETEENPQVYFCCCEGNMCNEKFSYFPE |
| | MEVTQPTS |
| 160 | GAILGRSETQECLFYNANWELDRTNQTGVEPCEGEKDKRLHCYATWRNIS |
| | GSIEIVKKGCWLDDINCYDRTDCVETKENPQVYFCCCEGNMCNEKFSYFPE |
| | MEVTQPTS |
| 161 | GAILGRSETQECLFYNANWELDRTNQTGVEPCEGEKDKRLHCYATWKNIS |
| | GSIEIVKKGCWLDDFNCYDRTDCVETKENPQVYFCCCEGNMCNEKFSYFP |
| | EMEVTQPTS |
| 162 | GAILGRSETQECLFYNANWELDRTNQTGVEPCEGEKDKRLHCYATWKNIS |
| | GSIEIVKKGCWLDDINCYDRTDCVETEENPQVYFCCCEGNMCNEKFSYFPE |
| | MEVTQPTS |
| 163 | GAILGRSETQECLFYNANWELDRTNQTGVEPCEGEKDKRLHCFATWRNIS |
| | GSIEIVKKGCWLDDFNCYDRTDCVETKENPQVYFCCCEGNMCNEKFSYFP |
| | EMEVTQPTS |
| 164 | GAILGRSETQECLFYNANWELDRTNQTGVEPCEGEKDKRLHCFATWRNIS |
| | GSIEIVKKGCWLDDINCYDRTDCVETEENPQVYFCCCEGNMCNEKFSYFPE |
| | MEVTQPTS |
| 165 | GAILGRSETQECLFYNANWELDRTNQTGVEPCEGEKDKRLHCFATWKNIS |
| | GSIEIVKKGCWLDDFNCYDRTDCVETEENPQVYFCCCEGNMCNEKFSYFPE |
| | MEVTQPTS |
| 166 | GAILGRSETQECLFYNANWELERTNQTGVERCEGEKDKRLHCYATWKNIS |
| | GSIEIVKKGCWLDDINCYDRTDCVETKENPQVYFCCCEGNMCNEKFSYFPE |
| | MEVTQPTS |
| 167 | GAILGRSETQECLFYNANWELERTNQTGVERCEGEKDKRLHCFATWRNIS |
| | GSIEIVKKGCWLDDINCYDRTDCVETKENPQVYFCCCEGNMCNEKFSYFPE |
| | MEVTQPTS |
| 168 | GAILGRSETQECLFYNANWELERTNQTGVERCEGEKDKRLHCFATWKNIS |
| | GSIEIVKKGCWLDDFNCYDRTDCVETKENPQVYFCCCEGNMCNEKFSYFP |
| | EMEVTQPTS |
| 169 | GAILGRSETQECLFYNANWELERTNQTGVERCEGEKDKRLHCFATWKNIS |
| | GSIEIVKKGCWLDDINCYDRTDCVETEENPQVYFCCCEGNMCNEKFSYFPE |
| | MEVTQPTS |
| 170 | GAILGRSETQECLFYNANWELERTNQTGVEPCEGEKDKRLHCYATWRNIS |
| | GSIEIVKKGCWLDDINCYDRTDCVETKENPQVYFCCCEGNMCNEKFSYFPE |
| | MEVTQPTS |
| 171 | GAILGRSETQECLFYNANWELERTNQTGVEPCEGEKDKRLHCYATWKNIS |
| | GSIEIVKKGCWLDDFNCYDRTDCVETKENPQVYFCCCEGNMCNEKFSYFP |
| | EMEVTQPTS |
| 172 | GAILGRSETQECLFYNANWELERTNQTGVEPCEGEKDKRLHCYATWKNIS |
| | GSIEIVKKGCWLDDINCYDRTDCVETEENPQVYFCCCEGNMCNEKFSYFPE |
| | MEVTQPTS |
| 173 | GAILGRSETQECLFYNANWELERTNQTGVEPCEGEKDKRLHCFATWRNIS |
| | GSIEIVKKGCWLDDFNCYDRTDCVETKENPQVYFCCCEGNMCNEKFSYFP |
| | EMEVTQPTS |
| 174 | GAILGRSETQECLFYNANWELERTNQTGVEPCEGEKDKRLHCFATWRNIS |
| | GSIEIVKKGCWLDDINCYDRTDCVETEENPQVYFCCCEGNMCNEKFSYFPE |
| | MEVTQPTS |
| 175 | GAILGRSETQECLFYNANWELERTNQTGVEPCEGEKDKRLHCFATWKNIS |
| | GSIEIVKKGCWLDDFNCYDRTDCVETEENPQVYFCCCEGNMCNEKFSYFPE |
| | MEVTQPTS |
| 176 | GAILGRSETQECLFYNANWELDRTNQTGVERCEGEKDKRLHCYATWRNIS |
| | GSIEIVKKGCWLDDINCYDRTDCVETKENPQVYFCCCEGNMCNEKFSYFPE |
| | MEVTQPTS |
| 177 | GAILGRSETQECLFYNANWELDRTNQTGVERCEGEKDKRLHCYATWKNIS |
| | GSIEIVKKGCWLDDFNCYDRTDCVETKENPQVYFCCCEGNMCNEKFSYFP |
| | EMEVTQPTS |
| 178 | GAILGRSETQECLFYNANWELDRTNQTGVERCEGEKDKRLHCYATWKNIS |
| | GSIEIVKKGCWLDDINCYDRTDCVETEENPQVYFCCCEGNMCNEKFSYFPE |
| | MEVTQPTS |
| 179 | GAILGRSETQECLFYNANWELDRTNQTGVERCEGEKDKRLHCFATWRNIS |
| | GSIEIVKKGCWLDDFNCYDRTDCVETKENPQVYFCCCEGNMCNEKFSYFP |
| | EMEVTQPTS |
| 180 | GAILGRSETQECLFYNANWELDRTNQTGVERCEGEKDKRLHCFATWRNIS |
| | GSIEIVKKGCWLDDINCYDRTDCVETEENPQVYFCCCEGNMCNEKFSYFPE |
| | MEVTQPTS |
| 181 | GAILGRSETQECLFYNANWELDRTNQTGVERCEGEKDKRLHCFATWKNIS |
| | GSIEIVKKGCWLDDFNCYDRTDCVETEENPQVYFCCCEGNMCNEKFSYFPE |
| | MEVTQPTS |
| 182 | GAILGRSETQECLFYNANWELDRTNQTGVEPCEGEKDKRLHCYATWRNIS |
| | GSIEIVKKGCWLDDFNCYDRTDCVETKENPQVYFCCCEGNMCNEKFSYFP |
| | EMEVTQPTS |
| 183 | GAILGRSETQECLFYNANWELDRTNQTGVEPCEGEKDKRLHCYATWRNIS |
| | GSIEIVKKGCWLDDINCYDRTDCVETEENPQVYFCCCEGNMCNEKFSYFPE |
| | MEVTQPTS |
| 184 | GAILGRSETQECLFYNANWELDRTNQTGVEPCEGEKDKRLHCYATWKNIS |
| | GSIEIVKKGCWLDDFNCYDRTDCVETEENPQVYFCCCEGNMCNEKFSYFPE |
| | MEVTQPTS |
| 185 | GAILGRSETQECLFYNANWELDRTNQTGVEPCEGEKDKRLHCFATWRNIS |
| | GSIEIVKKGCWLDDFNCYDRTDCVETEENPQVYFCCCEGNMCNEKFSYFPE |
| | MEVTQPTS |
| 186 | GAILGRSETQECLFYNANWELERTNQTGVERCEGEKDKRLHCYATWRNIS |
| | GSIEIVKKGCWLDDINCYDRTDCVETKENPQVYFCCCEGNMCNEKFSYFPE |
| | MEVTQPTS |
| 187 | GAILGRSETQECLFYNANWELERTNQTGVERCEGEKDKRLHCYATWKNIS |
| | GSIEIVKKGCWLDDFNCYDRTDCVETKENPQVYFCCCEGNMCNEKFSYFP |
| | EMEVTQPTS |
| 188 | GAILGRSETQECLFYNANWELERTNQTGVERCEGEKDKRLHCYATWKNIS |
| | GSIEIVKKGCWLDDINCYDRTDCVETEENPQVYFCCCEGNMCNEKFSYFPE |
| | MEVTQPTS |
| 189 | GAILGRSETQECLFYNANWELERTNQTGVERCEGEKDKRLHCFATWRNIS |
| | GSIEIVKKGCWLDDFNCYDRTDCVETKENPQVYFCCCEGNMCNEKFSYFP |
| | EMEVTQPTS |
| 190 | GAILGRSETQECLFYNANWELERTNQTGVERCEGEKDKRLHCFATWRNIS |
| | GSIEIVKKGCWLDDINCYDRTDCVETEENPQVYFCCCEGNMCNEKFSYFPE |
| | MEVTQPTS |
| 191 | GAILGRSETQECLFYNANWELERTNQTGVERCEGEKDKRLHCFATWKNIS |
| | GSIEIVKKGCWLDDFNCYDRTDCVETEENPQVYFCCCEGNMCNEKFSYFPE |
| | MEVTQPTS |
| 192 | GAILGRSETQECLFYNANWELERTNQTGVEPCEGEKDKRLHCYATWRNIS |
| | GSIEIVKKGCWLDDFNCYDRTDCVETKENPQVYFCCCEGNMCNEKFSYFP |
| | EMEVTQPTS |
| 193 | GAILGRSETQECLFYNANWELERTNQTGVEPCEGEKDKRLHCYATWRNIS |
| | GSIEIVKKGCWLDDINCYDRTDCVETEENPQVYFCCCEGNMCNEKFSYFPE |
| | MEVTQPTS |
| 194 | GAILGRSETQECLFYNANWELERTNQTGVEPCEGEKDKRLHCYATWKNIS |
| | GSIEIVKKGCWLDDFNCYDRTDCVETEENPQVYFCCCEGNMCNEKFSYFPE |
| | MEVTQPTS |
| 195 | GAILGRSETQECLFYNANWELERTNQTGVEPCEGEKDKRLHCFATWRNIS |
| | GSIEIVKKGCWLDDFNCYDRTDCVETEENPQVYFCCCEGNMCNEKFSYFPE |
| | MEVTQPTS |
| 196 | GAILGRSETQECLFYNANWELDRTNQTGVERCEGEKDKRLHCYATWRNIS |
| | GSIEIVKKGCWLDDFNCYDRTDCVETKENPQVYFCCCEGNMCNEKFSYFP |
| | EMEVTQPTS |
| 197 | GAILGRSETQECLFYNANWELDRTNQTGVERCEGEKDKRLHCYATWRNIS |
| | GSIEIVKKGCWLDDINCYDRTDCVETEENPQVYFCCCEGNMCNEKFSYFPE |
| | MEVTQPTS |
| 198 | GAILGRSETQECLFYNANWELDRTNQTGVERCEGEKDKRLHCYATWKNIS |
| | GSIEIVKKGCWLDDFNCYDRTDCVETEENPQVYFCCCEGNMCNEKFSYFPE |
| | MEVTQPTS |
| 199 | GAILGRSETQECLFYNANWELDRTNQTGVERCEGEKDKRLHCFATWRNIS |
| | GSIEIVKKGCWLDDFNCYDRTDCVETEENPQVYFCCCEGNMCNEKFSYFPE |
| | MEVTQPTS |
| 200 | GAILGRSETQECLFYNANWELDRTNQTGVEPCEGEKDKRLHCYATWRNIS |
| | GSIEIVKKGCWLDDFNCYDRTDCVETEENPQVYFCCCEGNMCNEKFSYFPE |
| | MEVTQPTS |
| 201 | GAILGRSETQECLFYNANWELERTNQTGVERCEGEKDKRLHCYATWRNIS |
| | GSIEIVKKGCWLDDFNCYDRTDCVETKENPQVYFCCCEGNMCNEKFSYFP |
| | EMEVTQPTS |
| 202 | GAILGRSETQECLFYNANWELERTNQTGVERCEGEKDKRLHCYATWRNIS |
| | GSIEIVKKGCWLDDINCYDRTDCVETEENPQVYFCCCEGNMCNEKFSYFPE |
| | MEVTQPTS |
| 203 | GAILGRSETQECLFYNANWELERTNQTGVERCEGEKDKRLHCYATWKNIS |
| | GSIEIVKKGCWLDDFNCYDRTDCVETEENPQVYFCCCEGNMCNEKFSYFPE |
| | MEVTQPTS |
| 204 | GAILGRSETQECLFYNANWELERTNQTGVERCEGEKDKRLHCFATWRNIS |
| | GSIEIVKKGCWLDDFNCYDRTDCVETEENPQVYFCCCEGNMCNEKFSYFPE |
| | MEVTQPTS |
| 205 | GAILGRSETQECLFYNANWELDRTNQTGVERCEGEKDKRLHCYATWRNIS |
| | GSIEIVKKGCWLDDFNCYDRTDCVETEENPQVYFCCCEGNMCNEKFSYFPE |
| | MEVTQPTS |
| 206 | GAILGRSETQECLFYNANWELERTNQTGVEPCEGEKDKRLHCYATWRNIS |
| | GSIEIVKKGCWLDDFNCYDRTDCVETEENPQVYFCCCEGNMCNEKFSYFPE |
| | MEVTQPTS |
| 207 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTS |
| 208 | GAILGRSETQECLYYNANWELERTNQTGVERCEGEQDKRLHCYATWRNIS |
| | GSIEIVKKGCWLDDFNCYDRTDCVETEENPQVYFCCCEGNMCNEKFSYFPE |
| | MEVTQPTS |
| 209 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTSNP |
| 210 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTSNPVTPK |

In some embodiments, a polypeptide disclosed herein comprises an extracellular ActRIIA variant polypeptide (e.g., any one of SEQ ID NOs: 139-210 (e.g., SEQ ID NOs: 144-210)) having an amino acid K at the position corresponding to X₁₇ in SEQ ID NO: 139 or SEQ ID NO: 140. In some embodiments, altering the amino acid at position X₁₇ can result in reduced activity. For example, an ActRIIA variant having the sequence

GAILGRSETQECLFYNANWELERTNQTGVERCEGEKDKRLHCYATWRNIS
GSIEIVAKGCWLDDFNCYDRTDCVETEENPQVYFCCCEGNMCNEKFSYFP
EMEVTQPTS

(SEQ ID NO: 283)has reduced activity in vivo, indicating that the substitution of A for K at X₁₇ is not tolerated.

In some embodiments, a polypeptide disclosed herein including an extracellular ActRIIA variant (e.g., any one of SEQ ID NOs: 139-210 (e.g., SEQ ID NOs: 144-210)) with the sequence TEEN at positions X₂₃, X₂₄, X₂₅, and X₂₆ can have a substitution of the amino acid K for the amino acid E at position X₂₄. In some embodiments, a polypeptide disclosed herein including an extracellular ActRIIA variant (e.g., any one of SEQ ID NOs: 139-210 (e.g., SEQ ID NOs: 144-210)) with the sequence TKEN at positions X₂₃, X₂₄, X₂₅, and X₂₆ can have a substitution of the amino acid E for the amino acid K at position X₂₄. In some embodiments, polypeptides having the sequence TEEN or TKEN at positions X₂₃, X₂₄, X₂₅, and X₂₆ have reduced binding to BMP9.

In some embodiments, a polypeptide disclosed herein including an extracellular ActRIIA variant (e.g., any one of SEQ ID NOs: 139-208 (e.g., SEQ ID NOs: 144-208)) may further include a C-terminal extension (e.g., additional amino acids at the C-terminus). In some embodiments, the C-terminal extension is amino acid sequence NP. For example, the sequence including the C-terminal extension is SEQ ID NO: 209 (e.g., SEQ ID NO: 207 with a C-terminal extension of NP). In some embodiments, the C-terminal extension is amino acid sequence NPVTPK (SEQ ID NO: 288). For example, the sequence including the C-terminal extension is SEQ ID NO: 210 (e.g., SEQ ID NO: 207 with a C-terminal extension of NPVTPK). The C-terminal extension can add one to six additional amino acids at the C-terminus (e.g., 1, 2, 3, 4, 5, 6 or more additional amino acids).

In some embodiments, Compositions that can be administered to a subject according to the methods described herein are provided in Table 6, below.

TABLE 6
Compositions that can be administed to a subject according to the methods
described herein.

| Row | Composition |
|---|---|
| 1 | A polypeptide comprising an extracellular activin receptor type IIa (ActRIIA) variant, |
| | the variant having a sequence of |
| | GAILGRSETQECLX₁X₂NANWX₃X₄X₅X₆TNQTGVEX₇CX₈GX₉X₁₀X₁₁X₁₂X₁₃X₁₄HC |
| | X₁₅ATWX₁₆NISGSIEIVX₁₇X₁₈GCX₁₉X₂₀X₂₁DX₂₂NCYDRTDCVEX₂₃X₂₄X₂₅X₂₆PX₂₇V |
| | YFCCCEGNMCNEKFSYFPEMEVTQPTS (SEQ ID NO: 139), wherein X₁ is F or Y; |
| | X₂ is F or Y; X₃ is E or A; X₄ is K or L; X₅ is D or E; X₆ is R or A; X₇ is P or R; X₈ is Y |
| | or E; X₉ is D or E; X₁₀ is K or Q; X₁₁ is D or A; X₁₂ is K or A; X₁₃ is R or A; X₁₄ is R or |
| | L; X₁₅ is F or Y; X₁₆ is K, R, or A; X₁₇ is K, A, Y, F, or I; X₁₈ is Q or K; X₁₉ is W or A; |
| | X₂₀ is L or A; X₂₁ is D, K, R, A, F, G, M, N, or I; X₂₂ is I, F, or A; X₂₃ is K or T; X₂₄ is |
| | K or E; X₂₅ is D or E; X₂₆ is S or N; and X₂₇ is E or Q, and wherein the variant has at |
| | least one amino acid substitution relative to a wild-type extracellular ActRIIA having |
| | the sequence of SEQ ID NO: 211 or an extracellular ActRIIA having any one of the |
| | sequences of SEQ ID NOs: 212-232. |
| 2 | A polypeptide comprising an extracellular activin receptor type IIa (ActRIIA) variant, |
| | the variant having a sequence of |
| | GAILGRSETQECLX₁X₂NANWX₃X₄X₅X₆TNQTGVEX₇CX₈GX₉X₁₀X₁₁X₁₂X₁₃X₁₄HC |
| | X₁₅ATWX₁₆NISGSIEIVX₁₇X₁₈GCX₁₉X₂₀X₂₁DX₂₂NCYDRTDCVEX₂₃X₂₄X₂₅X₂₆PX₂₇V |
| | YFCCCEGNMCNEKFSYFPEMEVTQPTS (SEQ ID NO: 139), wherein X₁ is F, A, V, |
| | I, L, M, F, Y or W; X₂ is F, A, V, I, L, M, F, Y or W; X₃ is D, E or A; X₄ is K or L; X₅ |
| | is D, E, or A; X₆ is R, H, K, or A; X₇ is P or R; X₈ is Y or E; X₉ is D, E, or A; X₁₀ is K |
| | or Q; X₁₁ is D, E, or A; X₁₂ is K, R, H, or A; X₁₃ is R, H, K, or A; X₁₄ is R or L; X₁₅ is |
| | F, A, V, I, L, M, F, Y or W; X₁₆ is K, R, H, or A; X₁₇ is K, A, Y, F, I, V, L, M, or W; |
| | X₁₈ is Q or K; X₁₉ is W or A; X₂₀ is L or A; X₂₁ is D, K, R, H, A, F, G, M, N, or I; X₂₂ is |
| | I, F, A, V, L, M, Y or W; X₂₃ is K or T; X₂₄ is K, R, H, D, or E; X₂₅ is D, E, or A; X₂₆ is |
| | S, N, T, or Q; and X₂₇ is E or Q, and wherein the variant has at least one amino acid |
| | substitution relative to a wild-type extracellular ActRIIA having the sequence of SEQ |
| | ID NO: 211 or an extracellular ActRIIA having any one of the sequences of SEQ ID |
| | NOs: 212-232. |
| 3 | The polypeptide of row 1 or 2, wherein the variant has a sequence of |
| | GAILGRSETQECLFX₂NANWX₃X₄X₅X₆TNQTGVEX₇CX₈GX₉KX₁₁X₁₂X₁₃X₁₄HCX₁₅ |
| | ATWX₁₆NISGSIEIVX₁₇X₁₈GCX₁₉X₂₀X₂₁DX₂₂NCYDRTDCVEX₂₃X₂₄X₂₅X₂₆PX₂₇VYF |
| | CCCEGNMCNEKFSYFPEMEVTQPTS (SEQ ID NO: 140). |
| 4 | The polypeptide of rows 1-3, wherein the variant has a sequence of |
| | GAILGRSETQECLFX₂NANWEX₄X₅RTNQTGVEX₇CX₈GX₉KDKRX₁₄HCX₁₅ATW |
| | X₁₆NISGSIEIVKX₁₈GCWLDDX₂₂NCYDRTDCVEX₂₃X₂₄X₂₅X₂₆X₂₇YFCCCEGNM |
| | CNEKFSYFPEMEVTQPTS (SEQ ID NO: 141). |
| 5 | The polypeptide of any one of rows 1-4, wherein the variant has a sequence of |
| | GAILGRSETQECLFX₂NANWEX₄DRTNQTGVEX₇CX₈GX₉KDKRX₁₄HCX₁₅ATWX₁₆ |
| | NISGSIEIVKX₁₈GCWLDDX₂₂NCYDRTDCVEX₂₃KX₂₅X₂₆PX₂₇VYFCCCEGNMCN |
| | EKFSYFPEMEVTQPTS (SEQ ID NO: 142). |
| 6 | The polypeptide of any one of rows 1-5, wherein the variant has a sequence of |
| | GAILGRSETQECLFX₂NANWEX₄DRTNQTGVEPCX₈GX₉KDKRX₁₄HCFATWKNI |
| | SGSIEIVKX₁₈GCWLDDINCYDRTDCVEX₂₃KX₂₅X₂₆PX₂₇VYFCCCEGNMCNEKFS |
| | YFPEMEVTQPTS (SEQ ID NO: 143). |
| 7 | The polypeptide of row 1 or 2, wherein X₁ is F. |
| 8 | The polypeptide of row 1 or 2, wherein X₁ is Y. |
| 9 | The polypeptide of row 1, 2, 7, or 8 wherein X₁₀ is K. |
| 10 | The polypeptide of row 1, 2, 7, or 8 wherein X₁₀ is Q. |
| 11 | The polypeptide of any one of rows 1-10, wherein X₂ is F. |
| 12 | The polypeptide of any one of rows 1-10, wherein X₂ is Y. |
| 13 | The polypeptide of any one of rows 1, 2, 3, and 7-12, wherein X₃ is E. |
| 14 | The polypeptide of any one of rows 1, 2, 3, and 7-12, wherein X₃ is A. |
| 15 | The polypeptide of any one of rows 1-14, wherein X₄ is K. |
| 16 | The polypeptide of any one of rows 1-14, wherein X₄ is L. |
| 17 | The polypeptide of any one of rows 1, 2, 3, 4, and 7-16, wherein X₅ is D. |
| 18 | The polypeptide of any one of rows 1, 2, 3, 4, and 7-16, wherein X₅ is E. |
| 19 | The polypeptide of any one of rows 1, 2, 3, and 7-18, wherein X₆ is R. |
| 20 | The polypeptide of any one of rows 1, 2, 3, and 7-18, wherein X₆ is A. |
| 21 | The polypeptide of any one of rows 1-5 and 7-20, wherein X₇ is P. |
| 22 | The polypeptide of any one of rows 1-5 and 7-20, wherein X₇ is R. |
| 23 | The polypeptide of any one of rows 1-22, wherein X₈ is Y. |
| 24 | The polypeptide of any one of rows 1-22, wherein X₈ is E. |
| 25 | The polypeptide of any one of rows 1-24, wherein X₉ is D. |
| 26 | The polypeptide of any one of rows 1-24, wherein X₉ is E |
| 27 | The polypeptide of any one of rows 1, 2, 3, and 7-26, wherein X₁₁ is D. |
| 28 | The polypeptide of any one of rows 1, 2, 3, and 7-26, wherein X₁₁ is A. |
| 29 | The polypeptide of any one of rows 1, 2, 3, and 7-28, wherein X₁₂ is K. |
| 30 | The polypeptide of any one of rows 1, 2, 3, and 7-28, wherein X₁₂ is A. |
| 31 | The polypeptide of any one of rows 1, 2, 3, and 7-30, wherein X₁₃ is R. |
| 32 | The polypeptide of any one of rows 1, 2, 3, and 7-30, wherein X₁₃ is A. |
| 33 | The polypeptide of any one of rows 1-32, wherein X₁₄ is R. |
| 34 | The polypeptide of any one of rows 1-32, wherein X₁₄ is L. |
| 35 | The polypeptide of any one of rows 1-5 and 7-34, wherein X₁₅ is F. |
| 36 | The polypeptide of any one of rows 1-5 and 7-34, wherein X₁₅ is Y. |
| 37 | The polypeptide of any one of rows 1-5 and 7-36, wherein X₁₆ is K. |
| 38 | The polypeptide of any one of rows 1-5 and 7-36, wherein X₁₆ is R. |
| 39 | The polypeptide of any one of rows 1-5 and 7-36, wherein X₁₆ is A. |
| 40 | The polypeptide of any one of rows 1, 2, 3, and 7-39, wherein X₁₇ is K. |
| 41 | The polypeptide of any one of rows 1, 2, 3, and 7-39, wherein X₁₇ is A. |
| 42 | The polypeptide of any one of rows 1, 2, 3, and 7-39, wherein X₁₇ is Y. |
| 43 | The polypeptide of any one of rows 1, 2, 3, and 7-39, wherein X₁₇ is F. |
| 44 | The polypeptide of any one of rows 1, 2, 3, and 7-39, wherein X₁₇ is 1. |
| 45 | The polypeptide of any one of rows 1-44, wherein X₁₈ is Q. |
| 46 | The polypeptide of any one of rows 1-44, wherein X₁₈ is K. |
| 47 | The polypeptide of any one of rows 1-3 and 7-46, wherein X₁₉ is W. |
| 48 | The polypeptide of any one of rows 1-3 and 7-46, wherein X₁₉ is A. |
| 49 | The polypeptide of any one of rows 1-3 and 7-48, wherein X₂₀ is L. |
| 50 | The polypeptide of any one of rows 1-3 and 7-48, wherein X₂₀ is A. |
| 51 | The polypeptide of any one of rows 1-3 and 7-50, wherein X₂₁ is D. |
| 52 | The polypeptide of any one of rows 1-3 and 7-50, wherein X₂₁ is K. |
| 53 | The polypeptide of any one of rows 1-3 and 7-50, wherein X₂₁ is R. |
| 54 | The polypeptide of any one of rows 1-3 and 7-50, wherein X₂₁ is A. |
| 55 | The polypeptide of any one of rows 1-3 and 7-50, wherein X₂₁ is F. |
| 56 | The polypeptide of any one of rows 1-3 and 7-50, wherein X₂₁ is G. |
| 57 | The polypeptide of any one of rows 1-3 and 7-50, wherein X₂₁ is M. |
| 58 | The polypeptide of any one of rows 1-3 and 7-50, wherein X₂₁ is N. |
| 59 | The polypeptide of any one of rows 1-3 and 7-50, wherein X₂₁ is 1. |
| 60 | The polypeptide of any one of rows 1-5 and 7-59, wherein X₂₂ is 1. |
| 61 | The polypeptide of any one of rows 1-5 and 7-59, wherein X₂₂ is F. |
| 62 | The polypeptide of any one of rows 1-5 and 7-59, wherein X₂₂ is A. |
| 63 | The polypeptide of any one of rows 1-62, wherein X₂₃ is K. |
| 64 | The polypeptide of any one of rows 1-62, wherein X₂₃ is T. |
| 65 | The polypeptide of any one of rows 1-4 and 7-64, wherein X₂₄ is K. |
| 66 | The polypeptide of any one of rows 1-4 and 7-64, wherein X₂₄ is E. |
| 67 | The polypeptide of any one of rows 1-66, wherein X₂₅ is D. |
| 68 | The polypeptide of any one of rows 1-66, wherein X₂₅ is E. |
| 69 | The polypeptide of any one of rows 1-68, wherein X₂₆ is S. |
| 70 | The polypeptide of any one of rows 1-68, wherein X₂₆ is N. |
| 71 | The polypeptide of any one of rows 1-70, wherein X₂₇ is E. |
| 72 | The polypeptide of any one of rows 1-70, wherein X₂₇ is Q. |
| 73 | The polypeptide of any one of rows 1-72, wherein X₂₃ is T, X₂₄ is E, X₂₅ is E, and X₂₆ is |
| | N. |
| 74 | The polypeptide of any one of rows 1-72, wherein X₂₃ is T, X₂₄ is E, X₂₅ is E, and X₂₆ is |
| | N. |
| 75 | The polypeptide of any one of rows 1-74, wherein X₁₇ is K. |
| 76 | The polypeptide of row 1 or 2, wherein the variant has the sequence of any one of SEQ |
| | ID NOs: 144-210. |
| 77 | The polypeptide of row 76, wherein the variant has the sequence of SEQ ID NO: 207. |
| 78 | The polypeptide of row 76, wherein the variant has the sequence of SEQ ID NO: 196. |
| 79 | The polypeptide of row 76, wherein the variant has the sequence of SEQ ID NO: 144. |
| 80 | The polypeptide of row 76, wherein the variant has the sequence of SEQ ID NO: 176. |
| 81 | The polypeptide of row 76, wherein the variant has the sequence of SEQ ID NO: 179. |
| 82 | The polypeptide of row 76, wherein the variant has the sequence of SEQ ID NO: 182. |
| 83 | The polypeptide of row 76, wherein the variant has the sequence of SEQ ID NO: 208. |
| 84 | The polypeptide of row 76, wherein the variant has the sequence of SEQ ID NO: 209. |
| 85 | The polypeptide of row 76, wherein the variant has the sequence of SEQ ID NO: 210. |
| 86 | The polypeptide of any one of rows 1-85, wherein the amino acid at position X₂₄ is |
| | replaced with the amino acid K. |
| 87 | The polypeptide of any one of rows 1-86, wherein the amino acid at position X₂₄ is |
| | replaced with the amino acid E. |
| 88 | The polypeptide of any one of rows 1-87, further comprising a C-terminal extension of |
| | one or more amino acids. |
| 89 | The polypeptide of row 88, wherein the C-terminal extension is NP. |
| 90 | The polypeptide of row 88, wherein the C-terminal extension is NPVTPK. |
| 91 | The polypeptide of any one of rows 1-90, further comprising an Fc domain monomer |
| | fused to the C-terminus of the polypeptide by way of a linker. |
| 92 | The polypeptide of row 91, wherein the Fc domain monomer comprises the sequence of |
| | SEQ ID NO: 233. |
| 93 | The polypeptide of any one of rows 1-90, further comprising a wild-type Fc domain |
| | fused to the C-terminus of the polypeptide by way of a linker. |
| 94 | The polypeptide of row 93, wherein the wild-type Fc domain comprises the sequence of |
| | SEQ ID NO: 284. |
| 95 | The polypeptide of any one of rows 1-90, further comprising an Fc domain with amino |
| | acid substitutions fused to the C-terminus of the polypeptide by way of a linker. |
| 96 | The polypeptide of row 95, wherein the Fc domain does not form a dimer. |
| 97 | The polypeptide of any one of rows 1-90, further comprising an albumin-binding |
| | peptide fused to the C-terminus of the polypeptide by way of a linker. |
| 98 | The polypeptide of row 97, wherein the albumin-binding peptide comprises the |
| | sequence of SEQ ID NO: 285. |
| 99 | The polypeptide of any one of rows 1-90, further comprising a fibronectin domain fused |
| | to the C-terminus of the polypeptide by way of a linker. |
| 100 | The polypeptide of row 99, wherein the fibronectin domain comprises the sequence of |
| | SEQ ID NO: 286. |
| 101 | The polypeptide of any one of rows 1-90, further comprising a human serum albumin |
| | fused to the C-terminus of the polypeptide by way of a linker. |
| 102 | The polypeptide of row 101, wherein the human serum albumin comprises the sequence |
| | of SEQ ID NO: 287. |
| 103 | The polypeptide of row 91 or 92, wherein the polypeptide forms a dimer. |
| 104 | The polypeptide of any one of rows 91-103, wherein the linker is an amino acid spacer. |
| 105 | The polypeptide of row 104, wherein the amino acid spacer is GGG, GGGA (SEQ ID |
| | NO: 234),GGGG (SEQ ID NO: 20), GGGAG (SEQ ID NO: 263), GGGAGG (SEQ ID |
| | NO: 264), or GGGAGGG (SEQ ID NO: 265). |

In some embodiments, an extracellular ActRIIA variant described herein does not have the sequence of any one of SEQ TD NOs: 212-232 shown in Table 7 below.

TABLE 7
Excluded Extracellular ActRIIA Variant polypeptides.

| SEQ ID | |
|---|---|
| NO: | Amino Acid Sequence |
| 2212 | GAILGRSETQECLFFNANWEKDRTNQTGVEPCYGDKDKRRHCFATWANISG |
| | SIEIVKQGCWLDDINCYDRTDCVEKKDSPEVYFCCCEGNMCNEKFSYFPEME |
| | VTQPTS |
| 2213 | GAILGRSETQECLFFNANWAKDRTNQTGVEPCYGDKDKRRHCFATWKNISG |
| | SIEIVKQGCWLDDINCYDRTDCVEKKDSPEVYFCCCEGNMCNEKFSYFPEME |
| | VTQPTS |
| 2214 | GAILGRSETQECLFFNANWEKDATNQTGVEPCYGDKDKRRHCFATWKNISG |
| | SIEIVKQGCWLDDINCYDRTDCVEKKDSPEVYFCCCEGNMCNEKFSYFPEME |
| | VTQPTS |
| 2215 | GAILGRSETQECLFFNANWEKDRTNQTGVEPCYGDKAKRRHCFATWKNISG |
| | SIEIVKQGCWLDDINCYDRTDCVEKKDSPEVYFCCCEGNMCNEKFSYFPEME |
| | VTQPTS |
| 2216 | GAILGRSETQECLFFNANWEKDRTNQTGVEPCYGDKDARRHCFATWKNISG |
| | SIEIVKQGCWLDDINCYDRTDCVEKKDSPEVYFCCCEGNMCNEKFSYFPEME |
| | VTQPTS |
| 2217 | GAILGRSETQECLFFNANWEKDRTNQTGVEPCYGDKDKARHCFATWKNISG |
| | SIEIVKQGCWLDDINCYDRTDCVEKKDSPEVYFCCCEGNMCNEKFSYFPEME |
| | VTQPTS |
| 2218 | GAILGRSETQECLFFNANWEKDRTNQTGVEPCYGDKDKRRHCFATWKNISG |
| | SIEIVAQGCWLDDINCYDRTDCVEKKDSPEVYFCCCEGNMCNEKFSYFPEME |
| | VTQPTS |
| 2219 | GAILGRSETQECLFFNANWEKDRTNQTGVEPCYGDKDKRRHCFATWKNISG |
| | SIEIVYQGCWLDDINCYDRTDCVEKKDSPEVYFCCCEGNMCNEKFSYFPEME |
| | VTQPTS |
| 2220 | GAILGRSETQECLFFNANWEKDRTNQTGVEPCYGDKDKRRHCFATWKNISG |
| | SIEIVFQGCWLDDINCYDRTDCVEKKDSPEVYFCCCEGNMCNEKFSYFPEME |
| | VTQPTS |
| 2221 | GAILGRSETQECLFFNANWEKDRTNQTGVEPCYGDKDKRRHCFATWKNISG |
| | SIEIVIQGCWLDDINCYDRTDCVEKKDSPEVYFCCCEGNMCNEKFSYFPEME |
| | VTQPTS |
| 2222 | GAILGRSETQECLFFNANWEKDRTNQTGVEPCYGDKDKRRHCFATWKNISG |
| | SIEIVKQGCALDDINCYDRTDCVEKKDSPEVYFCCCEGNMCNEKFSYFPEME |
| | VTQPTS |
| 2223 | GAILGRSETQECLFFNANWEKDRTNQTGVEPCYGDKDKRRHCFATWKNISG |
| | SIEIVKQGCWADDINCYDRTDCVEKKDSPEVYFCCCEGNMCNEKFSYFPEME |
| | VTQPTS |
| 2224 | GAILGRSETQECLFFNANWEKDRTNQTGVEPCYGDKDKRRHCFATWKNISG |
| | SIEIVKQGCWLKDINCYDRTDCVEKKDSPEVYFCCCEGNMCNEKFSYFPEME |
| | VTQPTS |
| 2225 | GAILGRSETQECLFFNANWEKDRTNQTGVEPCYGDKDKRRHCFATWKNISG |
| | SIEIVKQGCWLRDINCYDRTDCVEKKDSPEVYFCCCEGNMCNEKFSYFPEME |
| | VTQPTS |
| 2226 | GAILGRSETQECLFFNANWEKDRTNQTGVEPCYGDKDKRRHCFATWKNISG |
| | SIEIVKQGCWLADINCYDRTDCVEKKDSPEVYFCCCEGNMCNEKFSYFPEME |
| | VTQPTS |
| 2227 | GAILGRSETQECLFFNANWEKDRTNQTGVEPCYGDKDKRRHCFATWKNISG |
| | SIEIVKQGCWLFDINCYDRTDCVEKKDSPEVYFCCCEGNMCNEKFSYFPEME |
| | VTQPTS |
| 2228 | GAILGRSETQECLFFNANWEKDRTNQTGVEPCYGDKDKRRHCFATWKNISG |
| | SIEIVKQGCWLGDINCYDRTDCVEKKDSPEVYFCCCEGNMCNEKFSYFPEME |
| | VTQPTS |
| 2229 | GAILGRSETQECLFFNANWEKDRTNQTGVEPCYGDKDKRRHCFATWKNISG |
| | SIEIVKQGCWLMDINCYDRTDCVEKKDSPEVYFCCCEGNMCNEKFSYFPEME |
| | VTQPTS |
| 2230 | GAILGRSETQECLFFNANWEKDRTNQTGVEPCYGDKDKRRHCFATWKNISG |
| | SIEIVKQGCWLNDINCYDRTDCVEKKDSPEVYFCCCEGNMCNEKFSYFPEME |
| | VTQPTS |
| 2231 | GAILGRSETQECLFFNANWEKDRTNQTGVEPCYGDKDKRRHCFATWKNISG |
| | SIEIVKQGCWLIDINCYDRTDCVEKKDSPEVYFCCCEGNMCNEKFSYFPEME |
| | VTQPTS |
| 2232 | GAILGRSETQECLFFNANWEKDRTNQTGVEPCYGDKDKRRHCFATWKNISG |
| | SIEIVKQGCWLDDANCYDRTDCVEKKDSPEVYFCCCEGNMCNEKFSYFPEM |
| | EVTQPTS |

Furthermore, in some embodiments, a polypeptide described herein has a serum half-life of at least 7 days in humans. In some embodiments, the polypeptide may bind to bone morphogenetic protein 9 (BMP9) with a K_{D} of 200 pM or higher. In some embodiments, the polypeptide may bind to activin A with a K_{D} of 10 pM or higher. In some embodiments, the polypeptide does not bind to BMP9 or activin A. In some embodiments, the polypeptide binds to activin and/or myostatin and exhibits reduced binding to BMP9. In some embodiments, the polypeptide that has reduced binding to BMP9 has the sequence TEEN or TKEN at positions X₂₃, X₂₄, X₂₅, and X₂₆.

Additionally, in some embodiments, the polypeptide may bind to human BMP9 with a K_{D} of about 200 pM or higher (e.g., a K_{D} of about 200, 300, 400, 500, 600, 700, 800, or 900 pM or higher, e.g., a K_{D} of about 1, 2, 3, 4, 5, 6, 7, 8, 9, 10, 20, 30, 40, or 50 nM or higher, e.g., a K_{D} of between about 200 pM and about 50 nM). In some embodiments, the polypeptide does not substantially bind to human BMP9. In some embodiments, the polypeptide may bind to human activin A with a K_{D} of about 800 pM or less (e.g., a K_{D} of about 800, 700, 600, 500, 400, 300, 200,100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

To illustrate, one or more mutations may be selected that increase the selectivity of the altered ligand-binding domain for GDF11 and/or GDF8 over one or more ActRII-binding ligands such as activins (activin A, activin B, activin AB, activin C, and/or activin E), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

Amino acid residues of the ActRIIB proteins (e.g., E39, K55, Y60, K74, W78, L79, D80, and F101 with respect to SEQ ID NO: 1) are in the ActRIIB ligand-binding pocket and help mediate binding to its ligands including, for example, activin A, GDF11, and GDF8. Thus the present disclosure provides polypeptides comprising an altered-ligand binding domain (e.g., a GDF8/GDF11-binding domain) of an ActRIIB receptor which comprises one or more mutations at those amino acid residues.

As a specific example, the positively-charged amino acid residue Asp (D80) of the ligand-binding domain of ActRIIB can be mutated to a different amino acid residue to produce a polypeptide that preferentially binds to GDF8, but not activin. In some embodiments, the D80 residue with respect to SEQ ID NO: 1 is changed to an amino acid residue selected from the group consisting of: an uncharged amino acid residue, a negative amino acid residue, and a hydrophobic amino acid residue. As a further specific example, the hydrophobic residue L79 of SEQ ID NO: 1 can be altered to confer altered activin-GDF11/GDF8 binding properties. For example, an L79P substitution reduces GDF11 binding to a greater extent than activin binding. In contrast, replacement of L79 with an acidic amino acid [an aspartic acid or glutamic acid; an L79D or an L79E substitution] greatly reduces activin A binding affinity while retaining GDF11 binding affinity. In exemplary embodiments, the methods described herein utilize a polypeptide which is a variant ActRIIB polypeptide comprising an acidic amino acid (e.g., D or E) at the position corresponding to position 79 of SEQ ID NO: 1, optionally in combination with one or more additional amino acid substitutions, additions, or deletions.

In certain aspects, the disclosure relates ALK4 polypeptides and uses thereof. As used herein, the term “ALK4” refers to a family of activin receptor-like kinase-4 proteins from any species and variants derived from such ALK4 proteins by mutagenesis or other modification. Reference to ALK4 herein is understood to be a reference to any one of the currently identified forms. Members of the ALK4 family are generally transmembrane proteins, composed of a ligand-binding extracellular domain with a cysteine-rich region, a transmembrane domain, and a cytoplasmic domain with predicted serine/threonine kinase activity.

The term “ALK4 polypeptide” includes polypeptides comprising any naturally occurring polypeptide of an ALK4 family member as well as any variants thereof (including mutants, fragments, fusions, and peptidomimetic forms) that retain a useful activity. Numbering of amino acids for all ALK4-related polypeptides described herein is based on the numbering of the human ALK4 precursor protein sequence below (SEQ ID NO: 100), unless specifically designated otherwise.

A human ALK4 precursor protein sequence (NCBI Ref Seq NP_004293) is as follows:

(SEQ ID NO: 100)

| | |
|---|---|
| 1 | MAESAGASSE FPLVVLLLAG SGGSGPRGVQ ALLCACTSCL |
| | QANYTCETDG ACMVSIFNLD |
| 61 | GMEHHVRTCI PKVELVPAGK PFYCLSSEDL RNTHCCYTDY |
| | CNRIDLRVPS GHLKEPEHPS |
| 121 | MWGPVELVGI IAGPVFLLFL IIIIVFLVIN YHQRVYHNRQ |
| | RLDMEDPSCE MCLSKDKTLQ |
| 181 | DLVYDLSTSG SGSGLPLFVQ RTVARTIVLQ EIIGKGRFGE |
| | VWRGRWRGGD VAVKIFSSRE |
| 241 | ERSWFREAEI YQTVMLRHEN ILGFIAADNK DNGTWTQLWL |
| | VSDYHEHGSL FDYLNRYTVT |
| 301 | IEGMIKLALS AASGLAHLHM EIVGTQGKPG IAHRDLKSKN |
| | ILVKKNGMCA IADLGLAVRH |
| 361 | DAVTDTIDIA PNQRVGTKRY MAPEVLDETI NMKHFDSFKC |
| | ADIYALGLVY WEIARRCNSG |
| 421 | GVHEEYQLPY YDLVPSDPSI EEMRKVVCDQ KLRPNIPNWW |
| | QSYEALRVMG KMMRECWYAN |
| 481 | GAARLTALRI KKTLSQLSVQ EDVKI |

The signal peptide is indicated by a single underline and the extracellular domain is indicated in bold font.

A processed extracellular human ALK4 polypeptide sequence is as follows:

(SEQ ID NO: 101)
SGPRGVQALLCACTSCLQANYTCETDGACMVSIFNLDGMEHHVRTCIPKV
ELVPAGKPEYCLSSEDLRNTHCCYTDYCNRIDLRVPSGHLKEPEHPSMWG
PVE

A nucleic acid sequence encoding the ALK4 precursor protein is shown below (SEQ ID NO: 102), corresponding to nucleotides 78-1592 of Genbank Reference Sequence NM_004302.4. The signal sequence is underlined and the extracellular domain is indicated in bold font.

(SEQ ID NO: 102)
ATGGCGGAGTCGGCCGGAGCCTCCTCCTTCTTCCCCCTTGTTGTCCTCCT
GCTCGCCGGCAGCGGCGGGTCCGGGCCCCGGGGGGTCCAGGCTCTGCTGT
GTGCGTGCACCAGCTGCCTCCAGGCCAACTACACGTGTGAGACAGATGGG
GCCTGCATGGTTTCCATTTTCAATCTGGATGGGATGGAGCACCATGTGCG
CACCTGCATCCCCAAAGTGGAGCTGGTCCCTGCCGGGAAGCCCTTCTACT
GCCTGAGCTCGGAGGACCTGCGCAACACCCACTGCTGCTACACTGACTAC
TGCAACAGGATCGACTTGAGGGTGCCCAGTGGTCACCTCAAGGAGCCTGA
GCACCCGTCCATGTGGGGCCCGGTGGAGCTGGTAGGCATCATCGCCGGCC
CGGTGTTCCTCCTGTTCCTCATCATCATCATTGTTTTCCTTGTCATTAAC
TATCATGAGCGTGTCTATCACAACCGCCAGAGACTGGACATGGAAGATCC
CTCATGTGAGATGTGTCTCTCCAAAGACAAGACGCTCCAGGATCTTGTCT
ACGATCTCTCCACCTCAGGGTCTGGCTCAGGGTTACCCCTCTTTGTCCAG
CGCACAGTGGCCCGAACCATCGTTTTACAAGAGATTATTGGCAAGGGTCG
GTTTGGGGAAGTATGGCGGGGCCGCTGGAGGGGTGGTGATGTGGCTGTGA
AAATATTCTCTTCTCGTGAAGAACGGTCTTGGTTCAGGGAAGCAGAGATA
TACCAGACGGTCATGCTGCGCCATGAAAACATCCTTGGATTTATTGCTGC
TGACAATAAAGATAATGGCACCTGGACACAGCTGTGGCTTGTTTCTGACT
ATCATGAGCACGGGTCCCTGTTTGATTATCTGAACCGGTACACAGTGACA
ATTGAGGGGATGATTAAGCTGGCCTTGTCTGCTGCTAGTGGGCTGGCACA
CCTGCACATGGAGATCGTGGGCACCCAAGGGAAGCCTGGAATTGCTCATC
GAGACTTAAAGTCAAAGAACATTCTGGTGAAGAAAAATGGCATGTGTGCC
ATAGCAGACCTGGGCCTGGCTGTCCGTCATGATGCAGTCACTGACACCAT
TGACATTGCCCCGAATCAGAGGGTGGGGACCAAACGATACATGGCCCCTG
AAGTACTTGATGAAACCATTAATATGAAACACTTTGACTCCTTTAAATGT
GCTGATATTTATGCCCTCGGGCTTGTATATTGGGAGATTGCTCGAAGATG
CAATTCTGGAGGAGTCCATGAAGAATATCAGCTGCCATATTACGACTTAG
TGCCCTCTGACCCTTCCATTGAGGAAATGCGAAAGGTTGTATGTGATCAG
AAGCTGCGTCCCAACATCCCCAACTGGTGGCAGAGTTATGAGGCACTGCG
GGTGATGGGGAAGATGATGCGAGAGTGTTGGTATGCCAACGGCGCAGCCC
GCCTGACGGCCCTGCGCATCAAGAAGACCCTCTCCCAGCTCAGCGTGCAG
GAAGACGTGAAGATC

A nucleic acid sequence encoding the extracellular ALK4 polypeptide is as follows:

(SEQ ID NO: 103)
TCCGGGCCCCGGGGGGTCCAGGCTCTGCTGTGTGCGTGCACCAGCTGCCT
CCAGGCCAACTACACGTGTGAGACAGATGGGGCCTGCATGGTTTCCATTT
TCAATCTGGATGGGATGGAGCACCATGTGCGCACCTGCATCCCCAAAGTG
GAGCTGGTCCCTGCCGGGAAGCCCTTCTACTGCCTGAGCTCGGAGGACCT
GCGCAACACCCACTGCTGCTACACTGACTACTGCAACAGGATCGACTTGA
GGGTGCCCAGTGGTCACCTCAAGGAGCCTGAGCACCCGTCCATGTGGGGC
CCGGTGGAG

An alternative isoform of human ALK4 precursor protein sequence, isoform B (NCBI Ref Seq NP_064732.3), is as follows:

(SEQ ID NO: 104)

| | |
|---|---|
| 1 | MVSIFNLDGM EHHVRTCIPK VELVPAGKPF YCLSSEDLRN |
| | THCCYTDYCN RIDLRVPSGH |
| 61 | LKEPEHPSMW GPVELVGIIA GPVFLLFLII IIVFLVINYH |
| | QRVYHNRQRL DMEDPSCEMC |
| 121 | LSKDKTLQDL VYDLSTSGSG SGLPLFVQRT VARTIVLQEI |
| | IGKGRFGEVW RGRWRGGDVA |
| 181 | VKIFSSREER SWFREAEIYQ TVMLRHENIL GFIAADNKDN |
| | GTWTQLWLVS DYHEHGSLFD |
| 241 | YLNRYTVTIE GMIKLALSAA SGLAHLHMEI VGTQGKPGIA |
| | HRDLKSKNIL VKKNGMCAIA |
| 301 | DLGLAVRHDA VTDTIDIAPN QRVGTKRYMA PEVLDETINM |
| | KHFDSFKCAD IYALGLVYWE |
| 361 | IARRCNSGGV HEEYQLPYYD LVPSDPSIEE MRKVVCDQKL |
| | RPNIPNWWQS YEALRVMGKM |
| 421 | MRECWYANGA ARLTALRIKK TLSQLSVQED VKI |

The extracellular domain is indicated in bold font.

A processed extracellular ALK4 polypeptide sequence is as follows:

(SEQ ID NO: 105)

| | |
|---|---|
| 1 | MVSIFNLDGM EHHVRTCIPK VELVPAGKPF |
| | YCLSSEDLRN THCCYTDYCN RIDLRVPSGH |
| 61 | LKEPEHPSMW GPVE |

A nucleic acid sequence encoding the ALK4 precursor protein (isoform B) is shown below (SEQ ID NO: 106), corresponding to nucleotides 186-1547 of Genbank Reference Sequence NM_020327.3. The nucleotides encoding the extracellular domain are indicated in bold font.

(SEQ ID NO: 106)

| | |
|---|---|
| 1 | ATGGTTTCCA TTTTCAATCT GGATGGGATG |
| | GAGCACCATG TGCGCACCTG |
| 51 | CATCCCCAAA GTGGAGCTGG TCCCTGCCGG |
| | GAAGCCCTTC TACTGCCTGA |
| 101 | GCTCGGAGG ACCTGCGCAAC ACCCACTGCT |
| | GCTACACTGA CTACTGCAAC |
| 151 | AGGATCGACT TGAGGGTGCC CAGTGGTCAC |
| | CTCAAGGAGC CTGAGCACCC |
| 201 | GTCCATGTGG GGCCCGGTGG AGCTGGTAGG |
| | CATCATCGCC GGCCCGGTGT |
| 251 | TCCTCCTGTT CCTCATCATC ATCATTGTTT |
| | TCCTTGTCAT TAACTATCAT |
| 301 | CAGCGTGTCT ATCACAACCG CCAGAGACTG |
| | GACATGGAAG ATCCCTCATG |
| 351 | TGAGATGTGT CTCTCCAAAG ACAAGACGCT |
| | CCAGGATCTT GTCTACGATC |
| 401 | TCTCCACCTC AGGGTCTGGC TCAGGGTTAC |
| | CCCTCTTTGT CCAGCGCACA |
| 451 | GTGGCCCGAA CCATCGTTTT ACAAGAGATT |
| | ATTGGCAAGG GTCGGTTTGG |
| 501 | GGAAGTATGG CGGGGCCGCT GGAGGGGTGG |
| | TGATGTGGCT GTGAAAATAT |
| 551 | TCTCTTCTCG TGAAGAACGG TCTTGGTTCA |
| | GGGAAGCAGA GATATACCAG |
| 601 | ACGGTCATGC TGCGCCATGA AAACATCCTT |
| | GGATTTATTG CTGCTGACAA |
| 651 | TAAAGATAAT GGCACCTGGA CACAGCTGTG |
| | GCTTGTTTCT GACTATCATG |
| 701 | AGCACGGGTC CCTGTTTGAT TATCTGAACC |
| | GGTACACAGT GACAATTGAG |
| 751 | GGGATGATTA AGCTGGCCTT GTCTGCTGCT |
| | AGTGGGCTGG CACACCTGCA |
| 801 | CATGGAGATC GTGGGCACCC AAGGGAAGCC |
| | TGGAATTGCT CATCGAGACT |
| 851 | TAAAGTCAAA GAACATTCTG GTGAAGAAAA |
| | ATGGCATGTG TGCCATAGCA |
| 901 | GACCTGGGCC TGGCTGTCCG TCATGATGCA |
| | GTCACTGACA CCATTGACAT |
| 951 | TGCCCCGAAT CAGAGGGTGG GGACCAAACG |
| | ATACATGGCC CCTGAAGTAC |
| 1001 | TTGATGAAAC CATTAATATG AAACACTTTG |
| | ACTCCTTTAA ATGTGCTGAT |
| 1051 | ATTTATGCCC TCGGGCTTGT ATATTGGGAG |
| | ATTGCTCGAA GATGCAATTC |
| 1101 | TGGAGGAGTC CATGAAGAAT ATCAGCTGCC |
| | ATATTACGAC TTAGTGCCCT |
| 1151 | CTGACCCTTC CATTGAGGAA ATGCGAAAGG |
| | TTGTATGTGA TCAGAAGCTG |
| 1201 | CGTCCCAACA TCCCCAACTG GTGGCAGAGT |
| | TATGAGGCAC TGCGGGTGAT |
| 1251 | GGGGAAGATG ATGCGAGAGT GTTGGTATGC |
| | CAACGGCGCA GCCCGCCTGA |
| 1301 | CGGCCCTGCG CATCAAGAAG ACCCTCTCCC |
| | AGCTCAGCGT GCAGGAAGAC |
| 1351 | GTGAAGATCT AA |

A nucleic acid sequence encoding the extracellular ALK4 polypeptide (isoform B) is as follows:

(SEQ ID NO: 107)

| | |
|---|---|
| 1 | ATGGTTTCCA TTTTCAATCT GGATGGGATG |
| | GAGCACCATG TGCGCACCTG |
| 51 | CATCCCCAAA GTGGAGCTGG TCCCTGCCGG |
| | GAAGCCCTTC TACTGCCTGA |
| 101 | GCTCGGAGGA CCTGCGCAAC ACCCACTGCT |
| | GCTACACTGA CTACTGCAAC |
| 151 | AGGATCGACT TGAGGGTGCC CAGTGGTCAC |
| | CTCAAGGAGC CTGAGCACCC |
| 201 | GTCCATGTGG GGCCCGGTGG AGCTGGTAGG |

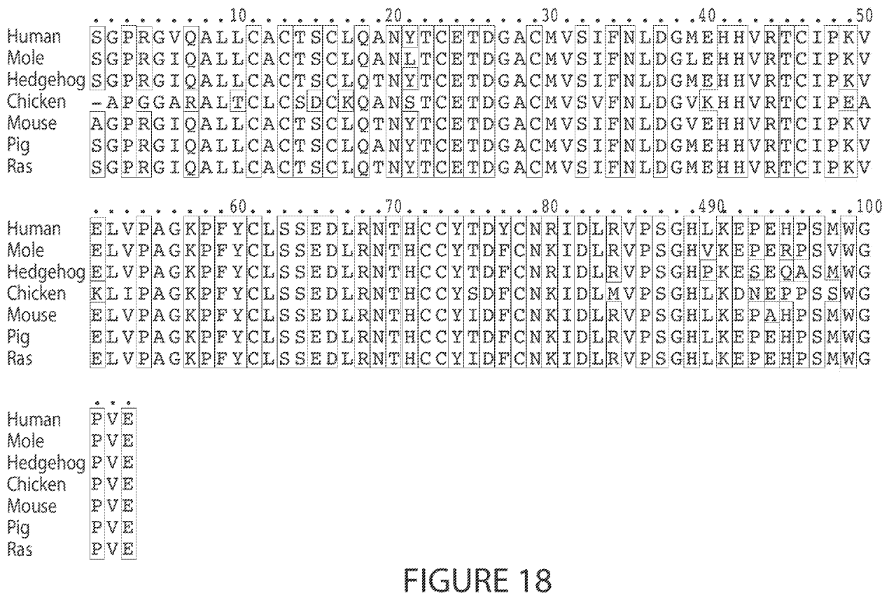

ALK4 is well-conserved among vertebrates, with large stretches of the extracellular domain completely conserved. For example, FIG. 18 depicts a multi-sequence alignment of a human ALK4 extracellular domain compared to various ALK4 orthologs. Many of the ligands that bind to ALK4 are also highly conserved. Accordingly, from these alignments, it is possible to predict key amino acid positions within the ligand-binding domain that are important for normal ALK4-ligand binding activities as well as to predict amino acid positions that are likely to be tolerant to substitution without significantly altering normal ALK4-ligand binding activities. Therefore, an active, human ALK4 variant polypeptide useful in accordance with the presently disclosed methods may include one or more amino acids at corresponding positions from the sequence of another vertebrate ALK4, or may include a residue that is similar to that in the human or other vertebrate sequences.

Without meaning to be limiting, the following examples illustrate this approach to defining an active ALK4 variant. As illustrated in FIG. 18, V6 in the human ALK4 extracellular domain (SEQ ID NO: 126) is isoleucine in Mus musculus ALK4 (SEQ ID NO: 130), and so the position may be altered, and optionally may be altered to another hydrophobic residue such as L, I, or F, or a non-polar residue such as A, as is observed in Gallus gallus ALK4 (SEQ ID NO: 129). E40 in the human extracellular domain is K in Gallus gallus ALK4, indicating that this site may be tolerant of a wide variety of changes, including polar residues, such as E, D, K, R, H, S, T, P, G, Y, and probably a non-polar residue such as A. S15 in the human extracellular domain is D in Gallus gallus ALK4, indicating that a wide structural variation is tolerated at this position, with polar residues favored, such as S, T, R, E, K, H, G, P, G and Y. E40 in the human extracellular domain is K in Gallus gallus ALK4, indicating that charged residues will be tolerated at this position, including D, R, K, H, as well as Q and N. R80 in the human extracellular domain is K in Condylura cristata ALK4 (SEQ ID NO: 127), indicating that basic residues are tolerated at this position, including R, K, and H. Y77 in the human extracellular domain is F in Sus scrofa ALK4 (SEQ ID NO: 131), indicating that aromatic residues are tolerated at this position, including F, W, and Y. P93 in the human extracellular domain is relatively poorly conserved, appearing as S in Erinaceus europaeus ALK4 (SEQ ID NO: 128) and N in Gallus gallus ALK4, thus essentially any amino acid should be tolerated at this position.

Moreover, ALK4 proteins have been characterized in the art in terms of structural and functional characteristics, particularly with respect to ligand binding [e.g., Harrison et al. (2003) J Biol Chem 278(23):21129-21135; Romano et al. (2012) J Mol Model 18(8):3617-3625; and Calvanese et al. (2009) 15(3):175-183]. In addition to the teachings herein, these references provide amply guidance for how to generate ALK4 variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ALK4, as demarcated by the outermost of these conserved cysteines, corresponds to positions 34-101 of SEQ ID NO: 100 (ALK4 precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 residues at the N-terminus and/or by 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ALK4 extracellular domains for N-terminal and/or C-terminal truncation include SEQ ID NOs: 101 and 105.

Accordingly, a general formula for an active portion (e.g., a ligand-binding portion) of ALK4 comprises amino acids 34-101 with respect to SEQ ID NO: 100. Therefore ALK4 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ALK4 beginning at a residue corresponding to any one of amino acids 24-34 (e.g., beginning at any one of amino acids 24, 25, 26, 27, 28, 29, 30, 31, 32, 33, or 34) of SEQ ID NO: 100 and ending at a position corresponding to any one amino acids 101-126 (e.g., ending at any one of amino acids 101, 102, 103, 104, 105, 106, 107, 108, 109, 110, 111, 112, 113, 114, 115, 116, 117, 118, 119, 120, 121, 122, 123, 124, 125, or 126) of SEQ ID NO: 100. Other examples include constructs that begin at a position from 24-34 (e.g., any one of positions 24, 25, 26, 27, 28, 29, 30, 31, 32, 33, or 34), 25-34 (e.g., any one of positions 25, 26, 27, 28, 29, 30, 31, 32, 33, or 34), or 26-34 (e.g., any one of positions 26, 27, 28, 29, 30, 31, 32, 33, or 34) of SEQ ID NO: 100 and end at a position from 101-126 (e.g., any one of positions 101, 102, 103, 104, 105, 106, 107, 108, 109, 110, 111, 112, 113, 114, 115, 116, 117, 118, 119, 120, 121, 122, 123, 124, 125, or 126), 102-126 (e.g., any one of positions 102, 103, 104, 105, 106, 107, 108, 109, 110, 111, 112, 113, 114, 115, 116, 117, 118, 119, 120, 121, 122, 123, 124, 125, or 126), 101-125 (e.g., any one of positions 101, 102, 103, 104, 105, 106, 107, 108, 109, 110, 111, 112, 113, 114, 115, 116, 117, 118, 119, 120, 121, 122, 123, 124, or 125), 101-124 (e.g., any one of positions 101, 102, 103, 104, 105, 106, 107, 108, 109, 110, 111, 112, 113, 114, 115, 116, 117, 118, 119, 120, 121, 122, 123, or 124), 101-121 (e.g., any one of positions 101, 102, 103, 104, 105, 106, 107, 108, 109, 110, 111, 112, 113, 114, 115, 116, 117, 118, 119, 120, or 121), 111-126 (e.g., any one of positions 111, 112, 113, 114, 115, 116, 117, 118, 119, 120, 121, 122, 123, 124, 125, or 126), 111-125 (e.g., any one of positions 111, 112, 113, 114, 115, 116, 117, 118, 119, 120, 121, 122, 123, 124, or 125), 111-124 (e.g., any one of positions 111, 112, 113, 114, 115, 116, 117, 118, 119, 120, 121, 122, 123, or 124), 121-126 (e.g., any one of positions 121, 122, 123, 124, 125, or 126), 121-125 (e.g., any one of positions 121, 122, 123, 124, or 125), 121-124 (e.g., any one of positions 121, 122, 123, or 124), or 124-126 (e.g., any one of positions 124, 125, or 126) of SEQ ID NO: 100. Variants within these ranges are also contemplated, particularly those having at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 100.

The variations described herein may be combined in various ways. In some embodiments, ALK4 variants comprise no more than 1, 2, 5, 6, 7, 8, 9, 10 or 15 conservative amino acid changes in the ligand-binding pocket. Sites outside the binding pocket, at which variability may be particularly well tolerated, include the amino and carboxy termini of the extracellular domain (as noted above).

In certain embodiments, the disclosure relates to ActRII antagonists that are heteromultimers comprising at least one ALK4 polypeptide, which includes fragments, functional variants, and modified forms thereof as well as uses thereof (e.g., treating, preventing, or reducing the severity of PAH or one or more complications of PAH). Preferably, ALK4 polypeptides are soluble (e.g., an extracellular domain of ALK4). In some embodiments, heteromultimers comprising an ALK4 polypeptide inhibit (e.g., Smad signaling) one or more TGFβ superfamily ligands [e.g., activin A, activin B, activin AB, activin AC, BMP6, BMP7, BMP9, BMP10, GDF3, GDF8, and/or GDF11]. In some embodiments, heteromultimers comprising an ALK4 polypeptide bind to one or more TGFβ superfamily ligands [e.g., activin A, activin B, activin AB, activin AC, BMP6, BMP7, BMP9, BMP10, GDF3, GDF8, and/or GDF11]. In some embodiments, heteromultimers comprise at least one ALK4 polypeptide that is at least 70%, 75%, 80%, 85%, 86%, 87%, 88%, 89%, 90%, 91%, 92%, 93%, 94%, 95%, 97%, 98%, 99%, 100% identical to amino acids 34-101 with respect to SEQ ID NO: 100. In some embodiments, heteromultimers comprise at least one ALK4 polypeptide that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 100, 101, 104, 105, 111, 113, 116, 117, 122, and 124. In some embodiments, heteromultimer comprise at least one ALK4 polypeptide that consist or consist essentially of at least one ALK4 polypeptide that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 100, 101, 104, 105, 111, 113, 116, 117, 122, and 124.

In certain aspects, the present disclosure relates to heteromultimer complexes comprising one or more ALK4 receptor polypeptides (e.g., SEQ ID NOs: 100, 101, 104, 105, 111, 113, 116, 117, 122, and 124 and variants thereof) and one or more ActRIIB receptor polypeptides (e.g., SEQ ID NOs: 1, 2, 3, 4, 5, 6, 58, 59, 60, 63, 64, 65, 66, 68, 69, 70, 71, 73, 77, 78, 108, 110, 114, 115, 118, 120, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407 and any variants thereof), which are generally referred to herein as “ALK4:ActRIIB heteromultimer complexes” or “ALK4:ActRIIB heteromultimers”, including uses thereof (e.g., increasing an immune response in a patient in need thereof and treating cancer). Preferably, ALK4:ActRIIB heteromultimers are soluble [e.g., a heteromultimer complex comprises a soluble portion (domain) of an ALK4 receptor and a soluble portion (domain) of an ActRIIB receptor]. In general, the extracellular domains of ALK4 and ActRIIB correspond to soluble portion of these receptors. Therefore, in some embodiments, ALK4:ActRIIB heteromultimers comprise an extracellular domain of an ALK4 receptor and an extracellular domain of an ActRIIB receptor. In some embodiments, ALK4:ActRIIB heteromultimers inhibit (e.g., Smad signaling) of one or more TGFβ superfamily ligands [e.g., activin A, activin B, activin AB, activin AC, BMP6, BMP7, BMP9, BMP10, GDF3, GDF8, and/or GDF11]. In some embodiments, ALK4:ActRIIB heteromultimers bind to one or more TGFβ superfamily ligands [e.g., activin A, activin B, activin AB, activin AC, BMP6, BMP7, BMP9, BMP10, GDF3, GDF8, and/or GDF11]. In some embodiments, ALK4:ActRIIB heteromultimers comprise at least one ALK4 polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 100, 101, 104, 105, 111, 113, 116, 117, 122, and 124. In some embodiments, ALK4:ActRIIB heteromultimer complexes of the disclosure comprise at least one ALK4 polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to a portion of ALK4 beginning at a residue corresponding to any one of amino acids 24-34, 25-34, or 26-34 of SEQ ID NO: 100 and ending at a position from 101-126, 102-126, 101-125, 101-124, 101-121, 111-126, 111-125, 111-124, 121-126, 121-125, 121-124, or 124-126 of SEQ ID NO: 100. In some embodiments, ALK4:ActRIIB heteromultimers comprise at least one ALK4 polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to amino acids 34-101 with respect to SEQ ID NO: 100. In some embodiments, ALK4-ActRIIB heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of any one of SEQ ID NOs: 1, 2, 3, 4, 5, 6, 58, 59, 60, 63, 64, 65, 66, 68, 69, 70, 71, 73, 77, 78, 108, 110, 114, 115, 118, 120, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 1. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 2. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 3. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 4. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 5. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 6. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 58. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 59. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 60. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 63. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 64. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 65. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 66. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 68. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 69. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 70. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 71. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 73. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 77. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 78. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 108. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 110. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 114. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 115. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 118. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 120. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 138. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 282. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 289. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 290. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 291. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 292. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 293. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 294. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 295. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 296. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 297. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 298. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 299. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 300. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 301. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 302. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 303. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 305. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 306. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 307. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 308. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 309. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 310. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 311. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 312. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 313. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 314. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 315. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 316. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 317. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 318. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 319. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 320. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 321. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 322. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 323. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 324. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 325. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 326. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 327. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 328. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 329. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 330. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 331. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 332. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 333. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 334. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 335. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 336. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 337. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 338. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 339. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 340. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 341. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 342. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 343. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 344. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 345. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 346. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 347. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 348. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 349. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 350. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 351. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 352. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 353. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 354. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 355. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 356. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 357. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 358. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 359. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 360. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 361. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 362. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 363. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 364. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 365. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 366. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 367. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 368. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 369. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 370. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 371. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 372. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 373. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 374. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 375. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 376. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 377. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 378. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 379. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 380. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 381. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 382. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 383. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 384. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 385. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 386. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 387. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 388. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 389. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 390. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 391. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 392. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 393. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 394. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 395. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 396. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 397. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 398. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 399. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 400. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 401. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 402. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 403. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 404. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 405. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 406. In some embodiments, ALK4-ActRIIB heteromultimers comprise at least one ActRIIB polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 407.

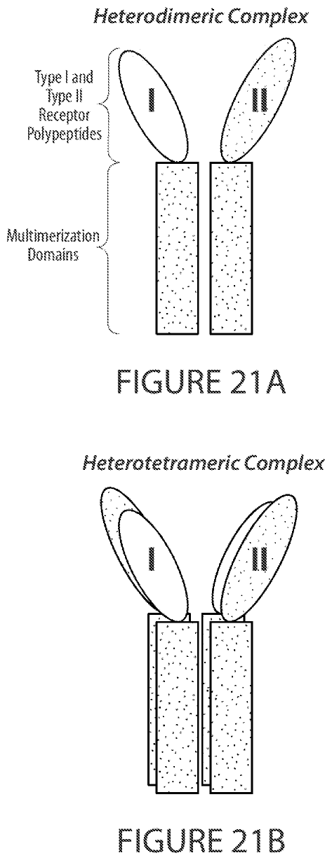

In some embodiments, ALK4:ActRIIB heteromultimer complexes of the disclosure comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to a portion of ActRIIB beginning at a residue corresponding to any one of amino acids 20-29, 20-24, 21-24, 22-25, or 21-29 and ending at a position from 109-134, 119-134, 119-133, 129-134, or 129-133 of SEQ ID NO: 1. In some embodiments, ALK4:ActRIIB heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to amino acids 29-109 of SEQ ID NO: 1. In some embodiments, ALK4:ActRIIB heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to amino acids 25-131 of SEQ ID NO: 1. In certain embodiments, ALK4:ActRIIB heteromultimer complexes of the disclosure comprise at least one ActRIIB polypeptide wherein the position corresponding to L79 of SEQ ID NO: 1 is not an acidic amino acid (i.e., not naturally occurring D or E amino acid residues or an artificial acidic amino acid residue). In some embodiments, the ActRIIB polypeptide comprises a leucine at the position corresponding to L79 of SEQ ID NO: 1. ALK4:ActRIIB heteromultimers of the disclosure include, e.g., heterodimers, heterotrimers, heterotetramers and further higher order oligomeric structures. See, e.g., FIGS. 21-23. In certain preferred embodiments, heteromultimer complexes of the disclosure are ALK4:ActRIIB heterodimers.

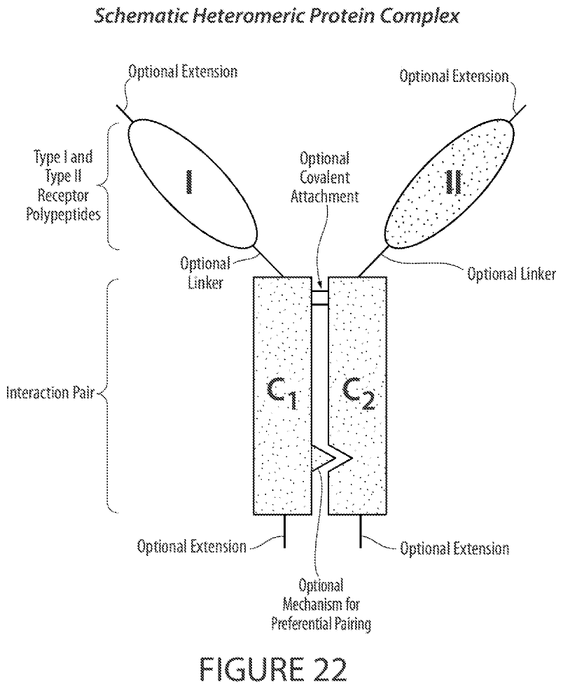

In certain aspects, the present disclosure relates to heteromultimer complexes comprising one or more ALK4 receptor polypeptides (e.g., SEQ ID NOs: 100, 101, 104, 105, 111, 113, 116, 117, 122, and 124 and variants thereof) and one or more ActRIIA receptor polypeptides (e.g., SEQ ID NOs: 9, 10, 11, 32, 36, 39, 93, 95, 96, 97, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409 and any variants thereof), which are generally referred to herein as “ALK4:ActRIIA heteromultimer complexes” or “ALK4:ActRIIA heteromultimers”, including uses thereof (e.g., increasing an immune response in a patient in need thereof and treating cancer). Preferably, ALK4:ActRIIA heteromultimers are soluble [e.g., a heteromultimer complex comprises a soluble portion (domain) of an ALK4 receptor and a soluble portion (domain) of an ActRIIA receptor]. In general, the extracellular domains of ALK4 and ActRIIA correspond to soluble portion of these receptors. Therefore, in some embodiments, ALK4:ActRIIA heteromultimers comprise an extracellular domain of an ALK4 receptor and an extracellular domain of an ActRIIA receptor. In some embodiments, ALK4:ActRIIA heteromultimers inhibit (e.g., Smad signaling) of one or more TGFβ superfamily ligands [e.g., activin A, activin B, activin AB, activin AC, BMP6, BMP7, BMP10, GDF3, GDF8, and/or GDF11]. In some embodiments, ALK4:ActRIIA heteromultimers bind to one or more TGFβ superfamily ligands [e.g., activin A, activin B, activin AB, activin AC, BMP6, BMP7, BMP10, GDF3, GDF8, and/or GDF11]. In some embodiments, ALK4:ActRIIA heteromultimers comprise at least one ALK4 polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 100, 101, 104, 105, 111, 113, 116, 117, 122, and 124. In some embodiments, ALK4:ActRIIA heteromultimer complexes of the disclosure comprise at least one ALK4 polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to a portion of ALK4 beginning at a residue corresponding to any one of amino acids 24-34, 25-34, or 26-34 of SEQ ID NO: 100 and ending at a position from 101-126, 102-126, 101-125, 101-124, 101-121, 111-126, 111-125, 111-124, 121-126, 121-125, 121-124, or 124-126 of SEQ ID NO: 100. In some embodiments, ALK4:ActRIIA heteromultimers comprise at least one ALK4 polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to amino acids 34-101 with respect to SEQ ID NO: 100. In some embodiments, ALK4-ActRIIA heteromultimers comprise at least one ActRIIA polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of any one of SEQ ID NOs: 9, 10, 11, 32, 36, 39, 93, 95, 96, 97, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 9. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 10. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 11. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 32. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 36. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 39. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 93. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 95. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 96. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 97. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 139. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 140. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 141. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 142. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 143. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 144. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 145. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 146. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 147. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 148. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 149. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 150. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 151. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 152. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 153. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 154. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 155. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 156. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 157. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 158. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 159. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 160. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 161. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 162. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 163. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 164. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 165. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 166. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 167. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 168. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 169. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 170. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 171. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 172. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 173. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 174. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 175. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 176. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 177. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 178. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 179. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 180. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 181. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 182. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 183. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 184. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 185. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 186. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 187. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 188. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 189. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 190. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 191. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 192. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 193. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 194. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 195. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 196. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 197. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 198. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 199. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 200. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 201. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 9l %, 92%, 93%, 94% 95%, 97%, 98%, 99%, or 100% identical to the amino acid sequence of SEQ ID NO: 202. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 203. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 204. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 205. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 206. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 207. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 208. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 209. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 210. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 211. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 283. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 304. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 408. In some embodiments, ALK4-ActRIIA heteromultimers comprise at least one ActRIIA polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 409. In some embodiments, ALK4:ActRIIA heteromultimer complexes of the disclosure comprise at least one ActRIIA polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to a portion of ActRIIA beginning at a residue corresponding to amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 9. In some embodiments, ALK4:ActRIIA heteromultimer complexes of the disclosure comprise at least one ActRIIA polypeptide that comprises, consists, or consists essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 9. In some embodiments, ALK4:ActRIIA heteromultimer complexes of the disclosure comprise at least one ActRIIA polypeptide that comprises, consists, or consists essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 9. ALK4:ActRIIA heteromultimers of the disclosure include, e.g., heterodimers, heterotrimers, heterotetramers and further higher order oligomeric structures. See, e.g., FIGS. 21-22. In certain preferred embodiments, heteromultimer complexes of the disclosure are ALK4:ActRIIA heterodimers.

In some embodiments, the present disclosure contemplates making functional variants by modifying the structure of an ActRII and/or ALK4 polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide or to a reference variant polypeptide, or to bind to one or more TGF-beta ligands including, for example, activin A, activin B, activin AB, activin AC, BMP6, BMP7, BMP9, BMP10, GDF3, GDF8, and GDF11.

In certain embodiments, the present disclosure contemplates specific mutations of an ActRII and/or ALK4 polypeptide so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of an ActRII and/or ALK4 polypeptide as well as truncation mutants. Pools of combinatorial mutants are especially useful for identifying functionally active (e.g., ligand binding) ActRII sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, ActRII and/or ALK4 variants, and heteromultimers comprising the same, may be screened for ability to bind to one or more TGF-beta ligands (e.g., activin A, activin B, activin AB, activin AC, BMP6, BMP7, BMP9, BMP10, GDF3, GDF8, and GDF11), to prevent binding of a TGF-beta ligand to an ActRII and/or ALK4 polypeptide, as well as heteromultimers thereof, and/or to interfere with signaling caused by a TGF-beta ligand. The activity of ActRII polypeptides, ALK4 polypeptides, ALK4:ActRIIB heteromultimers, and ALK4:ActRIIA heteromultimers may also be tested in a cell-based or in vivo assay. For example, the effect of an ActRII polypeptide, ALK4 polypeptide, ALK4:ActRIIB heteromultimer, or ALK4:ActRIIA heteromultimer on the expression of genes involved in PH pathogenesis, a kidney-associated disease (e.g., Alport syndrome, focal segmental glomerulosclerosis (FSGS), polycystic kidney disease, or chronic kidney disease), and/or an interstitial lung disease assessed. This may, as needed, be performed in the presence of one or more recombinant TGF-beta ligand proteins (e.g., activin A, activin B, activin AB, activin AC, BMP6, BMP7, BMP9, BMP10, GDF3, GDF8, and GDF11), and cells may be transfected so as to produce an ActRII polypeptide, ALK4 polypeptide, ALK4:ActRIIB heteromultimer, or ALK4:ActRIIA heteromultimer and optionally, aTGF-beta family ligand. Likewise, an ActRII polypeptide, ALK4 polypeptide, ALK4:ActRIIB heteromultimer, or ALK4:ActRIIA heteromultimer may be administered to a mouse or other animal and effects on PH pathogenesis, a kidney-associated disease (e.g., Alport syndrome, focal segmental glomerulosclerosis (FSGS), polycystic kidney disease, or chronic kidney disease), and/or an interstitial lung disease, may be assessed using art-recognized methods. Similarly, the activity of an ActRII polypeptide, ALK4 polypeptide, ALK4:ActRIIB heteromultimer, or ALK4:ActRIIA heteromultimer or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference ActRII polypeptide, ALK4 polypeptide, ALK4:ActRIIB heteromultimer, or ALK4:ActRIIA heteromultimer. Such variants, when expressed from recombinant DNA constructs, can be used in gene therapy protocols. Likewise, mutagenesis can give rise to variants which have intracellular half-lives dramatically different than the corresponding unmodified ActRII polypeptide, ALK4 polypeptide, ALK4:ActRIIB heteromultimer, or ALK4:ActRIIA heteromultimer. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the ActRII polypeptide, ALK4 polypeptide, ALK4:ActRIIB heteromultimer, or ALK4:ActRIIA heteromultimer.

A combinatorial library may be produced by way of a degenerate library of genes encoding a library of polypeptides which each include at least a portion of potential ActRII polypeptide, ALK4 polypeptide, ALK4:ActRIIB heteromultimer, or ALK4:ActRIIA heteromultimer sequences. For instance, a mixture of synthetic oligonucleotides can be enzymatically ligated into gene sequences such that the degenerate set of potential ActRII and/or or ALK4 encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, S A (1983) Tetrahedron 39:3; Itakura et al. (1981) Recombinant DNA, Proc. 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815].

Alternatively, other forms of mutagenesis can be utilized to generate a combinatorial library. For example, ActRII polypeptides, ALK4 polypeptides, ALK4:ActRIIB heteromultimers, and ALK4:ActRIIA heteromultimers of the disclosure can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, N.Y.; and Greener et al. (1994) Strategies in Mol Biol 7:32-34]. Linker scanning mutagenesis, particularly in a combinatorial setting, is an attractive method for identifying truncated (bioactive) forms of ActRII polypeptides, ALK4 polypeptides, ALK4:ActRIIB heteromultimers, or ALK4:ActRIIA heteromultimers.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRII polypeptides. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include TGF-beta ligand (e.g., activin A, activin B, activin AB, activin AC, BMP6, BMP7, BMP9, BMP10, GDF3, GDF8, and GDF11) binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of ActRII polypeptides, ALK4 polypeptides, ALK4:ActRIIB heteromultimers, or ALK4:ActRIIA heteromultimers that can be used as guidance for generating and using other variant ActRII polypeptides within the scope described herein.

In certain embodiments, functionally active fragments of ActRII polypeptides, ALK4 polypeptides, ALK4:ActRIIB heteromultimers, and ALK4:ActRIIA heteromultimers of the present disclosure can be obtained by screening polypeptides recombinantly produced from the corresponding fragment of the nucleic acid encoding an ActRII and/or ALK4 polypeptides. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII and/or ALK4 receptors and/or one or more TGF-beta ligands (e.g., activin A, activin B, activin AB, activin AC, BMP6, BMP7, BMP9, BMP10, GDF3, GDF8, and GDF11).

In certain embodiments, ActRII polypeptides, ALK4 polypeptides, ALK4:ActRIIB heteromultimers, and/or ALK4:ActRIIA heteromultimers of the present disclosure may further comprise post-translational modifications in addition to any that are naturally present in the ActRII polypeptide, ALK4 polypeptide, ALK4:ActRIIB heteromultimer, or ALK4:ActRIIA heteromultimer. Such modifications include, but are not limited to, acetylation, carboxylation, glycosylation, phosphorylation, lipidation, and acylation. As a result, the ActRII polypeptide, ALK4 polypeptide, ALK4:ActRIIB heteromultimer, or ALK4:ActRIIA heteromultimer may contain non-amino acid elements, such as polyethylene glycols, lipids, polysaccharide or monosaccharide, and phosphates. Effects of such non-amino acid elements on the functionality of a ligand trap polypeptide may be tested as described herein for other ActRII, ALK4, ALK4:ActRIIB, and ALK4:ActRIIA variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, W138, NIH-3T3 or HEK293) have specific cellular machinery and characteristic mechanisms for such post-translational activities and may be chosen to ensure the correct modification and processing of the ActRII polypeptides.

In certain aspects, ActRII and ALK4 polypeptides of the present disclosure include fusion proteins having at least a portion (domain) of an ActRII or ALK4 polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) (SEQ ID NO: 137) fusion partners. As another example, a fusion domain may be selected so as to facilitate detection of the ActRII or ALK4 polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (that confer an additional biological function) including, for example constant domains from immunoglobulins (e.g., Fc domains).

In certain aspects, ActRII and ALK4 polypeptides of the present disclosure contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising an ActRII polypeptide (or ALK4 polypeptide) domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol. In certain preferred embodiments, an ActRII polypeptide (or ALK4 polypeptide) is fused with a heterologous domain that stabilizes the polypeptide (a “stabilizer” domain), preferably a heterologous domain that increases stability of the polypeptide in vivo. Fusions with a constant domain of an immunoglobulin (e.g., a Fc domain) are known to confer desirable pharmacokinetic properties on a wide range of proteins. Likewise, fusions to human serum albumin can confer desirable properties.

An example of a native amino acid sequence that may be used for the Fc portion of human IgG1 (G1Fc) is shown below (SEQ ID NO: 14). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 14. Naturally occurring variants in G1Fc would include E134D and M136L according to the numbering system used in SEQ ID NO: 14 (see Uniprot P01857).

| (SEQ ID NO: 14) | |
|---|---|
| 1 | 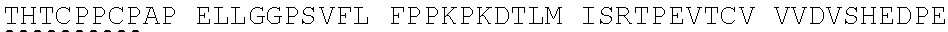 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

In some embodiments, the sequence that may be used for the Fc portion of human IgG1 (G1Fc) is shown below:

(SEQ ID NO: 233)
THTCPPCPAPELLGGPSVFLFPPKPKDTLMISRTPE
VTCVVVDVSHEDPEVKENWYVDGVEVHNAKTKPREE
QYNSTYRVVSVLTVLHQDWLNGKEYKCKVSNKALPV
PIEKTISKAKGQPREPQVYTLPPSREEMTKNQVSLT
CLVKGFYPSDIAVEWESNGQPENNYKTTPPVLDSDG
PFFLYSKLTVDKSRWQQGNVFSCSVMHEALHNHYTQ
KSLSLSPGK

In some embodiments, an Fc domain is from an IgG1 antibody and includes amino acid substitutions L12A, L13A, and G15A, relative to the sequence of SEQ ID NO: 233. In some embodiments, an Fc domain is from an IgG1 antibody and includes amino acid substitutions D43A, K1ODA, and N212A, relative to the sequence of SEQ ID NO: 233. In some embodiments, a polypeptide described herein (e.g., an ActRIIA, ActRIIB, or ALK1 polypeptide)) may be fused to the N- or C-terminus of an Fc domain monomer (e.g., SEQ ID NO: 233) through conventional genetic or chemical means, e.g., chemical conjugation. If desired, a linker (e.g., a spacer) can be inserted between the polypeptide and the Fc domain monomer. In some embodiments, the FcFc domain monomer can be fused to the N- or C-terminus (e.g., C-terminus) of the polypeptide.

In some embodiments, an Fc domain includes one or more of the following amino acid substitutions: T366W, T366Y, T394W, F405W, Y349T, Y349E, Y349V, L351T, L351H, L351N, L352K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y407I, K409E, K409D, K409T, and K409I, relative to the sequence of human IgG1. In some embodiments, an Fc domain includes the amino acid substitution T366W, relative to the sequence of human IgG1. The sequence of a wild-type Fc domain is shown in SEQ ID NO: 284.

An example of a native amino acid sequence that may be used for the Fc portion of human IgG2 (G2Fc) is shown below (SEQ ID NO: 15). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 15.

| (SEQ ID NO: 15) | |
|---|---|
| 1 | 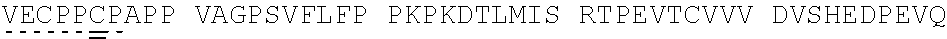 |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 16) contains a short hinge region consisting of a single 15-residue segment, whereas the second G3Fc sequence (SEQ ID NO: 17) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 16 and 17.

| (SEQ ID NO: 16) | |
|---|---|
| 1 | 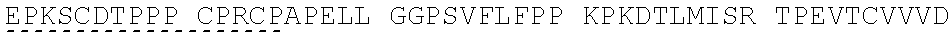 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| (SEQ ID NO:17) | |
| 1 | 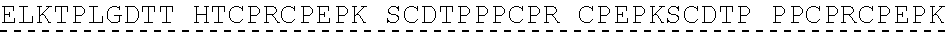 |
| 51 | 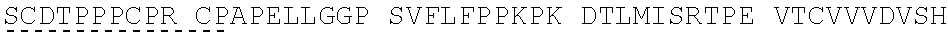 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 16, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 18). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 18.

| (SEQ ID NO: 18) | |
|---|---|
| 1 | 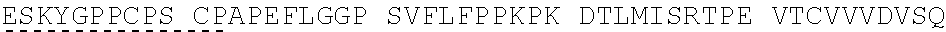 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

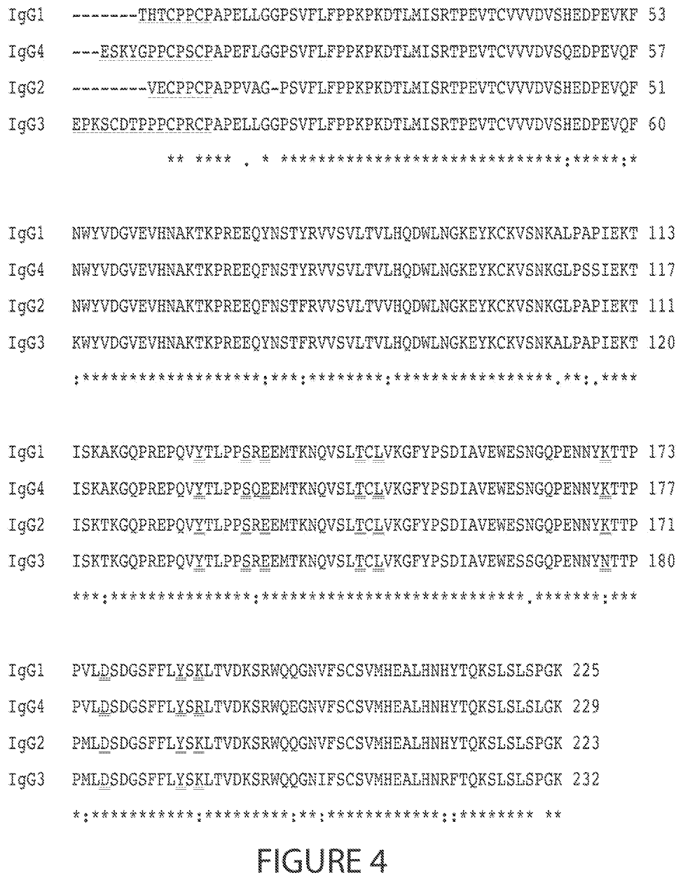

A variety of engineered mutations in the Fe domain are presented herein with respect to the G1Fc sequence (SEQ TD NO: 14), and analogous mutations in G2Fc, G3Fc, and G4Fc can be derived from their alignment with G1Fc in FIG. 4. Due to unequal hinge lengths, analogous Fc positions based on isotype alignment (FIG. 4) possess different amino acid numbers in SEQ TD NOs: 14, 15, 16, 17, and 18. It can also be appreciated that a given amino acid position in an immunoglobulin sequence consisting of hinge, C_{H}2, and C_{H}3 regions (e.g., SEQ ID NOs: 14, 15, 16, 17, and 18) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 14), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | IgG1 heavy chain |
|---|---|---|
| (Numbering begins | constant domain | (EU numbering |
| at first threonine | (Numbering | scheme of Kabat |
| in hinge region) | begins at C_{H}1) | et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

The application further provides Fc fusion proteins with engineered or variant Fc regions. Such Fc fusion proteins may be useful, for example, in modulating effector functions, such as, antigen-dependent cytotoxicity (ADCC) and complement-dependent cytotoxicity (CDC). Additionally, the modifications may improve the stability of the Fc fusion proteins. Amino acid sequence variants of the Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the Fc fusion proteins, such as changing the number or position of glycosylation sites. In some embodiments, Fc polypeptide domains of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a polypeptide selected from the group consisting of SEQ ID NOs: 14, 15, 16, 17, 18, 133, 134, 135, 136, 233, and 284.

In some embodiments, a polypeptide disclosed herein (e.g., an ActRIIA variant polypeptide, an ActRIIB variant polypeptide, an ALK4 polypeptide, or a heteromultimer comprising the same) may further include a moiety (e.g., Fc domain monomer, a wild-type Fc domain, an Fc domain with amino acid substitutions (e.g., one or more substitutions that reduce dimerization), an albumin-binding peptide, a fibronectin domain, or a human serum albumin), which may be fused to the N- or C-terminus (e.g., C-terminus) of the polypeptide by way of a linker or other covalent bonds. A polypeptide (e.g., an ActRIIA variant polypeptide, an ActRIIB variant polypeptide, an ALK4 polypeptide, or a heteromultimer comprising the same) fused to an Fc domain monomer may form a dimer (e.g., homodimer or heterodimer) through the interaction between two Fc domain monomers, which combine to form an Fc domain in the dimer.

Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26) and the P329G/L234A/L235A (P329G LALA) mutation described by Schlothauer et al. (see Schlothauer T., et al. Protein Eng Des Sel. 2016 October; 29(10):457-466). Thus in certain embodiments, Fc fusion proteins of the disclosure with mutations within the constant region including the Ala-Ala mutation or the P329G LALA mutation may be used to reduce or abolish effector function. According to these embodiments, Fc fusion proteins may comprise a mutation to an alanine at position 234 or a mutation to an alanine at position 235, or a combination thereof. In one embodiment, the Fc fusion protein comprises an IgG4 framework, wherein the Ala-Ala mutation would describe a mutation(s) from phenylalanine to alanine at position 234 and/or a mutation from leucine to alanine at position 235. In some embodiments, Fc fusion proteins may further comprise mutation from proline to glycine at position 329. In another embodiment, the Fc fusion protein comprises an IgG1 framework, wherein the Ala-Ala mutation would describe a mutation(s) from leucine to alanine at position 234 and/or a mutation from leucine to alanine at position 235. In some embodiments, the Fc fusion protein comprising an IgG1 framework further comprises a mutation from proline to glycine at position 329. The Fc fusion protein may alternatively or additionally carry other mutations, including the point mutation K322A in the CH2 domain (Hezareh et al. 2001 J Virol. 75: 12161-8).

In some embodiments, the Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & WinterNature 322: 738-40 (1988); U.S. Pat. Nos. 5,648,260; 5,624,821; and WO94/29351.

In certain aspects, the polypeptides disclosed herein may form protein complexes comprising at least one ALK4 polypeptide associated, covalently or non-covalently, with at least one ActRIIB polypeptide. Preferably, polypeptides disclosed herein form heterodimeric complexes, although higher order heteromultimeric complexes (heteromultimers) are also included such as, but not limited to, heterotrimers, heterotetramers, and further oligomeric structures (see, e.g., FIG. 21-23). In some embodiments, ALK4 and/or ActRIIB polypeptides comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides disclosed herein may be joined covalently or non-covalently to a multimerization domain. Preferably, a multimerization domain promotes interaction between a first polypeptide (e.g., an ALK4 polypeptide) and a second polypeptide (e.g., an ActRIIB polypeptide) to promote heteromultimer formation (e.g., heterodimer formation), and optionally hinders or otherwise disfavors homomultimer formation (e.g., homodimer formation), thereby increasing the yield of desired heteromultimer (see, e.g., FIG. 22).

Many methods known in the art can be used to generate ALK4:ActRIIB or ALK4:ActRIIA heteromultimers. For example, non-naturally occurring disulfide bonds may be constructed by replacing on a first polypeptide (e.g., an ALK4 polypeptide) a naturally occurring amino acid with a free thiol-containing residue, such as cysteine, such that the free thiol interacts with another free thiol-containing residue on a second polypeptide (e.g., an ActRIIB and/or ActRIIA polypeptide) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S. Pat. No. 8,592,562; coiled-coil interactions such as described in Christensen et al., U.S. 20120302737; leucine zippers such as described in Pack & Plueckthun, (1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form protein complexes comprising a first polypeptide covalently or non-covalently associated with a second polypeptide, wherein the first polypeptide comprises the amino acid sequence of an ALK4 polypeptide and the amino acid sequence of a first member of an interaction pair; and the second polypeptide comprises the amino acid sequence of an ActRIIB polypeptide and the amino acid sequence of a second member of an interaction pair. The interaction pair may be any two polypeptide sequences that interact to form a complex, particularly a heterodimeric complex although operative embodiments may also employ an interaction pair that can form a homodimeric complex. One member of the interaction pair may be fused to an ALK4 or ActRIIB polypeptide as described herein, including for example, a polypeptide sequence comprising, consisting essentially of, or consisting of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of any one of SEQ ID NOs: 2, 3, 5, 6, 101, and 103. An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric complex (see, e.g., FIG. 22). Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

As specific examples, the present disclosure provides fusion proteins comprising ALK4 or ActRIIB fused to a polypeptide comprising a constant domain of an immunoglobulin, such as a CH1, CH2, or CH3 domain derived from human IgG1, IgG2, IgG3, and/or IgG4 that has been modified to promote heteromultimer formation. A problem that arises in large-scale production of asymmetric immunoglobulin-based proteins from a single cell line is known as the “chain association issue”. As confronted prominently in the production of bispecific antibodies, the chain association issue concerns the challenge of efficiently producing a desired multi-chain protein from among the multiple combinations that inherently result when different heavy chains and/or light chains are produced in a single cell line [Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multi-chain fusion protein that incorporates only two different (asymmetric) heavy chains.

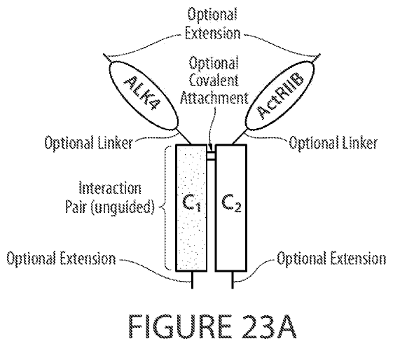

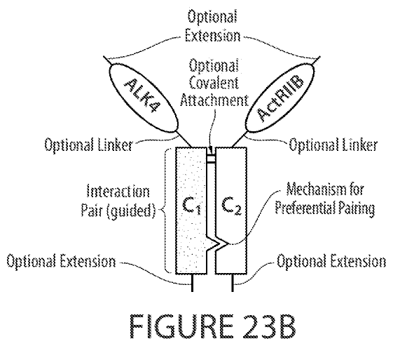

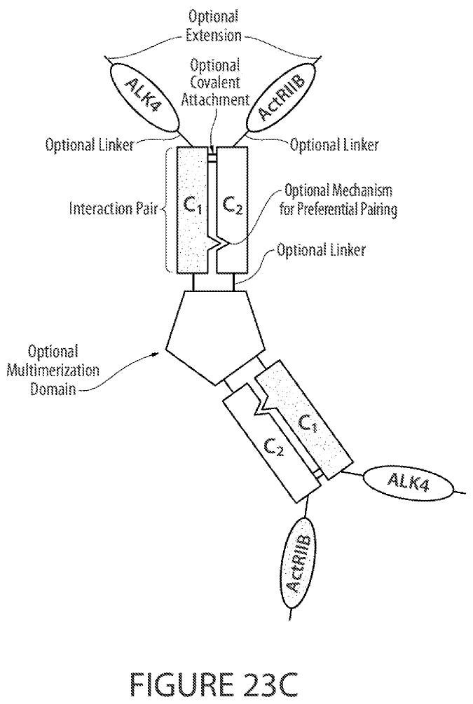

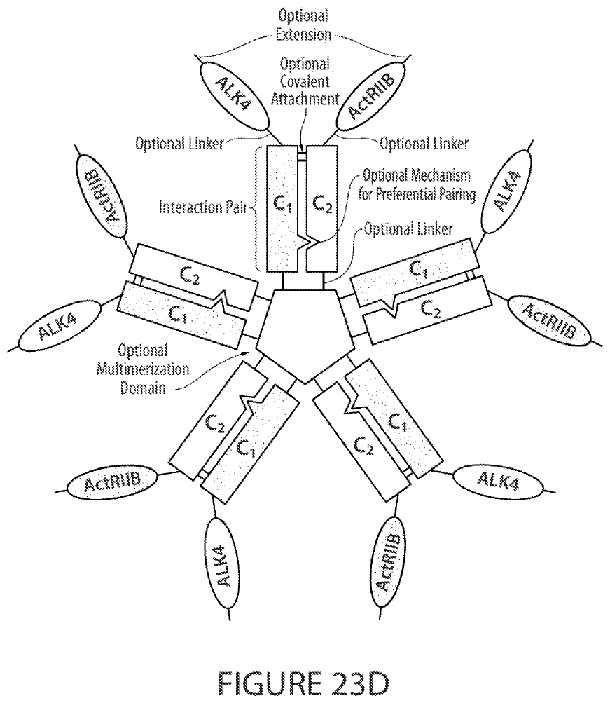

Various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing [Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605]. As described herein, these methods may be used to generate ALK4-Fc:ActRIIB-Fc heteromultimer complexes. See, e.g., FIG. 23.

ALK4:ActRIIB heteromultimers, ALK4:ActRIIA heteromultimers, and method of making such heteromultimers have been previously disclosed. See, for example, WO 2016/164497 and WO 2016/164089, the entire teachings of which are incorporated by reference herein.

In some embodiments, a polypeptide described herein may include an extracellular ActRIIA variant fused to a serum protein-binding peptide. Binding to serum protein peptides can improve the pharmacokinetics of protein pharmaceuticals.

As one example, albumin-binding peptides that can be used in the methods and compositions described here are generally known in the art. In one embodiment, the albumin binding peptide includes the sequence DICLPRWGCLW (SEQ ID NO: 285).

In some embodiments, albumin-binding peptides may be joined to the N- or C-terminus (e.g., C-terminus) of a polypeptide described herein (e.g., an ActRIIA, ActRIIB, or ALK4 polypeptide)) to increase the serum half-life of the extracellular ActRIIA variant. In some embodiments, an albumin-binding peptide is joined, either directly or through a linker, to the N- or C-terminus of the polypeptide.

In some embodiments, a polypeptide described herein (e.g., an ActRIIA, ActRIIB, or ALK4 polypeptide) may be fused to the N- or C-terminus of albumin-binding peptide (e.g., SEQ ID NO: 285) through conventional genetic or chemical means, e.g., chemical conjugation. If desired, a linker (e.g., a spacer) can be inserted between the polypeptide and the albumin-binding peptide. Without being bound to a theory, it is expected that inclusion of an albumin-binding peptide in an extracellular ActRIIA variant described herein may lead to prolonged retention of the therapeutic protein through its binding to serum albumin.

In some embodiments, a polypeptide described herein may include a polypeptide (e.g. an ActRIIA, ActRIIB, or ALK4 polypeptide) fused to fibronectin domains. Binding to fibronectin domains can improve the pharmacokinetics of protein pharmaceuticals.

A fibronectin domain is a high molecular weight glycoprotein of the extracellular matrix, or a fragment thereof, that binds to, e.g., membrane-spanning receptor proteins such as integrins and extracellular matrix components such as collagens and fibrins. In some embodiments, a fibronectin domain is joined to the N- or C-terminus (e.g., C-terminus) of a polypeptide described herein (e.g., an ActRIIA, ActRIIB, or ALK4 polypeptide) to increase the serum half-life of the polypeptide. A fibronectin domain can be joined, either directly or through a linker, to the N- or C-terminus of a polypeptide.

As one example, fibronectin domains that can be used in the methods and compositions described here are generally known in the art. In one embodiment, the fibronectin domain is a fibronectin type III domain (SEQ ID NO: 286) having amino acids 610-702 of the sequence of UniProt ID NO: P02751. In another embodiment, the fibronectin domain is an adnectin protein.

In some embodiments, a polypeptide described herein (e.g., an ActRIIA, ActRIIB, or ALK4 polypeptide) may be fused to the N- or C-terminus of a fibronectin domain (e.g., SEQ ID NO: 286) through conventional genetic or chemical means, e.g., chemical conjugation. If desired, a linker (e.g., a spacer) can be inserted between the polypeptide and the fibronectin domain. Without being bound to a theory, it is expected that inclusion of a fibronectin domain in a polypeptide described herein may lead to prolonged retention of the therapeutic protein through its binding to integrins and extracellular matrix components such as collagens and fibrins.

In some embodiments, a polypeptide described herein may include an ActRIIA, ActRIIB, or ALK4 polypeptide fused to serum albumin. Binding to serum albumins can improve the pharmacokinetics of protein pharmaceuticals.

Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In some embodiments, serum albumin is human serum albumin. In some embodiments, a human serum albumin is joined to the N- or C-terminus (e.g., C-terminus) of a polypeptide described herein (e.g., an ActRIIA, ActRIIB, or ALK4 polypeptide) to increase the serum half-life of the polypeptide. In some embodiments, the human serum albumin can be joined, either directly or through a linker, to the N- or C-terminus of a polypeptide disclosed herein.

As one example, serum albumins that can be used in the methods and compositions described herein are generally known in the art. In one embodiment, the serum albumin includes the sequence of UniProt ID NO: P02768 (SEQ ID NO: 287).

In some embodiments, a polypeptide described herein (e.g., an ActRIIA, ActRIIB, or ALK4 polypeptide) may be fused to the N- or C-terminus of a human serum albumin (e.g., SEQ ID NO: 287) through conventional genetic or chemical means, e.g., chemical conjugation. If desired, a linker (e.g., a spacer) can be inserted between the polypeptide and the human serum albumin. Without being bound to a theory, it is expected that inclusion of a human serum albumin in an ActRIIA, ActRIIB, or ALK4 polypeptide described herein may lead to prolonged retention of the therapeutic protein.

It is understood that different elements of the fusion proteins (e.g., immunoglobulin Fc fusion proteins) may be arranged in any manner that is consistent with desired functionality. For example, an ActRII polypeptide (or ALK4 polypeptide) domain may be placed C-terminal to a heterologous domain, or alternatively, a heterologous domain may be placed C-terminal to an ActRII polypeptide (or ALK4 polypeptide) domain. The ActRII polypeptide (or ALK4 polypeptide) domain and the heterologous domain need not be adjacent in a fusion protein, and additional domains or amino acid sequences may be included C- or N-terminal to either domain or between the domains.

For example, an ActRII (or ALK4) receptor fusion protein may comprise an amino acid sequence as set forth in the formula A-B-C. The B portion corresponds to an ActRII (or ALK4) polypeptide domain. The A and C portions may be independently zero, one, or more than one amino acid, and both the A and C portions when present are heterologous to B. The A and/or C portions may be attached to the B portion via a linker sequence. In certain embodiments, an ActRII (or ALK4) fusion protein comprises an amino acid sequence as set forth in the formula A-B-C, wherein A is a leader (signal) sequence, B consists of an ActRII (or ALK4) polypeptide domain, and C is a polypeptide portion that enhances one or more of in vivo stability, in vivo half-life, uptake/administration, tissue localization or distribution, formation of protein complexes, and/or purification. In certain embodiments, an ActRII (or ALK4) fusion protein comprises an amino acid sequence as set forth in the formula A-B-C, wherein A is a TPA leader sequence, B consists of an ActRII (or ALK4) receptor polypeptide domain, and C is an immunoglobulin Fc domain. Preferred fusion proteins comprise the amino acid sequence set forth in any one of SEQ ID NOs: 32, 36, 39, 40, 42, 45, 46, 48, 69, 74, 77, 78, 108, 110, 111, 113, 114, 115, 116, 117, 118, 120, 122, 124, 139, 140, 141, 142, 143, 144, 318, or 331.

In preferred embodiments, ActRII polypeptides, ALK4 polypeptides, ALK4:ActRIIB heteromultimers, and ALK4:ActRIIA heteromultimers to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptides, ALK4 polypeptides, ALK4:ActRIIB heteromultimers, and ALK4:ActRIIA heteromultimers to be used in accordance with the methods described herein are recombinant polypeptides.

ActRII polypeptides, ALK4 polypeptides, ALK4:ActRIIB heteromultimers, and ALK4:ActRIIA heteromultimers of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length ActRII polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ActRII or ALK4 polypeptides using chemical cleavage (e.g., cyanogen bromide, hydroxylamine, etc.).

### 3. Linkers

The disclosure provides for ActRII polypeptides, ALK4 polypeptides, ALK4:ActRIIB heteromultimers, ALK4:ActRIIA heteromultimers, and variants thereof (e.g., ActRIIA polypeptides, ActRIIB polypeptides, ALK4 polypeptides, ALK4:ActRIIB heteromultimer polypeptides, and ALK4:ActRIIA heteromultimer polypeptides), and in these embodiments, the polypeptide portion (e.g. ActRIIA polypeptide) is connected to the heterologous portion (e.g., Fc portion) by means of a linker. In some embodiments, the linkers are glycine and serine rich linkers. In some embodiments, the linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 19), GGGG (SEQ ID NO: 20), TGGGG (SEQ ID NO: 21), SGGGG (SEQ ID NO: 22), TGGG (SEQ ID NO: 23), SGGG (SEQ ID NO: 24), or GGGGS (SEQ ID NO: 25) singlets, or repeats. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In preferred embodiments, the linker comprises the amino acid sequence GlyGlyGlyGlySer (GGGGS) (SEQ ID NO: 25), or repetitions thereof (GGGGS)n, where n≥2. In particular embodiments n≥3, or n=3-10. In some embodiments, n≥4, or n=4-10. In some embodiments, n is not greater than 4 in a (GGGGS)n linker. In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS)n sequence also comprises an N-terminal threonine. In some embodiments, the linker is any one of the following:

(SEQ ID NO: 85)
GGGGSGGGGS
(SEQ ID NO: 86)
TGGGGSGGGGS
(SEQ ID NO: 87)
TGGGGSGGGGSGGGGS
(SEQ ID NO: 88)
TGGGGSGGGGSGGGGSGGGGS
(SEQ ID NO: 89)
TGGGGSGGGGSGGGGSGGGGSGGGGS
(SEQ ID NO: 90)
TGGGGSGGGGSGGGGSGGGGSGGGGSGGGGS
or
(SEQ ID NO: 91)
TGGGGSGGGGSGGGGSGGGGSGGGGSGGGGSGGGGS

In some embodiments, the linker comprises the amino acid sequence of TGGGPKSCDK (SEQ ID NO: 92). In some embodiments, the linker is any one of SEQ ID NOs: 85-92 lacking the N-terminal threonine. In some embodiments, the linker does not comprise the amino acid sequence of SEQ ID NO: 90 or 91.

In some embodiments, a polypeptide described (e.g., ActRIIA polypeptides, ActRIIB polypeptides, ALK4 polypeptides, ALK4:ActRIIB heteromultimer polypeptides, and ALK4:ActRIIA heteromultimer polypeptides) herein may include a polypeptide fused to a moiety by way of a linker. In some embodiments, the moiety increases stability of the polypeptide. In some embodiments, the moity is selected from the group consisting of an Fc domain monomer, a wild-type Fc domain, an Fc domain with amino acid substitutions (e.g., one or more substitutions that reduce dimerization), an albumin-binding peptide, a fibronectin domain, or a human serum albumin. In some embodiments, a linker between a moiety (e.g., an Fc domain monomer (e.g., the sequence of SEQ ID NO: 233), a wild-type Fc domain (e.g., SEQ ID NO: 284), an Fc domain with amino acid substitutions (e.g., one or more substitutions that reduce dimerization), an albumin-binding peptide (e.g., SEQ ID NO: 285), a fibronectin domain (e.g., SEQ ID NO: 286), or a human serum albumin (e.g., SEQ ID NO: 287)) and a polypeptide (e.g., ActRIIA polypeptides, ActRIIB polypeptides, ALK4 polypeptides, ALK4:ActRIIB heteromultimer polypeptides, and ALK4:ActRIIA heteromultimer polypeptides), can be an amino acid linker including 1-200 amino acids. Suitable peptide linkers are known in the art, and include, for example, peptide linkers containing flexible amino acid residues such as glycine, alanine, and serine. In some embodiments, a linker can contain motifs, e.g., multiple or repeating motifs, of GA, GS, GG, GGA, GGS, GGG, GGGA (SEQ ID NO: 234), GGGS (SEQ ID NO: 235), GGGG (SEQ ID NO: 20), GGGGA (SEQ ID NO: 236), GGGGS (SEQ ID NO: 25), GGGGG (SEQ ID NO: 237), GGAG (SEQ ID NO: 238), GGSG (SEQ ID NO: 239), AGGG (SEQ ID NO: 240), or SGGG (SEQ ID NO: 24). In some embodiments, a linker can contain 2 to 12 amino acids including motifs of GA or GS, e.g., GA, GS, GAGA (SEQ ID NO: 241), GSGS (SEQ ID NO: 242), GAGAGA (SEQ ID NO: 243), GSGSGS (SEQ ID NO: 244), GAGAGAGA (SEQ ID NO: 245), GSGSGSGS (SEQ ID NO: 246), GAGAGAGAGA (SEQ ID NO: 247), GSGSGSGSGS (SEQ ID NO: 248), GAGAGAGAGAGA (SEQ ID NO: 249), and GSGSGSGSGSGS (SEQ ID NO: 250). In some embodiments, a linker can contain 3 to 12 amino acids including motifs of GGA or GGS, e.g., GGA, GGS, GGAGGA (SEQ ID NO: 251), GGSGGS (SEQ ID NO: 252), GGAGGAGGA (SEQ ID NO: 253), GGSGGSGGS (SEQ ID NO: 254), GGAGGAGGAGGA (SEQ ID NO: 255), and GGSGGSGGSGGS (SEQ ID NO: 256). In some embodiments, a linker can contain 4 to 12 amino acids including motifs of GGAG (SEQ ID NO: 238), GGSG (SEQ ID NO: 239), GGAGGGAG (SEQ ID NO: 257), GGSGGGSG (SEQ ID NO: 258), GGAGGGAGGGAG (SEQ ID NO: 259), and GGSGGGSGGGSG (SEQ ID NO: 260). In some embodiments, a linker can contain motifs of GGGGA (SEQ ID NO: 236) or GGGGS (SEQ ID NO: 25), e.g., GGGGAGGGGAGGGGA (SEQ ID NO: 261) and GGGGSGGGGSGGGGS (SEQ ID NO: 262). In some embodiments, an amino acid linker between a moiety (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a polypeptide (e.g., ActRIIA polypeptides, ActRIIB polypeptides, ALK4 polypeptides, ALK4:ActRIIB heteromultimer polypeptides, and ALK4:ActRIIA heteromultimer polypeptides) may be GGG, GGGA (SEQ ID NO: 234), GGGG (SEQ ID NO: 20), GGGAG (SEQ ID NO: 263), GGGAGG (SEQ ID NO: 264), or GGGAGGG (SEQ ID NO: 265).

In some embodiments, a linker can also contain amino acids other than glycine, alanine, and serine, e.g., AAAL (SEQ ID NO: 266), AAAK (SEQ ID NO: 267), AAAR (SEQ ID NO: 268), EGKSSGSGSESKST (SEQ ID NO: 269), GSAGSAAGSGEF (SEQ ID NO: 270), AEAAAKEAAAKA (SEQ ID NO: 271), KESGSVSSEQLAQFRSLD (SEQ ID NO: 272), GENLYFQSGG (SEQ ID NO: 273), SACYCELS (SEQ ID NO: 274), RSIAT (SEQ ID NO: 275), RPACKIPNDLKQKVMNH (SEQ ID NO: 276), GGSAGGSGSGSSGGSSGASGTGTAGGTGSGSGTGSG (SEQ ID NO: 277), AAANSSIDLISVPVDSR (SEQ ID NO: 278), or GGSGGGSEGGGSEGGGSEGGGSEGGGSEGGGSGGGS (SEQ ID NO: 279). In some embodiments, a linker can contain motifs, e.g., multiple or repeating motifs, of EAAAK (SEQ ID NO: 280). In some embodiments, a linker can contain motifs, e.g., multiple or repeating motifs, of praline-rich sequences such as (XP)n, in which X may be any amino acid (e.g., A, K, or E) and n is from 1-5, and PAPAP(SEQ ID NO: 281).

The length of the peptide linker and the amino acids used can be adjusted depending on the two proteins involved and the degree of flexibility desired in the final protein fusion polypeptide. The length of the linker can be adjusted to ensure proper protein folding and avoid aggregate formation.

### 4. Nucleic Acids Encoding ActRII and ALK4 Polypeptides and Variants Thereof

In certain embodiments, the present disclosure provides isolated and/or recombinant nucleic acids encoding ActRII and/or ALK4 polypeptides (including fragments, functional variants, and fusion proteins thereof). For example, SEQ ID NO: 7 encodes a naturally occurring human ActRIIB precursor polypeptide (the R64 variant described above), while SEQ ID NO: 8 encodes the processed extracellular domain of ActRIIB (the R64 variant described above). The subject nucleic acids may be single-stranded or double-stranded. Such nucleic acids may be DNA or RNA molecules. These nucleic acids may be used, for example, in methods for making ActRII-based variant polypeptides as described herein.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRII or ALK4 polypeptides of the disclosure are understood to include nucleic acids that are variants of any one of SEQ ID NOs: 7, 8, 12, 13, 37, 43, 49, 70, 71, 72, 73, 75, 76, 80, 81, 82, 83, 84, 94, 102, 103, 106, 107, 109, 112, 119, 121, 123, and 125. Variant nucleotide sequences include sequences that differ by one or more nucleotide substitutions, additions, or deletions including allelic variants, and therefore, will include coding sequence that differ from the nucleotide sequence designated in any one of SEQ ID NOs: 7, 8, 12, 13, 37, 43, 49, 70, 71, 72, 73, 75, 76, 80, 81, 82, 83, 84, 94, 102, 103, 106, 107, 109, 112, 119, 121, 123, and 125.

In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to any one of SEQ ID NOs: 7, 8, 12, 13, 37, 43, 49, 70, 71, 72, 73, 75, 76, 80, 81, 82, 83, 84, 94, 102, 103, 106, 107, 109, 112, 119, 121, 123, and 125. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 7. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 8. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 12. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 13. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 37. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 43. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 49. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 70. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 71. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 72. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 73. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 75. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 76. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 80. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 81. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 82. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 83. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 84. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 94. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 102. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 103. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 106. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 107. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 109. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 112. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 119. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 121. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 123. In certain embodiments, ActRII or ALK4 polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 125. One of ordinary skill in the art will appreciate that nucleic acid sequences that are at least 70%, 75%, 80%, 85%, 90%, 91%, 92%, 93%, 94% 95%, 96%, 97%, 98%, 99%, or 100% identical to the sequences complementary to SEQ ID NOs: 7, 8, 12, 13, 37, 43, 49, 70, 71, 72, 73, 75, 76, 80, 81, 82, 83, 84, 94, 102, 103, 106, 107, 109, 112, 119, 121, 123, and 125, and variants thereof, are also within the scope of the present disclosure. In further embodiments, the nucleic acid sequences of the disclosure can be isolated, recombinant, and/or fused with a heterologous nucleotide sequence, or in a DNA library.

In other embodiments, nucleic acids of the present disclosure also include nucleotide sequences that hybridize under highly stringent conditions to the nucleotide sequence designated in SEQ ID NOs: 7, 8, 12, 13, 37, 43, 49, 70, 71, 72, 73, 75, 76, 80, 81, 82, 83, 84, 94, 102, 103, 106, 107, 109, 112, 119, 121, 123, and 125, complement sequences of SEQ ID NOs: 7, 8, 12, 13, 37, 43, 49, 70, 71, 72, 73, 75, 76, 80, 81, 82, 83, 84, 94, 102, 103, 106, 107, 109, 112, 119, 121, 123, and 125, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 7, 8, 12, 13, 37, 43, 49, 70, 71, 72, 73, 75, 76, 80, 81, 82, 83, 84, 94, 102, 103, 106, 107, 109, 112, 119, 121, 123, and 125 to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art and can be used in a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and can vary with the host cell used.

In certain aspects, the subject nucleic acid disclosed herein is provided in an expression vector comprising a nucleotide sequence encoding an ActRII and/or ALK4 polypeptide and operably linked to at least one regulatory sequence. Regulatory sequences are art-recognized and are selected to direct expression of the ActRII and/or ALK4 polypeptide. Accordingly, the term “regulatory sequence” includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding an ActRII and/or ALK4 polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRII and/or ALK4 polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In a preferred embodiment, a vector will be designed for production of the subject ActRII and/or ALK4 polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject ActRII polypeptides in cells propagated in culture, e.g., to produce proteins, including fusion proteins or variant proteins, for purification.

This disclosure also pertains to a host cell transfected with a recombinant gene including a coding sequence for one or more of the subject ActRII and/or ALK4 polypeptides. The host cell may be any prokaryotic or eukaryotic cell. For example, an ActRII and/or ALK4 polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject ActRII and/or ALK4 polypeptides. For example, a host cell transfected with an expression vector encoding an ActRII and/or ALK4 polypeptide can be cultured under appropriate conditions to allow expression of the ActRII and/or ALK4 polypeptide to occur. The polypeptide may be secreted and isolated from a mixture of cells and medium containing the polypeptide. Alternatively, the ActRII and/or ALK4 polypeptide may be retained cytoplasmically or in a membrane fraction and the cells harvested, lysed and the protein isolated. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRII and/or ALK4 polypeptides, and affinity purification with an agent that binds to a domain fused to the ActRII polypeptide (e.g., a protein A column may be used to purify an ActRII-Fc and/or ALK4-Fc fusion proteins). In some embodiments, the ActRII and/or ALK4 polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An ActRII and/or ALK4 protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRII and/or ALK4 polypeptide, can allow purification of the expressed fusion protein by affinity chromatography using a Ni²⁺ metal resin. The purification leader sequence can then be subsequently removed by treatment with enterokinase to provide the purified ActRII and/or ALK4 polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 5. Screening Assays

In certain aspects, the present disclosure relates to the use of the subject ActRII polypeptides and heteromultimers comprising the same to identify compounds (agents) which may be used to treat, prevent, or reduce the progression rate and/or severity of pulmonary hypertension (PH), a kidney-associated disease (e.g., Alport syndrome, focal segmental glomerulosclerosis (FSGS), polycystic kidney disease, or chronic kidney disease), and/or an interstitial lung disease (e.g., idiopathic pulmonary fibrosis), particularly treating, preventing or reducing the progression rate and/or severity of one or more PH-associated complications, kidney-associated disease, and/or interstitial lung disease.

There are numerous approaches to screening for therapeutic agents for treating PH, a kidney associated disease, and/or an interstitial lung disease by targeting signaling (e.g., Smad signaling) of one or more ligands. In certain embodiments, high-throughput screening of compounds can be carried out to identify agents that perturb ligand-mediated effects on a selected cell line. In certain embodiments, the assay is carried out to screen and identify compounds that specifically inhibit or reduce binding of a TGF-beta ligand (e.g., activin A, activin B, activin AB, activin AC, BMP6, BMP7, BMP9, BMP10, GDF3, GDF8, and/or GDF11) to its binding partner, such as an a type II receptor (e.g., ActRIIA and/or ActRIIB). Alternatively, the assay can be used to identify compounds that enhance binding of a ligand to its binding partner such as a type II receptor. In a further embodiment, the compounds can be identified by their ability to interact with a type II receptor.

A variety of assay formats will suffice and, in light of the present disclosure, those not expressly described herein will nevertheless be comprehended by one of ordinary skill in the art. As described herein, the test compounds (agents) disclosed herein may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatible crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a TGF-beta ligand (e.g., activin A, activin B, activin AB, activin AC, BMP6, BMP7, BMP9, BMP10, GDF3, GDF8, and/or GDF11) to its binding partner, such as an a type II receptor (e.g., ActRIIA and/or ActRIIB).

Merely to illustrate, in an exemplary screening assay of the present disclosure, the compound of interest is contacted with an isolated and purified ActRIIB polypeptide which is ordinarily capable of binding to an ActRIIB ligand, as appropriate for the intention of the assay. To the mixture of the compound and ActRIIB polypeptide is then added to a composition containing an ActRIIB ligand (e.g., GDF11). Detection and quantification of ActRIIB/ActRIIB-ligand complexes provides a means for determining the compound's efficacy at inhibiting (or potentiating) complex formation between the ActRIIB polypeptide and its binding protein. The efficacy of the compound can be assessed by generating dose-response curves from data obtained using various concentrations of the test compound. Moreover, a control assay can also be performed to provide a baseline for comparison. For example, in a control assay, isolated and purified ActRIIB ligand is added to a composition containing the ActRIIB polypeptide, and the formation of ActRIIB/ActRIIB ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between a ligand and its binding protein may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled ActRIIB polypeptide and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a ligand and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between a ligand and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., peptides) that dissociate interactions between a ligand and its binding protein [see, e.g., Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with a particularligand. The interaction between the compound and the ligand may be covalent or non-covalent. For example, such interaction can be identified at the protein level using in vitro biochemical methods, including photo-crosslinking, radiolabeled ligand binding, and affinity chromatography [see, e.g., Jakoby W B et al. (1974) Methods in Enzymology 46:1]. In certain cases, the compounds may be screened in a mechanism-based assay, such as an assay to detect compounds which bind to a particularligand. This may include a solid-phase or fluid-phase binding event. Alternatively, the gene encoding a ligand can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 6. Therapeutic Uses

In part, the present disclosure relates to methods of treating pulmonary hypertension (e.g., pulmonary arterial hypertension), a kidney-associated disease (e.g., Alport syndrome, focal segmental glomerulosclerosis (FSGS), polycystic kidney disease, or chronic kidney disease), and/or an interstitial lung disease (e.g., idiopathic pulmonary fibrosis) comprising administering to a patient in need thereof an effective amount of any of, or any combination of, the ActRII antagonist. In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. In some embodiments, the disclosure contemplates methods of treating one or more complications of pulmonary hypertension (e.g., smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis) comprising administering to a patient in need thereof an effective amount of a ActRII antagonist or a heteromultimer comprising the same. In some embodiments, the disclosure contemplates methods of preventing one or more complications of pulmonary hypertension comprising administering to a patient in need thereof an effective amount of an ActRII antagonist or a heteromultimer comprising the same. In some embodiments, the disclosure contemplates methods of reducing the progression rate of pulmonary hypertension comprising administering to a patient in need thereof an effective amount of an ActRII antagonist or a heteromultimer comprising the same. In some embodiments, the disclosure contemplates methods of reducing the progression rate of one or more complications of pulmonary hypertension comprising administering to a patient in need thereof an effective amount of an ActRII antagonist or a heteromultimer comprising the same. In some embodiments, the disclosure contemplates methods of reducing the severity of pulmonary hypertension comprising administering to a patient in need thereof an effective amount of a ActRII antagonist or a heteromultimer comprising the same. In some embodiments, the disclosure contemplates methods of reducing the severity of one or more complications of pulmonary hypertension comprising administering to a patient in need thereof an effective amount of an ActRII antagonist or a heteromultimer comprising the same. Optionally, methods disclosed herein for treating, preventing, or reducing the progression rate and/or severity of pulmonary hypertension, particularly treating, preventing, or reducing the progression rate and/or severity of one or more complications of pulmonary hypertension, may further comprise administering to the patient one or more supportive therapies or additional active agents for treating pulmonary hypertension. For example, the patient also may be administered one or more supportive therapies or active agents selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl]oleanolic acid; 3-O-[alpha-L-rhamnopyranosyl-(1-->3)-beta-D-glucuronopyranosyl]oleanolic acid 28-O-beta-D-glucopyranosyl ester; 28-β-O-D-glucopyranosyl-oleanolic acid; 3-β-O-D-glucopyranosyl (1→3)-β-D-glucopyranosiduronic acid (CS1); oleanolic acid 3-β-O-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate) lung and/or heart transplantation. In some embodiment, the patient may also be administered a BMP9 polypeptide. In some embodiments the BMP9 polypeptide is a mature BMP9 polypeptide. In some embodiments, the BMP9 polypeptide comprises a BMP9 prodomain polypeptide. In some embodiments, the BMP9 polypeptide is administered in a pharmaceutical preparation, which optionally may comprise a BMP9 prodomain polypeptide. In such BMP9 pharmaceutical preparations comprising a BMP9 prodomain polypeptide, the BMP9 polypeptide may be noncovalently associated with the BMP9 prodomain polypeptide. In some embodiments, BMP9 pharmaceutical preparations are substantially free, or does not comprise, of BMP9 prodomain polypeptide. BMP9 polypeptides (mature and pro-polypeptides), BMP9 prodomain polypeptides, pharmaceutical compositions comprising the same as well as method of generative such polypeptides and pharmaceutical compositions are described in, for example, WO 2013/152213, which is incorporated by reference herein in its entirety.

In some embodiments, the present disclosure relates to methods of treating an interstitial lung disease (e.g., idiopathic pulmonary fibrosis) comprising administering to a patient in need thereof an effective amount of any of the ActRII antagonists disclosed herein (e.g., an antagonist of one or more of activin A, activin B, activin AB, activin AC, BMP6, BMP7, BMP9, BMP10, GDF3, GDF8, GDF11, and one or more Smad proteins). In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. In some embodiments, the interstitial lung disease is pulmonary fibrosis. In some embodiments, the interstitial lung disease is caused by any one of the following: silicosis, asbestosis, berylliosis, hypersensitivity pneumonitis, drug use (e.g., antibiotics, chemotherapeutic drugs, antiarrhythmic agents, statins), systemic sclerosis, polymyositis, dermatomyositis, systemic lupus erythematosus, rheumatoid arthritis, an infection (e.g., atypical pneumonia, Pneumocystis pneumonia, tuberculosis, Chlamydia trachomatis, and/or respiratory syncytial virus), lymphangitic carcinomatosis, cigarette smoking, or developmental disorders. In some embodiments, the interstitial lung disease is idiopathic (e.g., sarcoidosis, idiopathic pulmonary fibrosis, Hamman-Rich syndrome, and/or antisynthetase syndrome). In particular embodiments, the interstitial lung disease is idiopathic pulmonary fibrosis. In some embodiments, the treatment for idiopathic pulmonary fibrosis is administered in combination with an additional therapeutic agent. In some embodiments, the additional therapeutic agent is selected from the group consisting of: pirfenidone, N-acetylcysteine, prednisone, azathioprine, nintedanib, derivatives thereof and combinations thereof.

The terms “treatment”, “treating”, “alleviation” and the like are used herein to generally mean obtaining a desired pharmacologic and/or physiologic effect, and may also be used to refer to improving, alleviating, and/or decreasing the severity of one or more clinical complication of a condition being treated. The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure is achieved by administering an ActRII antagonist in an effective amount. An effective amount of an agent refers to an amount effective, at dosages and for periods of time necessary, to achieve the desired therapeutic or prophylactic result. A therapeutically effective amount of an agent of the present disclosure may vary according to factors such as the disease state, age, sex, and weight of the individual, and the ability of the agent to elicit a desired response in the individual. A prophylactically effective amount refers to an amount effective, at dosages and for periods of time necessary, to achieve the desired prophylactic result.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

In certain aspects, the disclosure contemplates the use of an ActRII antagonist, in combination with one or more additional active agents or other supportive therapy for treating or preventing a disease or condition (e.g., pulmonary hypertension, pulmonary arterial hypertension, and ILD). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more ActRII antagonist of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the ActRII antagonist of the present disclosure with the additional active agent or therapy and/or the desired effect.

Pulmonary hypertension (PH) has been previously classified as primary (idiopathic) or secondary. Recently, the World Health Organization (WHO) has classified pulmonary hypertension into five groups: Group 1: pulmonary arterial hypertension (PAH); Group 2: pulmonary hypertension with left heart disease; Group 3: pulmonary hypertension with lung disease and/or hypoxemia; Group 4: pulmonary hypertension due to chronic thrombotic and/or embolic disease; and Group 5: miscellaneous conditions (e.g., sarcoidosis, histiocytosis X, lymphangiomatosis and compression of pulmonary vessels). See, for example, Rubin (2004) Chest 126:7-10.

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. In some embodiments, the method relates to pulmonary hypertension patients that have pulmonary arterial hypertension. In some embodiments, the method relates pulmonary hypertension patients that have pulmonary hypertension with left heart disease. In some embodiments, the method relates to pulmonary hypertension patients that have lung disease and/or hypoxemia. In some embodiments, the method relates to pulmonary hypertension patients that have chronic thrombotic and/or embolic disease. In some embodiments, the method relates to pulmonary hypertension patients that have sarcoidosis, histiocytosis X, or lymphangiomatosis and compression of pulmonary vessels.

Pulmonary arterial hypertension is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in pulmonary vascular resistance (PVR) and sustained elevations in pulmonary artery pressure (PAP), which ultimately lead to right ventricular failure and death. Patients diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of patients with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has resting pulmonary arterial pressure (PAP) of at least 25 mm Hg (e.g., 25, 30, 35, 40, 45, or 50 mm Hg). In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. In some embodiments, the method relates to patients having a resting PAP of at least 25 mm Hg. In some embodiments, the method relates to patients having a resting PAP of at least 30 mm Hg. In some embodiments, the method relates to patients having a resting PAP of at least 35 mm Hg. In some embodiments, the method relates to patients having a resting PAP of at least 40 mm Hg. In some embodiments, the method relates to patients having a resting PAP of at least 45 mm Hg. In some embodiments, the method relates to patients having a resting PAP of at least 50 mm Hg.

In some embodiments, the disclosure relates to methods of adjusting one or more hemodynamic parameters in the PH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. In some embodiments, the method relates to reducing PAP. In some embodiments, the method relates to reducing the patient's PAP by at least 3 mmHg. In certain embodiments, the method relates to reducing the patient's PAP by at least 5 mmHg. In certain embodiments, the method relates to reducing the patient's PAP by at least 7 mmHg. In certain embodiments, the method relates to reducing the patient's PAP by at least 10 mmHg. In certain embodiments, the method relates to reducing the patient's PAP by at least 12 mmHg. In certain embodiments, the method relates to reducing the patient's PAP by at least 15 mmHg. In certain embodiments, the method relates to reducing the patient's PAP by at least 20 mmHg. In certain embodiments, the method relates to reducing the patient's PAP by at least 25 mmHg. In some embodiments, the method relates to reducing pulmonary vascular resistance (PVR). In some embodiments, the method relate to increasing pulmonary capillary wedge pressure (PCWP). In some embodiments, the method relate to increasing left ventricular end-diastolic pressure (LVEDP).

In certain aspects, the disclosure relates to methods of treating, preventing, or reducing the progression rate and/or severity of one or more complications of pulmonary hypertension comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to treating, preventing, or reducing the progression rate and/or severity of cell proliferation in the pulmonary artery of a pulmonary hypertension patient. In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. In some embodiments, the method relates to treating, preventing, or reducing the progression rate and/or severity of smooth muscle and/or endothelial cells proliferation in the pulmonary artery of a pulmonary hypertension patient. In some embodiments, the method relates to treating, preventing, or reducing the progression rate and/or severity of angiogenesis in the pulmonary artery of a pulmonary hypertension patient. In some embodiments, the method relates to increasing physical activity of a patient having pulmonary hypertension. In some embodiments, the method relates to treating, preventing, or reducing the progression rate and/or severity of dyspnea in a pulmonary hypertension patient. In some embodiments, the method relates to treating, preventing, or reducing the progression rate and/or severity of chest pain in a pulmonary hypertension patient. In some embodiments, the method relates to treating, preventing, or reducing the progression rate and/or severity of fatigue in a pulmonary hypertension patient. In some embodiments, the method relates to treating, preventing, or reducing the progression rate and/or severity of pulmonary fibrosis in a pulmonary hypertension patient. In some embodiments, the method relates to treating, preventing, or reducing the progression rate and/or severity of fibrosis in a pulmonary hypertension patient. In some embodiments, the method relates to treating, preventing, or reducing the progression rate and/or severity of pulmonary vascular remodeling in a pulmonary hypertension patient. In some embodiments, the method relates to treating, preventing, or reducing the progression rate and/or severity of right ventricular hypertrophy in a pulmonary hypertension patient.

In certain aspects, the disclosure relates to methods of increasing exercise capacity in a patient having pulmonary hypertension comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. Any suitable measure of exercise capacity can be used. For example, exercise capacity in a 6-minute walk test (6MWT), which measures how far the subject can walk in 6 minutes, i.e., the 6-minute walk distance (6MWD), is frequently used to assess pulmonary hypertension severity and disease progression. The Borg dyspnea index (BDI) is a numerical scale for assessing perceived dyspnea (breathing discomfort). It measures the degree of breathlessness, for example, after completion of the 6MWT, where a BDI of 0 indicates no breathlessness and 10 indicates maximum breathlessness. In some embodiments, the method relates to increasing 6MWD by at least 10 meters in the patient having pulmonary hypertension. In some embodiments, the method relates to increasing 6MWD by at least 20 meters in the patient having pulmonary hypertension. In some embodiments, the method relates to increasing 6MWD by at least 30 meters in the patient having pulmonary hypertension. In some embodiments, the method relates to increasing 6MWD by at least 40 meters in the patient having pulmonary hypertension. In some embodiments, the method relates to increasing 6MWD by at least 50 meters in the patient having pulmonary hypertension. In some embodiments, the method relates to increasing 6MWD by at least 60 meters in the patient having pulmonary hypertension. In some embodiments, the method relates to increasing 6MWD by at least 70 meters in the patient having pulmonary hypertension. In some embodiments, the method relates to increasing 6MWD by at least 80 meters in the patient having pulmonary hypertension. In some embodiments, the method relates to increasing 6MWD by at least 90 meters in the patient having pulmonary hypertension. In some embodiments, the method relates to increasing 6MWD by at least 100 meters in the patient having pulmonary hypertension. In some embodiments, the method relate to lowering BDI by at least 0.5 index points in the patient having pulmonary hypertension. In some embodiments, the method relate to lowering BDI by at least 1 index points in the patient having pulmonary hypertension. In some embodiments, the method relate to lowering BDI by at least 1.5 index points in the patient having pulmonary hypertension. In some embodiments, the method relate to lowering BDI by at least 2 index points in the patient having pulmonary hypertension. In some embodiments, the method relate to lowering BDI by at least 2.5 index points in the patient having pulmonary hypertension. In some embodiments, the method relate to lowering BDI by at least 3 index points in the patient having pulmonary hypertension. In some embodiments, the method relate to lowering BDI by at least 3.5 index points in the patient having pulmonary hypertension. In some embodiments, the method relate to lowering BDI by at least 4 index points in the patient having pulmonary hypertension. In some embodiments, the method relate to lowering BDI by at least 4.5 index points in the patient having pulmonary hypertension. In some embodiments, the method relate to lowering BDI by at least 5 index points in the patient having pulmonary hypertension. In some embodiments, the method relate to lowering BDI by at least 5.5 index points in the patient having pulmonary hypertension. In some embodiments, the method relate to lowering BDI by at least 6 index points in the patient having pulmonary hypertension. In some embodiments, the method relate to lowering BDI by at least 6.5 index points in the patient having pulmonary hypertension. In some embodiments, the method relate to lowering BDI by at least 7 index points in the patient having pulmonary hypertension. In some embodiments, the method relate to lowering BDI by at least 7.5 index points in the patient having pulmonary hypertension. In some embodiments, the method relate to lowering BDI by at least 8 index points in the patient having pulmonary hypertension. In some embodiments, the method relate to lowering BDI by at least 8.5 index points in the patient having pulmonary hypertension. In some embodiments, the method relate to lowering BDI by at least 9 index points in the patient having pulmonary hypertension. In some embodiments, the method relate to lowering BDI by at least 9.5 index points in the patient having pulmonary hypertension. In some embodiments, the method relate to lowering BDI by at least 3 index points in the patient having pulmonary hypertension. In some embodiments, the method relate to lowering BDI by 10 index points in the patient having pulmonary hypertension.

Pulmonary hypertension at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Class I: pulmonary hypertension without resulting limitation of physical activity; ordinary physical activity does not cause undue dyspnea or fatigue, chest pain or near syncope; Class II: pulmonary hypertension resulting in slight limitation of physical activity; patient comfortable at rest; ordinary physical activity causes undue dyspnea or fatigue, chest pain or near syncope; Class III: pulmonary hypertension resulting in marked limitation of physical activity; patient comfortable at rest; less than ordinary activity causes undue dyspnea or fatigue, chest pain or near syncope; Class IV: pulmonary hypertension resulting in inability to carry out any physical activity without symptoms; patient manifests signs of right-heart failure; dyspnea and/or fatigue may be present even at rest; discomfort is increased by any physical activity.

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has Class I, Class II, Class III, or Class IV pulmonary hypertension as recognized by the WHO. In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. In some embodiments, the method relates to a patient that has Class I pulmonary hypertension as recognized by the WHO. In some embodiments, the method relates to a patient that has Class II pulmonary hypertension as recognized by the WHO. In some embodiments, the method relates to preventing or delaying patient progression from Class I pulmonary hypertension to Class II pulmonary hypertension as recognized by the WHO. In some embodiments, the method relates to promoting or increasing patient regression from Class II pulmonary hypertension to Class I pulmonary hypertension as recognized by the WHO. In some embodiments, the method relates to a patient that has Class III pulmonary hypertension as recognized by the WHO. In some embodiments, the method relates to preventing or delaying patient progression from Class II pulmonary hypertension to Class III pulmonary hypertension as recognized by the WHO. In some embodiments, the method relates to promoting or increasing patient regression from Class III pulmonary hypertension to Class II pulmonary hypertension as recognized by the WHO. In some embodiments, the method relates to promoting or increasing patient regression from Class III pulmonary hypertension to Class I pulmonary hypertension as recognized by the WHO. In some embodiments, the method relates to a patient that has Class IV pulmonary hypertension as recognized by the WHO. In some embodiments, the method relates to preventing or delaying patient progression from Class III pulmonary hypertension to Class IV pulmonary hypertension as recognized by the WHO. In some embodiments, the method relates to promoting or increasing patient regression from Class IV pulmonary hypertension to Class III pulmonary hypertension as recognized by the WHO. In some embodiments, the method relates to promoting or increasing patient regression from Class IV pulmonary hypertension to Class II pulmonary hypertension as recognized by the WHO. In some embodiments, the method relates to promoting or increasing patient regression from Class IV pulmonary hypertension to Class I pulmonary hypertension as recognized by the WHO.

There is no known cure for pulmonary hypertension; current methods of treatment focus on prolonging patient lifespan and enhancing patient quality of life. Current methods of treatment of pulmonary hypertension include administration of: vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of pulmonary hypertension has also been carried out using oxygen therapy, atrial septostomy, pulmonary thromboendarterectomy, and lung and/or heart transplantation. Each of these methods, however, suffers from one or multiple drawbacks which may include lack of effectiveness, serious side effects, low patient compliance, and high cost. In certain aspects, the method relate to treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins) in combination (e.g., administered at the same time or different times, but generally in such a manner as to achieve overlapping pharmacological/physiological effects) with one or more additional active agents and/or supportive therapies for treating pulmonary hypertension (e.g., vasodilators such as prostacyclin, epoprostenol, and sildenafil; endothelin receptor antagonists such as bosentan; calcium channel blockers such as amlodipine, diltiazem, and nifedipine; anticoagulants such as warfarin; diuretics; oxygen therapy; atrial septostomy; pulmonary thromboendarterectomy: and lung and/or heart transplantation); BMP9 polypeptides; BMP10 polypeptides; bardoxolone methyl or a derivative thereof; oleanolic acid or derivative thereof.

In some embodiments, any of the the ActRII antagonists disclosed herein (e.g., an antagonist of one or more of activin A, activin B, activin AB, activin AC, BMP6, BMP7, BMP9, BMP10, GDF3, GDF8, GDF11, and one or more Smad proteins) may be used, alone or in combination with one or more supportive therapies or active agents, to treat, prevent, or reduce the progression rate and/or severity of a kidney-associated disease or condition. As used herein, “kidney-associated disease or condition” can refer to any disease, disorder, or condition that affects the kidneys or the renal system. Examples of kidney-associated diseases or conditions include, but are not limited to, chronic kidney diseases (or failure), acute kidney diseases (or failure), primary kidney diseases, non-diabetic kidney diseases, glomerulonephritis, interstitial nephritis, diabetic kidney diseases, diabetic nephropathy, glomerulosclerosis, rapid progressive glomerulonephritis, renal fibrosis, Alport syndrome, IDDM nephritis, mesangial proliferative glomerulonephritis, membranoproliferative glomerulonephritis, crescentic glomerulonephritis, renal interstitial fibrosis, focal segmental glomerulosclerosis (FSGS), membranous nephropathy, minimal change disease, pauci-immune rapid progressive glomerulonephritis, IgA nephropathy, polycystic kidney disease (PKD), Dent's disease, nephrocytinosis, Heymann nephritis, autosomal dominant (adult) polycystic kidney disease (ADPKD), autosomal recessive (childhood) polycystic kidney disease (ARPKD), acquired cystic kidney disease (ACKD), polycystic kidney syndrome (PKS), acute kidney injury, nephrotic syndrome, renal ischemia, podocyte diseases or disorders, proteinuria, glomerular diseases, membranous glomerulonephritis, focal segmental glomerulonephritis, pre-eclampsia, eclampsia, kidney lesions, collagen vascular diseases, benign orthostatic (postural) proteinuria, IgM nephropathy, membranous nephropathy, sarcoidosis, diabetes mellitus, kidney damage due to drugs, Fabry's disease, aminoaciduria, Fanconi syndrome, hypertensive nephrosclerosis, interstitial nephritis, Sickle cell disease, hemoglobinuria, myoglobinuria, Wegener's Granulomatosis, Glycogen Storage Disease Type 1, chronic kidney disease, chronic renal failure, low Glomerular Filtration Rate (GFR), nephroangiosclerosis, lupus nephritis, ANCA-positive pauci-immune crescentic glomerulonephritis, chronic allograft nephropathy, nephrotoxicity, renal toxicity, kidney necrosis, kidney damage, glomerular and tubular injury, kidney dysfunction, nephritic syndrome, acute renal failure, chronic renal failure, proximal tubal dysfunction, acute kidney transplant rejection, chronic kidney transplant rejection, non-IgA mesangioproliferative glomerulonephritis, postinfectious glomerulonephritis, vasculitides with renal involvement of any kind, any hereditary renal disease, any interstitial nephritis, renal transplant failure, kidney cancer, kidney disease associated with other conditions (e.g., hypertension, diabetes, and autoimmune disease), Dent's disease, nephrocytinosis, Heymann nephritis, a primary kidney disease, a collapsing glomerulopathy, a dense deposit disease, a cryoglobulinemia-associated glomerulonephritis, an Henoch-Schonlein disease, a postinfectious glomerulonephritis, a bacterial endocarditis, a microscopic polyangitis, a Churg-Strauss syndrome, an anti-GBM-antibody mediated glomerulonephritis, amyloidosis, a monoclonal immunoglobulin deposition disease, a fibrillary glomerulonephritis, an immunotactoid glomerulopathy, ischemic tubular injury, a medication-induced tubulo-interstitial nephritis, a toxic tubulo-interstitial nephritis, an infectious tubulo-interstitial nephritis, a bacterial pyelonephritis, a viral infectious tubulo-interstitial nephritis which results from a polyomavirus infection or an HIV infection, a metabolic-induced tubulo-interstitial disease, a mixed connective disease, a cast nephropathy, a crystal nephropathy which may results from urate or oxalate or drug-induced crystal deposition, an acute cellular tubulo-interstitial allograft rejection, a tumoral infiltrative disease which results from a lymphoma or a post-transplant lymphoproliferative disease, an obstructive disease of the kidney, vascular disease, a thrombotic microangiopathy, a nephroangiosclerosis, an atheroembolic disease, a mixed connective tissue disease, a polyarteritis nodosa, a calcineurin-inhibitor induced-vascular disease, an acute cellular vascular allograft rejection, an acute humoral allograft rejection, early renal function decline (ERFD), end stage renal disease (ESRD), renal vein thrombosis, acute tubular necrosis, acute interstitial nephritis, established chronic kidney disease, renal artery stenosis, ischemic nephropathy, uremia, drug and toxin-induced chronic tubulointerstitial nephritis, reflux nephropathy, kidney stones, Goodpasture's syndrome, normocytic normochromic anemia, renal anemia, diabetic chronic kidney disease, IgG4-related disease, von Hippel-Lindau syndrome, tuberous sclerosis, nephronophthisis, medullary cystic kidney disease, renal cell carcinoma, adenocarcinoma, nephroblastoma, lymphoma, leukemia, hyposialylation disorder, chronic cyclosporine nephropathy, renal reperfusion injury, renal dysplasia, azotemia, bilateral arterial occlusion, acute uric acid nephropathy, hypovolemia, acute bilateral obstructive uropathy, hypercalcemic nephropathy, hemolytic uremic syndrome, acute urinary retention, malignant nephrosclerosis, postpartum glomerulosclerosis, scleroderma, non-Goodpasture's anti-GBM disease, microscopic polyarteritis nodosa, allergic granulomatosis, acute radiation nephritis, post-streptococcal glomerulonephritis, Waldenstrom's macroglobulinemia, analgesic nephropathy, arteriovenous fistula, arteriovenous graft, dialysis, ectopic kidney, medullary sponge kidney, renal osteodystrophy, solitary kidney, hydronephrosis, microalbuminuria, uremia, haematuria, hyperlipidemia, hypoalbuminaemia, lipiduria, acidosis, hyperkalemia, and edema.

In some embodiments, any of the the ActRII antagonists disclosed herein (e.g., an antagonist of one or more of activin A, activin B, activin AB, activin AC, BMP6, BMP7, BMP9, BMP10, GDF3, GDF8, GDF11, and one or more Smad proteins) may be used, alone or in combination with one or more supportive therapies or active agents, to treat, prevent, or reduce the progression rate and/or severity of chronic kidney disease (e.g., tissue damage, inflammation, and/or fibrosis). Chronic kidney disease (CKD), also known as chronic renal disease, is a progressive loss in renal function over a period of months or years. The symptoms of worsening kidney function may include feeling generally unwell and experiencing a reduced appetite. Often, chronic kidney disease is diagnosed as a result of screening of people known to be at risk of kidney problems, such as those with high blood pressure or diabetes and those with a blood relative with CKD. This disease may also be identified when it leads to one of its recognized complications, such as cardiovascular disease, anemia, or pericarditis. Recent professional guidelines classify the severity of CKD in five stages, with stage 1 being the mildest and usually causing few symptoms and stage 5 being a severe illness with poor life expectancy if untreated. Stage 5 CKD is often called end-stage kidney disease, end-stage renal disease, or end-stage kidney failure, and is largely synonymous with the now outdated terms chronic renal failure or chronic kidney failure; and usually means the patient requires renal replacement therapy, which may involve a form of dialysis, but ideally constitutes a kidney transplant. CKD is initially without specific symptoms and is generally only detected as an increase in serum creatinine or protein in the urine. As the kidney function decreases, various symptoms may manifest as described below. Blood pressure may be increased due to fluid overload and production of vasoactive hormones created by the kidney via the renin-angiotensin system, increasing one's risk of developing hypertension and/or suffering from congestive heart failure. Urea may accumulate, leading to azotemia and ultimately uremia (symptoms ranging from lethargy to pericarditis and encephalopathy). Due to its high systemic circulation, urea is excreted in eccrine sweat at high concentrations and crystallizes on skin as the sweat evaporates (“uremic frost”). Potassium may accumulate in the blood (hyperkalemia with a range of symptoms including malaise and potentially fatal cardiac arrhythmias). Hyperkalemia usually does not develop until the glomerular filtration rate falls to less than 20-25 ml/min/1.73 m2, at which point the kidneys have decreased ability to excrete potassium. Hyperkalemia in CKD can be exacerbated by acidemia (which leads to extracellular shift of potassium) and from lack of insulin. Erythropoietin synthesis may be decreased causing anemia. Fluid volume overload symptoms may occur, ranging from mild edema to life-threatening pulmonary edema. Hyperphosphatemia, due to reduced phosphate excretion, may occur generally following the decrease in glomerular filtration. Hyperphosphatemia is associated with increased cardiovascular risk, being a direct stimulus to vascular calcification. Hypocalcemia may manifest, which is generally caused by stimulation of fibroblast growth factor-23. Osteocytes are responsible for the increased production of FGF23, which is a potent inhibitor of the enzyme 1-alpha-hydroxylase (responsible for the conversion of 25-hydroxycholecalciferol into 1,25 dihydroxyvitamin D3). Later, this progresses to secondary hyperparathyroidism, renal osteodystrophy, and vascular calcification that further impairs cardiac function. Metabolic acidosis (due to accumulation of sulfates, phosphates, uric acid etc.) may occur and cause altered enzyme activity by excess acid acting on enzymes; and also increased excitability of cardiac and neuronal membranes by the promotion of hyperkalemia due to excess acid (acidemia). Acidosis is also due to decreased capacity to generate enough ammonia from the cells of the proximal tubule. Iron deficiency anemia, which increases in prevalence as kidney function decreases, is especially prevalent in those requiring haemodialysis. It is multifactoral in cause, but includes increased inflammation, reduction in erythropoietin, and hyperuricemia leading to bone marrow suppression. People with CKD suffer from accelerated atherosclerosis and are more likely to develop cardiovascular disease than the general population. Patients afflicted with CKD and cardiovascular disease tend to have significantly worse prognoses than those suffering only from the latter. In some embodiments, the chronic kidney disease is a chronic kidney disease mineral bone disorder, a broad syndrome of interrelated skeletal, cardiovascular, and mineral-metabolic disorders arising from kidney disease. CKD-MBD encompasses various skeletal pathologies often referred to as renal osteodystrophy (ROD), which is a preferred embodiment for treatment with any of the polypeptides disclosed herein, or combinations with one or more supportive therapies or active agents. Depending on the relative contribution of different pathogenic factors, ROD is manifested as diverse pathologic patterns of bone remodeling (Hruska et al., 2008, Chronic kidney disease mineral bone disorder (CKD-MBD); in Rosen et al. (ed) Primer on the Metabolic Bone Diseases and Disorders of Mineral Metabolism, 7th ed. American Society for Bone and Mineral Research, Washington D.C., pp 343-349). At one end of the spectrum is ROD with uremic osteodystrophy and low bone turnover, characterized by a low number of active remodeling sites, profoundly suppressed bone formation, and low bone resorption. At the other extreme is ROD with hyperparathyroidism, high bone turnover, and osteitis fibrosa.

In some embodiments, the disclosure contemplates methods of treating one or more complications of a kidney-associated disease (e.g., Alport syndrome, focal segmental glomerulosclerosis (FSGS), polycystic kidney disease, or chronic kidney disease) comprising administering to a patient in need thereof an effective amount of a ActRII antagonist or a heteromultimer comprising the same. In some embodiments, the disclosure contemplates methods of preventing one or more complications of a kidney-associated disease (e.g., Alport syndrome, focal segmental glomerulosclerosis (FSGS), polycystic kidney disease, or chronic kidney disease) comprising administering to a patient in need thereof an effective amount of an ActRII antagonist or a heteromultimer comprising the same. In some embodiments, the disclosure contemplates methods of reducing the progression rate of a kidney-associated disease (e.g., Alport syndrome, focal segmental glomerulosclerosis (FSGS), polycystic kidney disease, or chronic kidney disease) comprising administering to a patient in need thereof an effective amount of an ActRII antagonist or a heteromultimer comprising the same. In some embodiments, the disclosure contemplates methods of reducing the progression rate of one or more complications of a kidney-associated disease (e.g., Alport syndrome, focal segmental glomerulosclerosis (FSGS), polycystic kidney disease, or chronic kidney disease) comprising administering to a patient in need thereof an effective amount of an ActRII antagonist or a heteromultimer comprising the same. In some embodiments, the disclosure contemplates methods of reducing the severity of a kidney-associated disease (e.g., Alport syndrome, focal segmental glomerulosclerosis (FSGS), polycystic kidney disease, or chronic kidney disease) comprising administering to a patient in need thereof an effective amount of a ActRII antagonist or a heteromultimer comprising the same. In some embodiments, the disclosure contemplates methods of reducing the severity of one or more complications of a kidney-associated disease (e.g., Alport syndrome, focal segmental glomerulosclerosis (FSGS), polycystic kidney disease, or chronic kidney disease) comprising administering to a patient in need thereof an effective amount of an ActRII antagonist or a heteromultimer comprising the same. Optionally, methods disclosed herein for treating, preventing, or reducing the progression rate and/or severity of a kidney-associated disease (e.g., Alport syndrome, focal segmental glomerulosclerosis (FSGS), polycystic kidney disease, or chronic kidney disease), particularly treating, preventing, or reducing the progression rate and/or severity of one or more complications of a kidney-associated disease, may further comprise administering to the patient one or more supportive therapies or additional active agents for treating the kidney-associated disease.

In certain embodiments, the present disclosure provides methods for managing a patient that has been treated with, or is a candidate to be treated with, one or more one or more ActRII antagonists or heteromultimers comprising the same of the disclosure (e.g., variant polypeptides such as ActRIIA polypeptides, ActRIIB polypeptides, and ActRIIB:ALK4 polypeptides) by measuring one or more hematologic parameters in the patient. The hematologic parameters may be used to evaluate appropriate dosing for a patient who is a candidate to be treated with the antagonist of the present disclosure, to monitor the hematologic parameters during treatment, to evaluate whether to adjust the dosage during treatment with one or more antagonist of the disclosure, and/or to evaluate an appropriate maintenance dose of one or more antagonists of the disclosure. If one or more of the hematologic parameters are outside the normal level, dosing with one or more ActRII antagonists or heteromultimers comprising the same may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range or on the high side of normal in a patient who is a candidate to be treated with one or more ActRII antagonists or heteromultimers comprising the same, then onset of administration of the one or more antagonists of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels, then administration of the one or more antagonists of the disclosure may be delayed until the levels have returned to a normal or acceptable level.

In certain embodiments, if one or more hematologic parameters are outside the normal range or on the high side of normal in a patient who is a candidate to be treated with one or more ActRII antagonists or heteromultimers comprising the same, then the onset of administration may not be delayed. However, the dosage amount or frequency of dosing of the one or more antagonists of the disclosure may be set at an amount that would reduce the risk of an unacceptable increase in the hematologic parameters arising upon administration of the one or more antagonists of the disclosure. Alternatively, a therapeutic regimen may be developed for the patient that combines one or more ActRII antagonists or heteromultimers comprising the same with a therapeutic agent that addresses the undesirable level of the hematologic parameter. For example, if the patient has elevated blood pressure, then a therapeutic regimen may be designed involving administration of one or more ActRII antagonists or heteromultimers comprising the same and a blood pressure lowering agent. For a patient having lower than desired iron stores, a therapeutic regimen may be developed involving one or more ActRII antagonists or heteromultimers comprising the same of the disclosure and iron supplementation.

In one embodiment, baseline parameter(s) for one or more hematologic parameters may be established for a patient who is a candidate to be treated with one or more ActRII antagonists or heteromultimers comprising the same of the disclosure and an appropriate dosing regimen established for that patient based on the baseline value(s). Alternatively, established baseline parameters based on a patient's medical history could be used to inform an appropriate antagonist dosing regimen for a patient. For example, if a healthy patient has an established baseline blood pressure reading that is above the defined normal range it may not be necessary to bring the patient's blood pressure into the range that is considered normal for the general population prior to treatment with the one or more antagonist of the disclosure. A patient's baseline values for one or more hematologic parameters prior to treatment with one or more ActRII antagonists or heteromultimers comprising the same of the disclosure may also be used as the relevant comparative values for monitoring any changes to the hematologic parameters during treatment with the one or more antagonists of the disclosure.

In certain embodiments, one or more hematologic parameters are measured in patients who are being treated with one or more ActRII antagonists or heteromultimers comprising the same. The hematologic parameters may be used to monitor the patient during treatment and permit adjustment or termination of the dosing with the one or more antagonists of the disclosure or additional dosing with another therapeutic agent. For example, if administration of one or more ActRII antagonists or heteromultimers comprising the same results in an increase in blood pressure, red blood cell level, or hemoglobin level, or a reduction in iron stores, then the dose of the one or more antagonists of the disclosure may be reduced in amount or frequency in order to decrease the effects of the one or more antagonists of the disclosure on the one or more hematologic parameters. If administration of one or more ActRII antagonists or heteromultimers comprising the same results in a change in one or more hematologic parameters that is adverse to the patient, then the dosing of the one or more antagonists of the disclosure may be terminated either temporarily, until the hematologic parameter(s) return to an acceptable level, or permanently. Similarly, if one or more hematologic parameters are not brought within an acceptable range after reducing the dose or frequency of administration of the one or more antagonists of the disclosure, then the dosing may be terminated. As an alternative, or in addition to, reducing or terminating the dosing with the one or more antagonists of the disclosure, the patient may be dosed with an additional therapeutic agent that addresses the undesirable level in the hematologic parameter(s), such as, for example, a blood pressure lowering agent or an iron supplement. For example, if a patient being treated with one or more ActRII antagonists or heteromultimers comprising the same has elevated blood pressure, then dosing with the one or more antagonists of the disclosure may continue at the same level and a blood-pressure-lowering agent is added to the treatment regimen, dosing with the one or more antagonist of the disclosure may be reduced (e.g., in amount and/or frequency) and a blood-pressure-lowering agent is added to the treatment regimen, or dosing with the one or more antagonist of the disclosure may be terminated and the patient may be treated with a blood-pressure-lowering agent.

### 7. Pharmaceutical Compositions

The therapeutic agents described herein (e.g., ActRII antagonists or heteromultimers comprising the same) may be formulated into pharmaceutical compositions. Pharmaceutical compositions for use in accordance with the present disclosure may be formulated in conventional manner using one or more physiologically acceptable carriers or excipients. Such formulations will generally be substantially pyrogen-free, in compliance with most regulatory requirements.

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII antagonists or heteromultimers comprising the same which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII antagonists or heteromultimers comprising the same in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration

Further, the composition may be encapsulated or injected in a form for delivery to a target tissue site. In certain embodiments, compositions of the present invention may include a matrix capable of delivering one or more therapeutic compounds (e.g., ActRII antagonists or heteromultimers comprising the same) to a target tissue site, providing a structure for the developing tissue and optimally capable of being resorbed into the body. For example, the matrix may provide slow release of the ActRII antagonists or heteromultimers comprising the same. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods disclosed herein can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions disclosed herein may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII antagonists or heteromultimers comprising the same). The various factors include, but are not limited to, the patient's age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling.

In certain embodiments, the present invention also provides gene therapy for the in vivo production of ActRII antagonists or heteromultimers comprising the same. Such therapy would achieve its therapeutic effect by introduction of the ActRII antagonist (or heteromultimers comprising the same) polynucleotide sequences into cells or tissues having the disorders as listed above. Delivery of ActRII antagonist (or heteromultimers comprising the same) polynucleotide sequences can be achieved using a recombinant expression vector such as a chimeric virus or a colloidal dispersion system. Preferred for therapeutic delivery of ActRII antagonist (or heteromultimers comprising the same) polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the ActRII antagonist or heteromultimer of the same. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII antagonist (or heteromultimer of the same) polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### EXEMPLIFICATION

The invention now being generally described, it will be more readily understood by reference to the following examples, which are included merely for purposes of illustration of certain embodiments of the present invention, and are not intended to limit the invention.

### Example 1: ActRIIA-Fc Fusion Proteins

A soluble ActRIIA fusion protein was constructed that has the extracellular domain of human ActRIIa fused to a human or mouse Fc domain with a minimal linker in between. The constructs are referred to as ActRIIA-hFc and ActRIIA-mFc, respectively.

ActRIIA-hFc is shown below as purified from CHO cell lines SE ID NO: 32):

ILGRSETQECLFFNANWEKDRTNQTGVEPCYGDKD
KRRHCFATWKNISGSIEIVKQGCWLDDINCYDRTD
CVEKKDSPEVYFCCCEGNMCNEKFSYFPEMEVTQP
TSNPVTPKPPTGGGTHTCPPCPAPELLGGPSVFL
FPPKPKDTLMISRTPEVTCVVVDVSHEDPEVKFNW
YVDGVEVHNAKTKPREEQYNSTYRVVSVLTVLHQD
WLNGKEYKCKVSNKALPVPIEKTISKAKGQPREPQ
VYTLPPSREEMTKNQVSLTCLVKGFYPSDIAVEWE
SNGQPENNYKTTPPVLDSDGSFFLYSKLTVDKSRW
QQGNVFSCSVMHEALHNHYTQKSLSLSPGK

The ActRIIA-hFc and ActRIIA-mFc proteins were expressed in CHO cell lines. Three different leader sequences were considered:

(i) Honey bee mellitin (HBML):
(SEQ ID NO: 33)
MKFLVNVALVFMVVYISYIYA
(ii) Tissue plasminogen activator (TPA):
(SEQ ID NO: 34)
MDAMKRGLCCVLLLCGAVFVSP
(iii) Native:
(SEQ ID NO: 35)
MGAAAKLAFAVFLISCSSGA.

The selected form employs the TPA leader and has the following unprocessed amino acid sequence:

(SEQ ID NO: 36)
MDAMKRGLCCVLLLCGAVFVSPGAAILGRSETQEC
LFFNANWEKDRTNQTGVEPCYGDKDKRRHCFATWK
NISGSIEIVKQGCWLDDINCYDRTDCVEKKDSPEV
YFCCCEGNMCNEKFSYFPEMEVTQPTSNPVTPKPP
TGGGTHTCPPCPAPELLGGPSVFLFPPKPKDTLMI
SRTPEVTCVVVDVSHEDPEVKFNWYVDGVEVHNAK
TKPREEQYNSTYRVVSVLTVLHQDWLNGKEYKCKV
SNKALPVPIEKTISKAKGQPREPQVYTLPPSREEM
TKNQVSLTCLVKGFYPSDIAVEWESNGQPENNYKT
TPPVLDSDGSFFLYSKLTVDKSRWQQGNVFSCSVM
HEALHNHYTQKSLSLSPGK

This polypeptide is encoded by the following nucleic acid sequence:

(SEQ ID NO: 37)
ATGGATGCAATGAAGAGAGGGCTCTGCTGTGTG
CTGCTGCTGTGTGGAGCAGTCTTCGTTTCGCCC
GGCGCCGCTATACTTGGTAGATCAGAAACTCAG
GAGTGTCTTTTTTTAATGCTAATTGGGAAAAAG
ACAGAACCAATCAAACTGGTGTTGAACCGTGTT
ATGGTGACAAAGATAAACGGCGGCATTGTTTTG
CTACCTGGAAGAATATTTCTGGTTCCATTGAAT
AGTGAAACAAGGTTGTTGGCTGGATGATATCAA
CTGCTATGACAGGACTGATTGTGTAGAAAAAAA
AGACAGCCCTGAAGTATATTTCTGTTGCTGTGA
GGGCAATATGTGTAATGAAAAGTTTTCTTATTT
TCCGGAGATGGAAGTCACACAGCCCACTTCAAA
TCCAGTTACACCTAAGCCACCCACCGGTGGTGG
AACTCACACATGCCCACCGTGCCCAGCACCTGA
ACTCCTGGGGGGACCGTCAGTCTTCCTCTTCCC
CCCAAAACCCAAGGACACCCTCATGATCTCCCG
GACCCCTGAGGTCACATGCGTGGTGGTGGACGT
GAGCCACGAAGACCCTGAGGTCAAGTTCAACTG
GTACGTGGACGGCGTGGAGGTGCATAATGCCAA
GACAAAGCCGCGGGAGGAGCAGTACAACAGCAC
GTACCGTGTGGTCAGCGTCCTCACCGTCCTGCA
CCAGGACTGGCTGAATGGCAAGGAGTACAAGTG
CAAGGTCTCCAACAAAGCCCTCCCAGTCCCCAT
CGAGAAAACCATCTCCAAAGCCAAAGGGCAGCC
CCGAGAACCACAGGTGTACACCCTGCCCCCATC
CCGGGAGGAGATGACCAAGAACCAGGTCAGCCT
GACCTGCCTGGTCAAAGGCTTCTATCCCAGCGA
CATCGCCGTGGAGTGGGAGAGCAATGGGCAGCC
GGAGAACAACTACAAGACCACGCCTCCCGTGCT
GGACTCCGACGGCTCCTTCTTCCTCTATAGCAA
GCTCACCGTGGACAAGAGCAGGTGGCAGCAGGG
GAACGTCTTCTCATGCTCCGTGATGCATGAGGC
TCTGCACAACCACTACACGCAGAAGAGCCTCTC
CCTGTCTCCGGGTAAATGAGAATTC

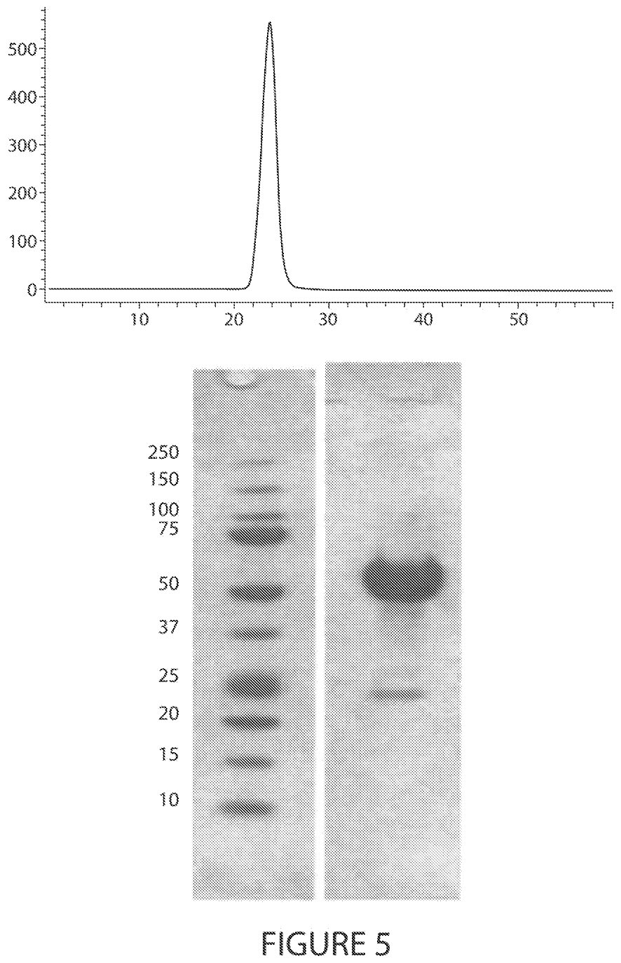

Both ActRIIA-hFc and ActRIIA-mFc were remarkably amenable to recombinant expression. As shown in FIG. 5, the protein was purified as a single, well-defined peak of protein. N-terminal sequencing revealed a single sequence of -ILGRSETQE (SEQ ID NO: 38). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

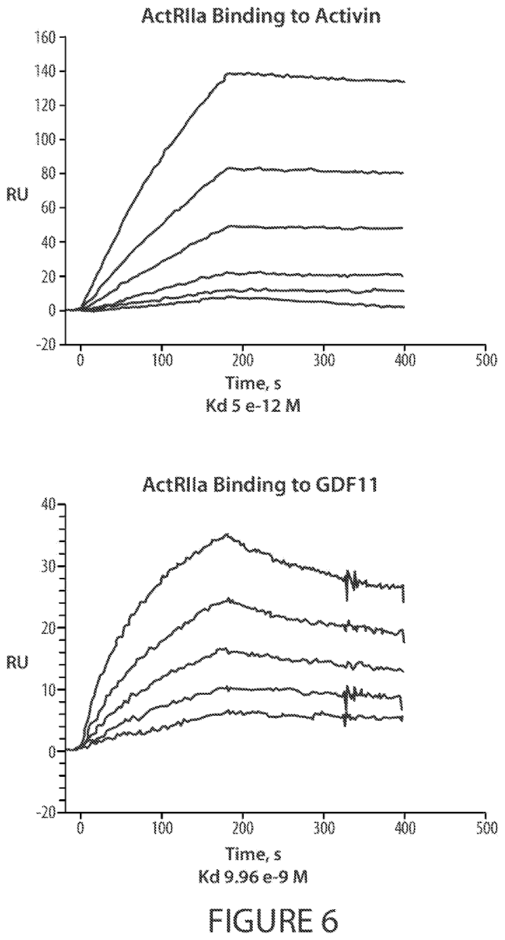

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10¹² and bound to GDF11 with a K_{D} of 9.96×10⁻⁹. See FIG. 6. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 g/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 on/enhancer, CMV promoter), using a tissue plasminogen leader sequence of SEQ ID NO: 34. The protein, purified as described above in Example 1, had a sequence of SEQ ID NO: 32. The Fc portion is a human IgG1 Fc sequence, as shown in SEQ ID NO: 32. Protein analysis reveals that the ActRIIA-hFc fusion protein is formed as a homodimer with disulfide bonding.

The CHO-cell-expressed material has a higher affinity for activin B ligand than that reported for an ActRIIa-hFc fusion protein expressed in human 293 cells [see, del Re et al. (2004) J Biol Chem. 279(51):53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

### Example 3: Alternative ActRIIA-Fc Proteins

A variety of ActRIIA variants that may be used according to the methods described herein are described in the International Patent Application published as WO2006/012627 (see e.g., pp. 55-58), incorporated herein by reference in its entirety. An alternative construct may have a deletion of the C-terminal tail (the final 15 amino acids of the extracellular domain of ActRIIA. The sequence for such a construct is presented below (Fc portion underlined) (SEQ ID NO: 39):

ILGRSETQECLFFNANWEKDRTNQTGVEPCYGD
KDKRRHCFATWKNISGSIEIVKQGCWLDDINCY
DRTDCVEKKDSPEVYFCCCEGNMCNEKFSYFPE
MTGGGTHTCPPCPAPELLGGPSVFLFPPKPKDT
LMISRTPEVTCVVVDVSHEDPEVKFNWYVDGVE
VHNAKTKPREEQYNSTYRVVSVLTVLHQDWLNG
KEYKCKVSNKALPVPIEKTISKAKGQPREPQVY
TLPPSREEMTKNQVSLTCLVKGFYPSDIAVEWE
SNGQPENNYKTTPPVLDSDGSFFLYSKLTVDKS
RWQQGNVFSCSVMHEALHNHYTQKSLSLSPGK

### Example 4: Generation of ActRIIB-Fc Fusion Proteins

Applicants constructed a soluble ActRIIB fusion protein that has the extracellular domain of human ActRIIB fused to a human or mouse Fc domain with a minimal linker in between. The constructs are referred to as ActRIIB-hFc and ActRIIB-mFc, respectively.

ActRIIB-hFc is shown below as purified from CHO cell lines (SEQ ID NO: 40):

GRGEAETRECIYYNANWELERTNQSGLERCEGE
QDKRLHCYASWRNSSGTIELVKKGCWLDDFNCY
DRQECVATEENPQVYFCCCEGNFCNERFTHLPE
AGGPEVTYEPPPTAPTGGGTHTCPPCPAPELLG
GPSVFLFPPKPKDTLMISRTPEVTCVVVDVSHE
DPEVKFNWYVDGVEVHNAKTKPREEQYNSTYRV
VSVLTVLHQDWLNGKEYKCKVSNKALPVPIEKT
ISKAKGQPREPQVYTLPPSREEMTKNQVSLTCL
VKGFYPSDIAVEWESNGQPENNYKTTPPVLDSD
GSFFLYSKLTVDKSRWQQGNVFSCSVMHEALHN
HYTQKSLSLSPGK

The ActRIIB-hFc and ActRIIB-mFc proteins were expressed in CHO cell lines. Three different leader sequences were considered: (i) Honey bee mellitin (HBML), ii) Tissue plasminogen activator (TPA), and (iii) Native: MGAAAKLAFAVFLISCSSGA (SEQ ID NO: 41).

The selected form employs the TPA leader and has the following unprocessed amino acid sequence (SEQ ID NO: 42):

MDAMKRGLCCVLLLCGAVFVSPGASGRGEAETR
ECIYYNANWELERTNQSGLERCEGEQDKRLHCY
ASWRNSSGTIELVKKGCWLDDFNCYDRQECVAT
EENPQVYFCCCEGNFCNERFTHLPEAGGPEVTY
EPPPTAPTGGGTHTCPPCPAPELLGGPSVFLFP
PKPKDTLMISRTPEVTCVVVDVSHEDPEVKFNW
YVDGVEVHNAKTKPREEQYNSTYRVVSVLTVLH
QDWLNGKEYKCKVSNKALPVPIEKTISKAKGQP
REPQVYTLPPSREEMTKNQVSLTCLVKGFYPSD
IAVEWESNGQPENNYKTTPPVLDSDGSFFLYSK
LTVDKSRWQQGNVFSCSVMHEALHNHYTQKSLS
LSPGK

This polypeptide is encoded by the following nucleic acid sequence (SEQ ID NO: 43):

A
TGGATGCAAT GAAGAGAGGG CTCTGCTGTG
TGCTGCTGCT GTGTGGAGCA GTCTTCGTTT
CGCCCGGCGC CTCTGGGCGT GGGGAGGCTG
AGACACGGGA GTGCATCTAC TACAACGCCA
ACTGGGAGCT GGAGCGCACC AACCAGAGCG
GCCTGGAGCG CTGCGAAGGC GAGCAGGACA
AGCGGCTGCA CTGCTACGCC TCCTGGCGCA
ACAGCTCTGG CACCATCGAG CTCGTGAAGA
AGGGCTGCTG GCTAGATGAC TTCAACTGCT
ACGATAGGCA GGAGTGTGTG GCCACTGAGG
AGAACCCCCA GGTGTACTTC TGCTGCTGTG
AAGGCAACTT CTGCAACGAG CGCTTCACTC
ATTTGCCAGA GGCTGGGGGC CCGGAAGTCA
CGTACGAGCC ACCCCCGACA GCCCCCACCG
GTGGTGGAAC TCACACATGC CCACCGTGCC
CAGCACCTGA ACTCCTGGGG GGACCGTCAG
TCTTCCTCTT CCCCCCAAAA CCCAAGGACA
CCCTCATGAT CTCCCGGACC CCTGAGGTCA
CATGCGTGGT GGTGGACGTG AGCCACGAAG
ACCCTGAGGT CAAGTTCAAC TGGTACGTGG
ACGGCGTGGA GGTGCATAAT GCCAAGACAA
AGCCGCGGGA GGAGCAGTAC AACAGCACGT
ACCGTGTGGT CAGCGTCCTC ACCGTCCTGC
ACCAGGACTG GCTGAATGGC AAGGAGTACA
AGTGCAAGGT CTCCAACAAA GCCCTCCCAG
TCCCCATCGA GAAAACCATC TCCAAAGCCA
AAGGGCAGCC CCGAGAACCA CAGGTGTACA
CCCTGCCCCC ATCCCGGGAG GAGATGACCA
AGAACCAGGT CAGCCTGACC TGCCTGGTCA
AAGGCTTCTA TCCCAGCGAC ATCGCCGTGG
AGTGGGAGAG CAATGGGCAG CCGGAGAACA
ACTACAAGAC CACGCCTCCC GTGCTGGACT
CCGACGGCTC CTTCTTCCTC TATAGCAAGC
TCACCGTGGA CAAGAGCAGG TGGCAGCAGG
GGAACGTCTT CTCATGCTCC GTGATGCATG
AGGCTCTGCA CAACCACTAC ACGCAGAAGA
GCCTCTCCCT GTCTCCGGGT AAATGA

N-terminal sequencing of the CHO-cell-produced material revealed a major sequence of -GRGEAE (SEQ ID NO: 44). Notably, other constructs reported in the literature begin with an -SGR . . . sequence.

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

ActRIIB-Fc fusion proteins were also expressed in HEK293 cells and COS cells. Although material from all cell lines and reasonable culture conditions provided protein with muscle-building activity in vivo, variability in potency was observed perhaps relating to cell line selection and/or culture conditions.

Applicants generated a series of mutations in the extracellular domain of ActRIIB and produced these mutant proteins as soluble fusion proteins between extracellular ActRIIB and an Fc domain. The background ActRIIB-Fc fusion has the sequence of SEQ ID NO: 40.

Various mutations, including N- and C-terminal truncations, were introduced into the background ActRIIB-Fc protein. Based on the data presented herien, it is expected that these constructs, if expressed with a TPA leader, will lack the N-terminal serine. Mutations were generated in ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNAs were isolated. For murine constructs (mFc), a murine IgG2a was substituted for the human IgG1. Sequences of all mutants were verified. All of the mutants were produced in HEK293T cells by transient transfection. In summary, in a 500 ml spinner, HEK293T cells were set up at 6×10⁵ cells/ml in Freestyle (Invitrogen) media in 250 ml volume and grown overnight. Next day, these cells were treated with DNA:PEI (1:1) complex at 0.5 ug/ml final DNA concentration. After 4 hrs, 250 ml media was added and cells were grown for 7 days. Conditioned media was harvested by spinning down the cells and concentrated.

Mutants were purified using a variety of techniques, including, for example, a protein A column, and eluted with low pH (3.0) glycine buffer. After neutralization, these were dialyzed against PBS.

Mutants were also produced in CHO cells by similar methodology. Mutants were tested in binding assays and/or bioassays described in WO 2008/097541 and WO 2006/012627 incorporated by reference herein. In some instances, assays were performed with conditioned medium rather than purified proteins. Additional variations of ActRIIB are described in U.S. Pat. No. 7,842,663.

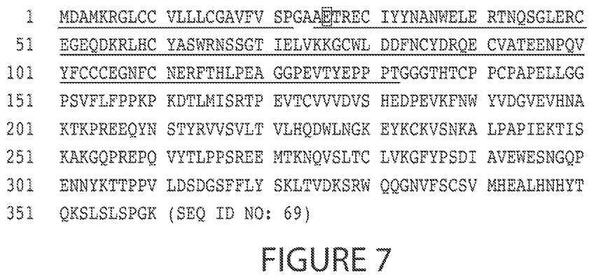

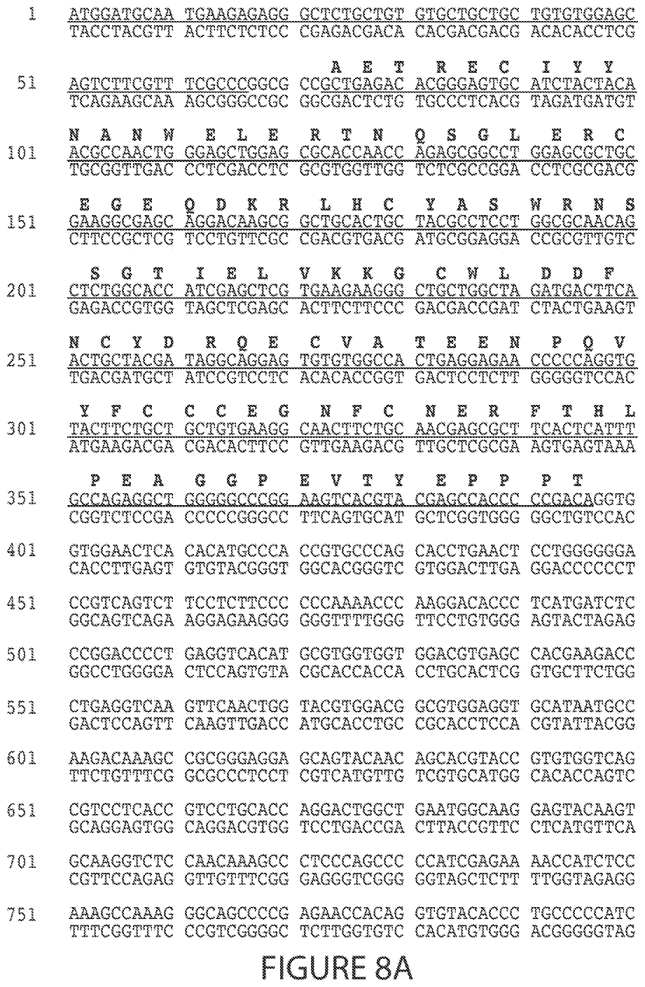

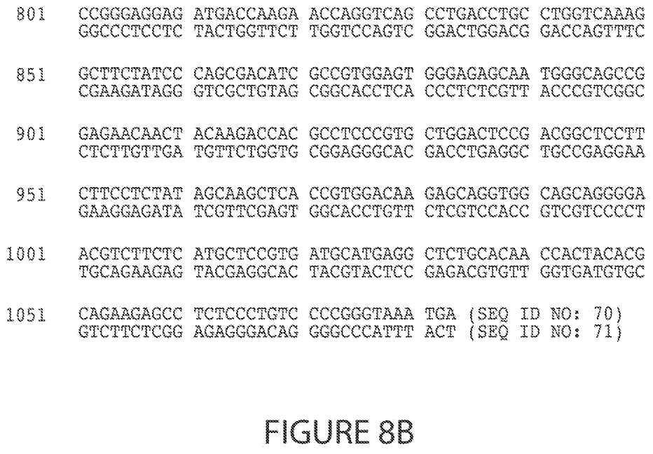

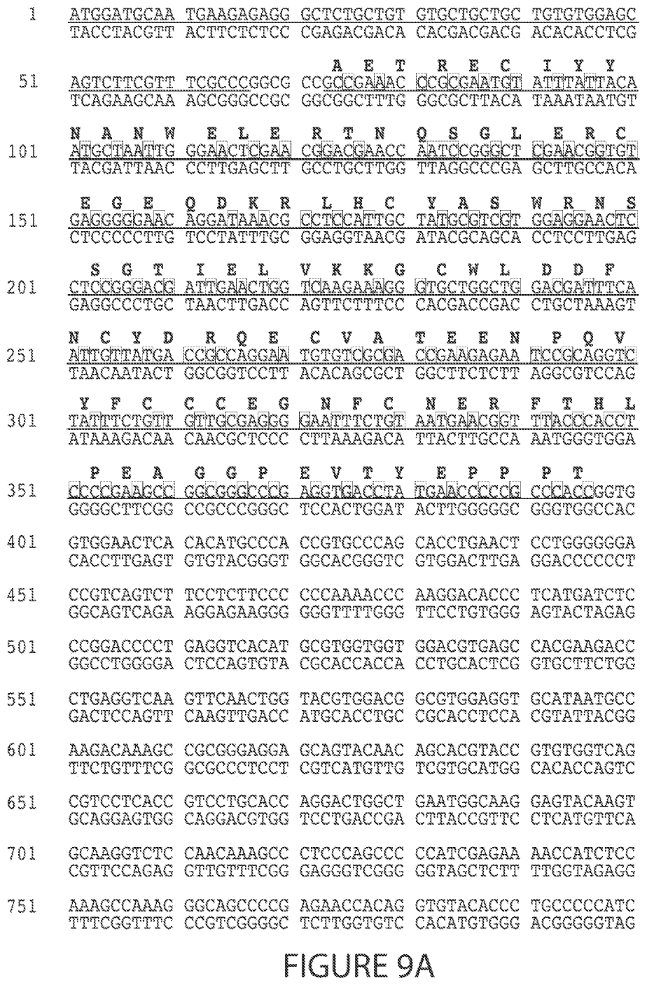

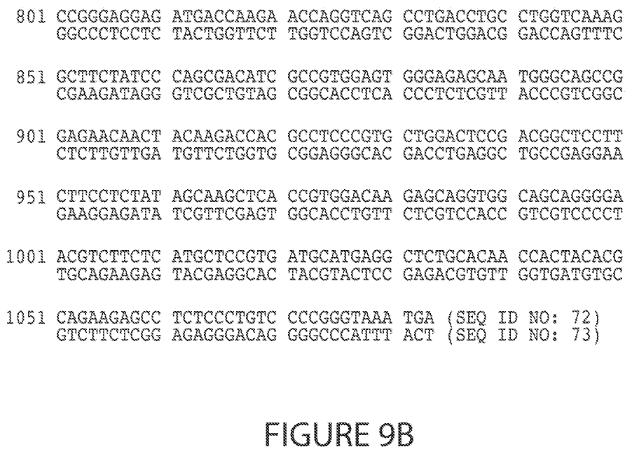

Applicant generated an ActRIIB(25-131)-hFc fusion protein, which comprises the human ActRIIB extracellular domain with N-terminal and C-terminal truncations (residues 25-131 of the native protein SEQ ID NO: 1) fused N-terminally with a TPA leader sequence substituted for the native ActRIIB leader and C-terminally with a human Fc domain via a minimal linker (three glycine residues) (FIG. 7). A nucleotide sequence encoding this fusion protein is shown in FIG. 8. Applicants modified the codons and found a variant nucleic acid encoding the ActRIIB(25-131)-hFc protein that provided substantial improvement in the expression levels of initial transformants (FIG. 9).

The mature protein has an amino acid sequence as follows (N-terminus confirmed by N-terminal sequencing)(SEQ ID NO: 45):

ETRECIYYNA NWELERTNQS GLERCEGEQD
KRLHCYASWR NSSGTIELVK KGCWLDDFNC
YDRQECVATE ENPQVYFCCC EGNFCNERFT
HLPEAGGPEV TYEPPPTGGG THTCPPCPAP
ELLGGPSVFL FPPKPKDTLM ISRTPEVTCV
VVDVSHEDPE VKFNWYVDGV EVHNAKTKPR
EEQYNSTYRV VSVLTVLHQD WLNGKEYKCK
VSNKALPAPI EKTISKAKGQ PREPQVYTLP
PSREEMTKNQ VSLTCLVKGF YPSDIAVEWE
SNGQPENNYK TTPPVLDSDG SFFLYSKLTV
DKSRWQQGNV FSCSVMHEAL HNHYTQKSLS
LSPGK

The expressed molecule was purified using a series of column chromatography steps, including for example, three or more of the following, in any order: Protein A chromatography, Q sepharose chromatography, phenylsepharose chromatography, size exclusion chromatography and cation exchange chromatography. The purification could be completed with viral filtration and buffer exchange.

Affinities of several ligands for ActRIIB(25-131)-hFc and its full-length counterpart ActRIIB(20-134)-hFc were evaluated in vitro with a Biacore™ instrument, and the results are summarized in Table 8 below. Kd values were obtained by steady-state affinity fit due to very rapid association and dissociation of the complex, which prevented accurate determination of kₒₙ and k_{off}. ActRIIB(25-131)-hFc bound, for example, activin A, activin B, and GDF11 with high affinity.

TABLE 8
Ligand Affinities of ActRIIB-hFc Forms

| | Activin A | Activin B | GDF11 |
|---|---|---|---|
| Fusion Construct | (e−11) | (e−11) | (e−11) |
| ActRIIB(20-134)-hFc | 1.6 | 1.2 | 3.6 |
| ActRIIB(25-131)-hFc | 1.8 | 1.2 | 3.1 |

### Example 5: Generation of a ActRIIB Variant Fc Fusion Polypeptide

An ActRIIB variant Fc fusion polypeptide was constructed as follows. A polypeptide having a modified extracellular domain of ActRIIB (amino acids 20-134 of SEQ ID NO: 1 with an L79D substitution) with greatly reduced activin A binding relative to GDF11 and/or myostatin (as a consequence of a leucine-to-aspartate substitution at position 79 in SEQ ID NO: 1) was fused to a human or mouse Fc domain with a minimal linker in between. The constructs are referred to as ActRIIB(L79D 20-134)-hFc and ActRIIB(L79D 20-134)-mFc, respectively. Alternative forms with a glutamate rather than an aspartate at position 79 performed similarly (L79E). Alternative forms with an alanine rather than a valine at position 226 with respect to SEQ ID NO: 64, below were also generated and performed equivalently in all respects tested. The aspartate at position 79 (relative to SEQ ID NO: 1) is indicated with double underlining below. The valine at position 226 relative to SEQ ID NO: 64 is also indicated by double underlining below.

The ActRIIB variant Fc fusion polypeptide ActRIIB(L79D 20-134)-hFc is shown below as purified from CHO cell lines (SEQ ID NO: 46).

GRGEAETRECIYYNANWELERTNQSGLERCEGEQD
KRLHCYASWRNSSGTIELVKKGCWDDDFNCYDRQE
CVATEENPQVYFCCCEGNFCNERFT
HLPEAGGPEVTYEPPPTAPT GGGTHTCPPCPAPE
LLGGPSVFLFPPKPKDTLMISRTPEVTCVVVDVSH
EDPEVKFNWYVDGVEVHNAKTKPREEQYNSTYRVV
SVLTVLHQDWLNGKEYKCKVSNKALPVPIEKTISK
AKGQPREPQVYTLPPSREEMTKNQVSLTCLVKGFY
PSDIAVEWESNGQPENNYKTTPPVLDSDGSFFLYS
KLTVDKSRWQQGNVFSCSVMHEALHNHYTQKSLSL
SPGK

The ActRIIB-derived portion of the ActRIIB variant Fc fusion polypeptide has an amino acid sequence set forth below (SEQ ID NO: 47), and that portion could be used as a monomer or as a non-Fc fusion protein as a monomer, dimer, or greater-order complex.

(SEQ ID NO: 47)
GRGEAETRECIYYNANWELERTNQSGLERCEGEQDKRLHCYASWRNSSG
TIELVKKGCWDDDFNCYDRQECVATEENPQVYFCCCEGNFCNERFTHLP
EAGGPEVTYEPPPTAPT

The ActRIIB variant Fc fusion polypeptide protein was expressed in CHO cell lines. Three different leader sequences were considered:

(i) Honey bee melittin (HBML), (ii) Tissue plasminogen activator (TPA), and (iii) Native.

The selected form employs the TPA leader and has the following unprocessed amino acid sequence:

(SEQ ID NO: 48)
MDAMKRGLCCVLLLCGAVFVSPGASGRGEAETRECIYYNANWELERTNQSG
LERCEGEQDKRLHCYASWRNSSGTIELVKKGCWDDDFNCYDRQECVATEEN
PQVYFCCCEGNFCNERFTHLPEAGGPEVTYEPPPTAPTGGGTHTCPPCPAP
ELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHY
TQKSLSLSPGK

This polypeptide is encoded by the following nucleic acid sequence (SEQ ID NO: 49):

A TGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GGACGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG TCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. In an example of a purification scheme, the cell culture medium is passed over a protein A column, washed in 150 mM Tris/NaCl (pH 8.0), then washed in 50 mM Tris/NaCl (pH 8.0) and eluted with 0.1 M glycine, pH 3.0. The low pH eluate is kept at room temperature for 30 minutes as a viral clearance step. The eluate is then neutralized and passed over a Q-sepharose ion-exchange column and washed in 50 mM Tris pH 8.0, 50 mM NaCl, and eluted in 50 mM Tris pH 8.0, with an NaCl concentration between 150 mM and 300 mM. The eluate is then changed into 50 mM Tris pH 8.0, 1.1 M ammonium sulfate and passed over a phenyl sepharose column, washed, and eluted in 50 mM Tris pH 8.0 with ammonium sulfate between 150 and 300 mM. The eluate is dialyzed and filtered for use.

Additional ActRIIB variant Fc fusion polypeptides (ActRIIB-Fc fusion proteins modified so as to reduce the ratio of activin A binding relative to myostatin or GDF11 binding) are described in WO 2008/097541 and WO 2006/012627, incorporated by reference herein.

### Example 6: Bioassay for GDF11- and Activin-Mediated Signaling

An A-204 reporter gene assay was used to evaluate the effects of ActRIIB-Fc proteins and ActRIIB variant Fc fusion polypeptides on signaling by GDF-11 and activin A. Cell line: human rhabdomyosarcoma (derived from muscle). Reporter vector: pGL3(CAGA)12 (described in Dennler et al, 1998, EMBO 17: 3091-3100). The CAGA12 motif is present in TGF-beta responsive genes (e.g., PAI-1 gene), so this vector is of general use for factors signaling through SMADs.

Day 1: Split A-204 cells into 48-well plate.

Day 2: A-204 cells transfected with 10 ug pGL3(CAGA)12 or pGL3(CAGA)12(10 ug)+pRLCMV (1 pg) and Fugene.

Day 3: Add factors (diluted into medium+0.1% BSA). Inhibitors need to be preincubated with factors for 1 hr before adding to cells. Six hrs later, cells were rinsed with PBS and lysed.

This is followed by a luciferase assay. In the absence of any inhibitors, activin A showed 10-fold stimulation of reporter gene expression and an ED50˜2 ng/ml. GDF-11: 16 fold stimulation, ED50: ˜1.5 ng/ml.

ActRIIB(20-134) is a potent inhibitor of, for example, activin A, GDF-8, and GDF-11 activity in this assay. As described below, ActRIIB variants were also tested in this assay.

### Example 7: ActRIIB-Fc Variants, Cell-Based Activity

Activity of ActRIIB-Fc proteins and ActRIIB variant Fc fusion polypeptides was tested in a cell-based assay as described above. Results are summarized in Table 9 below. Some variants were tested in different C-terminal truncation constructs. As discussed above, truncations of five or fifteen amino acids caused reduction in activity. The ActRIIB variant Fc fusion polypeptides (L79D and L79E variants) showed substantial loss of activin A inhibition while retaining almost wild-type inhibition of GDF11.

TABLE 9
Soluble ActRIIB-Fc binding to GDF11 and Activin A:

| | Portion of ActRIIB | GDF11 | Activin |
|---|---|---|---|
| ActRIIB-Fc | (corresponds to amino | Inhibition | Inhibition |
| Variations | acids of SEQ ID NO:1) | Activity | Activity |
| R64 | 20-134 | +++ | +++ |
| | | (approx. | (approx. |
| | | 10⁻⁸ M K_{I}) | 10⁻⁸ M K_{I}) |
| A64 | 20-134 | + | + |
| | | (approx. | (approx. |
| | | 10⁻⁶ M K_{I}) | 10⁻⁶ M K_{I}) |
| R64 | 20-129 | +++ | +++ |
| R64 K74A | 20-134 | ++++ | ++++ |
| R64 A24N | 20-134 | +++ | +++ |
| R64 A24N | 20-119 | ++ | ++ |
| R64 A24N K74A | 20-119 | + | + |
| R64 L79P | 20-134 | + | + |
| R64 L79P K74A | 20-134 | + | + |
| R64 L79D | 20-134 | +++ | + |
| R64 L79E | 20-134 | +++ | + |
| R64K | 20-134 | +++ | +++ |
| R64K | 20-129 | +++ | +++ |
| R64 P129S | 20-134 | +++ | +++ |
| P130A | | | |
| R64N | 20-134 | + | + |

+ Poor activity (roughly 1 × 10⁻⁶ K_{I})
++ Moderate activity (roughly 1 × 10⁻⁷ K_{I})
+++ Good (wild-type) activity (roughly 1 × 10⁻⁸ K_{I})
++++ Greater than wild-type activity

The A24N variant has activity in the cell-based assay (above) and that is equivalent to the wild-type molecule. The A24N variant, and any of the other variants tested above, may be combined with the ActRIIB variant Fc fusion polypeptides, such as the L79D or L79E variants.

### Example 8: GDF11 and Activin a Binding

Binding of certain ActRIIB-Fc proteins and ActRIIB variant Fc fusion polypeptides to ligands was tested in a Biacore™ assay.

The ActRIIB-Fc variants or wild-type protein were captured onto the system using an anti-hFc antibody. Ligands were injected and flowed over the captured receptor proteins. Results are summarized in the tables below.

TABLE 10
Ligand-Binding Specificity IIB Variants.

| Protein | Kon (1/Ms) | Koff (1/s) | KD (M) |
|---|---|---|---|
| | GDF11 | | |
| ActRIIB(20-134)-hFc | 1.34e−6 | 1.13e−4 | 8.42e−11 |
| ActRIIB(A24N 20-134)-hFc | 1.21e−6 | 6.35e−5 | 5.19e−11 |
| ActRIIB(L79D 20-134)-hFc | 6.7e−5 | 4.39e−4 | 6.55e−10 |
| ActRIIB(L79E 20-134)-hFc | 3.8e−5 | 2.74e−4 | 7.16e−10 |
| ActRIIB(R64K 20-134)-hFc | 6.77e−5 | 2.41e−5 | 3.56e−11 |
| | GDF8 | | |
| ActRIIB(20-134)-hFc | 3.69e−5 | 3.45e−5 | 9.35e−11 |
| ActRIIB(A24N 20-134)-hFc | | | |
| ActRIIB(L79D 20-134)-hFc | 3.85e−5 | 8.3e−4 | 2.15e−9 |
| ActRIIB(L79E 20-134)-hFc | 3.74e−5 | 9e−4 | 2.41e−9 |
| ActRIIB(R64K 20-134)-hFc | 2.25e−5 | 4.71e−5 | 2.1e−10 |
| ActRIIB(R64K 20-129)-hFc | 9.74e−4 | 2.09e−4 | 2.15e−9 |
| ActRIIB(P129S, P130R | 1.08e−5 | 1.8e−4 | 1.67e−9 |
| 20-134)-hFc | | | |
| ActRIIB(K74A 20-134)-hFc | 2.8e−5 | 2.03e−5 | 7.18e−11 |
| | Activin A | | |
| ActRIIB(20-134)-hFc | 5.94e6 | 1.59e−4 | 2.68e−11 |
| ActRIIB(A24N 20-134)-hFc | 3.34e6 | 3.46e−4 | 1.04e−10 |
| ActRIIB(L79D 20-134)-hFc | | | Low binding |
| ActRIIB(L79E 20-134)-hFc | | | Low binding |
| ActRIIB(R64K 20-134)-hFc | 6.82e6 | 3.25e−4 | 4.76e−11 |
| ActRIIB(R64K 20-129)-hFc | 7.46e6 | 6.28e−4 | 8.41e−11 |
| ActRIIB(P129S, P130R | 5.02e6 | 4.17e−4 | 8.31e−11 |
| 20-134)-hFc | | | |

These data obtained in a cell-free assay confirm the cell-based assay data, demonstrating that the A24N variant retains ligand-binding activity that is similar to that of the ActRIIB(20-134)-hFc molecule and that the L79D or L79E molecule retains myostatin and GDF11 binding but shows markedly decreased (non-quantifiable) binding to activin A.

Other variants have been generated and tested, as reported in WO2006/012627 (incorporated herein by reference in its entirety). See, e.g., pp. 59-60, using ligands coupled to the device and flowing receptor over the coupled ligands. Notably, K74Y, K74F, K74I (and presumably other hydrophobic substitutions at K74, such as K74L), and D80I, cause a decrease in the ratio of activin A (ActA) binding to GDF11 binding, relative to the wild-type K74 molecule. Table 11 showing data with respect to these variants is reproduced below:

TABLE 11
Soluble ActRIIB-Fc Variants Binding to GDF11
and Activin a (Biacore ™ Assay)

| ActRIIB | ActA | GDF11 |
|---|---|---|
| WT (64A) | KD = 1.8e−7 M | KD = 2.6e−7 M |
| | (+) | (+) |
| WT (64R) | na | KD = 8.6e−8 M |
| | | (+++) |
| +15tail | KD~2.6e−8 M | KD = 1.9e−8 M |
| | (+++) | (++++) |
| E37A | * | * |
| R40A | − | − |
| D54A | − | * |
| K55A | ++ | * |
| R56A | * | * |
| K74A | KD = 4.35e−9 M | KD = 5.3e−9 M |
| | +++++ | −++++ |
| K74Y | * | −− |
| K74F | * | −− |
| K74I | * | −− |
| W78A | * | * |
| L79A | + | * |
| D80K | * | * |
| D80R | * | * |
| D80A | * | * |
| D80F | * | * |
| D80G | * | * |
| D80M | * | * |
| D80N | * | * |
| D80I | * | −− |
| F82A | ++ | − |

* No observed binding
−− <⅕ WT binding
− ~½ WT binding
+ WT
++ <2x increased binding
+++ ~5x increased binding
++++ ~10x increased binding
+++++ ~40x increased binding

### Example 9: Generation of an ActRIIB Variant with Truncated ActRIIB Extracellular Domain

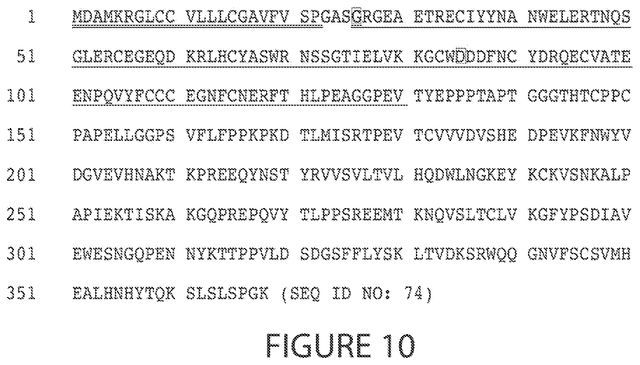

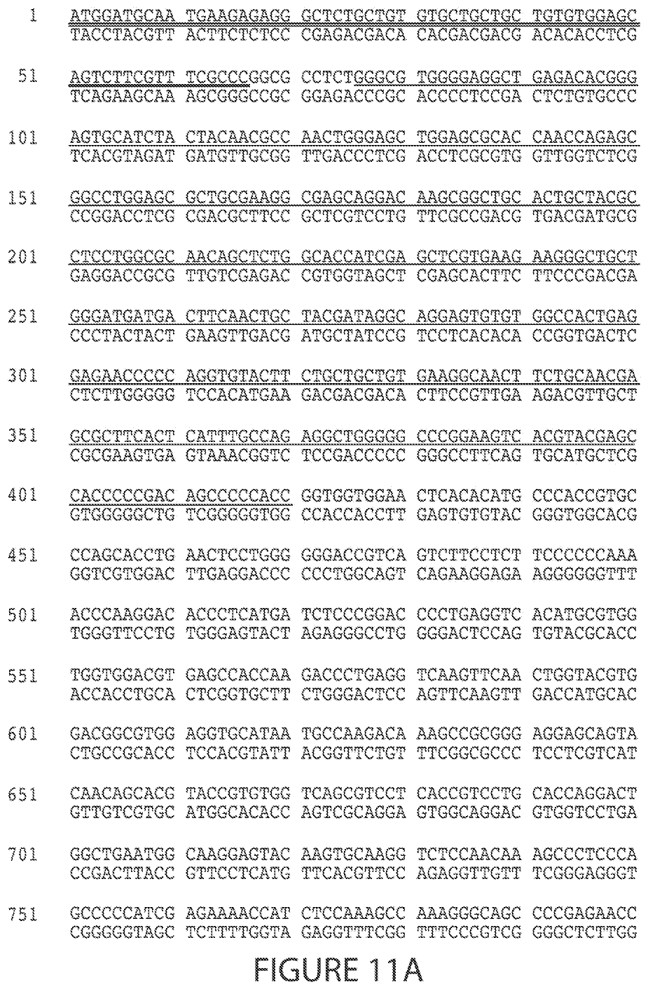

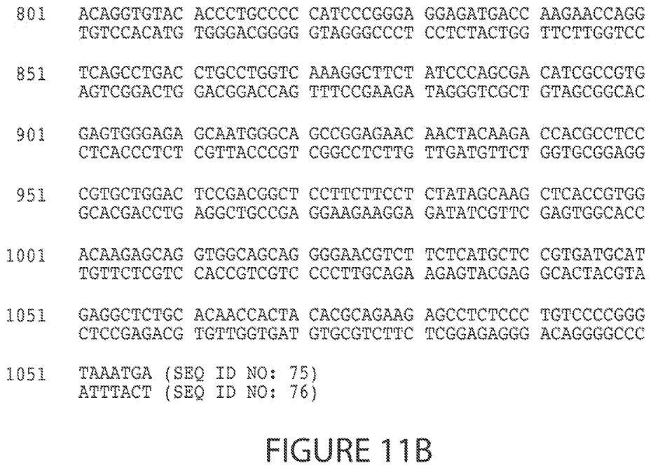

An ActRIIB variant referred to as ActRIIB(L79D 20-134)-hFc was generated by N-terminal fusion of TPA leader to the ActRIIB extracellular domain (residues 20-134 in SEQ ID NO: 1) containing a leucine-to-aspartate substitution (at residue 79 in SEQ ID NO: 1) and C-terminal fusion of human Fc domain with minimal linker (three glycine residues) (FIG. 10; SEQ ID NO: 74). A nucleotide sequence corresponding to this fusion protein is shown in FIG. 11 (SEQ ID NO: 75, sense strand; and SEQ ID NO: 76, antisense strand).

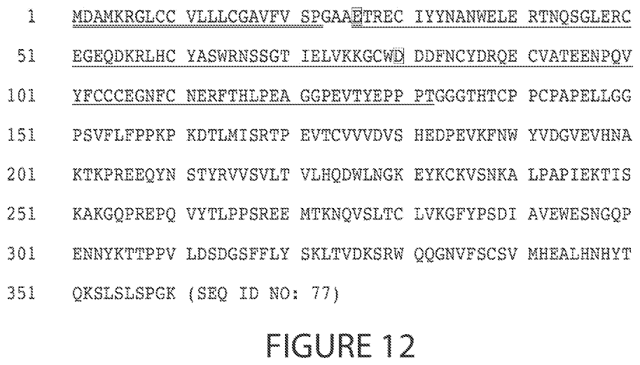

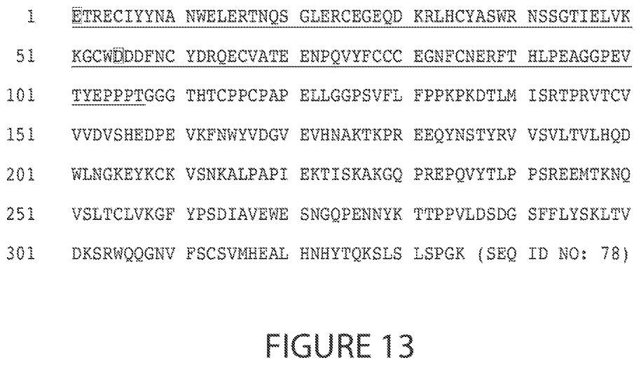

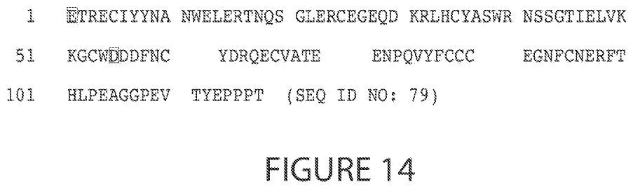

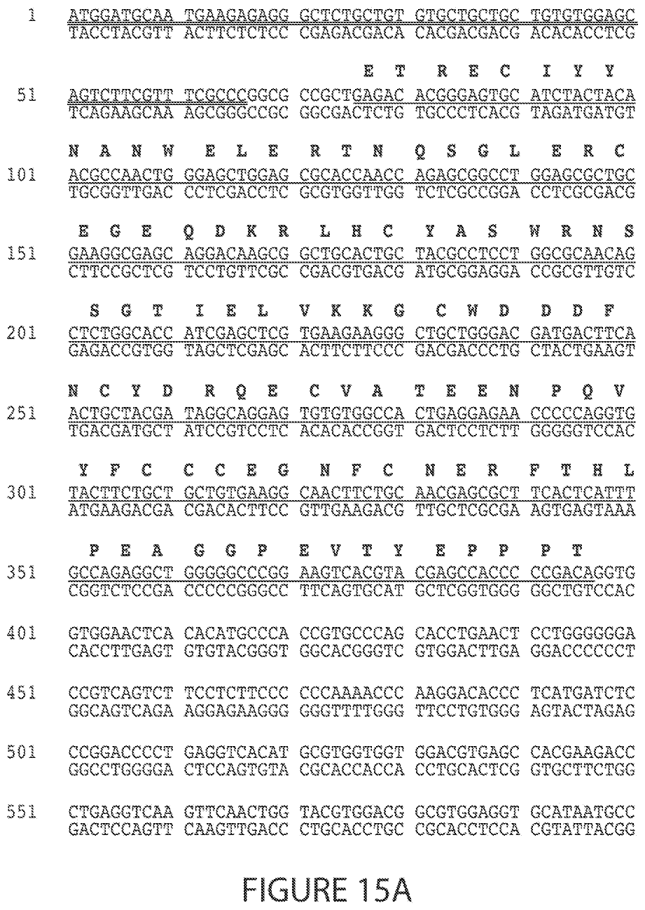

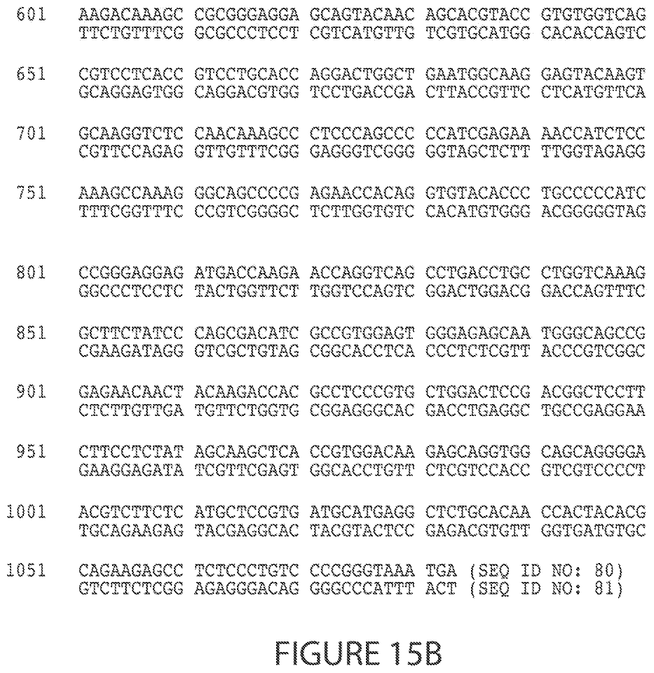

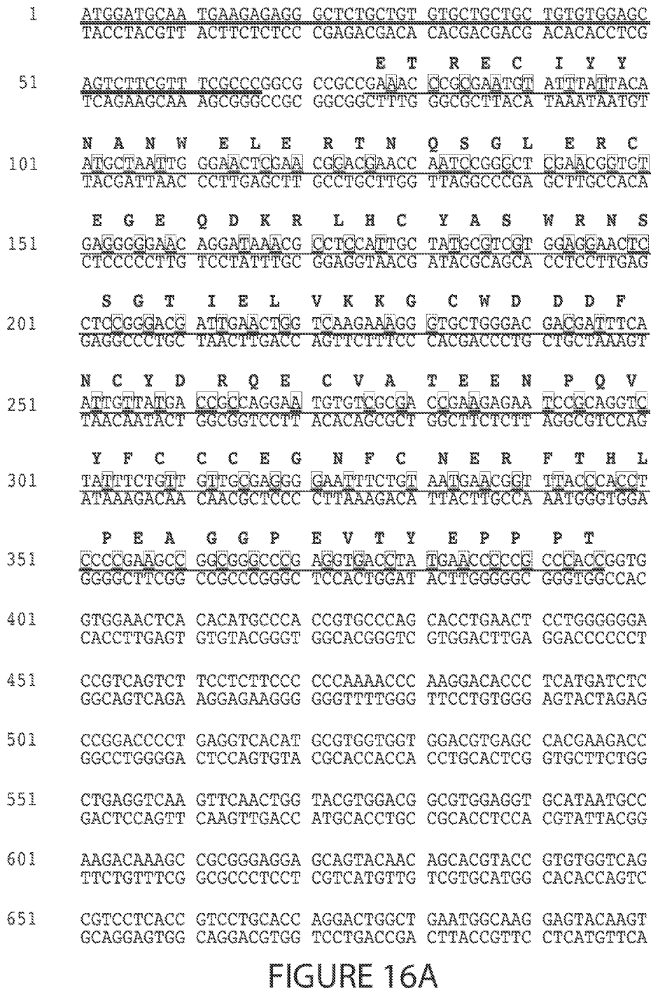

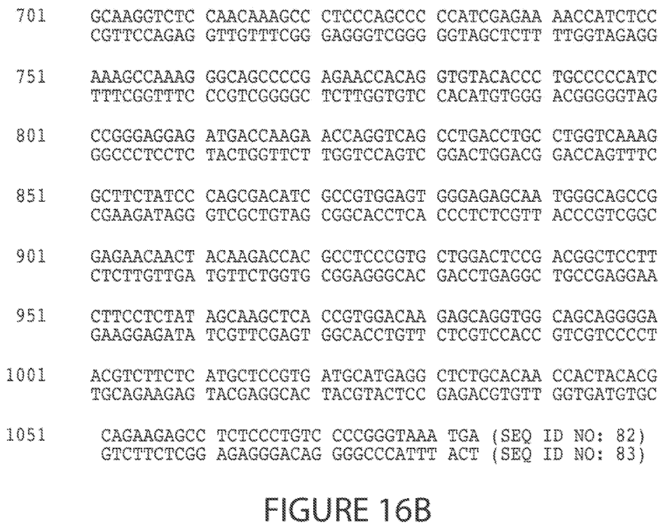

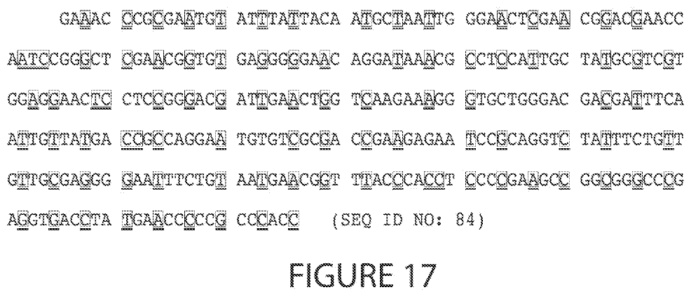

An ActRIIB variant with truncated ActRIIB extracellular domain, referred to as ActRIIB(L79D 25-131)-hFc, was generated by N-terminal fusion of TPA leader to truncated extracellular domain (residues 25-131 in SEQ ID NO:1) containing a leucine-to-aspartate substitution (at residue 79 in SEQ ID NO:1) and C-terminal fusion of human Fc domain with minimal linker (three glycine residues) (FIG. 12, SEQ ID NO: 77). The sequence of the cell purified form of ActRIIB(L79D 25-131)-hFc is presented in FIG. 13 (SEQ ID NO: 78). The sequence of the truncated ActRIIB(L79D 25-131) region without the leader, hFc domain, or linker is presented in FIG. 14 (SEQ ID NO: 79) One nucleotide sequence encoding the fusion protein is shown in FIG. 15 (SEQ ID NO: 80) along with its complementary sequence (SEQ ID NO: 81), and an alternative nucleotide sequence encoding exactly the same fusion protein (SEQ ID NO: 82) and its complementary sequence (SEQ ID NO: 83) is shown in FIGS. 16A and 16B. An alternative nucleotide sequence (SEQ ID NO: 84) encoding only the truncated ActRIIB extracellular domain (corresponding to residues 25-131 of SEQ ID NO: 1) with the L79D substitution (SEQ ID NO: 82) is shown in FIG. 17.

### Example 10: Selective Ligand Binding by ActRIIB Variants with Double-Truncated ActRIIB Extracellular Domain

The affinity of ActRIIB variants and other ActRIIB-hFc proteins for several ligands was evaluated in vitro with a Biacore™ instrument. Results are summarized in Table 12 below. Kd values were obtained by steady-state affinity fit due to the very rapid association and dissociation of the complex, which prevented accurate determination of kₒₙ and k_{off}.

TABLE 12
Ligand Selectivity of ActRIIB-hFc Variants:

| | Activin A | Activin B | GDF11 |
|---|---|---|---|
| Fusion Construct | (Kd e−11) | (Kd e−11) | (Kd e−11) |
| ActRIIB(L79 20-134)-hFc | 1.6 | 1.2 | 3.6 |
| ActRIIB(L79D 20-134)-hFc | 1350.0 | 78.8 | 12.3 |
| ActRIIB(L79 25-131)-hFc | 1.8 | 1.2 | 3.1 |
| ActRIIB(L79D 25-131)-hFc | 2290.0 | 62.1 | 7.4 |

The ActRIIB variant with a truncated extracellular domain, ActRIIB(L79D 25-131)-hFc, equaled or surpassed the ligand selectivity displayed by the longer variant, ActRIIB(L79D 20-134)-hFc, with pronounced loss of activin A binding, partial loss of activin B binding, and nearly full retention of GDF11 binding compared to ActRIIB-hFc counterparts lacking the L79D substitution. Note that truncation alone (without L79D substitution) did not alter selectivity among the ligands displayed here [compare ActRIIB(L79 25-131)-hFc with ActRIIB(L79 20-134)-hFc]. ActRIIB(L79D 25-131)-hFc also retains strong to intermediate binding to the Smad signaling ligands GDF8, BMP6, and BMP10.

### Example 11: ActRIIB5 Variant Derived from ActRIIB5

Others have reported an alternate, soluble form of ActRIIB (designated ActRIIB5), in which exon 4, including the ActRIIB transmembrane domain, has been replaced by a different C-terminal sequence (see, e.g., WO 2007/053775).

The sequence of native human ActRIIB5 without its leader is as follows:

(SEQ ID NO: 50)
GRGEAETRECIYYNANWELERTNQSGLERCEGEQDKRLHCYASWRNSSGTI
ELVKKGCWLDDFNCYDRQECVATEENPQVYFCCCEGNFCNERFTHLPEAGG
PEGPWASTTIPSGGPEATAAAGDQGSGALWLCLEGPAHE

An leucine-to-aspartate substitution, or other acidic substitutions, may be performed at native position 79 (underlined) as described to construct the variant ActRIIB5(L79D), which has the following sequence:

(SEQ ID NO: 51)
GRGEAETRECIYYNANWELERTNQSGLERCEGEQDKRLHCYASWRNSSGTI
ELVKKGCWDDDFNCYDRQECVATEENPQVYFCCCEGNFCNERFTHLPEAGG
PEGPWASTTIPSGGPEATAAAGDQGSGALWLCLEGPAHE

This variant may be connected to human Fc (double underline) with a TGGG linker (SEQ ID NO: 23) (single underline) to generate a human ActRIIB5(L79D)-hFc fusion protein with the following sequence:

(SEQ ID NO: 52)
GRGEAETRECIYYNANWELERTNQSGLERCEGEQDKRLHCYASWRNSSGTI
ELVKKGCWDDDFNCYDRQECVATEENPQVYFCCCEGNFCNERFTHLPEAGG
PEGPWASTTIPSGGPEATAAAGDQGSGALWLCLEGPAHETGGGTHTCPPCP
APELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHN
HYTQKSLSLSPGK.

This construct may be expressed in CHO cells.

### Example 12: Generation of an ALK4:ActRIIB Heterodimer

An ALK4-Fc:ActRIIB-Fc heteromeric complex was constructed comprising the extracellular domains of human ActRIIB and human ALK4, which are each separately fused to an Fc domain with a linker positioned between the extracellular domain and the Fc domain. The individual constructs are referred to as ActRIIB-Fc fusion polypeptide and ALK4-Fc fusion polypeptide, respectively, and the sequences for each are provided below.

A methodology for promoting formation of ALK4-Fc:ActRIIB-Fc heteromeric complexes, as opposed to ActRIIB-Fc or ALK4-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB-Fc and ALK4-Fc polypeptide sequences of SEQ ID NOs: 108 and 110 and SEQ ID NOs: 111 and 113, respectively, one Fc domain is altered to introduce cationic amino acids at the interaction face, while the other Fc domain is altered to introduce anionic amino acids at the interaction face. ActRIIB-Fc fusion polypeptide and ALK4-Fc fusion polypeptide each employ the tissue plasminogen activator (TPA) leader.

The ActRIIB-Fc polypeptide sequence (SEQ ID NO: 108) is shown below:

| (SEQ ID NO: 108) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSRKEMT KNQVSLTCLV KGFYPSDIAV |
| 301 | EWESNGQPEN NYKTTPPVLK SDGSFFLYSK LTVDKSRWQQ GNVFSCSVMH |
| 351 | EALHNHYTQK SLSLSPGK |

The leader (signal) sequence and linker are underlined. To promote formation of ALK4-Fc:ActRIIB-Fc heterodimer rather than either of the possible homodimeric complexes, two amino acid substitutions (replacing acidic amino acids with lysine) can be introduced into the Fc domain of the ActRIIB fusion protein as indicated by double underline above. The amino acid sequence of SEQ ID NO: 108 may optionally be provided with lysine (K) removed from the C-terminus.

This ActRIIB-Fc fusion protein is encoded by the following nucleic acid sequence (SEQ ID NO: 109):

| (SEQ ID NO: 109) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCGAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGAA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGAAG TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

A mature ActRIIB-Fc fusion polypeptide (SEQ ID NO: 110) is as follows, and may optionally be provided with lysine (K) removed from the C-terminus.

| (SEQ ID NO: 110) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | RKEMTKNQVS LTCLVKGFYP SDIAVEWESN GQPENNYKTT PPVLKSDGSF |
| 301 | FLYSKLTVDK SRWQQGNVFS CSVMHEALHN HYTQKSLSLS PGK |

A complementary form of ALK4-Fc fusion polypeptide (SEQ ID NO: 111) is as follows:

| (SEQ ID NO: 111) | |
|---|---|
| 1 | MDAMKRGLCC VLLLCGAVFV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPSREEMTKN QVSLTCLVKG FYPSDIAVEW ESNGQPENNY |
| 301 | DTTPPVLDSD GSFFLYSDLT VDKSRWQQGN VFSCSVMHEA LHNHYTQKSL |
| 351 | SLSPG |

The leader sequence and linker are underlined. To guide heterodimer formation with the ActRIIB-Fc fusion polypeptide of SEQ ID NOs: 108 and 110 above, two amino acid substitutions (replacing lysines with aspartic acids) can be introduced into the Fc domain of the ALK4-Fc fusion polypeptide as indicated by double underline above. The amino acid sequence of SEQ ID NO: 111 may optionally be provided with lysine (K) added at the C-terminus.

This ALK4-Fc fusion protein is encoded by the following nucleic acid (SEQ ID NO: 112):

| (SEQ ID NO: 112) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCCGGGCC CCGGGGGGTC CAGGCTCTGC |
| 101 | TGTGTGCGTG CACCAGCTGC CTCCAGGCCA ACTACACGTG TGAGACAGAT |
| 151 | GGGGCCTGCA TGGTTTCCAT TTTCAATCTG GATGGGATGG AGCACCATGT |
| 201 | GCGCACCTGC ATCCCCAAAG TGGAGCTGGT CCCTGCCGGG AAGCCCTTCT |
| 251 | ACTGCCTGAG CTCGGAGGAC CTGCGCAACA CCCACTGCTG CTACACTGAC |
| 301 | TACTGCAACA GGATCGACTT GAGGGTGCCC AGTGGTCACC TCAAGGAGCC |
| 351 | TGAGCACCCG TCCATGTGGG GCCCGGTGGA GACCGGTGGT GGAACTCACA |
| 401 | CATGCCCACC GTGCCCAGCA CCTGAACTCC TGGGGGGACC GTCAGTCTTC |
| 451 | CTCTTCCCCC CAAAACCCAA GGACACCCTC ATGATCTCCC GGACCCCTGA |
| 501 | GGTCACATGC GTGGTGGTGG ACGTGAGCCA CGAAGACCCT GAGGTCAAGT |
| 551 | TCAACTGGTA CGTGGACGGC GTGGAGGTGC ATAATGCCAA GACAAAGCCG |
| 601 | CGGGAGGAGC AGTACAACAG CACGTACCGT GTGGTCAGCG TCCTCACCGT |
| 651 | CCTGCACCAG GACTGGCTGA ATGGCAAGGA GTACAAGTGC AAGGTCTCCA |
| 701 | ACAAAGCCCT CCCAGCCCCC ATCGAGAAAA CCATCTCCAA AGCCAAAGGG |
| 751 | CAGCCCCGAG AACCACAGGT GTACACCCTG CCCCCATCCC GGGAGGAGAT |
| 801 | GACCAAGAAC CAGGTCAGCC TGACCTGCCT GGTCAAAGGC TTCTATCCCA |
| 851 | GCGACATCGC CGTGGAGTGG GAGAGCAATG GGCAGCCGGA GAACAACTAC |
| 901 | GACACCACGC CTCCCGTGCT GGACTCCGAC GGCTCCTTCT TCCTCTATAG |
| 951 | CGACCTCACC GTGGACAAGA GCAGGTGGCA GCAGGGGAAC GTCTTCTCAT |
| 1001 | GCTCCGTGAT GCATGAGGCT CTGCACAACC ACTACAGGCA GAAGAGCCTC |
| 1051 | TCCCTGTCTC CGGGT |

A mature ALK4-Fc fusion protein sequence (SEQ ID NO: 113) is as follows and may optionally be provided with lysine (K) added at the C-terminus.

| (SEQ ID NO: 113) | |
|---|---|
| 1 | SGPRGVQALL CACTSCLQAN YTCETDGACM VSIFNLDGME HHVRTCIPKV |
| 51 | ELVPAGKPFY CLSSEDLRNT HCCYTDYCNR IDLRVPSGHL KEPEHPSMWG |
| 101 | PVETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPSR EEMTKNQVSL |
| 251 | TCLVKGFYPS DIAVEWESNG QPENNYDTTP PVLDSDGSFF LYSDLTVDKS |
| 301 | RWQQGNVFSC SVMHEALHNH YTQKSLSLSP G |

The ActRIIB-Fc and ALK4-Fc proteins of SEQ ID NO: 110 and SEQ ID NO: 113, respectively, may be co-expressed and purified from a CHO cell line, to give rise to a heteromeric complex comprising ALK4-Fc:ActRIIB-Fc.

In another approach to promote the formation of heteromultimer complexes using asymmetric Fc fusion proteins the Fc domains are altered to introduce complementary hydrophobic interactions and an additional intermolecular disulfide bond as illustrated in the ActRIIB-Fc and ALK4-Fc polypeptide sequences of SEQ ID NOs: 114 and 115 and SEQ ID Nos: 116 and 117, respectively. The ActRIIB-Fc fusion polypeptide and ALK4-Fc fusion polypeptide each employ the tissue plasminogen activator (TPA) leader.

The ActRIIB-Fc polypeptide sequence (SEQ ID NO: 114) is shown below:

| (SEQ ID NO: 114) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPCREEMT KNQVSLWCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

The leader (signal) sequence and linker are underlined. To promote formation of the ALK4-Fc:ActRIIB-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 114 may optionally be provided with lysine (K) removed from the C-terminus.

A mature ActRIIB-Fc fusion polypeptide is as follows:

| (SEQ ID NO: 115) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPC |
| 251 | REEMTKNQVS LWCLVKGFYP SDIAVEWESN GQPENNYKTT PPVLDSDGSF |
| 301 | FLYSKLTVDK SRWQQGNVFS CSVMHEALHN HYTQKSLSLS PGK |

A complementary form of ALK4-Fc fusion polypeptide (SEQ ID NO: 116) is as follows and may optionally be provided with lysine (K) removed from the C-terminus.

| (SEQ ID NO: 116) | |
|---|---|
| 1 | MDAMKRGLCC VLLLCGAVFV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVCTL PPSREEMTKN QVSLSCAVKG FYPSDIAVEW ESNGQPENNY |
| 301 | KTTPPVLDSD GSFFLVSKLT VDKSRWQQGN VFSCSVMHEA LHNHYTQKSL |
| 351 | SLSPGK |

The leader sequence and the linker are underlined. To guide heterodimer formation with the ActRIIB-Fc fusion polypeptide of SEQ ID NOs: 114 and 115 above, four amino acid substitutions can be introduced into the Fc domain of the ALK4 fusion polypeptide as indicated by double underline above. The amino acid sequence of SEQ ID NO: 116 may optionally be provided with lysine (K) removed from the C-terminus.

A mature ALK4-Fc fusion protein sequence is as follows and may optionally be provided with lysine (K) removed from the C-terminus.

(SEQ ID NO: 117)

| | |
|---|---|
| 1 | SGPRGVQALL CACTSCLQAN YTCETDGACM |
| | VSIFNLDGME HHVRTCIPKV |
| 51 | ELVPAGKPFY CLSSEDLRNT HCCYTDYCNR |
| | IDLRVPSGHL KEPEHPSMWG |
| 101 | PVETGGGTHT CPPCPAPELL GGPSVFLFPP |
| | KPKDTLMISR TPEVTCVVVD |
| 151 | VSHEDPEVKF NWYVDGVEVH NAKTKPREEQ |
| | YNSTYRVVSV LTVLHQDWLN |
| 201 | GKEYKCKVSN KALPAPIEKT ISKAKGQPRE |
| | PQVCTLPPSR EEMTKNQVSL |
| 251 | SCAVKGFYPS DIAVEWESNG QPENNYKTTP |
| | PVLDSDGSFF LVSKLTVDKS |
| 301 | RWQQGNVFSC SVMHEALHNH YTQKSLSLSP |
| | GK |

ActRIIB-Fc and ALK4-Fc proteins of SEQ ID NO: 115 and SEQ ID NO: 117 respectively, may be co-expressed and purified from a CHO cell line, to give rise to a heteromeric complex comprising ALK4-Fc:ActRIIB-Fc.

Purification of various ALK4-Fc:ActRIIB-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

In another approach to promote the formation of heteromultimer complexes using asymmetric Fc fusion proteins, the Fc domains are altered to introduce complementary hydrophobic interactions, an additional intermolecular disulfide bond, and electrostatic differences between the two Fc domains for facilitating purification based on net molecular charge, as illustrated in the ActRIIB-Fc and ALK4-Fc polypeptide sequences of SEQ ID NOs: 118-121 and 122-125, respectively. The ActRIIB-Fc fusion polypeptide and ALK4-Fc fusion polypeptide each employ the tissue plasminogen activator (TPA) leader).

The ActRIIB-Fc polypeptide sequence (SEQ ID NO: 118) is shown below:

(SEQ ID NO: 118)

| | |
|---|---|
| 1 | MDAMKRGLCC VLLLCGAVFV SPGASGRGEA |
| | ETRECIYYNA NWELERTNQS |
| 51 | GLERCEGEQD KRLHCYASWR NSSGTIELVK |
| | KGCWLDDFNC YDRQECVATE |
| 101 | ENPQVYFCCC EGNFCNERFT HLPEAGGPEV |
| | TYEPPPTAPT GGGTHTCPPC |
| 151 | PAPELLGGPS VFLFPPKPKD TLMISRTPEV |
| | TCVVVDVSHE DPEVKFNWYV |
| 201 | DGVEVHNAKT KPREEQYNST YRVVSVLTVL |
| | HQDWLNGKEY KCKVSNKALP |
| 251 | APIEKTISKA KGQPREPQVY TLPPCREEMT |
| | ENQVSLWCLV KGFYPSDIAV |
| 301 | EWESNGQPEN NYKTTPPVLD SDGSFFLYSK |
| | LTVDKSRWQQ GNVFSCSVMH |
| 351 | EALHNHYTQD SLSLSPG |

The leader sequence and linker are underlined. To promote formation of the ALK4-Fc:ActRIIB-Fc heterodimer rather than either of the possible homodimeric complexes, two amino acid substitutions (replacing a serine with a cysteine and a threonine with a trytophan) can be introduced into the Fc domain of the fusion protein as indicated by double underline above. To facilitate purification of the ALK4-Fc:ActRIIB-Fc heterodimer, two amino acid substitutions (replacing lysines with acidic amino acids) can also be introduced into the Fc domain of the fusion protein as indicated by double underline above. The amino acid sequence of SEQ ID NO: 118 may optionally be provided with a lysine added at the C-terminus.

This ActRIIB-Fc fusion protein is encoded by the following nucleic acid (SEQ ID NO: 119):

(SEQ ID NO: 119)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCTCTGGGCG |
| | TGGGGAGGCT GAGACACGGG |
| 101 | AGTGCATCTA CTACAACGCC AACTGGGAGC |
| | TGGAGCGCAC CAACCAGAGC |
| 151 | GGCCTGGAGC GCTGCGAAGG GGAGCAGGAC |
| | AAGCGGCTGC ACTGCTACGC |
| 201 | CTCCTGGCGC AACAGCTCTG GCACCATCGA |
| | GCTCGTGAAG AAGGGCTGCT |
| 251 | GGCTAGATGA CTTCAACTGC TACGATAGGC |
| | AGGAGTGTGT GGCCACTGAG |
| 301 | GAGAACCCCC AGGTGTACTT CTGCTGCTGT |
| | GAAGGCAACT TCTGCAACGA |
| 351 | GCGCTTCACT CATTTGCCAG AGGCTGGGGG |
| | CCCGGAAGTC ACGTACGAGC |
| 401 | CACCCCCGAC AGCCCCCACC GGTGGTGGAA |
| | CTCACACATG CCCACCGTGC |
| 451 | CCAGCACCTG AACTCCTGGG GGGACCGTCA |
| | GTCTTCCTCT TCCCCCCAAA |
| 501 | ACCCAAGGAC ACCCTCATGA TCTCCCGGAC |
| | CCCTGAGGTC ACATGCGTGG |
| 551 | TGGTGGACGT GAGCCACGAA GACCCTGAGG |
| | TCAAGTTCAA CTGGTACGTG |
| 601 | GACGGCGTGG AGGTGCATAA TGCCAAGACA |
| | AAGCCGCGGG AGGAGCAGTA |
| 651 | CAACAGCAGG TACCGTGTGG TCAGCGTCCT |
| | CACCGTCCTG CACCAGGACT |
| 701 | GGCTGAATGG CAAGGAGTAC AAGTGCAAGG |
| | TCTCCAACAA AGCCCTCCCA |
| 751 | GCCCCCATCG AGAAAACCAT CTCCAAAGCC |
| | AAAGGGCAGC CCCGAGAACC |
| 801 | ACAGGTGTAC ACCCTGCCCC CATGCCGGGA |
| | GGAGATGACC GAGAACCAGG |
| 851 | TCAGCCTGTG GTGCCTGGTC AAAGGCTTCT |
| | ATCCCAGCGA CATCGCCGTG |
| 901 | GAGTGGGAGA GCAATGGGCA GCCGGAGAAC |
| | AACTACAAGA CCACGCCTCC |
| 951 | CGTGCTGGAC TCCGACGGCT CCTTCTTCCT |
| | CTATAGCAAG CTCACCGTGG |
| 1001 | ACAAGAGCAG GTGGCAGCAG GGGAACGTCT |
| | TCTCATGCTC CGTGATGCAT |
| 1051 | GAGGCTCTGC ACAACCACTA CAGGCAGGAC |
| | AGCCTCTCCC TGTCTCCGGG |
| 1101 | T |

The mature ActRIIB-Fc fusion polypeptide is as follows (SEQ ID NO: 120) and may optionally be provided with a lysine added to the C-terminus.

(SEQ ID NO: 120)

| | |
|---|---|
| 1 | GRGEAETREC IYYNANWELE RTNQSGLERC |
| | EGEQDKRLHC YASWRNSSGT |
| 51 | IELVKKGCWL DDFNCYDRQE CVATEENPQV |
| | YFCCCEGNFC NERFTHLPEA |
| 101 | GGPEVTYEPP PTAPTGGGTH TCPPCPAPEL |
| | LGGPSVFLFP PKPKDTLMIS |
| 151 | RTPEVTCVVV DVSHEDPEVK FNWYVDGVEV |
| | HNAKTKPREE QYNSTYRVVS |
| 201 | VLTVLHQDWL NGKEYKCKVS NKALPAPIEK |
| | TISKAKGQPR EPQVYTLPPC |
| 251 | REEMTENQVS LWCLVKGFYP SDIAVEWESN |
| | GQPENNYKTT PPVLDSDGSF |
| 301 | FLYSKLTVDK SRWQQGNVFS CSVMHEALHN |
| | HYTQDSLSLS PG |

This ActRIIB-Fc fusion polypeptide is encoded by the following nucleic acid (SEQ ID NO: 121):

(SEQ ID NO: 121)

| | |
|---|---|
| 1 | GGGCGTGGGG AGGCTGAGAC ACGGGAGTGC |
| | ATCTAGTACA ACGCCAACTG |
| 51 | GGAGCTGGAG CGCACCAACC AGAGCGGCCT |
| | GGAGCGCTGC GAAGGCGAGC |
| 101 | AGGACAAGCG GCTGCACTGC TACGCCTCCT |
| | GGCGCAACAG CTCTGGCACC |
| 151 | ATCGAGCTCG TGAAGAAGGG CTGCTGGCTA |
| | GATGACTTCA ACTGCTACGA |
| 201 | TAGGCAGGAG TGTGTGGCCA CTGAGGAGAA |
| | CCCCCAGGTG TACTTCTGCT |
| 251 | GCTGTGAAGG CAACTTCTGC AACGAGCGCT |
| | TCACTCATTT GCCAGAGGCT |
| 301 | GGGGGCCCGG AAGTCACGTA CGAGCCACCC |
| | CCGACAGCCC CCACCGGTGG |
| 351 | TGGAACTCAC ACATGCCCAC CGTGCCCAGC |
| | ACCTGAACTC CTGGGGGGAC |
| 401 | CGTCAGTCTT CCTCTTCCCC CCAAAACCCA |
| | AGGACAGCCT CATGATCTCC |
| 451 | CGGACCCCTG AGGTCACATG CGTGGTGGTG |
| | GACGTGAGCC ACGAAGACCC |
| 501 | TGAGGTCAAG TTCAACTGGT ACGTGGACGG |
| | CGTGGAGGTG CATAATGCCA |
| 551 | AGACAAAGCC GCGGGAGGAG CAGTACAACA |
| | GCACGTACCG TGTGGTCAGC |
| 601 | GTCCTCACCG TCCTGCACCA GGACTGGCTG |
| | AATGGCAAGG AGTACAAGTG |
| 651 | CAAGGTCTCC AACAAAGCCC TCCCAGCCCC |
| | CATCGAGAAA ACCATCTCCA |
| 701 | AAGCCAAAGG GCAGCCCCGA GAACCACAGG |
| | TGTACACCCT GCCCCCATGC |
| 751 | CGGGAGGAGA TGACCGAGAA CCAGGTCAGC |
| | CTGTGGTGCC TGGTCAAAGG |
| 801 | CTTCTATCCC AGCGACATCG CCGTGGAGTG |
| | GGAGAGCAAT GGGCAGCCGG |
| 851 | AGAACAACTA CAAGACCACG CCTCCCGTGC |
| | TGGACTCCGA CGGCTCCTTC |
| 901 | TTCCTCTATA GCAAGCTCAC CGTGGACAAG |
| | AGCAGGTGGC AGCAGGGGAA |
| 951 | CGTCTTCTCA TGCTCCGTGA TGCATGAGGC |
| | TCTGCACAAC CACTACACGC |
| 1001 | AGGACAGCCT CTCCCTGTCT CCGGGT |

The complementary form of ALK4-Fc fusion polypeptide (SEQ ID NO: 122) is as follows and may optionally be provided with lysine removed from the C-terminus.

(SEQ ID NO: 122)

| | |
|---|---|
| 1 | MDAMKRGLCC VLLLCGAVFV SPGASGPRGV |
| | QALLCACTSC LQANYTCETD |
| 51 | GACMVSIFNL DGMEHHVRTC IPKVELVPAG |
| | KPFYCLSSED LRNTHCCYTD |
| 101 | YCNRIDLRVP SGHLKEPEHP SMWGPVETGG |
| | GTHTCPPCPA PELLGGPSVF |
| 151 | LFPPKPKDTL MISRTPEVTC VVVDVSHEDP |
| | EVKFNWYVDG VEVHNAKTKP |
| 201 | REEQYNSTYR VVSVLTVLHQ DWLNGKEYKC |
| | KVSNKALPAP IEKTISKAKG |
| 251 | QPREPQVCTL PPSREEMTKN QVSLSCAVKG |
| | FYPSDIAVEW ESRGQPENNY |
| 301 | KTTPPVLDSR GSFFLVSKLT VDKSRWQQGN |
| | VFSCSVMHEA LHNHYTQKSL |
| 351 | SLSPGK |

The leader sequence and the linker are underlined. To guide heterodimer formation with the ActRIIB-Fc fusion polypeptide of SEQ ID NOs: 118 and 120 above, four amino acid substitutions (replacing a tyrosine with a cysteine, a threonine with a serine, a leucine with an alanine, and a tyrosine with a valine) can be introduced into the Fc domain of the ALK4 fusion polypeptide as indicated by double underline above. To facilitate purification of the ALK4-Fc:ActRIIB-Fc heterodimer, two amino acid substitutions (replacing an asparagine with an arginine and an aspartate with an arginine) can also be introduced into the Fc domain of the ALK4-Fc fusion polypeptide as indicated by double underline above. The amino acid sequence of SEQ ID NO: 122 may optionally be provided with lysine removed from the C-terminus.

This ALK4-Fc fusion polypeptide is encoded by the following nucleic acid (SEQ ID NO: 123):

(SEQ ID NO: 123)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCTCCGGGCC |
| | CCGGGGGGTC CAGGCTCTGC |
| 101 | TGTGTGCGTG CACCAGCTGC CTCCAGGCCA |
| | ACTACAGGTG TGAGACAGAT |
| 151 | GGGGCCTGCA TGGTTTCCAT TTTCAATCTG |
| | GATGGGATGG AGCACCATGT |
| 201 | GCGCACCTGC ATCCCCAAAG TGGAGCTGGT |
| | CCCTGCCGGG AAGCCCTTCT |
| 251 | ACTGCCTGAG CTCGGAGGAC CTGCGCAACA |
| | CCCACTGCTG CTACACTGAC |
| 301 | TACTGCAACA GGATCGACTT GAGGGTGCCC |
| | AGTGGTCACC TCAAGGAGCC |
| 351 | TGAGCACCCG TCCATGTGGG GCCCGGTGGA |
| | GACCGGTGGT GGAACTCACA |
| 401 | CATGCCCACC GTGCCCAGCA CCTGAACTCC |
| | TGGGGGGACC GTCAGTCTTC |
| 451 | CTCTTCCCCC CAAAACCCAA GGACACCCTC |
| | ATGATCTCCC GGACCCCTGA |
| 501 | GGTCACATGC GTGGTGGTGG AGGTGAGCCA |
| | CGAAGACCCT GAGGTCAAGT |
| 551 | TCAACTGGTA CGTGGACGGC GTGGAGGTGC |
| | ATAATGCCAA GACAAAGCCG |
| 601 | CGGGAGGAGC AGTACAACAG CACGTACCGT |
| | GTGGTCAGCG TCCTCACCGT |
| 651 | CCTGCACCAG GACTGGCTGA ATGGCAAGGA |
| | GTACAAGTGC AAGGTCTCCA |
| 701 | ACAAAGCCCT CCCAGCCCCC ATCGAGAAAA |
| | CCATCTCCAA AGCCAAAGGG |
| 751 | CAGCCCCGAG AACCACAGGT GTGCACCCTG |
| | CCCCCATGCC GGGAGGAGAT |
| 801 | GACCAAGAAC CAGGTCAGCC TGTCCTGCGC |
| | CGTCAAAGGC TTCTATCCCA |
| 851 | GCGACATCGC CGTGGAGTGG GAGAGCCGCG |
| | GGCAGCCGGA GAACAACTAC |
| 901 | AAGACCACGC CTCCCGTGCT GGACTCCCGC |
| | GGCTCCTTCT TCCTCGTGAG |
| 951 | CAAGCTCACC GTGGACAAGA GCAGGTGGCA |
| | GCAGGGGAAC GTCTTCTCAT |
| 1001 | GCTCCGTGAT GCATGAGGCT CTGCACAACC |
| | ACTACAGGCA GAAGAGCCTC |
| 1051 | TCCCTGTCTC CGGGTAAA |

The mature ALK4-Fc fusion polypeptide sequence is as follows (SEQ ID NO: 124) and may optionally be provided with lysine removed from the C-terminus.

(SEQ ID NO: 124)

| | |
|---|---|
| 1 | SGPRGVQALL CACTSCLQAN YTCETDGACM |
| | VSIFNLDGME HHVRTCIPKV |
| 51 | ELVPAGKPFY CLSSEDLRNT HCCYTDYCNR |
| | IDLRVPSGHL KEPEHPSMWG |
| 101 | PVETGGGTHT CPPCPAPELL GGPSVFLFPP |
| | KPKDTLMISR TPEVTCVVVD |
| 151 | VSHEDPEVKF NWYVDGVEVH NAKTKPREEQ |
| | YNSTYRVVSV LTVLHQDWLN |
| 201 | GKEYKCKVSN KALPAPIEKT ISKAKGQPRE |
| | PQVCTLPPSR EEMTKNQVSL |
| 251 | SCAVKGFYPS DIAVEWESRG QPENNYKTTP |
| | PVLDSRGSFF LVSKLTVDKS |
| 301 | RWQQGNVFSC SVMHEALHNH YTQKSLSLSP |
| | GK |

This ALK4-Fc fusion polypeptide is encoded by the following nucleic acid (SEQ ID NO: 125):

| | |
|---|---|
| 1 | TCCGGGCCCC GGGGGGTCCA GGCTCTGCTG |
| | TGTGCGTGCA CCAGCTGCCT |
| 51 | CCAGGCCAAC TACACGTGTG AGACAGATGG |
| | GGCCTGCATG GTTTCCATTT |
| 101 | TCAATCTGGA TGGGATGGAG CACCATGTGC |
| | GGAGGTGCAT CCCCAAAGTG |
| 151 | GAGCTGGTCC CTGCCGGGAA GCCCTTCTAC |
| | TGCCTGAGCT CGGAGGACCT |
| 201 | GCGCAACACC CACTGCTGCT ACACTGACTA |
| | CTGCAACAGG ATCGACTTGA |
| 251 | GGGTGCCCAG TGGTCACCTC AAGGAGCCTG |
| | AGCACCCGTC CATGTGGGGC |
| 301 | CCGGTGGAGA CCGGTGGTGG AACTCACACA |
| | TGCCCACCGT GCCCAGCACC |
| 351 | TGAACTCCTG GGGGGACCGT CAGTCTTCCT |
| | CTTCCCCCCA AAACCCAAGG |
| 401 | ACACCCTCAT GATCTCCCGG ACCCCTGAGG |
| | TCACATGCGT GGTGGTGGAC |
| 451 | GTGAGCCACG AAGACCCTGA GGTCAAGTTC |
| | AACTGGTACG TGGACGGCGT |
| 501 | GGAGGTGCAT AATGCCAAGA CAAAGCCGCG |
| | GGAGGAGCAG TACAACAGCA |
| 551 | CGTACCGTGT GGTCAGCGTC CTCACCGTCC |
| | TGCACCAGGA CTGGCTGAAT |
| 601 | GGCAAGGAGT ACAAGTGCAA GGTCTCCAAC |
| | AAAGCCCTCC CAGCCCCCAT |
| 651 | CGAGAAAACC ATCTCCAAAG CCAAAGGGCA |
| | GCCCCGAGAA CCACAGGTGT |
| 701 | GCACCCTGCC CCCATCCCGG GAGGAGATGA |
| | CCAAGAACCA GGTCAGCCTG |
| 751 | TCCTGCGCCG TCAAAGGCTT CTATCCCAGC |
| | GACATCGCCG TGGAGTGGGA |
| 801 | GAGCCGCGGG CAGCCGGAGA ACAACTACAA |
| | GACCACGCCT CCGGTGGTGG |
| 851 | ACTCCCGCGG CTCCTTCTTC CTCGTGAGCA |
| | AGCTCACCGT GGACAAGAGC |
| 901 | AGGTGGCAGC AGGGGAACGT CTTCTCATGC |
| | TCCGTGATGC ATGAGGCTCT |
| 951 | GCACAACCAC TACACGCAGA AGAGCCTCTC |
| | CCTGTCTCCG GGTAAA |

ActRIIB-Fc and ALK4-Fc proteins of SEQ ID NO: 120 and SEQ ID NO: 124, respectively, may be co-expressed and purified from a CHO cell line, to give rise to a heteromeric complex comprising ALK4-Fc:ActRIIB-Fc.

Purification of various ALK4-Fc:ActRIIB-Fc complexes could be achieved by a series of column chromatography steps, including, for example, three or more of the following, in any order: protein A chromatography, Q sepharose chromatography, phenylsepharose chromatography, size exclusion chromatography, cation exchange chromatography, epitope-based affinity chromatography (e.g., with an antibody or functionally equivalent ligand directed against an epitope on ALK4 or ActRIIB), and multimodal chromatography (e.g., with resin containing both electrostatic and hydrophobic ligands). The purification could be completed with viral filtration and buffer exchange.

### Example 13. Ligand Binding Profile of ALK4-Fc:ActRIIB-Fc Heterodimer Compared to ActRIIB-Fc Homodimer and ALK4-Fc Homodimer

A Biacore™-based binding assay was used to compare ligand binding selectivity of the ALK4-Fc:ActRIIB-Fc heterodimeric complex described above with that of ActRIIB-Fc and ALK4-Fc homodimer complexes. The ALK4-Fc:ActRIeB-Fc heterodimer, ActRIB-Fc homodimer, and ALK4-Fc homodimer were independently captured onto the system using an anti-Fc antibody. Ligands were injected and allowed to flow over the captured receptor protein. Results are summarized in Table 13 below, in which ligand off-rates (k_{d}) most indicative of effective polypeptides are denoted in bold.

TABLE 13
Ligand binding profile of ALK4-Fc:ActRIIB-Fc heterodimer
compared to ActRIIB-Fc homodimer and ALK4-Fc homodimer

| | ActRIIB-Fc | | | ALK4-Fc | | | ALK4-Fc:ActRIIB-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/MS) | (1/s) | (pM) | (1/Ms) | (1/s) | (pM) | (1/Ms) | (1/s) | (pM) |
| Activin A | 1.2 × 10⁷ | 2.3 × 10⁻⁴ | 19 | 5.8 × 10⁵ | 1.2 × 10⁻² | 20000 | 1.3 × 10⁷ | 1.5 × 10⁻⁴ | 12 |
| Activin B | 5.1 × 10⁶ | 1.0 × 10⁻⁴ | 20 | No binding | | | 7.1 × 10⁶ | 4.0 × 10⁻⁵ | 6 |
| BMP6 | 3.2 × 10⁷ | 6.8 × 10⁻³ | 190 | — | | | 2.0 × 10⁶ | 5.5 × 10⁻³ | 2700 |
| BMP9 | 1.4 × 10⁷ | 1.1 × 10⁻³ | 77 | — | | | Transient* | | 3400 |
| BMP10 | 2.3 × 10⁷ | 2.6 × 10⁻⁴ | 11 | — | | | 5.6 × 10⁷ | 4.1 × 10⁻³ | 74 |
| GDF3 | 1.4 × 10⁶ | 2.2 × 10⁻³ | 1500 | — | | | 3.4 × 10⁶ | 1.7 × 10⁻² | 4900 |
| GDF8 | 8.3 × 10⁵ | 2.3 × 10⁻⁴ | 280 | 1.3 × 10⁵ | 1.9 × 10⁻³ | 15000† | 3.9 × 10⁵ | 2.1 × 10⁻⁴ | 550 |
| GDF11 | 5.0 × 10⁷ | 1.1 × 10⁻⁴ | 2 | 5.0 × 10⁶ | 4.8 × 10⁻³ | 270† | 3.8 × 10⁷ | 1.1 × 10⁻⁴ | 3 |

*Indeterminate due to transient nature of interaction
†Very low signal
— Not tested

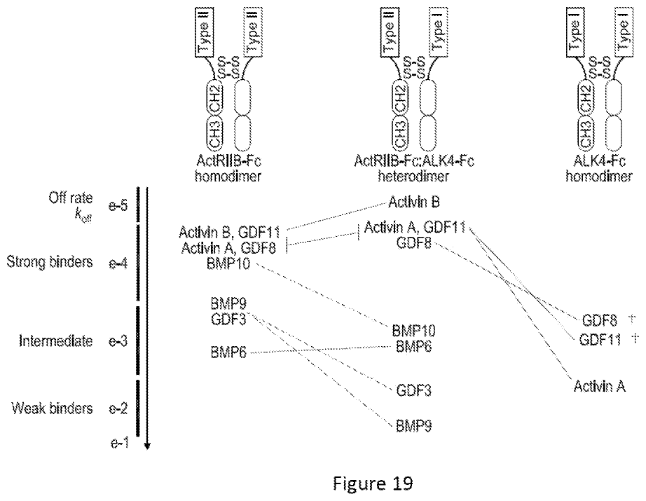

These comparative binding data demonstrate that ALK4-Fc:ActRIIB-Fc heterodimer has an altered binding profile/selectivity relative to either ActRIIB-Fc or ALK4-Fc homodimers. ALK4-Fc:ActRIIB-Fc heterodimer displays enhanced binding to activin B compared with either homodimer, retains strong binding to activin A, GDF8, and GDF11 as observed with ActRIIB-Fc homodimer, and exhibits substantially reduced binding to BMP9, BMP10, and GDF3. In particular, BMP9 displays low or no observable affinity for ALK4-Fc:ActRIIB-Fc heterodimer, whereas this ligand binds strongly to ActRIIB-Fc homodimer. Like the ActRIIB-Fc homodimer, the heterodimer retains intermediate-level binding to BMP6. See FIG. 19.

In addition, an A-204 Reporter Gene Assay was used to evaluate the effects of ALK4-Fc:ActRIIB-Fc heterodimer and ActRIIB-Fc:ActRIIB-Fc homodimer on signaling by activin A, activin B, GDF11, GDF8, BMP10, and BMP9. Cell line: Human Rhabdomyosarcoma (derived from muscle). Reporter vector: pGL3(CAGA)12 (as described in Dennler et al, 1998, EMBO 17: 3091-3100). The CAGA12 motif is present in TGFβ responsive genes (PAI-1 gene), so this vector is of general use for factors signaling through Smads. An exemplary A-204 Reporter Gene Assay is outlined below.
Day 1: Split A-204 cells into 48-well plate.
Day 2: A-204 cells transfected with 10 ug pGL3(CAGA)12 or pGL3(CAGA)12(10 ug)+pRLCMV (1 ug) and Fugene.
Day 3: Add factors (diluted into medium+0.1% BSA). Inhibitors need to be pre-incubated with Factors for about one hr before adding to cells. About six hrs later, cells are rinsed with PBS and then lysed.

Following the above steps, a Luciferase assay was performed.

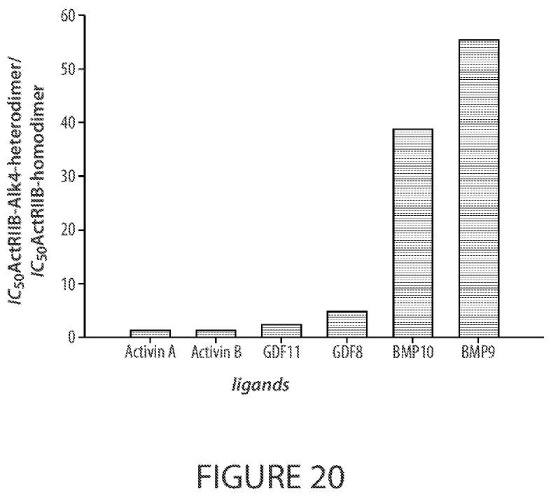

Both the ALK4-Fc:ActRIIB-Fc heterodimer and ActRIIB-Fc:ActRIIB-Fc homodimer were determined to be potent inhibitors of activin A, activin B, GDF11, and GDF8 in this assay. In particular, as can be seen in the comparative homodimer/heterodimer IC₅₀ data illustrated in FIG. 20, ALK4-Fc:ActRIIB-Fc heterodimer inhibits activin A, activin B, GDF8, and GDF11 signaling pathways similarly to the ActRIIB-Fc:ActRIIB-Fc homodimer. However, ALK4-Fc:ActRIIB-Fc heterodimer inhibition of BMP9 and BMP10 signaling pathways is significantly reduced compared to the ActRIIB-Fc:ActRIIB-Fc homodimer. This data is consistent with the above-discussed binding data in which it was observed that both the ALK4-Fc:ActRIIB-Fc heterodimer and ActRIIB-Fc:ActRIIB-Fc homodimer display strong binding to activin A, activin B, GDF8, and GDF11, but BMP10 and BMP9 have significantly reduced affinity for the ALK4-Fc:ActRIIB-Fc heterodimer compared to the ActRIIB-Fc:ActRIIB-Fc homodimer.

Together, these data therefore demonstrate that ALK4-Fc:ActRIIB-Fc heterodimer is a more selective antagonist of activin A, activin B, GDF8, and GDF11 compared to ActRIIB-Fc homodimer. Accordingly, an ALK4-Fc:ActRIIB-Fc heterodimer will be more useful than an ActRIIB-Fc homodimer in certain applications where such selective antagonism is advantageous. Examples include therapeutic applications where it is desirable to retain antagonism of one or more of activin A, activin B, activin AC, GDF8, and GDF11 but minimize antagonism of one or more of BMP9, BMP10, GDF3, and BMP6.

### Example 14: Effects of an ActRII Polypeptide and ALK4:ActRIIB Heterodimer on Pulmonary Hypertension in a Monocrotaline Rat Model

The effects of an ActRIIA-mFc fusion protein (ActRIIA-mFc homodimer as described in Example 1), an ALK4-Fc-ActRIIB-Fc heterodimer (as described in Examples 12 and 13), and sildenafil (a phosphodiesterase-5 inhibitor approved for the treatment of PAH) were examined in a rat model of pulmonary arterial hypertension (PAH). In this model, Sprague Dawley rats received a subcutaneous injection of monocrotaline (MCT) to induce PAH 24 hours prior to start of therapy.

Rats were separated into different treatment groups (10 mice per group): 1) treatment with MCT (60 mg/kg administered i.p. as a single dose at day 1 of study) and Tris buffered saline (i.p. as 1 ml/kg, every three days) (vehicle treatment group), 2) treatment with an ActRIIA-mFc polypeptide (10 mg/kg administered i.p. every three days) and MCT (60 mg/kg administered i.p. as a single dose at day 1 of study), 3) treatment with an ALK4-Fc:ActRIIB-Fc heterodimer (10 mg/kg administered i.p. every three days) and MCT (60 mg/kg administered i.p. as a single dose at day 1 of study), 4) treatment with sildenafil (30 mg/kg administered orally twice daily) and MCT (60 mg/kg administered i.p. as a single dose at day 1 of study), and 5) control rats (Tris buffered saline administered i.p. as 1 ml/kg, every three days). Rats were treated for 28 days. Body weights were recorded prior to first dose on Day 1 and then weekly throughout the study.

On day 28, rats were anesthetized by an intraperitoneal injection of ketamine/xylazine (80/10 mg/kg). An incision was made in the neck, and a jugular vein was isolated and ligated anteriorly. A fluid-filled pressure catheter was introduced into the right jugular vein to measure pulmonary artery pressure (PAP). Another incision was made in the inguinal region, and femoral artery was isolated and ligated anteriorly. A Millar pressure catheter was introduced into a femoral artery to measure systolic arterial pressure, diastolic pressure, and heart rate. Mean arterial pressure and right PAP were monitored using the Notocord HEM (Croissy sur Seine, Frnace) v3.5 data capture system for approximately 5-10 minutes until stable measurements were obtained. During the measurements, rats were maintained at approximately 37° C. on a heating pad and body temperature was monitored throughout the procedure with a rectal temperature probe. At the conclusion of the procedure, rats were euthanized, and the hearts and lungs were removed. The entire heart was weighed. Next, the atria were removed and the left ventricle with septum (LV+S) was separated from the right ventricle (RV). The ventricles were weighed separately. Hypertrophy was assessed, in part, by calculating RV/LV+S. The lungs were also weighed.

Compared to control animals, monocrotaline treated rats (vehicle treatment group) were observed to have decreased body weight, elevated PAP, right heart hypertrophy, and increased lung weight, indicating establishment of PAH. Sildenafil treated rats did not have any improvement in body weight compared to monocrotaline treated rats. However, sildenafil treatment did reduce elevated PAP by 30%, decrease right heart hypertrophy by 18.5%, and decrease lung weight by 10% compared to monocrotaline treated rats. Surprisingly, both ALK4-Fc:ActRIIB-Fc and ActRIIA-mFc were found have significantly greater effects in treating PAH in this model compared to sildenafil. For example, ALK4-Fc:ActRIIB-Fc treatment resulted in improvement in body weight (+5.1%), reduced elevated PAP by 44.6%, decreased right heart hypertrophy by 39.6%, and decreased lung weight by 19.0%. While ActRIIA-mFc treatment did not show improvement in body weight, it had significant effects in treating other complications of PAH. For example, ActRIIA-Fc treatment resulted in a reduction of elevated PAP by 68%, decreased right heart hypertrophy by 47.1%, and decreased lung weight by 18.4%.

Similar trends were observed on vessel muscularity based on histopathologic scoring. After staining tissue samples to detect αSMA/elastin, 100 pulmonary arterioles, between 10 μm and 50 m in size, per animal were categorized as non-muscularized, partially muscularized, or completely muscularized. Pulmonary arterioles from vehicle treated rats were determined to be 62.3% completely muscularized, 36.4% partially muscularized, and 1.4% non-muscularized. Sildenafil treatment had only a modest effect on decreasing vessel muscularity (e.g., pulmonary arterioles being 57.9% completely muscularized, 41.6% partially muscularized, and 0.9% non-muscularized). In contrast, ActRIIA-mFc treatment resulted in significant decreases in vessel muscularity compared to sildenafil treated animals (e.g., pulmonary arterioles being 25.8% completely muscularized, 66.9% partially muscularized, and 7.3% non-muscularized compared to vehicle treated animals). Histopathological scoring of smooth muscle hypertrophy of pulmonary arterioles were also recorded as follows: 0 (normal), 1 (minimal), 2 (mild), 3 (moderate), or 4 (marked). Vehicle treated rats had an average smooth muscle hypertrophy of moderate to marked (3.8 score). Again, sildenafil treatment was observed to have a modest effect on hypertrophy with an average score of 3 (moderate). While ActRIIA-mFc treated animals were observed to have significant reduction in smooth muscle hypertrophy (average score of 1.6) compared to both vehicle and sildenafil treated animals. Overall, ActRIIA-mFc treatment significantly reduced vessel muscularity and hypertrophy in this PAH model.

Together, these data demonstrate that both ActRIIA-mFc and ALK4-Fc:ActRIIB-Fc are effective in ameliorate various complications of PAH in this monocrotaline-induced model. In particular, both ActRIIA-mFc and ALK4-Fc:ActRIIB-Fc had a greater effect in reducing artery pressure, right heart hypertrophy, and vascular muscularization than was observed for sildenafil, which is an approved drug for the treatment of PAH. Furthermore, the data indicate that other ActRII antagonists (or heteromultimers comprising the same), particularly ones having activities similar to ActRIIA-mFc and ALK4-Fc:ActRIIB-Fc, may be useful in the treatment of PAH, particularly in preventing or reducing the severity various complications of PAH.

### Example 15: Effects of an ActRII Polypeptide and Sildenafil on Pulmonary Hypertension in the Sugen Hypoxia Rat Model

The effects of an ActRIIA-mFc fusion protein (ActRIIA-mFc homodimer as described in Example 1 and sildenafil (a phosphodiesterase-5 inhibitor approved for the treatment of PAH) were further examined the Sugen Hypoxia model of PAH. In this model, rats receive daily doses of semaxanib and are placed in a low oxygen environment (approximately 13% oxygen) to induce PAH 24 hours prior to start of therapy.

Rats were separated into different treatment groups (10 mice per group): 1) treatment with semaxanib (200 mg/kg administered s.c. as a single dose daily)/hypoxia and Tris buffered saline (administered i.p. as 1 ml/kg, every three days) (vehicle treatment group), 2) treatment with an ActRIIA-mFc polypeptide (10 mg/kg administered i.p. every three days) and semaxanib (200 mg/kg administered s.c. as a single dose daily)/hypoxia, 3) treatment with sildenafil (30 mg/kg administered orally twice daily) and semaxanib (200 mg/kg administered s.c. as a single dose daily)/hypoxia, and 4) control rats (Tris buffered saline administered i.p. as 1 ml/kg, every three days). Rats were treated for 28 days. Body weights were recorded prior to first dose on Day 1 and then weekly throughout the study.

On day 28, rats were anesthetized by an intraperitoneal injection of ketamine/xylazine (80/10 mg/kg). An incision was made in the neck, and a jugular vein was isolated and ligated anteriorly. A fluid-filled pressure catheter was introduced into the right jugular vein to measure pulmonary artery pressure (PAP). Another incision was made in the inguinal region, and femoral artery was isolated and ligated anteriorly. A Millar pressure catheter was introduced into a femoral artery to measure systolic arterial pressure, diastolic pressure, and heart rate. Mean arterial pressure and right PAP were monitored using the Notocord HEM (Croissy sur Seine, Frnace) v3.5 data capture system for approximately 5-10 minutes until stable measurements were obtained. During the measurements, rats were maintained at approximately 37° C. on a heating pad and body temperature was monitored throughout the procedure with a rectal temperature probe. At the conclusion of the procedure, rats were euthanized, and the hearts and lungs were removed. The entire heart was weighed. Next, the atria were removed and the left ventricle with septum (LV+S) was separated from the right ventricle (RV). The ventricles were weighed separately. Hypertrophy was assessed, in part, by calculating RV/LV+S. The lungs were also weighed.

Compared to control animals, semaxanib/hypoxia treated rats (vehicle treatment group) were observed to have decreased body weight, elevated PAP, right heart hypertrophy, and increased lung weight, indicating establishment of PAH. Sildenafil treatment reduced mean pulmonary arterial pressure by 22.4% and decreased right heart hypertrophy by 10% compared to vehicle treated animals. Again, ActRIIA-mFc treatment was found to have significantly greater effects in treating PAH in this model compared to sildenafil. For example, ActRIIA-mFc treatment resulted in a reduction of mean pulmonary arterial pressure by 51.3% and decreased right heart hypertrophy by 53.5% compared to vehicle treated animals.

Similar trends were observed on vessel muscularity based on histopathologic scoring. After staining tissue samples to detect αSMA/elastin, 100 pulmonary arterioles, between 10 m and 50 m in size, per animal were categorized as non-muscularized, partially muscularized, or completely muscularized. Pulmonary arterioles from vehicle treated rats were determined to be 72.5% completely muscularized, 27.4% partially muscularized, and 0.1% non-muscularized. Sildenafil treatment had only a modest effect on decreasing vessel muscularity (e.g., pulmonary arterioles being 67.4% completely muscularized, 31.6% partially muscularized, and 1.0% non-muscularized) compared to vehicle treated animals. In contrast, ActRIIA-mFc treatment resulted in significant decreases in vessel muscularity compared to sildenafil treated animals (e.g., pulmonary arterioles being 29.3% completely muscularized, 69.3% partially muscularized, and 1.4% non-muscularized compared to vehicle treated animals). Histopathological scoring of smooth muscle hypertrophy of pulmonary arterioles were also recorded as follows: 0 (normal), 1 (minimal), 2 (mild), 3 (moderate), or 4 (marked). Vehicle treated rats had an average smooth muscle hypertrophy of moderate to marked (3.6 score). Again, sildenafil treatment was observed to have a modest effect on hypertrophy with an average score of 3 (moderate). While ActRIIA-mFc treated animals were observed to have significant reduction in smooth muscle hypertrophy (average score of 1.4) compared to sildenafil treated animals. Overall, ActRIIA-mFc treatment significantly reduced vessel muscularity and hypertrophy in this PAH model.

Together, these data demonstrate that ActRIIA-mFc is effective in ameliorate various complications of PAH in the Sugen Hypoxia model. In particular, ActRIIA-mFc had a greater effect in reducing artery pressure, right heart hypertrophy, and vessel muscularization than was observed for sildenafil, which is an approved drug for the treatment of PAH. Furthermore, the data indicate that other ActRII antagonists, particularly ones having activities similar to ActRIIA-mFc may be useful in the treatment of PAH, particularly in preventing or reducing the severity various complications of PAH.

### Example 16. Generation of an ActRIIA-Fc:ALK4-Fc Heterodimer

Applicants constructed a soluble ActRIIA-Fc:ALK4-Fc heteromeric complex comprising the extracellular domains of human ActRIIA and human ALK4, which are each separately fused to an Fc domain with a linker positioned between the extracellular domain and the Fe domain. The individual constructs are referred to as ActRIIA-Fc fusion polypeptide and ALK4-Fc fusion polypeptide, respectively.

Formation of heteromeric ActRIIA-Fc:ALK4-Fc may be guided by approaches similar to those described in Example 12. In a first approach, one Fc domain is altered to introduce cationic amino acids at the interaction face, while the other Fc domain is altered to introduce anionic amino acids at the interaction face.

The ActRIIA-Fc polypeptide sequence (SEQ ID NO: 93) is shown below:

(SEQ ID NO: 93)

| | |
|---|---|
| 1 | MDAMKRGLCC VLLLCGAVFV SPGAAILGRS |
| | ETQECLFFNA NWEKDRTNQT |
| 51 | GVEPCYGDKD KRRHCFATWK NISGSIEIVK |
| | QGCWLDDINC YDRTDCVEKK |
| 101 | DSPEVYFCCC EGNMCNEKFS YFPEMEVTQP |
| | TSNPVTPKPP TGGGTHTCPP |
| 151 | CPAPELLGGP SVFLFPPKPK DTLMISRTPE |
| | VTCVVVDVSH EDPEVKFNWY |
| 201 | VDGVEVHNAK TKPREEQYNS TYRVVSVLTV |
| | LHQDWLNGKE YKCKVSNKAL |
| 251 | PAPIEKTISK AKGQPREPQV YTLPPSRKEM |
| | TKNQVSLTCL VKGEYPSDIA |
| 301 | VEWESNGQPE NNYKTTPPVL KSDGSFFLYS |
| | KLTVDKSRWQ QGNVFSCSVM |
| 351 | HEALHNHYTQ KSLSLSPGK |

The leader sequence and linker sequence are underlined. To promote formation of the ActRIIA-Fc:ALK4-Fc heterodimer rather than either of the possible homodimeric complexes, two amino acid substitutions (replacing acidic amino acids with lysine) can be introduced into the Fc domain of the ActRIIA fusion protein as indicated by double underline above. The amino acid sequence of SEQ ID NO: 93 may optionally be provided with the lysine removed from the C-terminus.

This ActRIIA-Fc fusion protein is encoded by the following nucleic acid sequence (SEQ ID NO: 94):

(SEQ ID NO: 94)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCGCTATACT |
| | TGGTAGATCA GAAACTCAGG |
| 101 | AGTGTCTTTT CTTTAATGCT AATTGGGAAA |
| | AAGACAGAAC CAATCAAACT |
| 151 | GGTGTTGAAC CGTGTTATGG TGACAAAGAT |
| | AAACGGCGGC ATTGTTTTGC |
| 201 | TACCTGGAAG AATATTTCTG GTTCCATTGA |
| | AATAGTGAAA CAAGGTTGTT |
| 251 | GGCTGGATGA TATCAACTGC TATGACAGGA |
| | CTGATTGTGT AGAAAAAAAA |
| 301 | GACAGGCCTG AAGTATATTT CTGTTGCTGT |
| | GAGGGCAATA TGTGTAATGA |
| 351 | AAAGTTTTCT TATTTTCCGG AGATGGAAGT |
| | CACACAGCCC ACTTCAAATC |
| 401 | GAGTTAGACC TAAGCCACCC ACCGGTGGTG |
| | GAACTCACAC ATGCCCACCG |
| 451 | TGCCCAGCAC CTGAACTCCT GGGGGGACCG |
| | TCAGTCTTCC TCTTCCCCCC |
| 501 | AAAACCCAAG GACACCCTCA TGATCTCCCG |
| | GACCCCTGAG GTCACATGCG |
| 551 | TGGTGGTGGA CGTGAGCCAC GAAGACCCTG |
| | AGGTCAAGTT CAACTGGTAC |
| 601 | GTGGACGGCG TGGAGGTGCA TAATGCCAAG |
| | ACAAAGCCGC GGGAGGAGCA |
| 651 | GTACAACAGC ACGTACCGTG TGGTCAGCGT |
| | CCTCACCGTC CTGCACCAGG |
| 701 | ACTGGCTGAA TGGCAAGGAG TACAAGTGCA |
| | AGGTCTCCAA CAAAGCCCTC |
| 751 | CCAGCCCCCA TCGAGAAAAC CATCTCCAAA |
| | GCCAAAGGGC AGCCCCGAGA |
| 801 | ACCACAGGTG TACACCCTGC CCCCATCCCG |
| | GAAGGAGATG ACCAAGAACC |
| 851 | AGGTCAGGCT GACCTGCCTG GTCAAAGGCT |
| | TCTATCCCAG CGACATCGCC |
| 901 | GTGGAGTGGG AGAGCAATGG GCAGCCGGAG |
| | AACAACTACA AGACCACGCC |
| 951 | TCCCGTGCTG AAGTCCGACG GCTCCTTCTT |
| | CCTCTATAGC AAGCTCACCG |
| 1001 | TGGACAAGAG CAGGTGGCAG CAGGGGAACG |
| | TCTTCTCATG CTCCGTGATG |
| 1051 | CATGAGGCTC TGCACAACCA CTACACGCAG |
| | AAGAGCCTCT CCCTGTCTCC |
| 1101 | GGGTAAA |

The mature ActRIIA-Fc fusion polypeptide (SEQ ID NO: 95) is as follows and may optionally be provided with the lysine removed from the C-terminus.

(SEQ ID NO: 95)

| | |
|---|---|
| 1 | ILGRSETQEC LFFNANWEKD RTNQTGVEPC |
| | YGDKDKRRHC FATWKNISGS |
| 51 | IEIVKQGCWL DDINCYDRTD CVEKKDSPEV |
| | YFCCCEGNMC NEKFSYFPEM |
| 101 | EVTQPTSNPV TPKPPTGGGT HTCPPCPAPE |
| | LLGGPSVFLF PPKPKDTLMI |
| 151 | SRTPEVTCVV VDVSHEDPEV KFNWYVDGVE |
| | VHNAKTKPRE EQYNSTYRVV |
| 201 | SVLTVLHQDW LNGKEYKCKV SNKALPAPIE |
| | KTISKAKGQP REPQVYTLPP |
| 251 | SRKEMTKNQV SLTCLVKGFY PSDIAVEWES |
| | NGQPENNYKT TPPVLKSDGS |
| 301 | FFLYSKLTVD KSRWQQGNVF SCSVMHEALH |
| | NHYTQKSLSL SPGK |

In this first approach, the polypeptide sequence of the complementary ALK4-Fc fusion protein and a nucleic acid sequence encoding it are provided above in Example 12 as SEQ ID NOs: 111-113.

The ActRIIA-Fc and ALK4-Fc proteins of SEQ ID NO: 95 and SEQ ID NO: 113, respectively, may be co-expressed and purified from a CHO cell line, to give rise to a heteromeric complex comprising ActRIIA-Fc:ALK4-Fc.

In a second approach to promote the formation of heteromultimer complexes using asymmetric Fc fusion proteins, the Fc domains are altered to introduce complementary hydrophobic interactions and an additional intermolecular disulfide bond.

The ActRIIA-Fc polypeptide sequence (SEQ ID NO: 96) is shown below:

(SEQ ID NO: 96)

| | |
|---|---|
| 1 | MDAMKRGLCC VLLLCGAVFV SPGAAILGRS |
| | ETQECLFFNA NWEKDRTNQT |
| 51 | GVEPCYGDKD KRRHCFATWK NISGSIEIVK |
| | QGCWLDDINC YDRTDCVEKK |
| 101 | DSPEVYFCCC EGNMCNEKFS YFPEMEVTQP |
| | TSNPVTPKPP TGGGTHTCPP |
| 151 | CPAPELLGGP SVFLFPPKPK DTLMISRTPE |
| | VTCVVVDVSH EDPEVKFNWY |
| 201 | VDGVEVHNAK TKPREEQYNS TYRVVSVLTV |
| | LHQDWLNGKE YKCKVSNKAL |
| 251 | PAPIEKTISK AKGQPREPQV YTLPPCREEM |
| | TKNQVSLWCL VKGEYPSDIA |
| 301 | VEWESNGQPE NNYKTTPPVL DSDGSFFLYS |
| | KLTVDKSRWQ QGNVFSCSVM |
| 351 | HEALHNHYTQ KSLSLSPGK |

The leader sequence and linker sequence are underlined. To promote formation of the ActRIIA-Fc:ALK4-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 96 may optionally be provided with the lysine removed from the C-terminus.

The mature ActRIIA-Fc fusion polypeptide (SEQ ID NO: 97) is as follows and may optionally be provided with the lysine removed from the C-terminus.

(SEQ ID NO: 97)

| | |
|---|---|
| 1 | ILGRSETQEC LFFNANWEKD RTNQTGVEPC |
| | YGDKDKRRHC FATWKNISGS |
| 51 | IEIVKQGCWL DDINCYDRTD CVEKKDSPEV |
| | YFCCCEGNMC NEKFSYFPEM |
| 101 | EVTQPTSNPV TPKPPTGGGT HTCPPCPAPE |
| | LLGGPSVFLF PPKPKDTLMI |
| 151 | SRTPEVTCVV VDVSHEDPEV KFNWYVDGVE |
| | VHNAKTKPRE EQYNSTYRVV |
| 201 | SVLTVLHQDW LNGKEYKCKV SNKALPAPIE |
| | KTISKAKGQP REPQVYTLPP |
| 251 | CREEMTKNQV SLWCLVKGFY PSDIAVEWES |
| | NGQPENNYKT TPPVLDSDGS |
| 301 | FFLYSKLTVD KSRWQQGNVF SCSVMHEALH |
| | NHYTQKSLSL SPGK |

In this second approach, the polypeptide sequence of the complementary ALK4-Fc fusion protein and a nucleic acid sequence encoding it are provided above in Example 12 as SEQ ID NOs: 116-117.

The ActRIIA-Fc and ALK4-Fc proteins of SEQ ID NO: 97 and SEQ ID NO: 117, respectively, may be co-expressed and purified from a CHO cell line, to give rise to a heteromeric complex comprising ActRIIA-Fc:ALK4-Fc.

Purification of various ActRIIA-Fc:ALK4-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 17. Ligand Binding Profile of ActRIIA-Fc:ALK4-Fc Heterodimer Compared to ActRIIA-Fc Homodimer and ALK4-Fc Homodimer

A Biacore™-based binding assay was used to compare ligand binding selectivity of the ActRIIA-Fc:ALK4-Fc heterodimeric complex described above with that of ActRIIA-Fc and ALK4-Fc homodimeric complexes. The ActRIIA-Fc:ALK4-Fc heterodimer, ActRIIA-Fc homodimer, and ALK4-Fc homodimer were independently captured onto the system using an anti-Fc antibody. Ligands were injected and allowed to flow over the captured receptor protein. Results are summarized in Table 14 below, in which ligand off-rates (k_{d}) most indicative of effective ligand traps are denoted in bold.

TABLE 14
Ligand binding profile of ActRIIA-Fc:ALK4-Fc heterodimer
compared to ActRIIA-Fc homodimer and ALK4-Fc homodimer

| | ActRIIA-Fc | | | ALK4-Fc | | | ActRIIA-Fc:ALK4-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |
| Activin A | 1.4 × 10⁷ | 6.2 × 10⁻⁴ | 45 | 5.8 × 10⁵ | 1.2 × 10⁻² | 20000 | 7.4 × 10⁶ | 2.4 × 10⁻⁴ | 32 |
| Activin B | 1.1 × 10⁷ | 1.1 × 10⁻⁴ | 10 | No binding | | | 9.5 × 10⁶ | 4.8 × 10⁻⁴ | 50 |
| Activin AB | 2.8 × 10⁷ | 2.6 × 10⁻⁴ | 9 | 1.8 × 10⁶ | 3.6 × 10⁻³ | 2000 | 1.8 × 10⁷ | 2.3 × 10⁻⁴ | 13 |
| Activin AC | 2.2 × 10⁷ | 7.9 × 10⁻³ | 360 | No binding | | | 3.2 × 10⁶ | 5.4 × 10⁻⁴ | 170 |
| BMP6 | 2.7 × 10⁸ | 2.2 × 10⁻² | 800 | No binding | | | 5.4 × 10⁶ | 1.2 × 10⁻² | 2200 |
| BMP7 | 8.9 × 10⁶ | 3.3 × 10⁻² | 3700 | No binding | | | 2.0 × 10⁷ | 7.2 × 10⁻² | 3500 |
| BMP9 | Transient* | | >10000 | — | | | No binding | | |
| BMP10 | 2.9 × 10⁷ | 2.5 × 10⁻³ | 85 | No binding | | | Transient* | | >6000 |
| GDF3 | 1.5 × 10⁶ | 3.6 × 10⁻³ | 2400 | — | | | 4.9 × 10⁷ | 4.8 × 10⁻³ | 9800 |
| GDF8 | 1.4 × 10⁶ | 1.4 × 10⁻³ | 99 | 1.3 × 10⁵ | 1.9 × 10⁻³ | 15000† | 1.8 × 10⁷ | 2.8 × 10⁻³ | 150 |
| GDF11 | 7.3 × 10⁷ | 9.2 × 10⁻⁴ | 13 | 5.0 × 10⁶ | 4.8 × 10⁻³ | 970† | 3.0 × 10⁷ | 6.5 × 10⁻⁴ | 22 |

*Indeterminate due to transient nature of interaction
†Very low signal
— Not tested

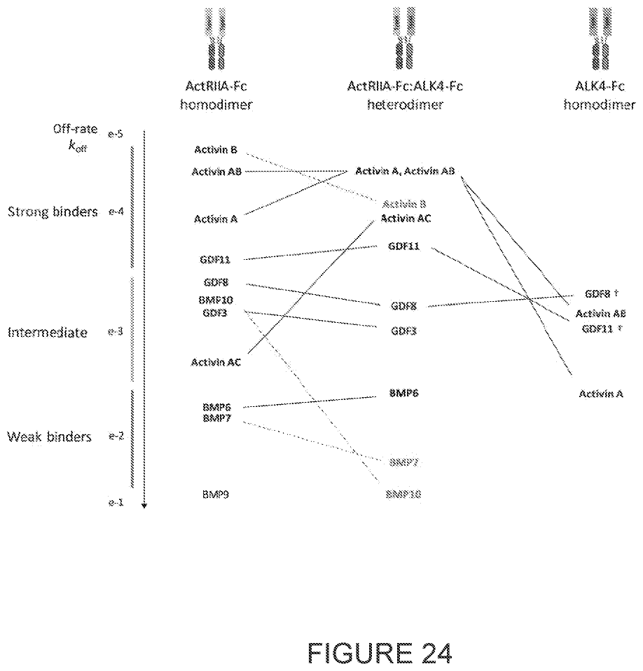

These comparative binding data demonstrate that the ActRIIA-Fc:ALK4-Fc heterodimer has an altered binding profile/selectivity relative to either the ActRIIA-Fc or ALK4-Fc homodimers. For example, the ActRIIA-Fc:ALK4-Fc heterodimer exhibits enhanced binding to activin A, and particularly enhanced binding to activin AC, compared to ActRIIA-Fc homodimer, while retaining strong binding to activin AB and GDF11. In addition, the ligand with highest affinity for ActRIIA-Fc homodimer, activin B, displays reduced affinity (albeit still within the high-affinity range) for the ActRIIA-Fc:ALK4-Fc heterodimer. The ActRIIA-Fc:ALK4-Fc heterodimer also exhibits markedly reduced binding to BMP10 compared to ActRIIA-Fc homodimer. See FIG. 24.

These results demonstrate that the ActRIIA-Fc:ALK4-Fc heterodimer is a more selective antagonist of activin A and activin AB over activin B than is ActRIIA-Fc homodimer. In addition, the ActRIIA-Fc:ALK4-Fc heterodimer has substantially increased affinity for activin AC and greatly reduced affinity for BMP10 compared to ActRIIA-Fc homodimer. Accordingly, an ActRIIA-Fc:ALK4-Fc heterodimer will be more useful than ActRIIA-Fc homodimer in certain applications where such selective antagonism is advantageous. Examples include therapeutic applications where it is desirable to antagonize activin A and/or activin AB preferentially over activin B, and to obtain strong inhibition of activin AC, while avoiding inhibition of BMP10.

TABLE 15
Amino Acid Sequences

| SEQ | |
|---|---|
| ID | |
| NO | Amino Acid Sequence |
| 211 | GAILGRSETQECLFFNANWEKDRTNQTGVE |
| | PCYGDKDKRRHCFATWKNISGSIEIVKQGC |
| | WLDDINCYDRTDCVEKKDSPEVYFCCCEGN |
| | MCNEKFSYFPEMEVTQPTS |
| 284 | MEWSWVFLFFLSVTTGVHSDKTHTCPPCPA |
| | PELLGGPSVFLFPPKPKDTLMISRTPEVTC |
| | VVVDVSHEDPEVKFNWYVDGVEVHNAKTKP |
| | REEQYNSTYRVVSVLTVLHQDWLNGKEYKC |
| | KVSNKALPAPIEKTISKAKGQPREPQVYTL |
| | PPSRDELTKNQVSLTCLVKGFYPSDIAVEW |
| | ESNGQPENNYKTTPPVLDSDGSFFLYSKLT |
| | VDKSRWQQGNVFSCSVMHEALHNHYTQKSL |
| | SLSPGK |
| 286 | GPVEVFITETPSQPNSHPIQWNAPQPSHIS |
| | KYILRWRPKNSVGRWKEATIPGHLNSYTIK |
| | GLKPGVVYEGQLISIQQYGHQEVTRFDFTT |
| | TST |
| 287 | MKWVTFISLLFLFSSAYSRGVFRRDAHKSE |
| | VAHRFKDLGEENFKALVLIAFAQYLQQCPF |
| | EDHVKLVNEVTEFAKTCVADESAENCDKSL |
| | HTLFGDKLCTVATLRETYGEMADCCAKQEP |
| | ERNECFLQHKDDNPNLPRLVRPEVDVMCTA |
| | FHDNEETFLKKYLYEIARRHPYFYAPELLF |
| | FAKRYKAAFTECCQAADKAACLLPKLDELR |
| | DEGKASSAKQRLKCASLQKFGERAFKAWAV |
| | ARLSQRFPKAEFAEVSKLVTDLTKVHTECC |
| | HGDLLECADDRADLAKYICENQDSISSKLK |
| | ECCEKPLLEKSHCIAEVENDEMPADLPSLA |
| | ADFVESKDVCKNYAEAKDVFLGMFLYEYAR |
| | RHPDYSVVLLLRLAKTYETTLEKCCAAADP |
| | HECYAKVFDEFKPLVEEPQNLIKQNCELFE |
| | QLGEYKFQNALLVRYTKKVPQVSTPTLVEV |
| | SRNLGKVGSKCCKHPEAKRMPCAEDYLSVV |
| | LNQLCVLHEKTPVSDRVTKCCTESLVNRRP |
| | CFSALEVDETYVPKEFNAETFTFHADICTL |
| | SEKERQIKKQTALVELVKHKPKATKEQLKA |
| | VMDDFAAFVEKCCKADDKETCFAEEGKKLV |
| | AASQAALGL |
| 305 | ETRECIYYNANWELERTNQSGLERCYGDKD |
| | KRRHCYASWRNSSGTIELVKKGCWLDDINC |
| | YDRTDCVEKKDSPEVYFCCCEGNFCNERFT |
| | HLPEAGGPEVTYEPPPTAPT |
| 306 | ETRECIYYNANWELERTNQSGLERCYGDKD |
| | KRRHCYASWRNSSGTIELVKKGCWLDDFNC |
| | YDRTDCVEKKDSPEVYFCCCEGNFCNERFT |
| | HLPEAGGPEVTYEPPPTAPT |
| 307 | ETRECIYYNANWELERTNQSGLERCEGEQD |
| | KRLHCYASWRNSSGTIELVKKGCWLDDINC |
| | YDRTDCVEKKDSPEVYFCCCEGNFCNERFT |
| | HLPEAGGPEVTYEPPPTAPT |
| 308 | ETRECIYYNANWELERTNQSGLERCEGEQD |
| | KRLHCYASWRNSSGTIELVKKGCWLDDINC |
| | YDRTDCVEKKDSPQVYFCCCEGNFCNERFT |
| | HLPEAGGPEVTYEPPPTAPT |
| 309 | ETRECIYYNANWELERTNQSGLERCEGEQD |
| | KRLHCYASWRNSSGTIELVKKGCWLDDFNC |
| | YDRTDCVEKKDSPQVYFCCCEGNFCNERFT |
| | HLPEAGGPEVTYEPPPTAPT |
| 310 | ETRECIYYNANWELERTNQSGLERCEGEQD |
| | KRLHCYASWRNSSGTIELVKKGCWLDDFNC |
| | YDRTDCVEKKDSPEVYFCCCEGNFCNERFT |
| | HLPEAGGPEVTYEPPPTAPT |
| 311 | ETRECIYYNANWELERTNQSGLERCEGEQD |
| | KRLHCYASWRNSSGTIELVKKGCWLDDFNC |
| | YDRQECVEKKDSPEVYFCCCEGNFCNERFT |
| | HLPEAGGPEVTYEPPPTAPT |
| 312 | ETRECIYYNANWELERTNQSGLERCEGEQD |
| | KRLHCYASWRNSSGTIELVKKGCWLDDFNC |
| | YDRQECVEKKDSPQVYFCCCEGNFCNERFT |
| | HLPEAGGPEVTYEPPPTAPT |
| 313 | ETRECIYYNANWELERTNQSGLERCEGEQD |
| | KRLHCYASWRNSSGTIELVKKGCWLDDINC |
| | YDRQECVEKKDSPEVYFCCCEGNFCNERFT |
| | HLPEAGGPEVTYEPPPTAPT |
| 314 | ETRECIYYNANWELERTNQSGLERCEGEQD |
| | KRLHCYASWRNSSGTIELVKKGCWLDDFNC |
| | YDRTDCVEKKDSPEVYFCCCEGNMCNERFT |
| | HLPEAGGPEVTYEPPPTAPT |
| 315 | ETRECIYYNANWELERTNQSGLERCEGDQD |
| | KRLHCYASWRNSSGTIELVKKGCWLDDFNC |
| | YDRQECVEKKDSPEVYFCCCEGNMCNERFT |
| | HLPEAGGPEVTYEPPPTAPT |
| 316 | ETQECIYYNANWEKDRTNQTGVEPCYGDKD |
| | KRRHCYASWRNSSGTIELVKKGCWLDDFNC |
| | YDRQECVATEENPQVYFCCCEGNFCNERFT |
| | HLPEAGGPEVTYEPPPTAPT |
| 317 | ETQECIYYNANWEKDRTNQTGVEPCEGDQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 318 | ETRECIYYNANWELERTNQSGLERCYGDKD |
| | KRRHCYASWRNSSGTIELVKKGCWLDDFNC |
| | YDRQECVATEENPQVYFCCCEGNFCNERFT |
| | HLPEAGGPEVTYEPPPTAPT |
| 319 | ETRECIYYNANWEKDRTNQTGVEPCEGDQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 320 | ETQECIYYNANWEKDRTNQTGVEPCEGDQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 321 | ETQECIYYNANWEKDRTNQTGLERCEGEQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 322 | ETQECIYYNANWEKDRTNQSGLERCEGEQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 323 | ETRECIYYNANWEKDRTNQSGLERCEGEQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 324 | ETQECIYYNANWELERTNQSGLERCEGEQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 325 | ETRECIYYNANWELERTNQSGLERCEGEQD |
| | KRLHCFATWRNSSGTIELVKQGCWLDDFNC |
| | YDRQECVATEENPQVYFCCCEGNFCNERFT |
| | HLPEAGGPEVTYEPPPTAPT |
| 326 | ETRECIYYNANWELERTNQSGLERCYGDKD |
| | KRRHCYASWRNSSGTIELVKKGCWLDDINC |
| | YDRQECVATEENPQVYFCCCEGNFCNERFT |
| | HLPEAGGPEVTYEPPPTAPT |
| 327 | ETRECIYYNANWELERTNQSGLERCEGEQD |
| | KRLHCYASWRNSSGTIELVKKGCWLDDFNC |
| | YDRQECVATKENPQVYFCCCEGNFCNERFT |
| | HLPEAGGPEVTYEPPPTAPT |
| 328 | ETRECIYYNANWELERTNQSGLERCEGEQD |
| | KRLHCYASWRNSSGTIELVKKGCWLDDINC |
| | YDRQECVATEENPQVYFCCCEGNFCNERFT |
| | HLPEAGGPEVTYEPPPTAPT |
| 329 | ETRECIYYNANWELERTNQSGLERCEGEQD |
| | KRLHCYASWRNSSGTIELVKKGCWLDDINC |
| | YDRQECVATKENPQVYFCCCEGNFCNERFT |
| | HLPEAGGPEVTYEPPPTAPT |
| 330 | ETRECIYYNANWELERTNQSGLERCEGDKD |
| | KRLHCYASWRNSSGTIELVKKGCWLDDINC |
| | YDRQECVATKENPQVYFCCCEGNFCNERFT |
| | HLPEAGGPEVTYEPPPTAPT |
| 331 | ETRECIYYNANWELERTNQSGLERCEGDQD |
| | KRLHCYASWRNSSGTIELVKKGCWLDDINC |
| | YDRQECVATKENPQVYFCCCEGNFCNERFT |
| | HLPEAGGPEVTYEPPPTAPT |
| 332 | ETRECIYYNANWELERTNQSGLERCEGDQD |
| | KRLHCYASWRNSSGTIELVKKGCWLDDFNC |
| | YDRQECVATKENPQVYFCCCEGNFCNERFT |
| | HLPEAGGPEVTYEPPPTAPT |
| 333 | ETRECIYYNANWELERTNQSGLERCEGDQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 334 | ETRECIYYNANWELERTNQSGLERCYGDKD |
| | KRRHCYASWRNSSGTIELVKKGCWLDDINC |
| | YDRQECVEKKDSPEVYFCCCEGNFCNERFT |
| | HLPEAGGPEVTYEPPPTAPT |
| 335 | ETRECIFFNANWEKDRTNQTGVEPCEGEQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 336 | ETRECIYYNANWELERTNQSGLERCEGEQD |
| | KRLHCFATWKNISGSIELVKQGCWLDDFNC |
| | YDRQECVATEENPQVYFCCCEGNFCNERFT |
| | HLPEAGGPEVTYEPPPTAPT |
| 337 | ETRECIYYNANWELERTNQSGLERCEGEQD |
| | KRLHCYASWRNSSGTIELVKKGCWLDDFNC |
| | YDRTDCVATEENPQVYFCCCEGNFCNERFT |
| | HLPEAGGPEVTYEPPPTAPT |
| 338 | ETRECIYYNANWELERTNQSGLERCEGEQD |
| | KRLHCYASWRNSSGTIELVKKGCWLDDFNC |
| | YDRQECVATEENPQVYFCCCEGNMCNERFT |
| | HLPEAGGPEVTYEPPPTAPT |
| 339 | ETRECIYYNANWELERTNQSGLERCEGDQD |
| | KRLHCYASWRNSSGTIELVKKGCWLDDINC |
| | YDRQECVATEENPQVYFCCCEGNFCNERFT |
| | HLPEAGGPEVTYEPPPTAPT |
| 340 | ETQECLFFNANWEKDRTNQSGVEPCYGDKD |
| | KRRHCYASWRNSSGTIELVKKGCWLDDFNC |
| | YDRQECVATEENPQVYFCCCEGNFCNERFT |
| | HLPEAGGPEVTYEPPPTAPT |
| 341 | ETQECLFFNANWEKDRTNQSGVEPCEGEQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 342 | ETRECLFFNANWEKDRTNQSGVEPCEGEQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 343 | ETQECLFFNANWEKDRTNQSGVEPCYGEQD |
| | KRLHCYASWRNSSGTIELVKKGCWLDDFNC |
| | YDRQECVATEENPQVYFCCCEGNFCNERFT |
| | HLPEAGGPEVTYEPPPTAPT |
| 344 | ETRECLFFNANWEKDRTNQSGVEPCYGDKD |
| | KRRHCYASWRNSSGTIELVKKGCWLDDFNC |
| | YDRQECVATEENPQVYFCCCEGNFCNERFT |
| | HLPEAGGPEVTYEPPPTAPT |
| 345 | ETRECLFFNANWEKDRTNQTGVEPCYGDKD |
| | KRRHCFATWKNISGSIEIVKQGCWLDDFNC |
| | YDRQECVATEENPQVYFCCCEGNFCNERFT |
| | HLPEAGGPEVTYEPPPTAPT |
| 346 | ETRECLFFNANWEKDRTNQTGVEPCYGDKD |
| | KRRHCFATWKNISGSIEIVKQGCWLDDINC |
| | YDRQECVATEENPQVYFCCCEGNFCNERFT |
| | HLPEAGGPEVTYEPPPTAPT |
| 347 | ETRECLFFNANWEKDRTNQTGVEPCYGDKD |
| | KRRHCFATWKNISGSIEIVKQGCWLDDINC |
| | YDRTDCVEKKDSPEVYFCCCEGNFCNERFT |
| | HLPEAGGPEVTYEPPPTAPT |
| 348 | ETQECIYYNANWELERTNQSGLERCYGDKD |
| | KRRHCFATWKNISGSIEIVKQGCWLDDINC |
| | YDRQECVATEENPQVYFCCCEGNFCNERFT |
| | HLPEAGGPEVTYEPPPTAPT |
| 349 | ETRECIYYNANWELERTNQSGLERCYGDKD |
| | KRRHCFATWKNISGSIEIVKQGCWLDDINC |
| | YDRTDCVEKKDSPEVYFCCCEGNFCNERFT |
| | HLPEAGGPEVTYEPPPTAPT |
| 350 | ETRECIYYNANWELERTNQSGLERCYGDKD |
| | KRRHCFATWKNISGSIEIVKQGCWLDDINC |
| | YDRTDCVEKKDSPEVYFCCCEGNMCNERFT |
| | HLPEAGGPEVTYEPPPTAPT |
| 351 | ETRECIYYNANWELERTNQSGLERCEGEQD |
| | KRLHCFATWKNISGSIEIVKQGCWLDDINC |
| | YDRTDCVEKKDSPEVYFCCCEGNFCNERFT |
| | HLPEAGGPEVTYEPPPTAPT |
| 352 | ETRECIYYNANWELERTNQSGLERCEGEQD |
| | KRLHCFATWKNISGSIEIVKQGCWLDDINC |
| | YDRTDCVATEENPQVYFCCCEGNFCNERFT |
| | HLPEAGGPEVTYEPPPTAPT |
| 353 | ETRECIYYNANWELERTNQSGLERCEGEQD |
| | KRLHCFATWKNISGSIEIVKQGCWLDDFNC |
| | YDRTDCVATEENPQVYFCCCEGNFCNERFT |
| | HLPEAGGPEVTYEPPPTAPT |
| 354 | ETRECIYYNANWELERTNQSGLERCEGEQD |
| | KRLHCFATWKNISGSIEIVKQGCWLDDFNC |
| | YDRQECVATEENPQVYFCCCEGNFCNERFT |
| | HLPEAGGPEVTYEPPPTAPT |
| 355 | ETQECIYYNANWELERTNQSGLERCYGDKD |
| | KRRHCYASWRNSSGTIELVKKGCWLDDINC |
| | YDRTDCVEKKDSPEVYFCCCEGNFCNERFT |
| | HLPEAGGPEVTYEPPPTAPT |
| 356 | ETQECIYYNANWELERTNQSGLERCYGDKD |
| | KRRHCYASWRNSSGTIELVKKGCWLDDINC |
| | YDRTDCVEKKDSPEVYFCCCEGNMCNERFT |
| | HLPEAGGPEVTYEPPPTAPT |
| 357 | ETQECIYYNANWELERTNQSGLERCYGDKD |
| | KRRHCFATWKNISGSIEIVKQGCWLDDINC |
| | YDRTDCVEKKDSPEVYFCCCEGNMCNERFT |
| | HLPEAGGPEVTYEPPPTAPT |
| 358 | ETQECIYYNANWELERTNQSGLERCYGDKD |
| | KRRHCFATWKNISGSIEIVKQGCWLDDINC |
| | YDRTDCVEKKDSPEVYFCCCEGNFCNERFT |
| | HLPEAGGPEVTYEPPPTAPT |
| 359 | ETRECLFFNANWEKDRTNQTGVEPCEGEQD |
| | KRLHCFATWKNISGSIEIVKQGCWLDDINC |
| | YDRTDCVEKKDSPEVYFCCCEGNFCNERFT |
| | HLPEAGGPEVTYEPPPTAPT |
| 360 | ETRECLFFNANWEKDRTNQSGVEPCEGEQD |
| | KRLHCYASWRNSSGTIELVKKGCWLDDINC |
| | YDRTDCVEKKDSPEVYFCCCEGNFCNERFT |
| | HLPEAGGPEVTYEPPPTAPT |
| 361 | ETRECLFFNANWEKDRTNQSGVEPCYGDKD |
| | KRRHCYASWRNSSGTIELVKKGCWLDDINC |
| | YDRTDCVEKKDSPEVYFCCCEGNFCNERFT |
| | HLPEAGGPEVTYEPPPTAPT |
| 362 | ETRECIYYNANWELERTNQSGLERCYGDKD |
| | KRRHCYASWRNSSGTIELVKKGCWLDDFNC |
| | YDRTDCVATEENPQVYFCCCEGNFCNERFT |
| | HLPEAGGPEVTYEPPPTAPT |
| 363 | ETRECIYYNANWELERTNQSGLERCYGDKD |
| | KRRHCYASWRNSSGTIELVKQGCWLDDFNC |
| | YDRTDCVATEENPQVYFCCCEGNFCNERFT |
| | HLPEAGGPEVTYEPPPTAPT |
| 364 | ETRECIYYNANWELERTNQSGLERCYGDKD |
| | KRRHCYASWRNSSGTIEIVKKGCWLDDFNC |
| | YDRTDCVATEENPQVYFCCCEGNFCNERFT |
| | HLPEAGGPEVTYEPPPTAPT |
| 365 | ETRECIYYNANWELERTNQSGLERCYGDKD |
| | KRRHCYASWRNSSGSIELVKKGCWLDDFNC |
| | YDRTDCVATEENPQVYFCCCEGNFCNERFT |
| | HLPEAGGPEVTYEPPPTAPT |
| 366 | ETRECIYYNANWELERTNQSGLERCYGDKD |
| | KRRHCYASWRNSSGSIEIVKKGCWLDDFNC |
| | YDRTDCVATEENPQVYFCCCEGNFCNERFT |
| | HLPEAGGPEVTYEPPPTAPT |
| 367 | ETRECIYYNANWELERTNQSGLERCYGDKD |
| | KRRHCYASWRNSSGSIEIVKQGCWLDDFNC |
| | YDRTDCVATEENPQVYFCCCEGNFCNERFT |
| | HLPEAGGPEVTYEPPPTAPT |
| 368 | ETRECIYYNANWELERTNQSGLERCEGEQD |
| | KRLHCYASWRNSSGTIELVKKGCWLDDFNC |
| | YDRTDCVATEENPQVYFCCCEGNFCNERFT |
| | HLPEAGGPEVTYEPPPTAPT |
| 369 | ETRECIYYNANWELERTNQSGLERCEGEQD |
| | KRLHCYASWRNSSGTIELVKKGCWLDDFNC |
| | YDRQECVATEENPQVYFCCCEGNFCNEKFS |
| | YFPEMEVTQPTSNPVTPKPP |
| 370 | ETRECIYYNANWELERTNQSGLERCEGEQD |
| | KRLHCYASWRNSSGTIELVKKGCWLDDFNC |
| | YDRQECVATEENPQVYFCCCEGNFCNEKFS |
| | YFPQMEVTQPTSNPVTPKPP |
| 371 | ETRECIYYNANWELERTNQSGLERCYGDKD |
| | KRRHCYASWRNSSGTIELVKKGCWLDDINC |
| | YDRTDCVEKKDSPEVYFCCCEGNFCNEKFS |
| | YFPEMEVTQPTSNPVTPKPP |
| 372 | ETRECIYYNANWELERTNQSGLERCYGDKD |
| | KRRHCYASWRNSSGTIELVKKGCWLDDINC |
| | YDRTDCVEKKDSPEVYFCCCEGNFCNEKFS |
| | YFPQMEVTQPTSNPVTPKPP |
| 373 | ETRECIYYNANWELERTNQSGLERCYGDKD |
| | KRRHCYASWRNSSGTIELVKKGCWLDDFNC |
| | YDRQECVATEENPQVYFCCCEGNFCNEKFS |
| | YFPQMEVTQPTSNPVTPKPP |
| 374 | ETRECIYYNANWELERTNQSGLERCYGDKD |
| | KRRHCYASWRNSSGTIELVKQGCWLDDFNC |
| | YDRQECVATEENPQVYFCCCEGNFCNEKFS |
| | YFPQMEVTQPTSNPVTPKPP |
| 375 | ETRECIYYNANWELERTNQSGLERCYGDKD |
| | KRRHCYASWRNSSGTIEIVKKGCWLDDFNC |
| | YDRQECVATKENPQVYFCCCEGNFCNEKFS |
| | YFPQMEVTQPTSNPVTPKPP |
| 376 | ETRECIYYNANWELERTNQSGLERCYGDKD |
| | KRRHCYASWRNSSGSIELVKKGCWLDDFNC |
| | YDRQECVATKENPQVYFCCCEGNFCNEKFS |
| | YFPQMEVTQPTSNPVTPKPP |
| 377 | ETRECIYYNANWELERTNQSGLERCEGEQD |
| | KRLHCYASWRNSSGSIELVKKGCWLDDFNC |
| | YDRQECVATEENPQVYFCCCEGNFCNEKFS |
| | YFPQMEVTQPTSNPVTPKPP |
| 378 | ETRECIYYNANWELERTNQSGLERCEGEQD |
| | KRLHCYASWRNSSGTIEIVKKGCWLDDFNC |
| | YDRQECVATEENPQVYFCCCEGNFCNEKFS |
| | YFPQMEVTQPTSNPVTPKPP |
| 379 | ETRECIYYNANWELERTNQSGLERCEGEQD |
| | KRLHCYASWRNSSGTIELVKQGCWLDDFNC |
| | YDRQECVATEENPQVYFCCCEGNFCNEKFS |
| | YFPQMEVTQPTSNPVTPKPP |
| 380 | ETRECIYYNANWELERTNQSGLERCEGEQD |
| | KRLHCYASWRNSSGTIELVKKGCWLDDFNC |
| | YDRQECVETEENPQVYFCCCEGNFCNEKFS |
| | YFPQMEVTQPTSNPVTPKPP |
| 381 | ETRECIYYNANWELERTNQSGLERCEGEQD |
| | KRLHCYASWRNSSGTIELVKKGCWLDDFNC |
| | YDRQECVAKEENPQVYFCCCEGNFCNEKFS |
| | YFPQMEVTQPTSNPVTPKPP |
| 382 | ETRECIYYNANWELERTNQSGLERCEGEQD |
| | KRLHCYASWRNSSGTIELVKKGCWLDDFNC |
| | YDRQECVATKENPQVYFCCCEGNFCNEKFS |
| | YFPQMEVTQPTSNPVTPKPP |
| 383 | ETRECIYYNANWELERTNQSGLERCEGEQD |
| | KRLHCYASWRNSSGTIELVKKGCWLDDFNC |
| | YDRQECVATEDNPQVYFCCCEGNFCNEKFS |
| | YFPQMEVTQPTSNPVTPKPP |
| 384 | ETRECIYYNANWELERTNQSGLERCEGEQD |
| | KRLHCYASWRNSSGTIELVKKGCWLDDFNC |
| | YDRQECVATEESPQVYFCCCEGNFCNEKFS |
| | YFPQMEVTQPTSNPVTPKPP |
| 385 | ETRECIYYNANWELERTNQSGLERCEGEQD |
| | KRLHCYASWRNSSGTIELVKKGCWLDDFNC |
| | YDRQECVATEENPEVYFCCCEGNFCNEKFS |
| | YFPQMEVTQPTSNPVTPKPP |
| 386 | ETRECIYYNANWELERTNQSGLERCEGDKD |
| | KRLHCYASWRNSSGTIELVKKGCWLDDFNC |
| | YDRQECVETEENPQVYFCCCEGNFCNEKFS |
| | YFPQMEVTQPTSNPVTPKPP |
| 387 | ETRECIYYNANWELERTNQSGLERCEGEKD |
| | KRLHCYASWRNSSGTIELVKKGCWLDDFNC |
| | YDRQECVAKEENPQVYFCCCEGNFCNEKFS |
| | YFPQMEVTQPTSNPVTPKPP |
| 388 | ETRECIYYNANWELERTNQSGLERCEGDQD |
| | KRLHCYASWRNSSGTIELVKKGCWLDDFNC |
| | YDRQECVATKENPQVYFCCCEGNFCNEKFS |
| | YFPQMEVTQPTSNPVTPKPP |
| 389 | ETRECIYYNANWELERTNQSGLERCEGDKD |
| | KRLHCYASWRNSSGTIELVKQGCWLDDFNC |
| | YDRQECVAKKENPQVYFCCCEGNFCNEKFS |
| | YFPQMEVTQPTSNPVTPKPP |
| 390 | ETRECIYYNANWELERTNQSGLERCEGDKD |
| | KRLHCYASWRNSSGTIEIVKQGCWLDDFNC |
| | YDRQECVAEKENPQVYFCCCEGNFCNEKFS |
| | YFPQMEVTQPTSNPVTPKPP |
| 391 | ETRECIYYNANWELERTNQSGLERCYGDQD |
| | KRLHCYASWRNSSGSIEIVKQGCWLDDFNC |
| | YDRQECVAKKENPQVYFCCCEGNFCNEKFS |
| | YFPQMEVTQPTSNPVTPKPP |
| 392 | ETRECIYYNANWELERTNQSGLERCEGEKD |
| | KRRHCYASWRNSSGTIEIVKKGCWLDDFNC |
| | YDRQECVATKENPQVYFCCCEGNFCNEKFS |
| | YFPQMEVTQPTSNPVTPKPP |
| 393 | ETRECIYYNANWELERTNQSGLERCYGDQD |
| | KRRHCYASWRNSSGTIELVKKGCWLDDFNC |
| | YDRQECVATEENPEVYFCCCEGNFCNEKFS |
| | YFPQMEVTQPTSNPVTPKPP |
| 394 | ETRECIYYNANWELERTNQSGLERCEGEQD |
| | KRRHCYASWRNSSGTIELVKKGCWLDDFNC |
| | YDRQECVATEENPQVYFCCCEGNFCNEKFS |
| | YFPQMEVTQPTSNPVTPKPP |
| 395 | ETRECIYYNANWELERTNQSGLERCYGEQD |
| | KRLHCYASWRNSSGSIEIVKKGCWLDDFNC |
| | YDRTDCVATEENPQVYFCCCEGNFCNEKFS |
| | YFPQMEVTQPTSNPVTPKPP |
| 396 | ETRECIYYNANWELERTNQSGLERCEGEQD |
| | KRRHCYASWRNSSGSIELVKKGCWLDDFNC |
| | YDRQECVAKEENPQVYFCCCEGNFCNEKFS |
| | YFPQMEVTQPTSNPVTPKPP |
| 397 | ETRECIYYNANWELERTNQSGLERCEGEQD |
| | KRRHCYASWRNSSGTIEIVKKGCWLDDFNC |
| | YDRQECVAKEENPQVYFCCCEGNFCNEKFS |
| | YFPQMEVTQPTSNPVTPKPP |
| 398 | ETRECIYYNANWELERTNQSGLERCEGEQD |
| | KRRHCYASWRNSSGSIEIVKKGCWLDDFNC |
| | YDRQECVATKENPQVYFCCCEGNFCNEKFS |
| | YFPQMEVTQPTSNPVTPKPP |
| 399 | ETRECIYYNANWELERTNQSGLERCEGDQD |
| | KRLHCYASWRNSSGTIEIVKKGCWLDDFNC |
| | YDRQECVATKENPQVYFCCCEGNFCNEKFS |
| | YFPQMEVTQPTSNPVTPKPP |
| 400 | ETRECIYYNANWELERTNQSGLERCEGDQD |
| | KRLHCYASWRNSSGSIELVKKGCWLDDFNC |
| | YDRQECVATKENPQVYFCCCEGNFCNEKFS |
| | YFPQMEVTQPTSNPVTPKPP |
| 401 | ETRECIYYNANWELERTNQSGLERCEGDQD |
| | KRLHCYASWRNSSGTIELVKKGCWLDDFNC |
| | YDRQECVATKENPQVYFCCCEGNFCNEKFS |
| | YFPQMEVTQPTSNPVTPKPP |
| 402 | ETRECIYYNANWELERTNQSGLERCEGEQD |
| | KRLHCYASWRNSSGTIEIVKKGCWLDDFNC |
| | YDRQECVATKENPQVYFCCCEGNFCNEKFS |
| | YFPQMEVTQPTSNPVTPKPP |
| 403 | ETRECIYYNANWELERTNQSGLERCEGEQD |
| | KRLHCYASWRNSSGTIELVKKGCWLDDFNC |
| | YDRQECVATKENPQVYFCCCEGNFCNEKFS |
| | YFPQMEVTQPTSNPVTPKPP |
| 404 | ETRECIYYNANWELERTNQSGLERCEGDQD |
| | KRLHCYASWRNSSGTIEIVKKGCWLDDFNC |
| | YDRQECVAKKENPQVYFCCCEGNFCNEKFS |
| | YFPQMEVTQPTSNPVTPKPP |
| 405 | ETQECLFFNANWEKDRTNQTGVEPCYGDKD |
| | KRRHCFATWKNISGSIEIVKQGCWLDDINC |
| | YDRTDCVEKKDSPEVYFCCCEGNMCNERFT |
| | HLPEAGGPEVTYEPPPTAPT |
| 406 | ETRECIYYNANWELERTNQSGLERCYGDKD |
| | KRRHCYASWRNSSGTIELVKKGCWLDDINC |
| | YDRQECVATKENPQVYFCCCEGNFCNERFT |
| | HLPEAGGPEVTYEPPPTAPT |
| 407 | SGRGEAETRECIYYNANWELERTNQSGLER |
| | CEGEQDKRLHCYASWRNSSGTIELVKKGCW |
| | LDDFNCYDRQECVATEENPQVYFCCCEGNF |
| | CNERFTHLPEAGGPEVTYEPPPTAPT |
| 408 | MGAATKLAFAVFLISCSSGAILGRSETQEC |
| | IYYNANWEKDKTNRSGIEPCYGDKDKRRHC |
| | FATWKNISGSIEIVKQGCWLDDINCYDRND |
| | CIEKKDSPEVFFCCCEGNMCNERFFYFPEM |
| | EVTQPTSNPVTPKPPLFNTLLYSLVPIMGI |
| | AVIVLFSFWMYRHHKLAYPPVLVPTQDPGP |
| | PPPSPLMGLKPLQLLEIKARGRFGCVVVKAQ |
| | LLNEYVAVKIFPIQDKQSWQNEYEIYSLPG |
| | MKHDNILQFIGAEKRGTSIDVDLWLITAFH |
| | EKGSLTDFLKANVVSWNELCHIAQTMARGL |
| | AYLHEDIPGLKDGHKPAISHRDIKSKNVLL |
| | KNNLTACIADFGLALKFEAGKSAGDTHGQV |
| | GTRRYMAPEVLEGAINFQRDAFLRIDMYAM |
| | GLVLWELASRCTASDGPVDEYMLPFEEEIG |
| | QHPSLEDMQEVVVHKKKRPVLRECWQKHSG |
| | MAMLCETIEECWDHDAEARLSAGCVEERII |
| | QMQKLTNIITTEDIVTVVTMVTNVDFPPKE |
| | SSL |
| 409 | AILGRSETQECLFFNANWEKDRTNQTGVEP |
| | CYGDKDKRRHCFATWKNISGSIEIVKQGCW |
| | LDDINCYDRTDCVEKKDSPEVYFCCCEGNM |
| | CNEKFSYFPEMEVTQPTSNPVTPKPP |

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1-115. (canceled)

116. A method of treating pulmonary arterial hypertension, comprising administering to a patient in need thereof an effective amount of an ActRIIB variant polypeptide, wherein the ActRIIB variant polypeptide comprises an amino acid sequence that is at least 90% identical the amino acid sequence of SEQ ID NO: 2, wherein the ActRIIB polypeptide comprises one or more substitutions selected from the group consisting of: Q69T, E70D, I11L, L27V, Q34K, T50S, I51L, L53I, and F89M, and wherein the patient has Functional Class II or Class III pulmonary arterial hypertension as recognized by the World Health Organization, and wherein the composition increases the 6-minute walk distance by at least 10 meters.

117. The method of claim 116, wherein the ActRIIB variant polypeptide is selected from the group consisting of:
a. an ActRIIB variant polypeptide that is at least 90% identical to the amino acid sequence of SEQ ID NO: 299;
b. an ActRIIB variant polypeptide that is at least 90% identical to the amino acid sequence of SEQ ID NO: 300;
c. an ActRIIB variant polypeptide that is at least 90% identical to the amino acid sequence of SEQ ID NO: 301; and
d. an ActRIIB variant polypeptide that is at least 90% identical to the amino acid sequence of SEQ ID NO: 302.

118. The method of claim 117, wherein the ActRIIB variant polypeptide comprises an amino acid sequence that is at least 95% identical to the amino acid sequence of SEQ ID NO: 301.

119. The method of claim 117, wherein the ActRIIB variant polypeptide comprises an amino acid sequence that is at least 99% identical to the amino acid sequence of SEQ ID NO: 301.

120. The method of claim 117, wherein the ActRIIB variant polypeptide comprises the amino acid sequence of SEQ ID NO: 301.

121. The method of claim 117, wherein the ActRIIB variant polypeptide comprises an amino acid sequence that is at least 95% identical to the amino acid sequence of SEQ ID NO: 302.

122. The method of claim 117, wherein the ActRIIB variant polypeptide comprises an amino acid sequence that is at least 99% identical to the amino acid sequence of SEQ ID NO: 302.

123. The method of claim 117, wherein the ActRIIB variant polypeptide comprises the amino acid sequence of SEQ ID NO: 302.

124. The method of claim 116, wherein the ActRIIB variant polypeptide is a fusion protein further comprising an Fc domain of an immunoglobulin.

125. The method of claim 124, wherein the Fc domain of the immunoglobulin is an Fc domain of an IgG1 immunoglobulin.

126. The method of claim 124, wherein the fusion protein comprises a linker domain positioned between the ActRIIB variant polypeptide and the Fc domain of the immunoglobulin.

127. The method of claim 126, wherein the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24).

128. The method of claim 116, wherein the ActRIIB variant polypeptide binds to one or more ligands selected from the group consisting of: activin A, activin B, and GDF11.

129. The method of claim 116, wherein the ActRIIB variant polypeptide has reduced BMP9 binding relative to wild type extracellular ActRIIB.

130. The method of claim 116, wherein the patient has resting pulmonary arterial pressure (PAP) of at least 25 mm Hg.
